(19) United States
(12) Patent Application Publication
(10) Pub. No.: US 2024/0191216 A1
(43) Pub. Date: Jun. 13, 2024

(54) RECOMBINANT HOST CELLS FOR THE PRODUCTION OF MALONATE

(71) Applicant: LYGOS, INC., BERKELEY, CA (US)

(72) Inventors: Jeffrey A. Dietrich, Berkeley, CA (US); Jeffrey L. Fortman, Berkeley, CA (US); Eric J. Steen, Berkeley, CA (US)

(73) Assignee: LYGOS, INC., BERKELEY, CA (US)

(21) Appl. No.: 18/123,840

(22) Filed: Mar. 20, 2023

Related U.S. Application Data
(63) Continuation of application No. 17/646,437, filed on Dec. 29, 2021, now abandoned.
Application No. 17/646,437 is a division of application No. 16/595,253, filed on Oct. 7, 2019, now Pat. No. 11,248,215.
Application No. 16/595,253 is a continuation of application No. 15/627,191, filed on Jun. 19, 2017, now Pat. No. 10,472,653.
Application No. 15/627,191 is a division of application No. 14/386,272, filed on Sep. 18, 2014, now Pat. No. 9,816,114.
(60) Provisional application No. 61/607,479, filed on Mar. 6, 2012.
(60) Provisional application No. 61/619,112, filed on Apr. 2, 2012.

(51) Int. Cl. C12N 9/16; C07C 51/38; C07C 67/08; C07C 67/31; C12P 7/46

(52) U.S. Cl. CPC C12N 9/16; C12P 7/46; C12Y 301/02004; C07C 51/38; C07C 67/08; C07C 67/31

(57) ABSTRACT

Systems and methods for the production of malonate in recombinant host cells.

### RELATED APPLICATIONS

The present application is a divisional of and claims priority to U.S. application Ser. No. 14/386,272 filed Sep. 18, 2014, which is a National Stage Application of PCT/US2013/029441 filed Mar. 6, 2013 and claims the benefit of U.S. Provisional Patent Application No. 61/607,479 filed Mar. 6, 2012 and U.S. Provisional Patent Application No. 61/619,112 filed Apr. 2, 2012, the entire disclosures of which are expressly incorporated herein by reference.

### GOVERNMENT INTEREST

This invention was made with government support under grant no. DE-SC0006469 awarded by the Department of Energy. The government has certain rights in this invention.

### REFERENCE TO SEQUENCE LISTING

The instant application contains a Sequence Listing XML which has been submitted electronically and is hereby incorporated by reference in its entirety. Said Sequence Listing XML copy, created on Feb. 20, 2024, is named “LYGOS00020304repST26.xml” and is 320,402 bytes in size.

### BACKGROUND OF THE INVENTION

The long-term economic and environmental concerns associated with the petrochemical industry has provided the impetus for increased research, development, and commercialization of processes for conversion of carbon feedstocks into chemicals that can replace those derived from petroleum feedstocks. One approach is the development of biorefining processes to convert renewable feedstocks into products that can replace petroleum-derived chemicals. Two common goals in improving a biorefining process include achieving a lower cost of production and reducing carbon dioxide emissions.

Propanedioic acid (“malonate”, CAS No. 141-82-2) is currently produced from non-renewable, petroleum feedstocks. Mono- or di-esterification of one or both carboxylic acid moieties of malonate with an alcohol (e.g. methanol or ethanol) yields the monoalkyl and dialkyl malonates, respectively. 2,2-dimethyl-1,3-dioxane-4,6-dione (“Meldrum's acid” CAS No. 2033-24-1) is produced from malonate using either acetone in acetic anhydride or isopropenyl acetate in acid.

Chemical synthesis is currently the preferred route for synthesis of malonate and malonate derived compounds. For example, dialkyl malonates are produced through either a hydrogen cyanide or carbon monoxide process. In the hydrogen cyanide process, sodium cyanide is reacted with sodium chloroacetate at elevated temperatures to produce sodium cyanoacetate, which is subsequently reacted with an alcohol/mineral acid mixture to produce the dialkyl malonate. Hildbrand et al. report yields of 75-85% (see “Malonic acid and Derivatives” In: Ullmann's Encyclopedia of Industrial Chemistry, Wiley-VCH, Weinheim, New York (2002)). In the carbon monoxide process, dialkyl malonates (also referred to herein as diester malonates) are produced through cobalt-catalyzed alkoxycarbonylation of chloroacetates with carbon monoxide in the presence of an alcohol at elevated temperatures and pressures.

The existing, petrochemical-based production routes to the malonate and malonate-derived compounds are low yielding, environmentally damaging, dependent upon non-renewable feedstocks, and require expensive treatment of wastewater and exhaust gas. Thus, there remains a need for methods and materials for biocatalytic conversion of renewable feedstocks into malonate, purification of biosynthetic malonate, and subsequent preparation of downstream chemicals and products.

### 1. SUMMARY OF THE INVENTION

The present invention provides recombinant host cells, materials, and methods for the biological production of malonate, methods for detecting the presence of malonate and determining the levels of malonate (referred to herein as “sensing malonate”) in malonate producing host cells, and methods for screening host cells for increased malonate production. In addition, the present invention provides methods for the purification of biologically produced malonate, and the methods for converting malonate to other industrially important chemicals.

In a first aspect, the invention provides recombinant host cells comprising a heterologous nucleic acid encoding an acyl-CoA hydrolase that catalyzes conversion of malonyl-CoA to malonic acid, as illustrated here:

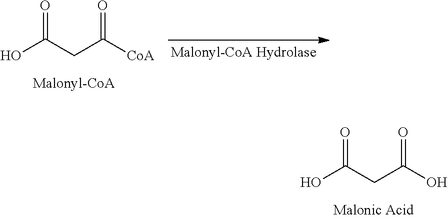

These recombinant host cells produce more malonate than counterpart cells that do not comprise such a heterologous hydrolase. In various embodiments, the host cells can produce at least 10 g/L malonate under appropriate fermentation conditions, and in various embodiments, productions levels can be as high as 50 g/L to 100 g/L or higher. In some embodiments, the heterologous nucleic acid encodes a mutated form of an endogenously expressed enzyme; thus, the present invention provides a variety of mutated acyl-CoA hydrolases, nucleic acids encoding them, and recombinant expression vectors comprising those nucleic acids. In other embodiments, the heterologous nucleic acid codes for the overexpression of an endogenous enzyme. Further, in some embodiments the heterologous nucleic acid encodes a wild-type or mutant enzyme of an acyl-CoA hydrolase heterologous to (not natively expressed in) the host cell. In some embodiments the host cell is a yeast cell. In other embodiments, the host cell is a bacterial cell.

Thus, in various embodiments, the heterologous nucleic acids provided by the invention encode a wild-type or mutated form of an acyl-CoA hydrolase. Non-limiting examples of acyl-CoA hydrolases encoded by the nucleic acids provided by the invention and suitable for malonyl-CoA hydrolysis include wild-type and modified enzymes selected from the group consisting of 3-hydroxyisobutyryl-CoA hydrolases (EC 3.1.2.4), 3-hydroxypropionyl-CoA hydrolases (EC 3.1.2.4), acetoacetyl-CoA hydrolases (EC 3.1.2.11), methylmalonyl-CoA hydrolases (EC 3.1.2.17), propionyl-CoA hydrolases (EC 3.1.2.18), succinyl-CoA hydrolases (EC 3.1.2.3), and malonyl-CoA:ACP transacylases (EC 2.3.1.39) mutated as provided herein to have malonyl-CoA hydrolase activity.

In various embodiments, the invention provides a malonyl-CoA hydrolase that is a mutant of a 3-hydroxyisobutyryl-CoA hydrolase (EC 3.1.2.4). Suitable 3-hydroxyisobutyryl-CoA hydrolases can be obtained from both eukaryotic and prokaryotic, including both Gram positive and Gram negative, organisms. In various embodiments, the 3-hydroxyisobutyryl-CoA hydrolase is obtained from a yeast strain, a Bacillus species, and a Pseudomonas species.

In additional embodiments, the invention provides a malonyl-CoA hydrolase that is a malonyl-CoA:ACP transacylase (EC 2.3.1.39) mutated as provided herein to have malonyl-CoA hydrolase activity, encoded by a prokaryote. In various embodiments, the prokaryote is a Gram-negative bacterium. In various embodiments of the invention, the Gram-negative bacterium is an Escherichia.

In a second aspect, the invention provides recombinant expression vectors encoding a wild-type or mutated acyl-CoA hydrolase that catalyzes conversion of malonyl-CoA to malonate. In some embodiments, the expression vector is a yeast expression vector; in other embodiments, the expression vector is a bacterial expression vector. In various embodiments, the bacterial expression vector is an Escherichia coli expression vector.

In a third aspect, the invention provides recombinant host cells suitable for the biosynthetic production of malonate at levels enabling its isolation and use as a starting material for chemical synthesis of other useful products. In some embodiments, the host cell is a eukaryote. In some embodiments, the host cell is a yeast cell. In various embodiments, the yeast is a Candida, Cryptococcus, Hansenula, Issatchenkia, Kluyveromyces, Komagataella, Lipomyces, Pichia, Rhodosporidium, Rhodotorula, Saccharomyces or Yarrowia species. In some embodiments, the eukaryotic host cell is a fungus. In some embodiments, the cell host is an algae.

In other embodiments, the host cell is a bacterial cell. In various embodiments, the host cell is a bacterial cell selected from the group consisting of Bacillus, Clostridium, Corynebacterium, Escherichia, Pseudomonas, and Streptomyces. In some embodiments, the host cell is an E. coli cell.

Generally, the recombinant host cells of the invention have been genetically modified for improved malonate yield, titer, and/or productivity. In various embodiments, the host cells have been modified for increased malonate biosynthesis through one or more host cell modifications selected from the group consisting of modifications that result in increased acetyl-CoA biosynthesis, increased malonyl-CoA biosynthesis, decreased malonyl-CoA utilization, decreased malonate catabolism, increased secretion of malonate into the fermentation broth, increased host cell tolerance to malonate in the fermentation broth, and/or increased host cell catabolism of carbon sources (e.g. acetate, alginate, ethanol, fatty acids, lignocellulosic biomass, methanol, pentose sugars, and syn gas).

In a fourth aspect, the invention provides methods for producing malonate in a recombinant host cell, which methods generally comprise culturing the recombinant host cell in fermentation broth under conditions that enable it to produce malonate. In some embodiments, the host cell has been engineered to express more or less of an endogenous enzyme that results in the production of more malonate than a corresponding cell that has not been so engineered. In some embodiments, the method comprises culturing a recombinant host cell expressing a heterologous (foreign or non-native) enzyme that results in the increased production of malonate. In some embodiments, the host cell used in the method comprises one or expression vectors comprising heterologous malonyl-CoA hydrolase enzymes. In some embodiments of these methods, the fermentation broth is supplemented with carbon sources promoting malonate production and selected from the group consisting of cellodextrins, 5 carbon sugars, 6 carbon sugars, carbon dioxide, ethanol, methanol, glycerol, acetate, and/or fatty acids.

In a fifth aspect, the invention provides biosensors comprising a malonate transcription factor and a promoter responsive to said transcription factor operably linked to a marker gene. The invention also provides methods for “sensing” malonate, malonate production, and malonate producing host cells and methods for screening for host cells with increased malonate production. In various embodiments, said methods comprise culturing a host cell expressing a malonate transcription factor and containing a promoter responsive to said transcription factor and operably linked to a marker gene, and selecting host cells with improved malonate production by screening for expression of the marker gene product and selecting those host cells that express higher levels of the marker gene product. In some embodiments, malonate is produced in one host cell, the fermentation broth from the first cell is contacted with (added to media containing) a second cell comprising a malonate transcription factor and a promoter responsive to said transcription factor operably linked to a marker gene, and host cells with improved malonate production are identified by identifying cells with the highest levels of expression of the marker gene product. In other embodiments, malonate is produced in a host cell comprising a malonate transcription factor and promoter responsive to malonate operably linked to a marker gene, and host cells with increased malonate production are screened for increased malonate production by screening for and identifying cells that express the highest levels of the marker gene product. In some embodiments, the transcription factor can bind malonate, which results in binding of the transcription factor to a cognate promoter and activation of the marker gene that is operably linked to the promoter. In some embodiments, the transcription factor is an MdcY transcription factor. In some embodiments, the method is practiced to screen or select for genetically modified host cells with improved malonate production relative to control cells.

In a sixth aspect, the invention provides purified malonate isolated from the fermentation broth of a host cell producing malonate, optionally a host cell of the invention. The invention also provides methods for purifying malonate from the fermentation broth of a host cell producing malonate, the methods generally comprising culturing a host cell in fermentation broth under conditions that enable the host cell to produce malonate, and purifying the malonate from the fermentation broth. In some embodiments of the invention, the concentration of malonate in the broth is increased by dewatering the fermentation broth during the purification process. In various embodiments of the invention, the dewatering is achieved by reverse osmosis processing, evaporation, or a combination of the two. In various embodiments, the purification is achieved by adding one or more of the following: a divalent cation, a monovalent cation, ammonium, a monosubstituted amine, a disubstituted amine, a trisubstituted amine, a cationic purification resin, or an acid. In various embodiments of the invention, these agents are added in conjunction with one or more organic solvents. In some embodiments of the invention, a hydrophobic solvent is used in a liquid-liquid extraction of the fermentation broth. In other embodiments, malonate is purified from the fermentation broth by reactive extraction or distillation with an acid catalyst and an alcohol.

In a seventh aspect, the invention provides methods of making compounds derived from malonate and compounds produced by such methods. The methods generally comprise reacting malonate with one or more substrates to produce a compound. In some embodiments of these methods, chemicals with established synthetic routes from malonate are produced using biologically derived malonate. In other embodiments of these methods, new synthetic routes for the production of useful chemicals are provided that are suitable for use with either a synthetically or biologically derived malonate. In some embodiments, monoalkyl malonate esters are synthesized from biologically derived malonate. In other embodiments, dialkyl malonate esters are synthesized from biologically derived malonate. In some embodiments, an acrylate is synthesized from malonate or malonic acid. In other embodiments, acrylate is synthesized from malonate monoesters or diesters. In other embodiments, dicarboxylic acids are produced from malonate. Illustrative dicarboxylic acids that can be produced in accordance with the methods of the invention include those selected from the group consisting of pentanedioic acid, hexanedioic acid, heptanedioic acid, octanedioic acid, nonanedioic acid, decanedioic acid, undecanedioic acid, dodecanedioic acid, and the corresponding monoalkyl and dialkyl esters of each. In other embodiments of the invention, dicarboxylic acids are produced from a malonate-derived compound. In other embodiments of the invention, ε-caprolactam is produced from malonate. In other embodiments of the invention, δ-valerolactam is produced from malonate.

These and other aspects and embodiments of the invention are illustrated in the accompanying drawings and described in more detail below.

### BRIEF DESCRIPTION OF THE FIGURES

FIG. 1 shows a dose-response curve for an E. coli MdcY malonate biosensor of the invention that utilizes the promoter P_{MdcL}. The X-axis is the concentration of exogenous malonate added to the fermentation broth; the Y-axis is the cell culture density (OD₆₀₀) after 12 hours growth in medium with 25 μg/ml tetracycline. E. coli transformed with plasmid S14, comprising an MdcY transcription factor and a tetA gene under control of a P_{MdcL} promoter, produced the tetracycline resistance protein TetA upon exogenous addition of malonate. The biosensor displayed malonate-dependent increases in tetracycline resistance as measured by the increase in OD₆₀₀ with the increase in concentration exogenously added malonate as described in additional detail in Example 21.

FIG. 2 is an HPLC chromatogram trace showing separation and detection of acrylic acid production from malonate according to the methods of the invention as described in Example 28. The X-axis shows elution of acrylic acid at approximately 17.5 minutes, and the Y-axis shows arbitrary units derived from detection of acrylic acid via a UV detector monitoring 210 nm. Acrylic acid produced from malonate according to the methods of the invention (dashed line) exhibited the same retention time as an authentic acrylic acid standard (solid line).

### DETAILED DESCRIPTION OF THE INVENTION

The present invention provides recombinant host cells, materials, and methods for the biological production of malonate, screening malonate producing host cells for improved malonate production, purification of biologically produced malonate, and the synthetic conversion of malonate to industrially important chemicals.

While the present invention is described herein with reference to aspects and specific embodiments thereof, those skilled in the art will recognize that various changes may be made and equivalents may be substituted without departing from the invention. The present invention is not limited to particular nucleic acids, expression vectors, enzymes, host microorganisms, or processes, as such may vary. The terminology used herein is for purposes of describing particular aspects and embodiments only, and is not to be construed as limiting. In addition, many modifications may be made to adapt a particular situation, material, composition of matter, process, process step or steps, in accordance with the invention. All such modifications are within the scope of the claims appended hereto.

All patents, patent applications, and publications cited herein are incorporated herein by reference in their entireties.

### Section 1: Definitions

In this specification and in the claims that follow, reference will be made to a number of terms that shall be defined to have the following meanings.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

Amino acids in a protein coding sequence are identified herein by the following abbreviations and symbols. Specific amino acids are identified by a single-letter abbreviation, as follows: A is alanine, R is arginine, N is asparagine, D is aspartic acid, C is cysteine, Q is glutamine, E is glutamic acid, G is glycine, H is histidine, L is leucine, I is isoleucine, K is lysine, M is methionine, F is phenylalanine, P is proline, S is serine, T is threonine, W is tryptophan, Y is tyrosine, and V is valine. A dash (−) in a consensus sequence indicates that there is no amino acid at the specified position. A plus (+) in a consensus sequence indicates any amino acid may be present at the specified position. Thus, a plus in a consensus sequence herein indicates a position at which the amino acid is generally non-conserved; a homologous enzyme sequence, when aligned with the consensus sequence, can have any amino acid at the indicated “+” position. At positions in a consensus sequence where one of a subset of amino acids can be present, the following abbreviations are used: B represents that one of the amino acids R, K, or H is present at the indicated position; J represents that one of the amino acids D or E is present at the indicated position; O represents that one of the amino acids I, L, or V is present at the indicated position; U represents that one of the amino acids S or T is present at the indicated position; and X₁ represents that one of the amino acids A, D, R, H, K, S, T, N, Q, or Y (or a subset of those amino acids) is present at the indicated position. Illustrative subsets of X₁ include X₁ is A, D, K, S, T, N, or Y and X₁ is S or N. Specific amino acids in a protein coding sequence are identified by their respective single-letter abbreviation followed by the amino acid position in the protein coding sequence where 1 corresponds to the amino acid (typically methionine) at the N-terminus of the protein. For example, E124 in S. cerevisiae wild type EHD3 refers to the glutamic acid at position 124 from the EHD3 N-terminal methionine (i.e., M1). Amino acid substitutions (i.e., point mutations) are indicated by identifying the mutated (i.e., progeny) amino acid after the single-letter code and number in the parental protein coding sequence; for example, E124A in S. cerevisiae EHD3 refers to substitution of alanine for glutamic acid at position 124 in the EHD3 protein coding sequence. The mutation may also be identified in parentheticals, for example EHD3 (E124A). Multiple point mutations in the protein coding sequence are separated by a backslash (/); for example, EHD3 E124A/Y125A indicates that mutations E124A and Y125A are both present in the EHD3 protein coding sequence. The number of mutations introduced into some examples has been annotated by a dash followed by the number of mutations, preceeding the parenthetical identification of the mutation (e.g. A5W8H3-1 (E95Q)). The Uniprot IDs with and without the dash and number are used interchangeably herein (i.e. A5W8H3-1 (E95Q)=A5W8H3 (E95Q)).

As used herein, the term “express”, when used in connection with a nucleic acid encoding an enzyme or an enzyme itself in a cell, means that the enzyme, which may be an endogenous or exogenous (heterologous) enzyme, is produced in the cell. The term “overexpress”, in these contexts, means that the enzyme is produced at a higher level, i.e., enzyme levels are increased, as compared to the wild-type, in the case of an endogenous enzyme. Those skilled in the art appreciate that overexpression of an enzyme can be achieved by increasing the strength or changing the type of the promoter used to drive expression of a coding sequence, increasing the strength of the ribosome binding site or Kozak sequence, increasing the stability of the mRNA transcript, altering the codon usage, increasing the stability of the enzyme, and the like.

The terms “expression vector” or “vector” refer to a nucleic acid and/or a composition comprising a nucleic acid that can be introduced into a host cell, e.g., by transduction, transformation, or infection, such that the cell then produces (“expresses”) nucleic acids and/or proteins other than those native to the cell, or in a manner not native to the cell, that are contained in or encoded by the nucleic acid so introduced. Thus, an “expression vector” contains nucleic acids (ordinarily DNA) to be expressed by the host cell. Optionally, the expression vector can be contained in materials to aid in achieving entry of the nucleic acid into the host cell, such as the materials associated with a virus, liposome, protein coating, or the like. Expression vectors suitable for use in various aspects and embodiments of the present invention include those into which a nucleic acid sequence can be, or has been, inserted, along with any preferred or required operational elements. Thus, an expression vector can be transferred into a host cell and, typically, replicated therein (although, one can also employ, in some embodiments, non-replicable vectors that provide for “transient” expression). In some embodiments, an expression vector that integrates into chromosomal, mitochondrial, or plastid DNA is employed. In other embodiments, an expression vector that replicates extrachromosomally is employed. Typical expression vectors include plasmids, and expression vectors typically contain the operational elements required for transcription of a nucleic acid in the vector. Such plasmids, as well as other expression vectors, are described herein or are well known to those of ordinary skill in the art.

The terms “ferment”, “fermentative”, and “fermentation” are used herein to describe culturing microbes under conditions to produce useful chemicals, including but not limited to conditions under which microbial growth, be it aerobic or anaerobic, occurs.

The term “heterologous” as used herein refers to a material that is non-native to a cell. For example, a nucleic acid is heterologous to a cell, and so is a “heterologous nucleic acid” with respect to that cell, if at least one of the following is true: (a) the nucleic acid is not naturally found in that cell (that is, it is an “exogenous” nucleic acid); (b) the nucleic acid is naturally found in a given host cell (that is, “endogenous to”), but the nucleic acid or the RNA or protein resulting from transcription and translation of this nucleic acid is produced or present in the host cell in an unnatural (e.g., greater or lesser than naturally present) amount; (c) the nucleic acid comprises a nucleotide sequence that encodes a protein endogenous to a host cell but differs in sequence from the endogenous nucleotide sequence that encodes that same protein (having the same or substantially the same amino acid sequence), typically resulting in the protein being produced in a greater amount in the cell, or in the case of an enzyme, producing a mutant version possessing altered (e.g. higher or lower or different) activity; and/or (d) the nucleic acid comprises two or more nucleotide sequences that are not found in the same relationship to each other in the cell. As another example, a protein is heterologous to a host cell if it is produced by translation of RNA or the corresponding RNA is produced by transcription of a heterologous nucleic acid; a protein is also heterologous to a host cell if it is a mutated version of an endogenous protein, and the mutation was introduced by genetic engineering.

The terms “host cell” and “host microorganism” are used interchangeably herein to refer to a living cell that can be (or has been) transformed via insertion of an expression vector. A host microorganism or cell as described herein may be a prokaryotic cell (e.g., a microorganism of the kingdom Eubacteria) or a eukaryotic cell. As will be appreciated by one of skill in the art, a prokaryotic cell lacks a membrane-bound nucleus, while a eukaryotic cell has a membrane-bound nucleus.

The terms “isolated” or “pure” refer to material that is substantially, e.g. greater than 50% or greater than 75%, or essentially, e.g. greater than 90%, 95%, 98% or 99%, free of components that normally accompany it in its native state, e.g. the state in which it is naturally found or the state in which it exists when it is first produced.

A carboxylic acid as described herein can be a salt, acid, base, or derivative depending on the structure, pH, and ions present. The terms “malonate” and “malonic acid” are used interchangeably herein. Malonic acid is also called propanedioic acid (C3H404; CAS #141-82-2).

The term “malonate-derived compounds” as used herein refers to mono-alkyl malonate esters, including, for example and without limitation, mono-methyl malonate (also referred to as monomethyl malonate, CAS #16695-14-0), mono-ethyl malonate (also referred to as monoethyl malonate, CAS #1071-46-1), mono-propyl malonate, mono-butyl malonate, mono-tert-butyl malonate (CAS #40052-13-9), and the like; di-alkyl malonate esters, for example and without limitation, dimethyl malonate (CAS #108-59-8), diethyl malonate (CAS #105-53-3), dipropyl malonate (CAS #1117-19-7), dibutyl malonate (CAS #1190-39-2), and the like, and Meldrum's acid (CAS #2033-24-1). The malonate-derived compounds can be produced synthetically from malonate and are themselves valuable compounds but are also useful substrates in the chemical synthesis of a number of other valuable compounds.

As used herein, the term “nucleic acid” and variations thereof shall be generic to polydeoxyribonucleotides (containing 2-deoxy-D-ribose) and to polyribonucleotides (containing D-ribose). “Nucleic acid” can also refer to any other type of polynucleotide that is an N-glycoside of a purine or pyrimidine base, and to other polymers containing nonnucleotidic backbones, provided that the polymers contain nucleobases in a configuration that allows for base pairing and base stacking, as found in DNA and RNA. As used herein, the symbols for nucleotides and polynucleotides are those recommended by the IUPAC-IUB Commission of Biochemical Nomenclature (Biochem. 9:4022, 1970). A “nucleic acid” may also be referred to herein with respect to its sequence, the order in which different nucleotides occur in the nucleic acid, as the sequence of nucleotides in a nucleic acid typically defines its biological activity, e.g., as in the sequence of a coding region, the nucleic acid in a gene composed of a promoter and coding region, which encodes the product of a gene, which may be an RNA, e.g. a rRNA, tRNA, or mRNA, or a protein (where a gene encodes a protein, both the mRNA and the protein are “gene products” of that gene).

The term “operably linked” refers to a functional linkage between a nucleic acid expression control sequence (such as a promoter, ribosome-binding site, and transcription terminator) and a second nucleic acid sequence, the coding sequence or coding region, wherein the expression control sequence directs or otherwise regulates transcription and/or translation of the coding sequence.

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

As used herein, “recombinant” refers to the alteration of genetic material by human intervention. Typically, recombinant refers to the manipulation of DNA or RNA in a cell or virus or expression vector by molecular biology (recombinant DNA technology) methods, including cloning and recombination. Recombinant can also refer to manipulation of DNA or RNA in a cell or virus by random or directed mutagenesis. A “recombinant” cell or nucleic acid can typically be described with reference to how it differs from a naturally occurring counterpart (the “wild-type”). In addition, any reference to a cell or nucleic acid that has been “engineered” or “modified” and variations of those terms, is intended to refer to a recombinant cell or nucleic acid.

As used herein, the term “transcription factor biosensor” refers to a system to detect a substance, e.g., malonate, by activating expression of a “marker” or “reporter” gene where reporter gene expression is mediated by a transcription factor that is capable of binding to a promoter and activating transcription upon binding of that substance, e.g., malonate. For example, malonate may bind to a transcription factor (e.g., MdcY) and activate transcription from a promoter (e.g., P_{MdcL}). A “malonate transcription factor” is a transcription factor that, when bound to malonate, can activate a promoter. Thus, MdcY is a malonate transcription factor.

The terms “transduce”, “transform”, “transfect”, and variations thereof as used herein refers to the introduction of one or more nucleic acids into a cell. For practical purposes, the nucleic acid must be stably maintained or replicated by the cell for a sufficient period of time to enable the function(s) or product(s) it encodes to be expressed for the cell to be referred to as “transduced”, “transformed”, or “transfected”. As will be appreciated by those of skill in the art, stable maintenance or replication of a nucleic acid may take place either by incorporation of the sequence of nucleic acids into the cellular chromosomal DNA, e.g., the genome, as occurs by chromosomal integration, or by replication extrachromosomally, as occurs with a freely-replicating plasmid. A virus can be stably maintained or replicated when it is “infective”: when it transduces a host microorganism, replicates, and (without the benefit of any complementary virus or vector) spreads progeny expression vectors, e.g., viruses, of the same type as the original transducing expression vector to other microorganisms, wherein the progeny expression vectors possess the same ability to reproduce.

### Section 2: Malonyl-CoA Hydrolase Enzymes

In accordance with one aspect of the invention, malonate is produced through the action of a malonyl-CoA hydrolase catalyzing the conversion of malonyl-CoA to malonate. To date, no wild-type malonyl-CoA hydrolase gene has been identified, although the presence of a small amount of malonate in the fermentation media of non-engineered strains indicates that a wild-type enzyme with this activity may exist. The present invention provides a number of genes that are counterparts to wild type genes that have been mutated to confer malonyl-CoA hydrolase activity. The host cell making the malonyl-CoA hydrolase is a recombinant host cell; in many embodiments, the host cell has been genetically modified to comprise heterologous nucleic acid(s) encoding malonyl-CoA hydrolase enzyme(s) catalyzing hydrolysis of malonyl-CoA to malonate. In some embodiments, the recombinant host cell is a eukaryote. In various embodiments, the eukaryote is a yeast strain selected from the non-limiting example genera: Candida, Cryptococcus, Hansenula, Issatchenkia, Kluyveromyces, Komagataella, Lipomyces, Pichia, Rhodosporidium, Rhodotorula, Saccharomyces, or Yarrowia. Those skilled in the art will recognize that these genera broadly encompass yeast, including those distinguished as oleaginous yeast. In some embodiments, the host cell is Saccharomyces cerevisiae. In other embodiments, the host cell is Pichia kudriavzevii. In other embodiments of the invention, the eukaryotic host cell is a fungus or algae. In yet other embodiments, the recombinant host cell is a prokaryote selected from the non-limited example genera: Bacillus, Clostridium, Corynebacterium, Escherichia, Pseudomonas, Rhodobacter, and Streptomyces. In some embodiments, the host cell is E. coli.

The present invention results in part from the discovery that various acyl-CoA hydrolases and transacylases can be engineered to have malonyl-CoA hydrolase activity and so be useful for biological production of malonate. Non-limiting examples of acyl-CoA hydrolases suitable for modification for malonyl-CoA hydrolysis include any of those from the group consisting of 3-hydroxyisobutyryl-CoA hydrolases (EC 3.1.2.4), 3-hydroxypropionyl-CoA hydrolases (EC 3.1.2.4), acetoacetyl-CoA hydrolases (EC 3.1.2.11), methylmalonyl-CoA hydrolases (EC 3.1.2.17), propionyl-CoA hydrolases (EC 3.1.2.18), succinyl-CoA hydrolases (EC 3.1.2.3), and malonyl-CoA:ACP transacylases (EC 2.3.1.39) mutated as provided herein to have malonyl-CoA hydrolase activity.

In some embodiments, the malonyl-CoA hydrolase used to produce malonate in accordance with the invention is a mutated S. cerevisiae EHD3 acyl-CoA hydrolase (see SEQ ID NO:1 for the wild-type EHD3 amino acid sequence). One such mutant with altered substrate specificity is the E124V mutant (see Rouhier, “Characterization of YDR036C from Saccharomyces cerevisiae.” Dissertation, Miami University, Miami University and OhioLINK (2011)), which, while previously reported, was not reported to have malonyl-CoA hydrolase activity. In some embodiments of the invention, an E. coli host cell expressing the E124V mutant is used to produce malonate, which is then purified from the cell or fermentation broth. In other embodiments of the invention, a yeast cell expressing the E124V mutant is used to produce malonate in accordance with the invention. In yet another embodiment, an oleaginous yeast cell expressing the E124V mutant is used to produce malonate in accordance with the invention.

Prior attempts to produce the E124A mutant of EHD3 resulted in cell death upon induction of protein expression from a pET28a expression vector; the protein was unable to be purified (see Rouhier, supra). The present invention provides expression vectors for the E124A mutant that can be used in E. coli host cells, rendering them capable of producing malonate. These E. coli expression vectors are characterized in that, relative to the pET28a vector of Rouhier, the E124A mutant is produced at a lower, non-toxic, level. This is achieved, for example by employing expression vectors with a lower copy number or weaker promoter than used by Rouhier. One skilled in the art will also appreciate that translation can be modulated by the affinity of the ribosome binding site (RBS), or Kozak sequence, for the ribosome. Thus a weaker RBS or Kozak sequence can also be employed to reduce gene expression. Examples of lower copy number expression vectors include, but are not limited to pSC101 origin expression vectors, p15a origin expression vectors, and expression vectors that integrate into the chromosomal DNA. Examples of weaker promoters than the T7 promoter used by Rouhier include, but are not limited to the P_{LacO1}, P_{TRC}, and P_{BAD} promoters. In some embodiments, the vector has a pSC101 origin of replication. In other embodiments, the promoter used for expression of the EHD3 E124A mutant coding sequence is the P_{lacO1} promoter. Additionally, the present invention provides vectors for yeast host cells that code for the expression of the E124A mutant. The genetically modified S. cerevisiae EHD3 E124A expression vectors of the invention can be used in vivo for the production of malonate in E. coli and S. cerevisiae, and the methods of the invention provide means for the subsequent purification of malonate from fermentation broth of these strains, and the synthetic conversion of malonate into derivative small-molecule compounds.

The present invention also provides the E124S mutant of EHD3 for use as a malonyl-CoA hydrolase, vectors for expressing this mutant, and host cells that express this mutant and produce malonate (see Example 31). Wild-type S. cerevisiae EHD3 catalyzes the hydrolysis of 3-hydroxypropionyl-CoA (3HPA-CoA) and 3-hydroxyisobutyryl-CoA (3HIBA-CoA), and while this invention is not to be limited by theory, E124 is predicted to interact with the terminal hydroxyl moiety on 3HPA-CoA, stabilizing the substrate in the EHD3 active site (see Rouhier, supra). Certain aspects of this invention arise from the discovery that specific E124 point mutations increase enzyme hydrolysis of malonyl-CoA, producing malonate. Mutation of E124 to a nucleophilic amino acid (e.g., S or T), basic amino acid (e.g., H, K, or R), or amide amino acid (e.g., N or Q) improves the binding of malonyl-CoA in the EHD3 active site over 3-hydroxypropionyl-CoA and increases malonate production (relative to the unmutated counterpart enzyme). The E124S, E124T, E124N, E124Q, E124H, E124K, and E124R mutations also decrease production of byproducts (e.g., acetate, propionate, isobutyrate, and succinate) due to decreased hydrolysis of endogenous host cell acyl-CoA molecules. The E124S point mutation places a hydroxyl moiety in a position that promotes hydrogen bonding between the serine residue and the terminal carboxylate group of malonyl-CoA. The E124Q point mutation places the glutamine amide group in a position near the terminal carboxylate group of malonyl-CoA. The E124K point mutation places the lysine amine group in a position that promotes hydrogen bonding between the lysine residue and the terminal carboxylate group of malonyl-CoA. In contrast to the nucleophilic, amide, and basic E124 point mutations described above, mutations E124A and E124V remove the presence of a charged amino acid at position 124; these mutations both eliminate hydrogen bonding between the terminal carboxylate on malonate and the EHD3 124 amino acid sidechain and open the EHD3 active site to promiscuous activity, increasing undesirable byproduct formation and decreasing malonate production.

In some embodiments of the invention, an E. coli host cell expressing the E124S mutant is used to produce malonate. In other embodiments of the invention, a yeast host cell expressing the E124S mutant is used to produce malonate. In other embodiments, an oleaginous yeast host cell expressing the E124S mutant is used to produce malonate. In some embodiments of the invention, an E. coli host cell expressing the E124Q mutant is used to produce malonate. In other embodiments of the invention, a yeast host cell expressing the E124Q mutant is used to produce malonate. In other embodiments, an oleaginous yeast host cell expressing the E124Q mutant is used to produce malonate. In some embodiments of the invention, an E. coli host cell expressing the E124K mutant is used to produce malonate. In other embodiments of the invention, a yeast host cell expressing the E124K mutant is used to produce malonate. In other embodiments, an oleaginous yeast host cell expressing the E124K mutant is used to produce malonate. In some embodiments of the invention, an E. coli host cell expressing the E124H mutant is used to produce malonate. In other embodiments of the invention, a yeast host cell expressing the E124H mutant is used to produce malonate. In other embodiments of the invention, an oleaginous yeast host cell expressing the E124H mutant is used to produce malonate. In some embodiments of the invention, an E. coli host cell expressing the E124R mutant is used to produce malonate. In other embodiments of the invention, a yeast host cell expressing the E124R mutant is used to produce malonate. In other embodiments of the invention, an oleaginous yeast host cell expressing the E124R mutant is used to produce malonate. In other embodiments, a recombinant host cell expressing an EHD3 E124 nucleophilic amino acid point mutation (i.e., E124S or E124T) is used to produce malonate. In other embodiments, a recombinant host cell expressing an EHD3 E124 basic amino acid point mutation (i.e., E124H, E124K, or E124R) is used to produce malonate. In other embodiments, a recombinant host cell expressing an EHD3 E124 amide amino acid point mutation (i.e., E124N or E124Q) is used to produce malonate.

The present invention also provides a mutated EHD3 comprising a mutated active site, vectors for expressing the mutant, and host cells that express the mutant and produce malonate. Certain aspects of the present invention arose, in part, from the discovery that specific amino acids (i.e., F121, and F177) are important for acyl-CoA substrate binding, and introduction of specific point mutations increase malonyl-CoA hydrolysis and production of malonate. Introduction of mutation F121I or F121L increases malonyl-CoA access to the active site. Similarly, introduction of mutation F177I or F177L increases malonyl-CoA access to the active site. One or more point mutations at amino acid positions F121 or F177 can be introduced alone, or along with an E124 point mutation. In various embodiments, a F121 and/or F177 point mutation is introduced along with an E124 point mutation. In some embodiments, a recombinant host cell expressing an EHD3 F121I or F121L mutant is used to produce malonate. In other embodiments, a recombinant host cell expressing an EHD3 F177I or F178L mutant is used to produce malonate. In these embodiments, the recombinant host cell can be, without limitation, an E. coli or yeast, including but not limited to S. cerevisiae or other yeast, host cell.

The present invention also provides mutated EHD3 comprising a mutated mitochondrial targeting sequence, vectors for expressing the mutant, and host cells that express the mutant and produce malonate. In an S. cerevisiae host, wild-type EHD3 is localized in the mitochondria. Malonyl-CoA is found in both the mitochondria and the cytosol; EHD3 catalyzed hydrolysis of cytosolic malonyl-CoA requires localization of an EHD3 to the cytosol. Certain aspects of the present invention arose from the discovery that mutations of the EHD3 mitochondrial targeting sequence can increase production of malonate. The EHD3 amino acids important for mitochondrial targeting include R3, K7, K14, K18, and R22, and mutation of one or more of these basic amino acids to a hydrophobic amino acid (i.e., A or V) abrogates mitochondrial targeting. In some embodiments, a recombinant host comprising an EHD3 consisting of one or more mutations to A or V at amino acids selected from the group consisting of R3, K7, K14, K18, and R22 is used to produce malonate. In some embodiments, the recombinant host is a yeast strain. In other embodiments, the host is S. cerevisiae. In still further embodiments, the recombinant host cell contains one or more copies of an EHD3 with the mitochondrial targeting sequence unaltered (i.e., wild-type) and one or more copies of an EHD3 with the mitochondrial targeting sequence mutated. Additional examples of mitochondrial targeting sequences useful in this aspect of the invention are: WT COX4, SynA1, SynA2, Syn B1, and Syn B2, as outline for other applications by Allison & Schatz (Allison & Schatz (1986) PNAS 83:9011-9015). In further embodiments of the invention, peroxisomal targeting signal (e.g. PTS1, PTS2), most often containing a Ser-Lys-Leu motif, is fused to the C-terminus of the malonyl-CoA hydrolase to result in localization of this protein to the peroxisome.

Thus, in one aspect of the invention, the recombinant host cell comprises a heterologous nucleic acid encoding a mutant S. cerevisiae EHD3 that results in increased production of malonate relative to host cells not comprising the mutant EHD3. In some embodiments, the mutant EHD3 is heterologously expressed in E. coli. In other embodiments, the mutant EHD3 is heterologously expressed in S. cerevisiae. In other embodiments, the mutant EHD3 is heterologously expressed in an oleaginous yeast cell. In some embodiments, the mutant EHD3 contains a point mutation at position E124. In some embodiments, the point mutation at residue E124 is either E124A or E124V. In some embodiments, the point mutation at E124 is E124S or E124T. In some embodiments, the point mutation at E124 is E124S. In some embodiments, the point mutation at E124 is a basic amino acid selected from the group consisting of E124H, E124K, and E124R In some embodiments, the point mutation at E124 is E124H. In some embodiments, the point mutation at E124 is E124K. In some embodiments, the point mutation at E124 is E124R In some embodiments, the point mutation at residue E124 is E124N or E124Q. In some embodiments, the point mutation at residue E124 is E124Q. In some embodiments, one or more EHD3 amino acids selected from the group consisting of F121 and F177 are mutated to I or L. In some embodiments, one or more EHD3 amino acids selected from the group consisting of R3, K7, K14, K18, and R22 are mutated to either A or V.

In another aspect of the invention, an enzyme other than, or in addition to, EHD3 is utilized as a malonyl-CoA hydrolase to produce malonate in accordance with the invention. In some embodiments, Haemophilus influenzae YciA is heterologously expressed in a heterologous host to produce malonate in accordance with the invention (see Zhuang et al. Biochemistry 47: 2789-2796 (2008)). In other embodiments, the malonyl-CoA hydrolase is an acyl-CoA hydrolase endogenous to Rattus norvegicus (see Kovachy et al., J. Biol. Chem. 258:11415-11421 (1983)). In other embodiments, the malonyl-CoA hydrolase is the acyl-CoA hydrolase from brown adipose tissue mitochondrial protein fraction from Mesocricetus auratus (see Alexson et al., J. Biol. Chem. 263:13564-13571 (1988)).

Thus, in accordance with the invention acyl-CoA hydrolases other than, or in addition to, EHD3 (from S. cerevisiae or homologous enzymes from other organisms) can be used for biological synthesis of malonate in a recombinant host. In some embodiments, the recombinant host is S. cerevisiae. In other embodiments, the recombinant host is E. coli. In other embodiments, the recombinant host is a yeast other than S. cerevisiae as described in additional detail below. In various embodiments, the host is modified to express a mutated enzyme selected from the group consisting of S. albicans EHD3, H. sapiens HIBCH (UniProt: Q6NVY1), A. thaliana CHY1 (UniProt: Q9LKJ1), R. norvegicus HIBCH (UniProt: Q5XIE6), M. musculus HIBCH (UniProt: Q8QZS1), G. gallus HIBCH (UniProt: Q5ZJ60), B. taurus HIBCH (UniProt: Q2HJ73), D. rerio HIBCH (UniProt: Q58EB4), B. cereus Bch, P. aeruginosa Hich, E. coli YciA, H. influenzae YciA, M. musculus ACOT4, M. musculus ACOT8, S. enterica SARI_01218, A. pernix K1, C. hutchinsonii Chut02003666, S. solfataricus P2 SS02287, S. acidocaldarius DSM 639 Saci_0145, P. aerophilum str. IM2 PAE3404, D. melanogaster CG1635, P. carbinolicus DSM 2380 Pcar_1366, A. dehalogenans 2CP-C 110, G. gallus ACO9, and X. laevis MGC114623.

Those of skill in the art will appreciate that one or multiple suitably mutated acyl-CoA hydrolases can be used in accordance with the invention to convert malonyl-CoA to malonate in a host cell. Moreover, acyl-CoA hydrolases other than those specifically disclosed herein can be utilized in mutated or heterologously expressed form, and it will be well understood to those skilled in the art in view of this disclosure how other appropriate enzymes can be identified, modified, and expressed to achieve the desired malonyl-CoA hydrolase activity as disclosed herein.

### Consensus Sequences

Malonyl-CoA hydrolases of the invention include those that are homologous to consensus sequences provided by the invention. As noted above, any enzyme substantially homologous to an enzyme specifically described herein can be used in a host cell of the invention. One enzyme is homologous to another (the “reference enzyme”) when it exhibits the same activity of interest and can be used for substantially similar purposes. Generally, homologous enzymes share substantial sequence identity. Sets of homologous enzymes generally possess one or more specific amino acids that are conserved across all members of the consensus sequence protein class.

The percent sequence identity of an enzyme relative to a consensus sequence is determined by aligning the enzyme sequence against the consensus sequence. Those skilled in the art will recognize that various sequence alignment algorithms are suitable for aligning an enzyme with a consensus sequence. See, for example, Needleman, S B, et al “A general method applicable to the search for similarities in the amino acid sequence of two proteins.” Journal of Molecular Biology 48 (3): 443-53 (1970). Following alignment of the enzyme sequence relative to the consensus sequence, the percentage of positions where the enzyme possesses an amino acid (or dash) described by the same position in the consensus sequence determines the percent sequence identity. When a degenerate amino acid (i.e. B, J, O, U, “+”) is present in a consensus sequence any of the amino acids described by the degenerate amino acid may be present in the enzyme at the aligned position for the enzyme to be identical to the consensus sequence at the aligned position. When a dash is present in a consensus sequence the enzyme must not have an amino acid present in the aligned position for the enzyme to be identical to the consensus sequence at the aligned position.

The present invention provides consensus sequences useful in identifying and constructing malonyl-CoA hydrolases of the invention. In various embodiments, these malonyl-CoA hydrolase consensus sequences contain active site amino acid residues believed to be necessary (although the invention is not to be limited by any theory of mechanism of action) for formation of an oxyanion hole responsible for stabilizing the enolate anion intermediate derived from a malonyl-CoA substrate as well as the amino acid residues important for malonyl-CoA binding, as described below. A malonyl-CoA hydrolase enzyme encompassed by a consensus sequence provided herein has an enzymatic activity that is identical, or essentially identical, or at least substantially similar with respect to ability to hydrolyze malonyl-CoA to that of one of the enzymes exemplified herein. A malonyl-CoA hydrolase enzyme may be found in nature or, more typically, is an engineered mutant of a wild-type enzyme modified in accordance with the invention to have malonyl-CoA hydrolase activity. A malonyl-CoA hydrolase enzyme may be identified or constructed from another enzyme by mutating the sequence of the other enzyme to create a sequence encompassed by a consensus sequence herein; if an enzyme shares substantial homology to a consensus sequence herein but has suboptimal, including no, malonyl-CoA hydrolase activity, then, in accordance with the invention, it is mutated to conform to a consensus sequence provided herein to provide a malonyl-CoA hydrolase of the invention.

The invention provides four malonyl-CoA hydrolase consensus sequences: (i) malonyl-CoA hydrolase based on EHD3 EC 3.1.2.4 (SEQ ID NO:7), (ii) malonyl-CoA hydrolase based on Bacillus EC 3.1.2.4 (SEQ ID NO:8), (iii) malonyl-CoA hydrolase based on Pseudomonas EC 3.1.2.4 (SEQ ID NO:9), and (iv) malonyl-CoA hydrolase based on both Bacillus and Pseudomonas EC 3.1.2.4 ((SEQ ID NO:10). The consensus sequences provide a sequence of amino acids in which each position identifies the amino acid (if a specific amino acid is identified) or a subset of amino acids (if a position is identified as variable) most likely to be found at a specified position in a malonyl-CoA hydrolase of that class. Those of skill in the art will recognize that fixed amino acids and conserved amino acids in these consensus sequences are identical to (in the case of fixed amino acids) or consistent with (in the case of conserved amino acids) with the wild-type sequence(s) on which the consensus sequence is based. A dash in a consensus sequences indicates that suitable enzymes for mutation in accordance with the invention can be found in nature that may have an additional amino acid at the location of the dash in the sequence, but typically no amino acid is present at the location of a dash.

### Malonyl-CoA Hydrolase Consensus Sequence Based on EHD3 EC 3.1.2.4 Enzymes

The invention provides a malonyl-CoA hydrolase consensus sequence based on EHD3 EC 3.1.2.4 enzymes (SEQ ID NO:7), and in various embodiments, suitable malonyl-CoA hydrolases for use in the methods of the invention have at least 63% identity to this malonyl-CoA hydrolase consensus sequence. In various embodiments, enzymes suitable for mutation of the key glutamic acid residue to X₁ in accordance with the methods of the invention to confer malonyl-CoA hydrolase activity have 65%, 70%, 80%, 90%, or 95% or more identity to SEQ ID NO:7. Proteins having significant homology to this consensus sequence include UniProt ID: C5DE94 (63% identity), UniProt ID: Q6CJH2 (64% identity), UniProt ID: G2WAE2 (66% identity), UniProt ID: J8Q6P9 (66% identity), UniProt ID: G8COHO (68% identity), UniProt ID: C5DX08 (68% identity), UniProt ID: P28817 (69% identity), UniProt ID: A7TTD5 (69% identity), UniProt ID: J7S9J9 (70% identity), UniProt ID: Q6FM09 (71% identity), UniProt ID: I2H4L2 (71% identity), UniProt ID: H2AME2 (73% identity), UniProt ID: G8ZTJ4 (77% identity), UniProt ID: GOW4I8 (77% identity), UniProt ID: G0V818 (78% identity), and UniProt ID: J5S5X3 (79% identity). In some embodiments, a malonyl-CoA hydrolase with equal to or greater than 63% identity to the consensus sequence SEQ ID NO:7 is expressed in a recombinant host cell and used to produce malonate in accordance with the invention.

In mutant and wild-type enzymes homologous to this consensus sequence (SEQ ID NO: 7), amino acids that are highly conserved are V101, R110, L114, R116, K119, L120, N121, A122, L123, L135, E137, Y138, K140, S141, S151, R156, C159, G161, G162, D163, V164, A168, F185, E188, Y189, S190, N192, A196, T197, K200, M206, G208, I209, T210, M211, G212, G213, G214, V215, G216, H220, P222, F223, R224, T227, E228, T230, M234, P235, E236, D238, 1239, G240, F242, P243, D244, V245, F249, P252, Q263, Y267, L268, T271, G272, G277, G284, S287, H288, Y289, L298, R301, L302, E304, E333, F334, L352, V354, I355, F359, L374, F391, L399, K402, S403, S406, N417, D429, L430, T432, A433, E449, F450, K457, L458, K461, W468, L494, T502, Y506, P507, L514, P515, and K561. In various embodiments, malonyl-CoA hydrolase enzymes homologous to this consensus sequence (SEQ ID NO:7) contain at least 25% of these conserved amino acids, often a majority (greater than 50%) of these conserved amino acids, and sometimes all of these conserved amino acids.

Some amino acids in this consensus sequence (SEQ ID NO:7) are essential for activity and conserved across all members of the class. Malonyl-CoA hydrolase enzymes encompassed by the EHD3 EC 3.1.2.4 based consensus sequence contain six active site residues important for hydrolase activity: (i) three active site amino acid residues (G161, G162, G213) in the consensus sequence believed to be necessary (although the invention is not to be limited by any theory of mechanism of action) for formation of an oxyanion hole responsible for stabilizing the enolate anion intermediate derived from the malonyl-CoA substrate; (ii) two amino acid residues (E236, D244) of the consensus sequence necessary for acyl-CoA hydrolysis; and (iii) an amino acid residue at position 188 (of SEQ ID NO:7) believed to be necessary for malonyl-CoA substrate binding. Of these six residues, then, five are present in the consensus sequence (SEQ ID NO:7) and in all malonyl-CoA hydrolases encompassed by that sequence, and the sixth, at position 188 (amino acid X₁ in the consensus) is selected from the group consisting of polar or positively charged amino acids (R, H, K, S, T, N, Q, Y), as well as A and D, to provide a malonyl-CoA hydrolase of the invention capable of producing malonate in a recombinant host cell. The six essential residues from the consensus sequence (G161, G162, G213, E236, D244, X₁188) correspond to G99, G100, G149, E172, D180, and E124 (typically mutated to X₁), respectively, in S. cerevisiae EHD3 used to illustrate the invention in example 31.

### Malonyl-CoA Hydrolase Consensus Sequence Based on Bacillus EC 3.1.2.4 Enzymes

The invention provides a malonyl-CoA hydrolase consensus sequence (SEQ ID NO:8) based on Bacillus EC 3.1.2.4 enzymes, and in various embodiments, suitable malonyl-CoA hydrolases for use in the methods of the invention have at least 86% identity to this malonyl-CoA hydrolase consensus sequence. In various embodiments, enzymes suitable for mutation of the key glutamic acid residue to X₁ in accordance with the methods of the invention to confer malonyl-CoA hydrolase activity have 90%, or 95% or more identity to SEQ ID NO:8. Proteins having significant homology to this consensus sequence include UniProt ID: C2TX63 (92% identity), UniProt ID: C2UV40 (91% identity), UniProt ID: C2QBT2 (93% identity), UniProt ID: C2XTU0 (93% identity), UniProt ID: C2PVQ0 (93% identity), UniProt ID: C3A5N3 (93% identity), UniProt ID: C2SJV4 (93% identity), UniProt ID: C2Z7U1 (92% identity), UniProt ID: C2VTI4 (97% identity), UniProt ID: B3Z9Y3 (97% identity), UniProt ID: B7JNH7 (97% identity), UniProt ID: Q63BK8 (97% identity), UniProt ID: B0Q3Q4 (97% identity), UniProt ID: B0AQX0 (97% identity), UniProt ID: B3YSW2 (97% identity), UniProt ID: C2NHG5 (97% identity), UniProt ID: B3ZIZ8 (97% identity), UniProt ID: C2QSV2 (97% identity), UniProt ID: C3C255 (97% identity), UniProt ID: B5UZZ1 (96% identity), UniProt ID: C2MKL7 (95% identity), UniProt ID: B9IZZ9 (95% identity), UniProt ID: F0PNG8 (95% identity), UniProt ID: Q738L0 (97% identity), UniProt ID: C2PEV7 (95% identity), UniProt ID: C2YRH7 (96% identity), UniProt ID: Q4MU30 (95% identity), UniProt ID: Q81DR3 (96% identity), UniProt ID: C2W7W8 (89% identity), and UniProt ID: A7GPH6 (86% identity). In various embodiments, a malonyl-CoA hydrolase with equal to or greater than 86% identity to the consensus sequence SEQ ID NO:8 is expressed in a recombinant host cell and used to produce malonate in accordance with the invention. Sequences for B9IZZ9 (SEQ ID NO:46), C3ALI3 (SEQ ID NO:47), F0PNG8 (SEQ ID NO:49), Q63BK8 (SEQ ID NO:51), and Q81DR3 (SEQ ID NO:52) containing X₁ at the position of the key glutamic acid residue that is mutated in accordance with the invention to confer malonyl-CoA hydrolase activity are provided in the sequence listing.

In mutant and wildtype enzymes homologous to this consensus sequence (SEQ ID NO:8) amino acids that are highly conserved are M1, T2, E3, V5, L6, F7, S8, G13, V14, A15, I17, T18, L19, N20, R21, P22, K23, A24, L25, N26, S27, L28, S29, Y30, M32, L33, I36, G37, K39, L40, K41, E42, W43, E44, I49, I52, V53, L54, K55, G56, A57, G58, K60, G61, F62, C63, A64, G65, G66, D67, I68, K69, T70, L71, Y72, E73, A74, R75, S76, N77, E78, A80, L81, Q82, A84, E85, F87, F88, E90, E91, Y92, I94, D95, T96, Y99, Y101, K103, P104, I105, I106, A107, C108, L109, D10, G111, I112, V113, M114, G115, G116, G117, V118, G119, L120, T121, N122, G123, A124, R127, I128, V129, T130, T133, K134, W135, A136, M137, P138, E139, M140, N141, I142, G143, F144, F145, P146, D147, V148, G149, A150, A151, Y152, F153, L154, N155, A157, P158, G159, G162, V165, A166, L167, A169, L172, K173, A174, D176, V177, L178, I180, A182, A183, D184, L192, F195, L196, W204, V210, L214, K215, L231, E236, H241, F242, E248, I250, I251, S253, L254, E255, F261, L269, L270, S271, K272, S273, P274, S276, L277, K278, V279, T280, L281, K282, Q283, G287, K290, S291, E293, C295, F296, A297, T298, D299, L300, L302, A303, K304, N305, F306, M307, R308, H309, D311, F312, F313, E314, G315, V316, R317, S318, V320, D322, K323, D324, Q325, N326, P327, Y329, K330, Y331, D336, V337, V342, N343, F345, F346, L348, and L349. In various embodiments, malonyl-CoA hydrolase enzymes homologous to this consensus sequence (SEQ ID NO:8) contain at least 25% of these conserved amino acids, often a majority (greater than 50%) of these conserved amino acids, and sometimes all of these conserved amino acids.

Some amino acids in this consensus sequence (SEQ ID NO:8) are essential for activity and conserved across all members of the class. Malonyl-CoA hydrolase enzymes encompassed by the Bacillus EC 3.1.2.4 based consensus sequence contain six active site residues important for hydrolase activity: (i) three active site amino acid residues (G65, G66, G116) of the consensus sequence believed to be necessary (although the invention is not to be limited by any theory of mechanism of action) for formation of an oxyanion hole responsible for stabilizing the enolate anion intermediate derived from the malonyl-CoA substrate; (ii) two amino acid residues (E139, D147) of the consensus sequence necessary for acyl-CoA hydrolysis; and (iii) a mutated amino acid (X₁91) (of SEQ ID NO:8) believed to be necessary for malonyl-CoA substrate binding. Of these six residues, then, five are present in the consensus sequence (SEQ ID NO:8) and in all malonyl-CoA hydrolases encompassed by that sequence, and the sixth, X₁91 is necessary to provide a malonyl-CoA hydrolase of the invention capable of producing malonate in a recombinant host cell. The six essential residues from the consensus sequence (G65, G66, G116, E139, D147, X₁91) correspond to G65, G66, G116, E139, D147, and E91 (typically mutated to X₁), respectively, in Bacillus thuringiensis subsp. finitimus (strain YBT-020) F0PNG8 used to illustrate the invention in example 31 (See SEQ ID NO: 49 containing mutation E91S).

Non-limiting examples of enzymes suitable for malonyl-CoA hydrolysis homologous to the consensus sequence (SEQ ID NO:8) and encoded by cloned or synthesized nucleic acids provided by the invention include mutant enzymes containing at least one mutation illustrated by the group of mutant enzymes consisting of Bacillus cereus (strain Q1) B9IZZ9 (E91S), B9IZZ9 (E91A), B9IZZ9 (E91H), B9IZZ9 (E91K), B9IZZ9 (E91R), B9IZZ9 (E91Q), B9IZZ9 (E91T), B9IZZ9 (E91N), B9IZZ9 (E91Y), B9IZZ9 (E91D); Bacillus thuringiensis subsp. finitimus (strain YBT-020) F0PNG8 (E91S), F0PNG8 (E91A), F0PNG8 (E91H), F0PNG8 (E91K), F0PNG8 (E91R), F0PNG8 (E91Q), F0PNG8 (E91T), F0PNG8 (E91N), F0PNG8 (E91Y), F0PNG8 (E91D); Bacillus cereus (strain ATCC 14579/DSM 31) Q81DR3, Q81DR3 (E91S), Q81DR3 (E91A), Q81DR3 (E91H), Q81DR3 (E91K), Q81DR3 (E91R), Q81DR3 (E91Q), Q81DR3 (E91T), Q81DR3 (E91N), Q81DR3 (E91Y), Q81DR3 (E91D); Bacillus cereus (strain ZK/E33L) Q63BK8, Q63BK8 (E91S), Q63BK8 (E91A), Q63BK8 (E91H), Q63BK8 (E91K), Q63BK8 (E91R), Q63BK8 (E91Q), Q63BK8 (E91T), Q63BK8 (E91N), Q63BK8 (E91Y), Q63BK8 (E91D).

### Malonyl-CoA Hydrolase Consensus Sequence Based on Pseudomonas EC 3.1.2.4 Enzymes

The invention provides a malonyl-CoA hydrolase consensus sequence based on Pseudomonas EC 3.1.2.4 enzymes (SEQ ID NO:9), and in various embodiments, suitable malonyl-CoA hydrolases for use in the methods of the invention have at least 75% identity to this malonyl-CoA hydrolase consensus sequence. In various embodiments, enzymes suitable for mutation of the key glutamic acid residue to X₁ in accordance with the invention to confer malonyl-CoA hydrolase activity have 80%, 90%, or 95% or more identity to SEQ ID NO:9. Proteins having significant homology to this consensus sequence include: UniProt ID: F5KBQ4 (80% identity), UniProt ID: A6VAN3 (81% identity), UniProt ID: A4XS22 (81% identity), UniProt ID: F6AA82 (75% identity), UniProt ID: E2XN63 (84% identity), UniProt ID: F2KE35 (85% identity), UniProt ID: C3KDS5 (83% identity), UniProt ID: F8G3B7 (86% identity), UniProt ID: G8PYD2 (85% identity), UniProt ID: Q4KGS1 (82% identity), UniProt ID: Q3KGL5 (85% identity), UniProt ID: B0KV51 (86% identity), UniProt ID: B1J4J2 (86% identity), UniProt ID: A5W8H3 (86% identity), UniProt ID: Q88N06 (86% identity), UniProt ID: Q1I5T5 (84% identity), UniProt ID: F8H1A4 (77% identity), UniProt ID: A4VIV7 (77% identity), and UniProt ID: Q9I5I5 (81% identity). In some embodiments, a malonyl-CoA hydrolase with equal to or greater than 75% identity to the consensus sequence SEQ ID NO:9 is expressed in a recombinant host cell and used to produce malonate in accordance with the invention. Sequences for A4XS22 (SEQ ID NO:45), F6AA82 (SEQ ID NO:50), and E2XN63 (SEQ ID NO:48), each containing X₁ at the position of the key glutamic acid residue that is mutated in accordance with the invention, are included in the sequence listing.

Highly conserved amino acids in this consensus sequence (SEQ ID NO:9) are M1, E6, G13, R15, I16, A19, L21, D22, A23, L27, N28, A29, L30, L32, P33, M34, I35, L38, W45, A46, C53, V54, L56, R57, G58, N59, G60, K62, A63, F64, C65, A66, G67, G68, V70, L73, C77, P81, G82, P85, L87, A88, F91, F92, Y96, R97, L98, H103, P106, K107, P108, C111, W112, H114, G115, V117, G119, G120, G121, M122, G123, L124, Q126, R131, I132, V133, T134, P135, R138, L139, M141, P142, E143, I146, G147, L148, D151, V152, G153, S155, F157, L158, R160, P162, G163, L165, G166, L167, F168, L171, N177, D180, A181, D183, L184, L186, A187, D188, R189, Q195, Q196, L199, L203, Q205, N207, W208, E210, Q215, L216, S218, L219, A222, P232, L237, R239, R240, D244, L247, D248, A258, D267, L269, G280, P282, V288, W289, Q291, R294, R296, L298, S299, L300, E307, Y308, S311, L312, N313, C314, R316, H317, P318, F320, E322, G323, V324, R325, A326, R327, L328, D330, D332, P335, W337, W339, P346, A352, H353, and F354. In various embodiments, malonyl-CoA hydrolase enzymes homologous to this consensus sequence (SEQ ID NO:9) contain at least 25% of these conserved amino acids, often a majority (greater than 50%) of these conserved amino acids, and sometimes all of these conserved amino acids.

Some amino acids in this consensus sequence (SEQ ID NO:9) are essential for activity and conserved across all members of the class. Malonyl-CoA hydrolase enzymes encompassed by the Pseudomonas EC 3.1.2.4 based consensus sequence contain six conserved active site residues necessary for hydrolase activity (i) three active site amino acid residues (G67, G68, G120) of the consensus sequence believed to be necessary (although the invention is not to be limited by any theory of mechanism of action) for formation of an oxyanion hole responsible for stabilizing the enolate anion intermediate derived from an acyl-CoA substrate; (ii) two amino acid residues (E143, D151) of the consensus sequence believed to be necessary for acyl-CoA hydrolysis; and (iii) amino acid X₁95 (of SEQ ID NO:9) is believed to be necessary for malonyl-CoA substrate binding. Of these six residues, then, five are present in the consensus sequence (SEQ ID NO:9) and in all malonyl-CoA hydrolases encompassed by that sequence, and the sixth, X₁95 is necessary to provide a malonyl-CoA hydrolase of the invention capable of producing malonate in a recombinant host cell. In various embodiments of the invention, the key wild-type glutamic acid residue (E95) is (has been) mutated to a polar or positively charged amino acid (i.e. R, H, K, S, T, N, Q, Y), or A or D, to produce X₁95 and provide a malonyl-CoA hydrolase of the invention capable of producing malonate in a recombinant host cell. In some embodiments of the invention, amino acid E95 is (has been mutated to) an amino acid selected from the group consisting of K, S, T, N, Y, A, and D. In some embodiments of the invention, amino acid E95 is S or N. The six essential residues from the consensus sequence (G67, G68, G120, E143, D151, X₁95) correspond to G67, G68, G120, E143, D151, and E95 (typically mutated to X₁), respectively, in Pseudomonas fulva (12-X) F6AA82-2 used to illustrate the invention in example 31 (see SEQ ID NO:50 containing mutations E95S/Q348A).

Non-limiting examples of enzymes suitable for malonyl-CoA hydrolysis homologous to the consensus sequence (SEQ ID NO:9) and encoded by cloned or synthesized nucleic acids provided by the invention include mutant enzymes containing at least one mutation illustrated by the group of mutant enzymes consisting of Pseudomonas fulva (strain 12-X) F6AA82 (E95S), F6AA82 (E95N), F6AA82 (E95A), F6AA82 (E95H), F6AA82 (E95K), F6AA82 (E95R), F6AA82 (E95Q), F6AA82 (E95D), F6AA82 (E95T), F6AA82 (E95Y) as demonstrated in example 43; Pseudomonas fluorescens WH6 E2XN63 (E95S), E2XN63 (E95N), E2XN63 (E95A), E2XN63 (E95H), E2XN63 (E95K), E2XN63 (E95R), E2XN63 (E95Q), E2XN63 (E95D), E2XN63 (E95T), E2XN63 (E95Y); Pseudomonas mendocina (strain ymp) A4XS22 (E95S), A4XS22 (E95N), A4XS22 (E95A), A4XS22 (E95H), A4XS22 (E95K), A4XS22 (E95R), A4XS22 (E95Q), A4XS22 (E95D), A4XS22 (E95T), E2XN63 (E95Y).

In various embodiments of the invention the malonyl-CoA hydrolase is F6AA82 (E95S) from Pseudomonas fulva (strain 12-X), E2XN63 (E95S) from Pseudomonas fluorescens WH6, A4XS22 (E95S) from Pseudomonas mendocina (strain ymp), as illustrated in Example 31.

As illustrated in Example 43, F6AA82 E95X₁ mutations resulted in malonyl-CoA hydrolase activity. F6AA82 proteins containing mutations E95S, E95Y, E95T, E95N, E96K, E95A, and E95D produced significantly (t-test, p<0.05) more malonate than the wild type F6AA82 protein. F6AA82 proteins containing these mutations are suitable for use as malonyl-CoA hydrolases and production of malonate. Of the F6AA82 enzymes containing an X₁ mutation conferring malonyl-CoA hydrolase activity, mutations E95S or E95N are preferred. Of the F6AA82 enzymes containing an X₁ mutation conferring malonyl-CoA hydrolase activity, mutations E95A, E95T, E95K, E95Y and E95D are suitable. In various embodiments of the invention the malonyl-CoA hydrolase is F6AA82 (E95S). In other embodiments of the invention the malonyl-CoA hydrolase is F6AA82 (E95N). F6AA82 proteins containing mutations E95H, E95Q, or E95R did not result in increased malonate production under test conditions employed as described in the examples.

Where an enzyme with substantial homology to a consensus sequence herein has suboptimal or no malonyl-CoA hydrolase activity, then, in accordance with the invention, it can be mutated to conform to a consensus sequence provided herein to provide a malonyl-CoA hydrolase of the invention. For example, protein A5W8H3 shows 86% identity to malonyl-CoA hydrolase consensus SEQ ID NO:9 but does not exhibit malonyl-CoA hydrolase activity (example 31). One or more amino acids that differ between A5W8H3 and consensus SEQ ID NO:9 may be mutated to introduce malonyl-CoA hydrolase activity. Specifically A5W8H3 mutations T2N, C5F, V7J, L8B, G10U, D12B, P24J, A26U, N31U, Q41B, A72B, A740, Q75J, S83J, S90B, A94J, F100B, A101B, N129U, A159U, F169O, P175B, G185J, G192B, A198J, A213J, N217B, Q224J, C228B, A229J, W236O, H241Q, E242B, Q245J, A252O, R261A, Q264J, D272B, G274A, Q275B, Y297B, Q302J, Q305B, M310O, N333B, A347J, A355J, A357O, and/or G368U may be used to impart malonyl-CoA hydrolase activity.

### Malonyl-CoA Hydrolase Consensus Sequence Based on Bacterial EC 3.1.2.4 Enzymes

Despite Bacillus and Pseudomonas being evolutionarily distant (i.e. Bacillus is Gram-positive and Pseudomonas is Gram-negative), there is significant sequence conservation between the Bacillus EC 3.1.2.4 and Pseudomonas EC 3.1.2.4 enzymes. The present invention provides a malonyl-CoA hydrolase consensus sequence based on these bacterial EC 3.1.2.4 acyl-CoA hydrolases (SEQ ID NO:10). Malonyl-CoA hydrolase enzymes encompassed by this consensus sequence typically possess at least 25% (or a majority or all) of the highly conserved amino acids from this sequence, which conserved amino acids are selected from the group consisting of L53, L59, N60, L62, M66, L88, F97, C98, A99, G100, G101, F124, F125, Y129, K140, P141, G148, G152, G153, G154, G156, L157, T167, M174, P175, E176, 1179, G180, D184, V185, G186, L191, L210, D219, A226, P333, N364, F375, E377, D385, and P390. A suitable malonyl-CoA hydrolase of the invention homologous to this consensus sequence includes the active site amino acids necessary for malonyl-CoA hydrolysis (G100, G101, G153, E176, and D184) of the consensus sequence, as well as a X₁128, where the key wild-type glutamic acid residue (E128) is (has been) mutated to a polar or charged amino acid (i.e. R, H, K, S, T, N, Q, Y), or D or A, and is capable of producing malonate in a recombinant host cell.

### Malonyl-CoA Hydrolases Based on Malonyl-CoA:ACP Transacylases

In yet other embodiments of the invention, the malonyl-CoA hydrolase is a mutated malonyl-CoA:ACP transacylase (EC 2.3.1.39). The invention provides an E. coli FabD sequence (SEQ ID NO:53), and in various embodiments, suitable malonyl-CoA hydrolases for use in the methods of the invention have at least 50%, 60%, 70%, 80%, 90%, 95%, or more sequence identity when aligned relative to SEQ ID NO:53 and contain one or more of the following amino acid mutations at the aligned positions: S92C, H201N, R117D, R117E, R117N, R117Y, R117G, R117H, Q1ID, Q11E, Q11N, Q11Y, Q11G, Q11H, L93A, L93V, L93I, L93F, L93S, L93G.

In some embodiments of this invention the malonyl-CoA hydrolase is a mutated E. coli FabD malonyl-CoA:ACP transacylase (see SEQ ID NO:53 for wild type sequence) with one or more mutation selected from the group consisting of S92C, H201N, R117D, R117E, R117N, R117Y, R117G, R117H, Q1ID, Q11E, Q11N, Q11Y, Q11G, Q11H, L93A, L93V, L93I, L93F, L93S, L93G. Example 35 illustrates recombinant yeast cells expressing a mutated E. coli FabD enzyme containing one of the following combinations of mutations S92C/L91V/R117H, L91I/R117Y/A246E, Q80L/L91S/R117G, and L91I/R117Y, and producing malonate at levels higher than wild type yeast without a mutated FabD enzyme.

### Section 3: Expression Vectors

In various aspects of the present invention, the recombinant host cell has been modified by “genetic engineering” to produce a recombinant malonyl-CoA hydrolase enzyme and malonate. The host cell is typically engineered via recombinant DNA technology to express heterologous nucleic acids that encode a malonyl-CoA hydrolase, which is either a mutated version of a naturally occurring acyl-CoA hydrolase or transacylase or a non-naturally occurring malonyl-CoA hydrolase prepared in accordance with one of the consensus sequences provided herein or is a naturally occurring acyl-CoA hydrolase with malonyl-CoA hydrolase activity that is either overexpressed in the cell in which it naturally occurs or is heterologously expressed in a cell in which it does not naturally occur.

Nucleic acid constructs of the present invention include expression vectors that comprise nucleic acids encoding one or more malonyl-CoA hydrolase enzymes. The nucleic acids encoding the enzymes are operably linked to promoters and optionally other control sequences such that the subject enzymes are expressed in a host cell containing the expression vector when cultured under suitable conditions. The promoters and control sequences employed depend on the host cell selected for the production of malonate. Thus, the invention provides not only expression vectors but also nucleic acid constructs useful in the construction of expression vectors. Methods for designing and making nucleic acid constructs and expression vectors generally are well known to those skilled in the art and so are only briefly reviewed herein.

Nucleic acids encoding the malonyl-CoA hydrolase enzymes can be prepared by any suitable method known to those of ordinary skill in the art, including, for example, direct chemical synthesis and cloning. Further, nucleic acid sequences for use in the invention can be obtained from commercial vendors that provide de novo synthesis of the nucleic acids.

A nucleic acid encoding the desired enzyme can be incorporated into an expression vector by known methods that include, for example, the use of restriction enzymes to cleave specific sites in an expression vector, e.g., plasmid, thereby producing an expression vector of the invention. Some restriction enzymes produce single stranded ends that may be annealed to a nucleic acid sequence having, or synthesized to have, a terminus with a sequence complementary to the ends of the cleaved expression vector. The ends are then covalently linked using an appropriate enzyme, e.g., DNA ligase. DNA linkers may be used to facilitate linking of nucleic acids sequences into an expression vector.

A set of individual nucleic acid sequences can also be combined by utilizing polymerase chain reaction (PCR)-based methods known to those of skill in the art. For example, each of the desired nucleic acid sequences can be initially generated in a separate PCR Thereafter, specific primers are designed such that the ends of the PCR products contain complementary sequences. When the PCR products are mixed, denatured, and reannealed, the strands having the matching sequences at their 3′ ends overlap and can act as primers for each other. Extension of this overlap by DNA polymerase produces a molecule in which the original sequences are “spliced” together. In this way, a series of individual nucleic acid sequences may be joined and subsequently transduced into a host cell simultaneously. Thus, expression of each of the plurality of nucleic acid sequences is effected.

A typical expression vector contains the desired nucleic acid sequence preceded and optionally followed by one or more control sequences or regulatory regions, including a promoter and, when the gene product is a protein, ribosome binding site, e.g., a nucleotide sequence that is generally 3-9 nucleotides in length and generally located 3-11 nucleotides upstream of the initiation codon that precede the coding sequence, which is followed by a transcription terminator in the case of E. coli or other prokaryotic hosts. See Shine et al., Nature 254:34 (1975) and Steitz, in Biological Regulation and Development: Gene Expression (ed. R F. Goldberger), vol. 1, p. 349 (1979) Plenum Publishing, N.Y. In the case of eukaryotic hosts like yeast a typical expression vector contains the desired nucleic acid coding sequence preceded by one or more regulatory regions, along with a Kozak sequence to initiate translation and followed by a terminator. See Kozak, Nature 308:241-246 (1984).

Regulatory regions or control sequences include, for example, those regions that contain a promoter and an operator. A promoter is operably linked to the desired nucleic acid coding sequence, thereby initiating transcription of the nucleic acid sequence via an RNA polymerase. An operator is a sequence of nucleic acids adjacent to the promoter, which contains a protein-binding domain where a transcription factor can bind. Transcription factors activate or repress transcription initiation from a promoter. In this way, control of transcription is accomplished, based upon the particular regulatory regions used and the presence or absence of the corresponding transcription factor. Non-limiting examples for prokaryotic expression include lactose promoters (LacI repressor protein changes conformation when contacted with lactose, thereby preventing the LacI repressor protein from binding to the operator) and tryptophan promoters (when complexed with tryptophan, TrpR repressor protein has a conformation that binds the operator; in the absence of tryptophan, the TrpR repressor protein has a conformation that does not bind to the operator). Non-limiting examples of promoters to use for eukaryotic expression include pTDH3, pTEF1 (as illustrated in Example 31), pTEF2, pRNR2, pRPL18B, pREV1, pGAL1, pGAL10, pGAPDH, pCUP1, pMET3, pPGK1, pPYK1, pHXT7, pPDC1, pFBA1, pTDH2, pPGI1, pPDC1, pTPI1, pENO2, pADH1, and pADH2. As described in Example 44, the promoters for the genes HSP150, PGK1, PHO5, SCT1, PRB1, TPI1, ACH1, HXK2, ACO1, JEN1, MDH2, POX1, CIT1, ALD4, ADH1, TDH3, ADH2, and SDH1 from S. cerevisiae strain BY4741 were demonstrated to be useful for the production malonic acid in accordance with the invention. As will be appreciated by those of ordinary skill in the art, a variety of expression vectors and components thereof may be used in the present invention.

Although any suitable expression vector may be used to incorporate the desired sequences, readily available expression vectors include, without limitation: plasmids, such as pESC, pTEF, p414CYC1, p414GALS, pSC101, pBR322, pBBR1MCS-3, pUR, pEX, pMR100, pCR4, pBAD24, pUC19, pRS series; and bacteriophages, such as M13 phage and λ phage. Of course, such expression vectors may only be suitable for particular host cells or for expression of particular malonyl-CoA hydrolases. One of ordinary skill in the art, however, can readily determine through routine experimentation whether any particular expression vector is suited for any given host cell or protein. For example, the expression vector can be introduced into the host cell, which is then monitored for viability and expression of the sequences contained in the vector. In addition, reference may be made to the relevant texts and literature, which describe expression vectors and their suitability to any particular host cell. In addition to the use of expression vectors, strains are built where expression cassettes are directly integrated into the host genome.

The expression vectors are introduced or transferred, e.g. by transduction, transfection, or transformation, into the host cell. Such methods for introducing expression vectors into host cells are well known to those of ordinary skill in the art. For example, one method for transforming E. coli with an expression vector involves a calcium chloride treatment wherein the expression vector is introduced via a calcium precipitate.

For identifying whether a nucleic acid has been successfully introduced or into a host cell, a variety of methods are available. For example, a culture of potentially transformed host cells may be separated, using a suitable dilution, into individual cells and thereafter individually grown and tested for expression of a desired gene product of a gene contained in the introduced nucleic acid. For example, an often-used practice involves the selection of cells based upon antibiotic resistance that has been conferred by antibiotic resistance-conferring genes in the expression vector, such as the beta lactamase (amp), aminoglycoside phosphotransferase (neo), and hygromycin phosphotransferase (hyg, hph, hpt) genes.

Typically, a host cell of the invention will have been transformed with at least one expression vector. When only a single expression vector is used, the vector will typically contain a malonyl-CoA hydrolase gene. Once the host cell has been transformed with the expression vector, the host cell is cultured in a suitable medium containing a carbon source, such as a sugar (e.g., glucose). As the host cell is cultured, expression of the enzyme(s) for producing malonate occurs. Once expressed, the enzyme(s) catalyzes the hydrolysis of the thioester bond of malonyl-CoA, thus releasing malonate and CoA.

If a host cell of the invention is to include more than one heterologous gene, the multiple genes can be expressed from one or more vectors. For example, a single expression vector can comprise one, two, or more genes encoding one, two, or more malonyl-CoA hydrolase enzyme(s) and/or other proteins providing some useful function, e.g. improved malonate yield, titer, and/or productivity. The heterologous genes can be contained in a vector replicated episomally or in a vector integrated into the host cell genome, and where more than one vector is employed, then all vectors may replicate episomally (extrachromosomally), or all vectors may integrate, or some may integrate and some may replicate episomally. Chromosomal integration is typically used for cells that will undergo sustained propagation, e.g., cells used for production of malonate for industrial applications. Example 45 illustrates the benefits of genomic integration of a malonyl-CoA hydrolase gene provided by the invention; in that example, a single integrated copy of the gene is shown to result in higher malonic acid titers than achieved when the same gene was expressed from a plasmid. This example also serves to demonstrate modulation of malonic acid production as provided by the invention, as an increase in the number of copies of the malonyl-CoA hydrolase encoding gene per cell results in an increase of malonic acid in the fermentation media. While a “gene” is generally composed of a single promoter and a single coding sequence, in certain host cells, two or more coding sequences may be controlled by one promoter in an operon. In some embodiments, a two or three operon system is used.

In some embodiments, the coding sequences employed have been modified, relative to some reference sequence, to reflect the codon preference of a selected host cell. Codon usage tables for numerous organisms are readily available and can be used to guide sequence design. The use of prevalent codons of a given host organism generally improves translation of the target sequence in the host cell. As one non-limiting example, in some embodiments the subject nucleic acid sequences will be modified for yeast codon preference (see, for example, Bennetzen et al., J. Biol. Chem. 257: 3026-3031 (1982)). In some embodiments, the nucleotide sequences will be modified for E. coli codon preference (see, for example, Nakamura et al., Nucleic Acids Res. 28:292 (2000)). In other embodiments, the nucleotide sequences are modified to include codons optimized for S. cerevisiae codon preference (see Example 42).

Nucleic acids can be prepared by a variety of routine recombinant techniques. Briefly, the subject nucleic acids can be prepared from genomic DNA fragments, cDNAs, and RNAs, all of which can be extracted directly from a cell or recombinantly produced by various amplification processes including but not limited to PCR and rt-PCR. Subject nucleic acids can also be prepared by a direct chemical synthesis.

The nucleic acid transcription levels in a host microorganism can be increased (or decreased) using numerous techniques. For example, the copy number of the nucleic acid can be increased through use of higher copy number expression vectors comprising the nucleic acid sequence, or through integration of multiple copies of the desired nucleic acid into the host microorganism's genome, as demonstrated in Example 45. Non-limiting examples of integrating a desired nucleic acid sequence onto the host chromosome include recA-mediated recombination, lambda phage recombinase-mediated recombination and transposon insertion. Nucleic acid transcript levels can be increased by changing the order of the coding regions on a polycistronic mRNA or breaking up a polycistronic operon into multiple poly- or mono-cistronic operons each with its own promoter. RNA levels can be increased (or decreased) by increasing (or decreasing) the strength of the promoter to which the protein-coding region is operably linked. Illustrative techniques for plasmid design and assembly to afford malonate production are provided in Examples 1, 3, 31, and 35.

The translation level of a desired polypeptide sequence in a host microorganism can also be increased in a number of ways. Non-limiting examples include increasing the mRNA stability, modifying the ribosome binding site (or Kozak) sequence, modifying the distance or sequence between the ribosome binding site (or Kozak sequence) and the start codon of the nucleic acid sequence coding for the desired polypeptide, modifying the intercistronic region located 5′ to the start codon of the nucleic acid sequence coding for the desired polypeptide, stabilizing the 3′-end of the mRNA transcript, modifying the codon usage of the polypeptide, altering expression of low-use/rare codon tRNAs used in the biosynthesis of the polypeptide. Determination of preferred codons and low-use/rare codon tRNAs can be based on a sequence analysis of genes derived from the host microorganism.

The polypeptide half-life, or stability, can be increased through mutation of the nucleic acid sequence coding for the desired polypeptide, resulting in modification of the desired polypeptide sequence relative to the control polypeptide sequence. When the modified polypeptide is an enzyme, the activity of the enzyme in a host may be altered due to increased solubility in the host cell, improved function at the desired pH, removal of a domain inhibiting enzyme activity, improved kinetic parameters (lower Km or higher Kcat values) for the desired substrate, removal of allosteric regulation by an intracellular metabolite, and the like. Altered/modified enzymes can also be isolated through random mutagenesis of an enzyme, such that the altered/modified enzyme can be expressed from an episomal vector or from a recombinant gene integrated into the genome of a host microorganism.

### Section 4: Recombinant Host Cells

In one aspect, the invention provides recombinant host cells suitable for biological production of malonate. Any suitable host cell may be used in practice of the methods of the present invention. In some embodiments, the host cell is a recombinant host microorganism in which nucleic acid molecules have been inserted, deleted or modified (i.e., mutated; e.g., by insertion, deletion, substitution, and/or inversion of nucleotides), either to produce malonate, or to increase yield, titer, and/or productivity of malonate relative to a “control cell” or “reference cell”. A “control cell” can be used for comparative purposes, and is typically a wild-type or recombinant parental cell that does not contain one or more of the modification(s) made to the host cell of interest.

In an important embodiment, the present invention provides recombinant yeast cells suitable for the production of malonate at levels sufficient for subsequent purification and use as described herein. Yeast host cells are excellent host cells for construction of recombinant metabolic pathways comprising heterologous enzymes catalyzing production of small molecule products. There are established molecular biology techniques and nucleic acids encoding genetic elements necessary for construction of yeast expression vectors, including, but not limited to, promoters, origins of replication, antibiotic resistance markers, auxotrophic markers, terminators, and the like. Second, techniques for integration of nucleic acids into the yeast chromosome are well established. Yeast also offers a number of advantages as an industrial fermentation host. Yeast can tolerate high concentrations of organic acids and maintain cell viability at low pH and can grow under both aerobic and anaerobic culture conditions, and there are established fermentation broths and fermentation protocols. The ability of a strain to propagate and/or produce desired product under low pH provides a number of advantages with regard to the present invention. First, this characteristic provides tolerance to the environment created by the production of malonic acid. Second, from a process standpoint, the ability to maintain a low pH environment limits the number of organisms that are able to contaminate and spoil a batch. Third, this characteristic also eliminates or at least reduces the need to add additional acid to facilitate purification of malonic acid by some methods provided by the invention (see Example 37).

In some embodiments of the invention, the recombinant host cell comprising a heterologous nucleic acid encoding a malonyl-CoA hydrolase is a eukaryote. In various embodiments, the eukaryote is a yeast selected from the non-limiting list of genera; Candida, Cryptococcus, Hansenula, Issatchenki, Kluyveromyces, Komagataella, Lipomyces, Pichia, Rhodosporidium, Rhodotorula, Saccharomyces or Yarrowia species. In various embodiments, the yeast is of a species selected from the group consisting of Candida albicans, Candida ethanolica, Candida krusei, Candida methanosorbosa, Candida sonorensis, Candida tropicalis, Cryptococcus curvatus, Hansenula polymorpha, Issatchenkia orientalis, Kluyveromyces lactis, Kluyveromyces marxianus, Kluyveromyces thermotolerans, Komagataella pastoris, Lipomyces starkeyi, Pichia angusta, Pichia deserticola, Pichia galeiformis, Pichia kodamae, Pichia kudriavzevii, Pichia membranaefaciens, Pichia methanolica, Pichia pastoris, Pichia salictaria, Pichia stipitis, Pichia thermotolerans, Pichia trehalophila, Rhodosporidium toruloides, Rhodotorula glutinis, Rhodotorula graminis, Saccharomyces bayanus, Saccharomyces boulardi, Saccharomyces cerevisiae, Saccharomyces kluyveri, and Yarrowia lipolytica. One skilled in the art will recognize that this list encompasses yeast in the broadest sense, including both oleaginous and non-oleaginous strains. Examples 46 and 47 illustrate the use of Pichia kudriavzevii strain Y-134 in accordance with the invention.

Alternative recombinant host cells are provided by the invention for biological production of malonate. Illustrative examples include eukaryotic, prokaryotic, and archaea cells. Illustrative examples of eukaryotic cells include, but are not limited to: Aspergillus niger, Aspergillus oryzae, Crypthecodinium cohnii, Cunninghamella japonica, Entomophthora coronata, Mortierella alpina, Mucor circinelloides, Neurospora crassa, Pythium ultimum, Schizochytrium limacinum, Thraustochytrium aureum, Trichoderma reesei and Xanthophyllomyces dendrorhous. In general, if a eukaryotic cell is used, a non-pathogenic strain is employed. Illustrative examples of non-pathogenic strains include, but are not limited to: Pichia pastoris and Saccharomyces cerevisiae. In addition, certain strains, including Saccharomyces cerevisiae, have been designated by the Food and Drug Administration as Generally Regarded As Safe (or GRAS) and so can be conveniently employed in various embodiments of the methods of the invention.

Illustrative examples of recombinant prokaryotic host cells provided by the invention include, but are not limited to, Bacillus subtilis, Brevibacterium ammoniagenes, Clostridium beijerinckii, Enterobacter sakazakii, Lactobacillus acidophilus, Lactococcus lactis, Mesorhizobium loti, Pseudomonas aeruginosa, Pseudomonas putida, Rhodobacter capsulatus, Rhodobacter sphaeroides, Salmonella enterica, Salmonella typhi, Salmonella typhimurium, Shigella flexneri, Staphylococcus aureus, Streptomyces ambofaciens, Streptomyces aureofaciens, Streptomyces aureus, Streptomyces fungicidicus, Streptomyces griseochromogenes, Streptomyces griseus, Streptomyces lividans, Streptomyces olivogriseus, Streptomyces rameus, Streptomyces tanashiensis, and Streptomyces vinaceus. Certain of these cells, including Bacillus subtilis, Lactobacillus acidophilus, have been designated by the Food and Drug Administration as Generally Regarded As Safe (or GRAS) and so are employed in various embodiments of the methods of the invention. While desirable from public safety and regulatory standpoints, GRAS status does not impact the ability of a host strain to be used in the practice of this invention; hence, non-GRAS and even pathogenic organisms are included in the list of illustrative host strains suitable for use in the practice of this invention.

Escherichia coli is also an excellent prokaryotic host cell for metabolic pathway construction, and E. coli is also well utilized in industrial fermentation of small-molecule products. Unlike most wild type yeast strains, wild type E. coli can catabolize both pentose and hexose sugars as carbon sources. E. coli also has a shorter doubling time relative to yeast, enabling experiments to be conducted more rapidly. The present invention provides a wide variety of E. coli host cells suitable for the production of malonate as described herein. In various embodiments of the methods of the invention, the recombinant host cell comprising a heterologous nucleic acid encoding a malonyl-CoA hydrolase is an E. coli cell.

### Section 5: Additional Modifications and Fermentation Conditions for Improved Malonate Production

In other aspects of the invention, increased malonate yield, titer, and/or productivity is achieved by employing host cells provided by the invention that have been genetically modified in ways other than, or in addition to, introduction of a heterologous malonyl-CoA hydrolase and/or by employing fermentation conditions provided by certain methods of the invention. In brief, the recombinant host cell of the invention comprise genetic modifications that increase acetyl-CoA biosynthesis, increase malonyl-CoA biosynthesis, decrease malonate catabolism, increase secretion of malonate from the host cell, increase host cell tolerance to malonate, and increase catabolism of various carbon sources.

Genetic Modifications and Fermentation Conditions that Increase Acetyl-CoA Biosynthesis

In accordance with the invention, increased malonate titer, yield, and/or productivity can be achieved by genetic modifications that increase acetyl-CoA biosynthesis, and the invention provides enzymes that increase acetyl-CoA biosynthesis, vectors for expressing enzymes that increase acetyl-CoA biosynthesis, host cells expressing enzymes that increase acetyl-CoA biosynthesis and increase malonate titer, yield, and/or productivity, and methods relating thereto. As described above, malonate is produced by hydrolysis of malonyl-CoA, which, can be produced from acetyl-CoA; thus, increases in acetyl-CoA biosynthesis can improve malonate production.

One route by which acetyl-CoA is produced is by an acetyl-CoA synthetase (EC 6.2.1.1), which catalyzes the formation of acetyl-CoA from acetate and coenzyme A (CoA). The invention provides recombinant host cells suitable for producing malonate in accordance with the methods of the invention comprising one or more heterologous acetyl-CoA synthetase (ACS) enzymes that increase malonate titer, yield, and/or productivity relative to a host cell not comprising a heterologous acetyl-CoA synthetase. Non-limiting examples of suitable ACS enzymes are S. cerevisiae ACS1 (GenBank: AAC04979.1) and ACS2 (GenBank: CAA97725.1). In some embodiments, a recombinant host cell comprising S. cerevisiae acetyl-CoA synthetase ACS1 and/or ACS2 is used to increase malonate titer, yield, and/or productivity. In other embodiments, a recombinant host cell comprising an acetyl-CoA synthetase selected from the group consisting of Salmonella enterica Acs, Escherichia coli AcsA, and Bacillus subtilis AcsA is used to increase malonate yield, titer, and/or productivity. Those skilled in the art appreciate that other acetyl-CoA synthetases can be expressed in a recombinant host cell producing malonate in accordance with the invention to increase malonate yield, titer, and/or productivity. This modification is illustrated in Examples 9 and 46.

A second route through which acetyl-CoA is produced is by a pyruvate dehydrogenase complex, which catalyzes the formation of acetyl-CoA from pyruvate. The invention provides recombinant host cells suitable for producing malonate in accordance with the methods of the invention that comprise one or more heterologous pyruvate dehydrogenase complex enzymes that increase malonate titer, yield, and/or productivity relative to a host cell not comprising a heterologous pyruvate dehydrogenase complex enzyme. Non-limiting examples of suitable pyruvate dehydrogenase complex enzymes include S. cerevisiae PDA1, PDB1, LAT1, LPD1, and PDX1. In some embodiments of the invention, malonate yield, titer, and/or productivity are increased in a recombinant host cell used to produce malonate by expressing one or more pyruvate dehydrogenase enzymes selected from the group consisting of S. cerevisiae PDA1, PDB1, LAT1, LPD1, and PDX1. Those skilled in the art appreciate that other pyruvate dehydrogenase enzymes can be expressed in a recombinant host cell producing malonate in accordance with the invention to increase malonate yield, titer, and/or productivity. This modification is illustrated in Example 10.

A third route through which acetyl-CoA is produced is by a heterologous ethanol catabolic pathway comprising enzymes catalyzing the conversion of ethanol to acetyl-CoA. Compared to malonate, ethanol is a less expensive chemical, and host cells producing malonate and expressing an ethanol catabolic pathway can convert ethanol to malonate. An alcohol dehydrogenase (EC 1.1.1.1) catalyzes conversion of ethanol to acetaldehyde. Non-limiting examples of suitable alcohol dehydrogenase enzymes include those selected from the group consisting of S. cerevisiae ADH2, E. coli AdhP, H. sapiens ADH1A, H. sapiens ADH1B, and H. sapiens ADH1C. In addition to the alcohol dehydrogenase, an ethanol catabolic pathway also comprises either an acetaldehyde dehydrogenase (acylating; EC 1.2.1.10), or an aldehyde dehydrogenase (EC 1.2.1.3) and an acetyl-CoA synthetase (EC 6.2.1.1). An acetaldehyde dehydrogenase (acylating) catalyzes the conversion of acetaldehyde to acetyl-CoA, an aldehyde dehydrogenase catalyzes the conversion of acetaldehyde to acetate, and an acetyl-CoA synthase, as described above, catalyzes the formation of acetyl-CoA from acetate and CoA. Non-limiting examples of suitable acetaldehyde dehydrogenases (acylating) include those selected from the group consisting of E. coli MhpF, E. coli AdhE, Pseudomonas sp CF600 DmpF, and Pseudomonas putida TodL. Non-limiting examples of aldehyde dehydrogenases include S. cerevisiae ALD2, ALD3, ALD4, ALD5, and ALD6; and H. sapiens ALD1, ALD2, ALD4, and ALD10. Non-limiting examples of acetyl-CoA synthetase enzymes include S. cerevisiae ACS1, S. cerevisiae ACS2, and E. coli Acs.

The present invention provides recombinant host cells suitable for producing malonate in accordance with the methods of the invention comprising one or more heterologous ethanol catabolic pathway enzymes that increase malonate yield, titer, and/or productivity relative to host cells not comprising the heterologous ethanol catabolic pathway enzyme(s). In some embodiments, the heterologous ethanol catabolic pathway enzymes are an ethanol dehydrogenase and an acetaldehyde dehydrogenase (acylating). In some embodiments, the heterologous ethanol catabolic pathway enzymes are S. cerevisiae ADH2 ethanol dehydrogenase and E. coli MhpF acetaldehyde dehydrogenase (acylating). In some embodiments, a heterologous S. cerevisiae ADH2 and E. coli MhpF are expressed in recombinant E. coli expressing a heterologous S. cerevisiae EHD3 malonyl-CoA hydrolase. In other embodiments, a heterologous S. cerevisiae ADH2 and E. coli MhpF are expressed in recombinant S. cerevisiae expressing a heterologous S. cerevisiae EHD3 malonyl-CoA hydrolase. In other embodiments, a heterologous S. cerevisiae ADH2 and E. coli MhpF are expressed in a recombinant oleaginous yeast expressing a heterologous S. cerevisiae EHD3 malonyl-CoA hydrolase. In other embodiments, the heterologous ethanol catabolic pathway enzymes are S. cerevisiae ADH2 ethanol dehydrogenase and Pseudomonas sp. CF600 DmpF acetaldehyde dehydrogenase (acylating). In some embodiments, a heterologous S. cerevisiae ADH2 and Pseudomonas sp. CF600 DmpF are expressed in recombinant E. coli expressing a heterologous S. cerevisiae EHD3 malonyl-CoA hydrolase. In some embodiments, a heterologous S. cerevisiae ADH2 and Pseudomonas sp. CF600 DmpF are expressed in recombinant S. cerevisiae expressing a S. cerevisiae EHD3 malonyl-CoA hydrolase. In some embodiments, a heterologous S. cerevisiae ADH2 and Pseudomonas sp. CF600 DmpF are expressed in a recombinant oleaginous yeast expressing a heterologous S. cerevisiae EHD3 malonyl-CoA hydrolase. In other embodiments, the heterologous ethanol catabolic pathway enzymes are S. cerevisiae ADH2 ethanol dehydrogenase and Pseudomonas putida TodL acetaldehyde dehydrogenase (acylating). In some embodiments, a heterologous S. cerevisiae ADH2 and Pseudomonas putida TodL are expressed in recombinant E. coli expressing a heterologous S. cerevisiae EHD3 malonyl-CoA hydrolase. In some embodiments, a heterologous S. cerevisiae ADH2 and Pseudomonas putida TodL are expressed in recombinant S. cerevisiae expressing a heterologous S. cerevisiae EHD3 malonyl-CoA hydrolase. In some embodiments, a heterologous S. cerevisiae ADH2 and Pseudomonas putida TodL are expressed in a recombinant oleaginous yeast expressing a heterologous S. cerevisiae EHD3 malonyl-CoA hydrolase. In other embodiments, the heterologous ethanol catabolic pathway enzymes are one or more alcohol dehydrogenase selected from the group containing S. cerevisiae ADH2, E. coli AdhP, H. sapiens ADH1A, H. sapiens ADH1B, and/or H. sapiens ADH1C and one or more acetaldehyde dehydrogenase (acylating) selected from the group containing E. coli MhpF, E. coli AdhE, Pseudomonas sp CF600 DmpF, and Pseudomonas putida TodL. Those skilled in the art appreciate that other alcohol dehydrogenase enzymes and acetaldehyde dehydrogenase (acylating) enzymes can be expressed in a recombinant host cell suitable for producing malonate in accordance with the methods of the invention to increase malonate yield, titer, and/or productivity.

In other embodiments, the heterologous ethanol catabolic pathway enzymes are an ethanol dehydrogenase, an aldehyde dehydrogenase, and an acetyl-CoA synthetase. In some embodiments, the heterologous ethanol catabolic pathway enzymes are a S. cerevisiae ALD2 alcohol dehydrogenase, a S. cerevisiae ALD2 aldehyde dehydrogenase, and a S. cerevisiae ACS1 acetyl-CoA synthetase. In other embodiments, the heterologous ethanol catabolic pathway enzymes are a S. cerevisiae ALD2 alcohol dehydrogenase, a S. cerevisiae ALD2 aldehyde dehydrogenase, and a S. cerevisiae ACS2 acetyl-CoA synthetase. In other embodiments, the heterologous ethanol catabolic pathway enzymes are a S. cerevisiae ALD2 alcohol dehydrogenase, a S. cerevisiae ALD6 aldehyde dehydrogenase, and a S. cerevisiae ACS1 acetyl-CoA synthetase. In other embodiments, the heterologous ethanol catabolic pathway enzymes are a S. cerevisiae ALD2 alcohol dehydrogenase, a S. cerevisiae ALD6 aldehyde dehydrogenase, and a S. cerevisiae ACS2 acetyl-CoA synthetase. In other embodiments, the heterologous ethanol catabolic pathway enzymes are one or more alcohol dehydrogenases selected from the group containing S. cerevisiae ADH2, E. coli AdhP, H. sapiens ADH1A, H. sapiens ADH1B, and/or H. sapiens ADH1C, one or more aldehyde dehydrogenases selected from the group containing S. cerevisiae ALD2, S. cerevisiae ALD3, S. cerevisiae ALD4, S. cerevisiae ALD5, S. cerevisiae ALD6, H. sapiens H. sapiens ALD1, H. sapiens ALD2, H. sapiens ALD4, and/or H. sapiens ALD10, and one or more acetyl-CoA synthetases selected from the group containing S. cerevisiae ACS1, S. cerevisiae ACS2, and/or E. coli Acs.

In some embodiments, recombinant host cells suitable for producing malonate according to the methods of the invention comprise a heterologous ethanol catabolic pathway enzyme and convert endogenously produced ethanol into acetyl-CoA and increase malonate yield, titer, and/or productivity. In other embodiments, ethanol is exogenously added to the fermentation broth and recombinant host cells suitable for producing malonate according to the methods of the invention comprise a heterologous ethanol catabolic pathway enzyme and convert exogenously added ethanol into acetyl-CoA and increase malonate yield, titer, and/or productivity. When exogenously added to the fermentation broth, ethanol is added to obtain a minimal concentration of 1% ethanol volume/volume, and is typically added to the fermentation broth to obtain a concentration between 1-15% volume/volume. Ethanol catabolism for improving malonate production in accordance with the invention is illustrated in Example 11.

Increased cytosolic pools of acetyl-CoA is a fourth route to increase malonate biosynthesis; in numerous plant and animal cells, but not S. cerevisiae, ATP citrate lyase (EC 2.3.3.8) is the primary enzyme responsible for cytosolic acetyl-CoA biosynthesis. In more detail, acetyl-CoA in the mitochondrion is condensed with oxaloacetate to form citrate through the activity of citrate synthase. Subsequently, citrate is transported from the mitochondrion into the cytosol where ATP citrate lyase catalyzes the formation of acetyl-CoA, oxaloacetate, and ADP. While S. cerevisiae does not contain a native ATP citrate lyase, suitable heterologous ATP citrate lyase enzymes have been described in oleaginous yeast strains (see, for example, Boulton et al., J. Gen. Microbiol. 127:169-176 (1981)). The present invention provides recombinant host cells comprising one or more heterologous nucleic oleaginous yeast ATP citrate lyase enzymes. Non-limiting examples of oleaginous yeast ATP citrate lyase enzymes include those selected from the group of oleaginous yeasts consisting of Candida curvata, Cryptococcus albidus, Lipomyces lipofer, Rhodospiridium toruloides, Rhodotorula glutanis, Trichosporon cutaneum, Yarrowia lipolytica, and the like. In various embodiments, the recombinant host cell comprises a heterologous nucleic acid encoding an ATP citrate lyase. In various embodiments, the ATP citrate lyase is from an organism selected from the group consisting of Candida curvata, Cryptococcus albidus, Lipomyces lipofer, Rhodospiridium toruloides, Rhodotorula glutanis, Trichosporon cutaneum, Yarrowia lipolytica. This modification is illustrated in Example 12.

Acetyl-CoA biosynthesis can also be increased in accordance with the invention by altering expression of one or more nucleic acids encoding proteins affecting fatty acid storage or catabolism. The present invention provides host cells comprising genetic modifications of one or more nucleic acids encoding proteins affecting fatty acid storage and catabolism. In Saccharomyces cerevisiae, these proteins include SNF2, IRA2, PRE9, PHO90, SPT21, POX1, ANT1, FOX3, PAS1, PAS3, ARE1, ARE2, DGA1, LRO1, ACL1, MAE1, GLC3, GLG1, GLG2, PAT1, and PEX11. This modification is illustrated in Example 13.

In some embodiments of the invention, the host cell comprises genetic modifications affecting expression and/or activity of proteins involved in fatty acid catabolism. For example, most host cells will naturally degrade fatty acids, hydroxy fatty acids and many diacids through beta-oxidation pathways. Beta-oxidation occurs, in most cases, by activating free fatty acid groups to CoA thioesters with acyl-CoA ligases. The acyl-CoA intermediate is further oxidized and degraded—proceeding through a 2,3 enoyl-CoA, 3-hydroxyacyl-CoA, and 3-ketoacyl-CoA—and subsequent cleavage results in production of acetyl-CoA and an acyl-CoA shortened by two carbons relative to the initial substrate. The enzymatic activities required for beta-oxidation are known. The present invention provides host cells that possess increased catabolic pathway activity for medium (C4-C8)- and long (>C8)-chain fatty acids, hydroxyl fatty acids, and diacids compared to control host cells. For example, in yeast (e.g., Saccharomyces cerevisiae), beta-oxidation occurs in the peroxisome; exemplary, non-limiting, nucleic acid products affecting peroxisomal beta-oxidation are Saccharomyces cerevisiae PAT1 and PEX11. In some embodiments of the invention, a host cell modified for increased expression of PAT1 and/or PEX11 is provided for use in the methods herein for the production of malonate. This modification is illustrated in Example 14.

Geneic Modifications and Fermentation Conditions that Increase Malonyl-CoA Biosynthesis

In accordance with the invention, increased malonate titer, yield, and/or productivity can be achieved through increased malonyl-CoA biosynthesis, and the invention provides host cells, vectors, enzymes, and methods relating thereto. Malonyl-CoA is produced in host cells through the activity of an acetyl-CoA carboxylase (EC 6.4.1.2) catalyzing the formation of malonyl-CoA from acetyl-CoA and carbon dioxide. The invention provides recombinant host cells for producing malonate that express a heterologous acetyl-CoA carboxylase (ACC). In some embodiments, the host cell is a S. cerevisiae cell comprising a heterologous S. cerevisiae acetyl-CoA carboxylase ACC1 or an enzyme homologous thereto. In some embodiments, the host cell modified for heterologous expression of an ACC such as S. cerevisiae ACC1 is further modified to eliminate ACC1 post-translational regulation by genetic modification of S. cerevisiae SNF1 protein kinase or an enzyme homologous thereto. The invention also provides a recombinant host cell suitable for producing malonate in accordance with the invention that is an E. coli cell that comprises a heterologous nucleic acid coding for expression of E. coli acetyl-CoA carboxylase complex proteins AccA, AccB, AccC and AccD or one or more enzymes homologous thereto. In accordance with the invention, additional acetyl-CoA carboxylases can be heterologously expressed to increase malonyl-CoA and malonate biosynthesis. This modification is illustrated in Example 15.

In various embodiments of the invention, expression of BirA, biotin-[acetylCoA carboxylase] holoenzyme synthetase, is coexpressed with E. coli acetyl-CoA carboxylase complex proteins AccA, AccB, AccC and AccD to enhance the activity of the ACC complex and result in an increase in malonyl-CoA and malonate production. In various embodiments of the invention, S. cerevisiae ACC1 is further modified to eliminate ACC1 post-translational regulation by introducing serine to alanine mutations at any, all, or any combination of the following residues; S10, S233, S430, S1114, S1145, S1148, S1157, S1159, S1162, S1163, S1169. In some embodiments of the invention, the acetyl-CoA carboxylase used is from Yarrowia lipolytica CLIB122, herein referred to as Y1ACC. Example 42 illustrates how the inclusion of this enzyme in a malonate-producing host strain provided by the invention results in a doubling of the titer of malonate from that host after 120 hours of fermentation, relative to the same host fermented without this enzyme. In additional embodiments of the invention, this enzyme is coexpressed with a biotin-[acetyl-CoA carboxylase] holoenzyme synthetase, also derived from this organism (BPL1). In additional embodiments of the invention, the acetyl-CoA carboxylases and biotin-[acetylCoA carboxylase] holoenzyme synthetase encoding genes are dtsR1 accBC and derived from Corynebacterium glutamicum. In additional embodiments of the invention, these genes are derived from a yeast strain including, but not limited to those of the genera, Candida, Pichia, or any of the other yeast herein. In various embodiments of the invention, the host cell producing malonate expresses any combination of these acetyl-CoA carboxylases and biotin-[acetyl-CoA carboxylase] holoenzyme synthetase enzymes.

In some embodiments of the invention, a host cell suitable for producing malonate according to the methods of the invention comprises genetic modifications affecting expression and/or activity of proteins involved in fatty acid biosynthesis. Malonyl-CoA is naturally a substrate in the biosynthesis of fatty acids, and diversion of malonyl-CoA to fatty acid production decreases the ability for the host cell to produce malonate. The invention provides recombinant host cells for producing malonate that express a heterologous fatty acid synthase (FAS) multienzyme complex. Temperature sensitive mutations of S. cerevisiae fatty acid synthase complex are known (see, Knobling et al., Eur. J. Biochem., 59:415-421 (1975)). Expression of a heterologous, temperature sensitive fatty acid synthase complex allows diversion of malonyl-CoA to fatty acid biosynthesis to be controlled by the temperature at which the host cell is cultured. In some embodiments, the host cell is a S. cerevisiae cell comprising S. cerevisiae fatty acid synthases FAS1 and FAS2 or enzymes homologous thereto. In some embodiments of the invention, FAS 1 and FAS2 enzymes are temperature-sensitive FAS1 or FAS2 enzymes.

In addition to genetic modification of the host cell, fatty acid biosynthesis can be decreased through addition of a FAS inhibitor to the cell culture media. For example, the FAS inhibitor cerulenin forms a covalent bond with the active site cysteine C1305 in the S. cerevisiae ketoacyl synthase domain of the FAS complex, inhibiting enzyme activity (Johansson et al., PNAS, 105:12803-12808 (2008)). Cerulenin is not only effective in inhibiting S. cerevisiae FAS activity, but is generally an inhibitor of FAS complexes containing a Cys-His-His or Cys-His-Asn catalytic triad in the ketoacyl synthase domain. In some embodiments, cerulenin is added to the fermentation broth to a final concentration between 5 mg/l and 100 mg/l to inhibit fatty acid biosynthesis and increase malonate production in recombinant host cells producing malonate in accordance with the methods of the invention. In various embodiments of a method of the invention, a FAS inhibitor is added to fermentation broth containing recombinant host cells producing malonate. In some embodiments of a method of the invention, the FAS inhibitor is cerulenin, as illustrated in Example 16. In some embodiments of the method of the invention, cerulenin is supplemented in the fermentation broth at a concentration between 5 mg/l and 100 mg/l. In other embodiments of a method of the invention, the fatty acid synthase complex inhibitor is selected from a group consisting of platensimycin, thiolactomycin, and triclosan.

One of the substrates for acetyl-CoA carboxylase is carbon dioxide, and increasing the carbon dioxide partial pressure in the fermentation broth promotes formation of malonyl-CoA. The fermentation broth should contain a minimum dissolved carbon dioxide pressure of 0.01 atmospheres, and an increase in dissolved carbon dioxide partial pressure above this threshold is desirable. The fermentation broth should typically contain between 0.1 and 1 atmospheres dissolved carbon dioxide partial pressure. The dissolved carbon dioxide partial pressure in the fermentation broth may be increased to above saturating conditions, or above 1 atmosphere dissolved carbon dioxide. In some embodiments of a method of the invention, the dissolved carbon dioxide partial pressure in the fermentation broth is increased to between 0.1 and 1 atmospheres. In some embodiments of the method of the invention, carbon dioxide partial pressure is increased through addition of carbonates or bicarbonates to fermentation broth. For example, and without limitation, calcium carbonate can be added to the fermentation broth to increase dissolved carbon dioxide partial pressure. In other embodiments of the method of the invention, the fermentation is run in a pressurized vessel that contains carbon dioxide at above atmospheric pressure. In other embodiments of the method of the invention, carbon dioxide gas is sparged into the fermentation broth. The gas mixture being sparged may contain other gases if the added components do not interfere with host cell growth or malonate production. It may be advantageous to co-localize the source of the carbon dioxide gas with the malonate fermentation. For example, and without limitation, gaseous carbon dioxide resulting from various fermentation processes (e.g., ethanol, isobutanol, 3-hydroxypropionate, etc.), chemical processes (e.g., downstream malonate synthetic chemistry), or energy generation (e.g., coal or natural gas powerplants) may be pumped into fermentation broth from malonate producing host cells to increase the carbon dioxide partial pressure, as illustrated in Example 17.

Genetic Modifications that Decrease Malonate Catabolism

In accordance with the invention, increased malonate titer, yield, and/or productivity can be achieved by decreasing malonate catabolism, and the invention provides host cells, vectors, enzymes, and methods relating thereto. One metabolic pathway by which malonate is catabolized in a host cell is through the activity of an acyl-CoA synthetase catalyzing the conversion malonate and Coenzyme A to malonyl-CoA. In some embodiments of the invention, a recombinant host cell suitable for producing malonate in accordance with the methods of the invention comprises a genetic modification resulting in the deletion, attenuation, or modification of one or more nucleic acids encoding for an acyl-CoA synthetase. In some embodiments of the invention, the recombinant host cell is yeast and the one or more acyl-CoA synthetases are selected from the group consisting of FAA1, FAA2, FAA3, FAA4, LSC1, and LSC2. In other embodiments of the invention, the recombinant host cell is E. coli and the one or more acyl-CoA synthetases are selected from the group consisting of FadD, FadK, FadI, SucC, SucD, and YahF. This aspect of the invention is illustrated in Example 18.

Genetic Modifications that Increase Malonate Secretion from the Host Cell

In accordance with the invention, increased malonate titer, yield, and/or productivity can be achieved by increasing malonate transport into the fermentation broth, and the invention provides host cells, materials, and methods relating thereto. In some embodiments of the invention, the recombinant host cell suitable for use in the methods of the invention is a S. cerevisiae cell that comprises a heterologous nucleic acid coding for expression of an S. cerevisiae transport protein selected from the group consisting of PDR5, PDR10, PDR11, PDR12, PDR15 and PDR18. In some embodiments of the invention, the recombinant host cell suitable for producing malonate in accordance with the methods of the invention is an E. coli cell that comprises a heterologous nucleic acid coding for expression of E. coli DcuC. This aspect of the invention is illustrated in Example 19.

Genetic Modifications that Increase Host Cell Tolerance to Malonate

In accordance with the invention, increased malonate titer, yield, and/or productivity can be achieved by increasing host cell tolerance to malonate, and the invention provides host cells, materials, and methods relating thereto. High concentrations of malonate can competitively inhibit succinate dehydrogenase (EC 1.3.5.1) activity (see Slater, Methods Enzymol. 10:48-57 (1967)). The present invention is based, in part, on the discovery that mutant succinate dehydrogenase enzymes exhibit a lower competitive inhibition by malonate. For example S. cerevisiae succinate dehydrogenase SDH1 residues E300, R331, and R442 are important for substrate (e.g., succinate) recognition. Increasing the size of the SDH1 active site decreases competitive inhibition by malonate while still allowing the enzyme to maintain activity toward the native substrate, succinate. In specific, introduction of one or more mutations selected from the group consisting of E300D, R331K or R331H, and R442K and R442H decreases competitive inhibition of SDH1 by malonate. In some embodiments, a recombinant host cell expressing an SDH1 with point mutation R300D is used to produce malonate in accordance with the invention. In other embodiments, a recombinant host cell expressing an SDH1 with point mutation R331K or R331H is used to produce malonate in accordance with the invention. In other embodiments, a recombinant host cell expressing an SDH1 with point mutation R442K or R442H is used to produce malonate in accordance with the invention. This aspect of the invention is illustrated in Example 20.

Genetic Modifications that Increase Catabolism of Various Carbon Sources

In the methods of the invention, carbon feedstocks are utilized for production of malonate. Suitable carbon sources include, without limitation, those selected from the group consisting of purified sugars (e.g., dextrose, sucrose, xylose, arabinose, lactose, etc.); plant-derived, mixed sugars (e.g., sugarcane, sweet sorghum, molasses, cornstarch, potato starch, beet sugar, wheat, etc.), plant oils, fatty acids, glycerol, cellulosic biomass, alginate, ethanol, carbon dioxide, methanol, and synthetic gas (“syn gas”). A given host cell may catabolize a particular feedstock efficiently or inefficiently. If a host cell inefficiently catabolizes a feedstock, then one can modify the host cell to enhance or create a catabolic pathway for that feedstock. For example, Example 47 shows production of malonate, in accordance with the invention, from a number of different carbon sources using Pichia kudriavzevii as the host strain. While this organism does not catabolize sucrose under the conditions tested, the invention provides the introduction of a sucrose invertase (beta-fructofuranoside, EC 3.2.1.26) to facilitate the use of this carbon source. Example 30 also demonstrates the production of malonic acid in accordance with the methods of the invention using a variety of carbon sources. Additional embodiments of the invention include the use of methanol catabolizing host strains. In some embodiments, the host is a yeast strain. In some embodiments, the host is selected from the Komagataella pastoris, Pichia methanolica, or Pichia pastoris.

The invention provides host cells comprising genetic modifications that increase malonate titer, yield, and/or productivity through the increased ability to catabolize non-native carbon sources. Wild type S. cerevisiae cells are unable to catabolize pentose sugars, lignocellulosic biomass, or alginate feedstocks. In some embodiments, the invention provides a an S. cerevisiae cell comprising a heterologous nucleic acid encoding enzymes enabling catabolism of pentose sugars useful in production of malonate as described herein. In other embodiments, the heterologous nucleic acid encodes enzymes enabling catabolism of lignocellulosic feedstocks. In yet other embodiments of the invention, the heterologous nucleic acid encodes enzymes increasing catabolism of alginate feedstocks.

Those skilled in the art will recognize that the individual manipulations to increase malonate production from a given host cell can be used in virtually unlimited combination and often confer simulations benefits resulting in a much higher malonate output than a single manipulations and in cases, the sum of the individual manipulations. Thus, the invention encompasses not only the single manipulations described herein, it also is embodied by any combination of these perturbations resulting in the increase of malonic acid produced the host strain.

### Section 6: Detecting Malonate Producing Host Cells and Screening Host Cells for Improved Malonate Production

The present invention also provides a transcription factor biosensor system that can be used for the accurate sensing of malonate in liquid media. In many applications, this system is used to detect malonate produced within a host cell by sensing the presence of malonate in the fermentation media containing the malonate producing host cell. “Accurate sensing” refers to detecting the presence of and/or directly or indirectly determining the concentration of malonate; therefore, by accurately sensing malonate, this aspect of the invention has application in strain improvement, i.e., for increasing malonate production in host cells. In this system, malonate binds to a protein moiety present on a transcription factor. Binding of malonate to the malonate binding moiety results in either binding of the transcription factor to the promoter that is activated by the transcription factor, or in some embodiments, results in derepression of the promoter by the malonate-bound transcription factor.

Any number of transcription factors that bind to malonate, or are activated to bind to a promoter in response to a signal generated by binding of malonate to a binding moiety, are suitable for use in this system.

In some embodiments, the transcription factor can bind malonate, which results in binding of the transcription factor to a cognate promoter and activation of a gene that is operably linked to the promoter. Non-limiting examples of transcription factors that bind malonate include the transcription factors Acinetobacter calcoaceticus MdcY (SEQ ID NO:3), Rhizobium leguminosarum MatR (SEQ ID NO:4), Klebsiella pneumoniae MauR (SEQ ID NO:5), and homologs thereof.

In some embodiments, a transcription factor used in the invention is an MdcY transcription factor. An MdcY transcription factor can directly bind malonate and regulate transcription mediated by promoters such as P_{MdcL}. MdcY and MdcY-responsive promoters are known in the art (see, e.g., Koo et al., J Bacteriol. 182:6382-6390 (2000)).

An example of an MdcY polypeptide sequence from Acinetobacter calcoaceticus is provided in SEQ ID NO:3. MdcY polypeptides that can be employed in accordance with the invention include variants and homologs of the MdcY polypeptide sequence set forth in SEQ ID NO:3. Thus, a MdcY transcription factor polypeptide may have an amino acid sequence that has at least 60% identity, typically at least 75%, 90%, 95%, 99% or greater amino acid sequence identity, preferably over a region of at least 100 or more amino acids, or at least 200 or more amino acids, or over the length of the entire polypeptide, to an amino acid sequence of SEQ ID NO:3. One of skill in the art understands in view of this disclosure that variants can also be employed, e.g., using the known sequences as guidance for selecting amino acid substitutions that will not result in loss of function.

In some embodiments, the MdcY transcription factor for use in the invention is naturally present in a host cell. In other embodiments, a host cell is genetically modified to express a foreign transcription factor by introducing a heterologous nucleic acid encoding the malonate transcription factor into the host cell. In some embodiments, the genetically modified host cell comprising a heterologous nucleic acid encoding the malonate transcription factor is an E. coli host cell.

The MdcY transcription factor can bind to a number of promoters and activate expression of a gene operably linked to the promoter. An example of a MdcY-responsive promoter suitable for use in accordance with the invention is provided in SEQ ID NO:6. In some embodiments, an MdcY-responsive promoter for use in the invention, typically comprise an operator sequence ATTGTATACAAT. In some embodiments, the promoter is at least 75% identical to the promoter sequence shown in SEQ ID NO:6. In some embodiments, the promoter comprises a subsequence of SEQ ID NO:6 comprising 10, 20, 25, 30, 35, or more, contiguous nucleotides of SEQ ID NO:6.

Thus, in one aspect, the present invention provides a method for accurately sensing malonate. In some embodiments, the sensing step comprises detecting, e.g., measuring the amount of a gene product of a reporter or marker gene (e.g. a fluorescent reporter gene). In some embodiments, the gene product of the reporter gene influences the growth rate of a host cell comprising the components of a malonate transcription factor biosensor of the invention. In some embodiments, the gene product of the reporter gene causes the modified host cell to become resistant or sensitive to a compound. For example, in some embodiments, the reporter gene is an antibiotic resistance gene (e.g. a tetA gene) where the presence of malonate in the culture medium induces antibiotic resistance such that the host cell exhibits improved growth in the presence of malonate when the antibiotic is present, as illustrated in Example 21. In some embodiments, a host cell that comprises the components of a transcription factor biosensor of the invention is a host cell that is capable of producing malonate. Example 36 illustrates practice of this aspect of the invention using both antibiotic resistance (tetA) and a colorimetric signal (lacZ) as outputs that are correlated to malonic acid concentration, produced in accordance with other aspects of the invention, in fermentation media.

Generally, then, the present invention provides for a method for screening or selecting a host cell that produces malonate comprising: (a) providing a modified host cell of the present invention, (b) culturing the host cell, and (c) screening or selecting the host cell based on the expression of the reporter gene by the host cell.

In some embodiments of the present invention, the method for screening or selecting a host cell that produces malonate comprises: (a) providing a plurality of modified host cells of the present invention wherein the modified host cells of different modification are in separate cultures, (b) culturing each separate culture of host cell, (c) screening or selecting the host cell based the expression of the reporter gene by the host cell, and (d) comparing the expression of the reporter genes of the separate cultures. In some embodiments of the present invention, step (d) comprises identifying one or more cultures, and/or the corresponding host cell, that have an increased expression of the gene product of the reporter gene. It will be recognized by those skilled in the art that these cultures arise from a single transformant and represent a clonal population.

In some embodiments, the method of the invention is a method for selecting a host cell that produces malonate, wherein the selection is a positive selection. In a positive selection, the selecting step selects for host cells that have a higher expression of a reporter gene that increases the probability of remaining viable and doubling, and thus have a higher probability of remaining viable and doubling. For example, host cells producing malonate will remain viable and propagate at a rate faster than host cells not that do not produce malonate. Similarly, host cells with increased malonate production as compared to a control strain will propagate at a rate faster than the control strain.

In some embodiments of the present invention, the method for selecting an E. coli host cell that produces malonate comprises: (a) providing a plurality of modified E. coli host cells of the present invention wherein the modified host cells of different modification are in separate cultures, (b) culturing each separate culture of host cell, (c) positively selecting the host cell based the expression of the reporter gene by the host cell, and (d) comparing the expression of the reporter genes of the separate cultures.

In other embodiments of the present invention, the method for selecting an E. coli host cell that produces malonate comprises: (a) providing a plurality of modified E. coli host cells of the present invention wherein the modified host cells of different modification are in the same culture, (b) culturing the heterogenous mixture of modified host cells in growth medium containing a positive selecting agent such that the host cells exhibiting a higher production of malonate than the plurality of host cells will propagate at a rate faster than host cells exhibiting lower production of malonate and (d) isolating the host cells exhibiting the highest production of malonate.

### Section 7: Malonate Purification

In a sixth aspect, the invention provides methods for purifying malonate from fermentation broth, the methods comprising: (a) culturing a host cell under conditions suitable for production of malonate, (b) and recovering (i.e., purifying) the malonate from fermentation broth. The invention also provides purified malonate produced in accordance with the methods of the invention. Biosynthesized malonate can be produced intracellularly and/or secreted into the culture medium. Intracellularly produced malonate is typically secreted into the culture medium using a membrane transporter, as described above. Malonate not secreted can be removed from the host cell by chemical, enzymatic, or mechanical cell lysis. Malonate can be recovered from the cells, from the fermentation broth, or both. If the cell is engineered to secrete malonate, one can opt to recover the malonate only from the fermentation broth or one can opt to recover it both from the media and from the cell (i.e., by lysing the cell). If the cell is not engineered to secrete malonate one can lyse the host cell to isolate the malonate therein.

The present invention provides methods to isolate malonate produced biologically. As used herein, “isolate”, “purify”, and “recover” are used to refer to separation of the malonate from other substances present. “Isolation”, “purification”, or “recovery” as used in this context is intended to convey a preparation of malonate that is enriched in malonate relative to the cell or fermentation broth that produced it but that may or may not be substantially (i.e., more than 50%) pure on a weight/weight (w/w) basis. Isolating malonate in accordance with these methods involves separating the malonate produced from at least part or all of the fermentation medium, host cells, and parts thereof, from which malonate is produced. Malonate may be purified, i.e., to more than 50% purity on a w/w basis, in accordance with the invention from the fermentation broth and/or from the producing cell in which any naturally occurring or recombinant host cell (e.g., E. coli, S. cerevisiae, oleaginous yeast, and the like) producing malonate is grown, i.e., the host cell is not limited to a recombinant host cell of the invention. The isolated malonate may be free or essentially free of impurities from the host cells. The malonate is isolated or purified to a degree such that any impurities present do not interfere in the subsequent use of the malonate. For example, if the subsequent use is as an industrial chemical, such as a chemical to be used in a polymerization reaction, then the malonate is essentially free of impurities when any remaining impurities would not interfere with the use of the malonate in a polymerization reaction. Typically malonate used for polymerization reactions has a purity of at least 95% w/w or higher. If the malonate is to be used as a fuel, such as a fuel to be used in a combustion reaction, then the compound is essentially free of impurities when any impurities remaining would not interfere with the use of the malonate as a fuel. If the malonate is used as an animal feed, then the malonate is essentially free of impurities when any impurities remaining would not interfere with the use of the material as animal feed. When malonate is used as an animal feed, one may opt to recover the biomass containing malonate from the fermentation broth and use the biomass as animal feed.

In some embodiments of the purification methods of invention, the fermentation broth is concentrated to increase the working concentration of malonate and decrease the volume of liquid that requires processing. In various embodiments of the purification methods of the invention, this concentration is achieved by evaporation, including vacuum and heat, reverse osmosis, “high pass” membrane dewatering, and/or thin film evaporation.

In some embodiments, the purification methods of the invention comprise the step of recovering the malonate produced, wherein the recovering step is concurrent or subsequent to the culturing step. In some embodiments, the malonate is purified from the fermentation broth and the host cells. In other embodiments, the host cells are separated from the fermentation broth, lysed, and then malonate is recovered from the host cells. In other embodiments, the host cells are lysed in the fermentation broth and malonate is recovered from the lysed cells and fermentation broth. One method for recovering malonate from the fermentation broth provided by the invention is precipitation of malonate with a cation. In some embodiments this is a monovalent cation, in other embodiments it is a divalent cation. Typically the cation is added to the fermentation broth (or lysate) as a salt. For example, precipitation of calcium malonate from an aqueous solution, which may be fermentation broth or a cell lysate or a mixture of both, containing malonate in accordance with the invention is accomplished by the addition of a calcium salt (Weiner, Org. Synth. 18:50 (1938); Weiner, Org. Synth. Coll. 2:376 (1943)). Various calcium salts (e.g., calcium hydroxide, calcium carbonate, calcium chloride) can be used in accordance with the invention to precipitate malonate from fermentation broth.

A calcium malonate salt forms when the when two malonate carboxylic acids are unprotonated and calcium is present in the fermentation broth. Calcium malonate is insoluble in aqueous solutions, e.g., fermentation broth, will precipitate, and can then be recovered from the fermentation broth. The pKₐ values of the two malonate carboxylic acid moieties are 2.83 and 5.69; thus, when the fermentation broth pH is below 5.7, calcium malonate does not form and malonate remains dissolved in the fermentation broth. When the fermentation broth pH is above 5.7, calcium malonate can form and will precipitate from the fermentation broth. Carboxylic acids are weak acids, and the accumulation of malonate in the fermentation broth will decrease the pH, and if the pH falls below 5.7 the addition of calcium cations does not result in the formation of calcium malonate. One method of the invention to purify malonate from fermentation broth is to add a calcium salt where the calcium salt anion is a base (i.e., calcium carbonate and calcium hydroxide) and addition of the calcium salt raises and/or maintains the fermentation broth pH above 5.7. In some embodiments of a method of the invention, calcium hydroxide is added to the fermentation broth to reach/maintain a pH between 5.69 and 7.5 and precipitate calcium malonate. In a second embodiment of a method of the invention, calcium carbonate is added to the fermentation medium to reach/maintain a pH between 5.69 and 7.5 and precipitate calcium malonate. In a third embodiment of the invention, calcium chloride is added to the fermentation broth when the fermentation broth pH is between 5.69 and 7.5 and precipitate calcium malonate. In accordance with the methods of the invention, other calcium salts can be added to the fermentation broth to precipitate malonate. Addition of calcium carbonate has the additional advantage of increasing the carbon dioxide partial pressure in the fermentation broth, promoting formation of malonyl-CoA through the activity of acetyl-CoA carboxylase, described above. Example 23 illustrates this aspect of the invention by describing the purification of malonate using a calcium salt.

One may add the calcium salt at the beginning of the fermentation (i.e., before substantial malonate has accumulated), during the fermentation, or at the end of the fermentation. In some embodiments, calcium carbonate or calcium hydroxide is added to the fermentation broth before the host cells have begun producing malonate. In other embodiments, calcium carbonate or calcium hydroxide is added to the fermentation broth before malonate concentrations exceed 5, 10, 15, 20, 25, 30, 40, or 50 g/l. In other embodiments, calcium carbonate or calcium hydroxide is added the fermentation broth at the end of the fermentation. One may choose to recover the calcium malonate precipitate from the fermentation broth concurrent or subsequent to the fermentation.

Because calcium malonate monohydrate solubility decreases with increasing temperature, recovery of malonate from the fermentation broth may be increased by raising the temperature to between 50° C. and 100° C. during the recovery step. In some embodiments of the method of the invention, a calcium salt (i.e., calcium carbonate, calcium hydroxide, and/or calcium chloride) is added to fermentation broth, the temperature is increased to between 50° C. and 100° C., and malonate is recovered from the fermentation broth. In additional embodiments of the invention, the temperature is increased above 100° C. However, thermal decomposition of malonic acid can occur at temperatures of about 100° C., accordingly, increasing the temperature to or above 100° C. may negatively impact yield.

In some embodiments of the invention, malonate is purified from the production media by precipitation with a monovalent cation. Typically the monovalent cation is added to the fermentation broth (or lysate) as a salt. For example, precipitation of sodium malonate from an aqueous solution, which may be fermentation broth or a cell lysate or a mixture of both, containing malonate in accordance with the invention is accomplished by the addition of a sodium salt. Various sodium salts (e.g., sodium hydroxide, sodium carbonate, sodium chloride) can be used in accordance with the invention to precipitate malonate from fermentation broth. A sodium malonate salt forms when the when two malonate carboxylic acids are unprotonated and sodium is present in the fermentation broth. Sodium malonate is soluble in aqueous solutions, e.g., fermentation broth, but can be forced out of solution by increasing the pH of the media above the higher pKa of malonic acid (5.69); see Example 32. Additional precipitation of the sodium malonate is achieved by increasing the concentration of the ions by evaporation of the solvent (water). As with the aspects of the invention associated with calcium malonate precipitation, the addition of sodium salts containing a base (e.g. sodium hydroxide or sodium carbonate) can be used to maintain the pH of the fermentation broth above 5.7, thus facilitating precipitation of the sodium salt. Separation of the precipitate from the fermentation broth provides purified malonate in accordance with the invention.

Another method of the invention for isolating malonate from the fermentation broth is through reactive extraction with an aliphatic primary, secondary, or tertiary amine or a primary, secondary, or tertiary alcohol. This method arises in part from the discovery that both primary alcohols and tertiary amines are highly effective agents for the selective removal of malonate from fermentation broth as is illustrated in Examples 24-26, 36-41. In some embodiments of the invention, malonate is purified from the fermentation broth by reactive extraction with trioctylamine in organic solvent. In other embodiments of in the invention, malonate is purified from the fermentation broth using a tertiary amine selected from the group consisting of triethylamine, tripropylamine, tributylamine, tripentylamine, trihexylamine, triheptylamine, trinonylamine, and tridecylamine. In additional embodiments of the invention, the amine compound is ammonia. In yet other embodiments, it is a dialkylamine including, but not limited to diethylamine, dipropylamine, diisopropylamine, dibutylamine, dipentylamine, dihexylamine, diheptylamine, dioctylamine, dinonylamine, didecylamine. Example 33 demonstrates the isolation of malonate from fermentation broth using diethylamine. In various embodiments of the invention the amino compound is added in conjunction with an organic solvent or mixture of solvents including, but not limited to: pentanol, hexanol, heptanol, octanol, nonanol, decanol, hexane, decane, kerosene, ethers, ethylacetate or any combination thereof. In various embodiments, the solvent is a branched or unbranched, alkane, alcohol, diol, ester, ether, diether, or lactone containing four to twenty carbons In various embodiments, addition of solvents facilitates the transfer of amino compounds to the aqueous phase and the amine-coordinated malonates to the organic phase, thus improving purification.

A number of parameters can be adjusted in accordance with the invention to improve malonate extraction. Length of alkyl groups, ionic strength of the fermentation broth, pH, molar equivalents of trialkylamines, temperature, and cosolvent each have an impact on the extraction efficiency of trialkylamines. The impact of alkyl group length is illustrated in example 36. Tripropyl-, trihexyl-, and trioctyl-amine are each capable of isolating malonic acid from the aqueous phase and solubilizing it into the 1-octanol organic phase in accordance with the invention. The extraction efficiency of trioctylamine>trihexylamine>tripropylamine, indicating the longer of these chain lengths is the preferable embodiment (for high-yield) of the invention at low pH, with a 1-octanol cosolvent.

As with salt precipitation, pH has an impact on malonic acid extraction from an aqueous phase when using trialkylamines. Trialkylamine extraction has the highest efficiency when both acid moieties are carboxylated. Example 37 shows the extraction efficiencies of trioctylamine over a range of pH values. The highest level of extraction occurs at pH 1.5 and diminishes as pH is increased. Above the second pKa of malonic acid (5.69) there is virtually no extraction. While the invention provides malonate extraction from an aqueous phase using trialkylamines without limitation on pH, embodiments at pH of 2.0 or lower are preferred for maximum efficiency. In some embodiments of the invention, host cell growth and production of malonate at a low pH, as illustrate by Example 48, facilitates protonation of malonate to enhance extraction with trialkylamines. In other embodiments of the invention, exogenous acid is added to facilitate this protonation.

In various embodiments of the invention, different concentrations of trialkylamine are used. As reported in Example 38, linear relationship between extraction efficiency and trioctylamine:malonic acid mol fraction was observed; specifically the linear relationship existed between trioctylamine:malonic acid mol fractions of 0 to 1. Above 1 molar ratio, 100% of the malonic acid was extracted into the organic phase. Thus, to maximize extraction efficiency the amount of trialkylamine in the organic phase must be equimolar to the amount of malonic acid in the aqueous phase (e.g., fermentation broth). Ideally a greater than equimolar amount of trialkylamine will be added to the organic phase to compensate for decreased extraction efficiencies due to other organic acids and anions in the fermentation broth.

As illustrated in Example 39, trialkylamine extraction efficiency of malonic acid is negatively impacted by increasing the ionic strength (salt concentration) of the aqueous phase. Desalting of various ions can be achieved through a number of methods, including: addition of other agents to generate an insoluble salt, pH adjustment, and/or passage through various membranes.

In various embodiments of the invention, the reactive extraction is Fischer esterification of malonate at one or both carboxylic acids with an alcohol in the presence of an acid catalyst to result in the formation of monoalkyl and dialkyl malonate esters, respectively. For example, diethyl malonate can be produced by Fischer esterification of malonate using ethanol. The monoalkyl and dialkyl malonate esters have low solubility in aqueous solutions and low volatility, enabling the malonate esters to be separated from water and other volatile components in the fermentation broth by distillation. In various embodiments of the invention, separation of the esters is achieved by phase partitioning into an organic solvent. In general, a primary or secondary aliphatic alcohol is suitable for Fischer esterification with malonate; for example, and without limitation, the alcohol may be selected from the group consisting of methanol (CAS #67-56-1), ethanol (CAS #64-17-5), 1-propanol (CAS #71-23-8), 1-butanol (CAS #71-36-3), isopropanol (CAS #67-64-0) and isobutanol (CAS #78-83-1), among others. A number of acid catalysts are suitable for Fischer esterification of an alcohol with malonate; for example, and without limitation, the acid catalyst may be selected from the group consisting of sulfuric acid, tofic acid, scandium(III) triflate, N,N′-dicyclohexylcarbodiimide and tetrabutyl ammonium tribromide. In various embodiments of the invention, malonate is purified from fermentation broth by reactive distillation using ethanol to convert malonate to diethyl malonate. In other embodiments of the invention, malonate is purified from the fermentation broth by reactive distillation using methanol to convert malonate to dimethyl malonate. In various embodiments of the invention, the acid catalyst is sulfuric acid.

As is illustrated in Examples 41 Fischer esterification to ethanol using sulfuric acid as a catalyst can result in >99% purification of malonic acid from an aqueous phase. In various embodiments of the invention, the excess ethanol used in this process is recovered under reduced pressure and recycled for use in subsequent fermentation batches. In various other embodiments of the invention, the catalyst is matrix based (e.g. amberlite resins) and is recovered, recharged, and reused in subsequent batches.

The economics of malonate purification by Fischer esterification can be improved in accordance with the invention by co-localization of the malonate biorefinery with an alcohol biorefinery or chemical refinery (used herein to refer to production of alcohol from renewable or petroleum feedstocks using synthetic chemistry and not a fermentation process). In some embodiments of the invention, a malonate biorefinery is co-localized with an alcohol biorefinery or chemical refinery to reduce the cost of monoalkyl or dialkyl malonate ester synthesis. In some embodiments of the invention, the alcohol resulting from the biorefinery or chemical refinery is of lower purity than typically sold and distributed for fuel and/or chemical applications.

In other embodiments, the method of the invention for isolating malonate from the fermentation broth involves liquid-liquid extraction using an organic solvent. The pKₐ values of the two malonate carboxylic acid moieties are 2.83 and 5.69; thus, when the pH of the media is reduced below 2.8, greater than 50% of the molecules are not ionize. The affinity of these species is higher for organic solvent than for the aqueous fermentation broth. The separation of these molecules into the organic phase serves to drive the equilibrium toward the protonated species and thus progressively into the organic solvent. In some embodiments of the invention, the acid pH of the broth is the result of fermentation conditions. In other embodiments of the invention, the pH is lowered to the appropriate acidity by the addition of an acid. In various embodiments of the invention, the solvent is selected from, but not limited to, the group consisting of ethylacetate, dichloromethane, dichloroethane, decane, dodecane, hexanes, octanol, pentanol, or mixtures of thereof. In some embodiments of the invention, host cell growth and production of malonate at a low pH facilitates protonation of malonate to enhance solubility of the malonate in the organic solvent. In other embodiments of the invention, exogenous acid is added to facilitate this protonation. In some embodiments of the invention, the solvent is removed (wholly or partially) by vacuum distillation. In various embodiments of the invention, the concentrated malonic acid is esterified to an alcohol, as described elsewhere, and the ester purified by distillation.

In additional embodiments of the invention, malonate is removed from the fermentation broth by binding to an ion exchange resin. In various embodiments of the invention, the resin is selected from, but not limited to, the following: Lewatit® VP OC 1065, Lewatit® MP-64 chloride form, Toyopearl®, Dowex® 66 free base, Amberlite® IRA-67 free base, Amberlite® IRA-96 free base, Amberjet® 4200 chloride form, Lewatit® MonoPlus M 500 chloride form, Dowex® 1X8 chloride form, Amberlyst A26 hydroxide form, Amberlite® IRA958 chloride form. In various embodiments of the invention, malonic acid is eluted from the resin using a pH or salt gradient. In additional embodiments of the invention, one or more of these resins is used to remove impurities from the concentrated malonic acid

While the purification methods of the present invention can be used as single-step purification methods for purification of malonate from the fermentation broth, two or more purification methods can be used in series in accordance with the invention. For example, and without limitation, precipitation of malonate with calcium hydroxide may be followed by Fischer esterification of malonate with ethanol and further purification of the resulting diethyl malonate using distillation.

### Section 8: Chemistry for Using Malonate to Synthesize Other Chemicals

Malonate is a chemical precursor in a large number of important industrial reactions. The present invention enables, for the first time, the use of biologically derived malonate in these reactions. In any known reaction that utilizes malonate as a reactant, biologically derived malonate can be used. In any reaction using a monoalkyl malonate, dialkyl malonate, or Meldrum's acid, biologically derived malonate can be used following conversion to the appropriate substrate for the reaction. Furthermore, the invention provides new chemical synthesis methods in which malonate is used as a starting material. For these reactions, one can use malonate derived from any means, including biological production of malonate through enzyme catalyzed oxidation of malonate semialdehyde, synthetic oxidation of biologically- or synthetically-derived 3-hydroxypropionate, or synthetic oxidation of biologically- or synthetically-derived 1,3-propanediol.

Thus, in addition to providing biologically derived malonate for use in known chemical reactions, the invention provides new methods for using malonate and malonate-derived compounds to produce other useful compounds.

The invention arises in part from the fact that malonate and malonate-derived compounds can be used as substrates in the synthesis of acrylates (i.e., acrylate, methyl acrylate, ethyl acrylate, propyl acrylate, and butyl acrylate) by reaction with formaldehyde. The acrylates represent billions of dollars per year in sales and are used in products as wide ranging as diapers to shampoo to films and coatings. The invention provides two basic syntheses for production of an acrylate by reacting malonate or malonate-diesters with formaldehyde. In one method, malonate-diester is reacted with formaldehyde in the presence of an appropriate base (e.g. diethylamine) to produce the diester of 2-methylenemalonate. Subsequent saponification of the esters and heating results in the production of one part acrylate, one part CO₂, and two parts alcohol. The composition of the ester portion of the malonate diester, the base, and the solvent used can vary widely. For example, and without limitation, dimethyl, diethyl, dipropyl, dibutyl, diisopropyl, or dihexyl esters can be utilized. Similarly, the base used can be one of many. In various embodiments, the base is a tri-substituted amine. Non-limiting examples of tri-substituted amines suitable for use according the methods of the invention include those selected from the group containing piperidine, trimethylamine, triethylamine, tripropylamine, triisopropylamine, tributylamine, or longer chain trialkylamine. In some embodiments of this aspect of the invention, diethyl malonate is reacted with formaldehyde in pyrrolidine to form acrylate in a Knoevenagel condensation and subsequently saponified and heated to yield one part acrylate, one part CO₂, and two parts ethanol. In other embodiments of the method of the invention, dimethyl malonate is reacted with formaldehyde in diethylamine to form dimethyl 2-methylenemalonate which is subsequently converted to acrylate, CO₂, and methanol.

In other embodiments, a magnesium salt catalyzes the condensation of a malonate-diester with formaldehyde to form a diester 2-methylenemalonate which is then worked up in the described manner. For example, and without limitation, magnesium-silicate is a suitable magnesium salt for this reaction. In some embodiments, diethyl-malonate is reacted with formaldehyde in the presence of a magnesium-silicate catalyst to form diethyl 2-methylenemalonate. In other embodiments, diisopropyl malonate is reacted with formaldehyde in the presence of a magnesium-silicate catalyst to form diisopropyl 2-methylenemalonate. A general scheme using a malonate mono-ester is illustrated here:

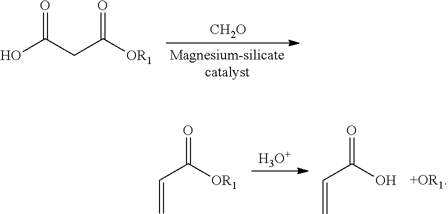

In this case, R₁ is selected from the non-limiting examples: H, CH₃, CH₃CH₂, CH₃(CH₂)₂, CH₃(CH₂)₃, (CH₃)₂CH₂, or (CH₃)₂CH₂CH₂.

In another embodiment, a Doebner modification of the Knoevenagel condensation is employed and malonate is reacted with formaldehyde in pyridine to produce acrylate. Example 28 illustrates the production of acrylate from malonate in accordance with this embodiment of the invention. In other embodiments, monomethyl malonate is reacted with formaldehyde in pyridine to form methyl acrylate. In the scheme illustrated here, a malonate mono-ester serves as the starting material in a Doebner modification of the Knoevenagel condensation

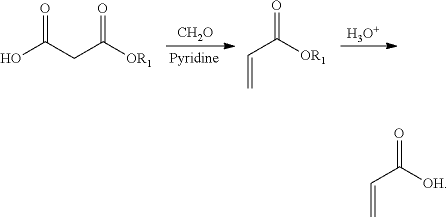

R₁ is selected from the non-limiting examples: H, CH₃, CH₃CH₂, CH₃(CH₂)₂, CH₃(CH₂)₃, (CH₃)₂CH₂, or (CH₃)₂CH₂CH₂. In other embodiments, monoethyl malonate is reacted with formaldehyde in pyridine to form ethyl acrylate.

In additional embodiments of the invention, malonate and malonate-derived compounds can be used as substrates in the synthesis of unsaturated dicarboxylic acids through reaction with various substrates, including, but not limited to, aldehydes, alkyl halides, dialdehydes, alkyl dihalides, terminal olefins, and combinations of the above (i.e., difunctional substrates). In the case of reaction of malonate or malonate-derived compound with an aldehyde, an alkene is formed that can be subsequently hydrogenated to yield the saturated product. Generally, malonate and malonate-derived compounds can be used to synthesize C5-C12 straight chain, saturated or unsaturated diacids (i.e., pentanedioic acid (CAS 110-94-1), hexanedioic acid (CAS 124-04-9), heptanedioic acid (CAS 111-16-0), octanedioic acid (CAS 505-48-6), nonanedioic acid (CAS 123-99-9), decanedioic acid (CAS 111-20-6), undecanedioic acid (CAS 1852-04-6), dodecanedioic acid (CAS 693-23-2)) and their corresponding dialkyl esters. In various embodiments of the invention, malonate is used as a substrate in the chemical synthesis of a C5-C12 straight chain, saturated diacid. In various embodiments of the invention, malonate or a malonate-derived compound is used as a substrate in the chemical synthesis of pentanedioic acid, hexanedioic acid, heptanedioic acid, octanedioic acid, nonanedioic acid, decanedioic acid, undecanedioic acid, or dodecanedioic acid.

In other embodiments of the invention, or a compound derived from malonate provided by, or not provided by, the invention is used as a substrate in the chemical synthesis of pentanedioic acid. Pentanedioic acid can be formed in accordance with the invention through reaction of synthetically derived diethyl malonate and formaldehyde (see Ahluwalia et al., “Organic reaction mechanisms,” 2nd ed. Alpha Science International: Harrow (2005) pgs 340-341); synthetically derived diethyl malonate and dichloromethane (see Perkin et al., J Am Chem Soc 59:990-995 (1891)); and synthetically derived Meldrum's acid and formaldehyde (see Hedge et al., J. Org. Chem. 26:3166-3170 (1961)).

In other embodiments of the invention, malonate or a malonate-derived compound is used as a substrate in the chemical synthesis of hexanedioic acid. Hexane dioic acid is one of two compounds used to produce Nylon 6,6 and represents billions of dollars in annual raw material sales. Nylon 6,6 is use in a wide range of durable consumer goods including, carpet, airbags for automobiles, and rope. Hexanedioic acid can be formed in accordance with the invention through reaction of magnesium-chelated malonate monoesters and 1,2-dichloroethane. Alkylation of dichloroethane and dimethyl malonate has been described (U.S. Pat. No. 6,262,298); however the undesired product dimethyl cyclopropane-1,1-dicarboxylate is the only product reported at 99.3% purity and 83% theoretical yield. Formation of cyclopropane-1,1-dicarboxylate results from intramolecular alkylation following the initial alkylation of 1,2-dichloroethane. In contrast, the present invention provides materials and methods for using a magnesium-chelated malonate monoester that does not allow the undesired intramolecular alkylation to the cyclopropane. A magnesium-chelated malonate monoester reacts with 1,2-dichloroethane through decarboxylation-facilitated enolate generation; the second intramolecular reaction (i.e., to the cyclopropane dicarboxylate) is unable to proceed following decarboxylation. An exemplary catalyst for use in accordance with this embodiment of the invention is magnesium-silicate. Those skilled in the art appreciate that various dihaloalkanes (e.g. 1,3-dihalopropane, 1,2-dihalopropane) can also be used with a magnesium-chelated malonate monoester to form a dicarboxylic acid product and that those halide groups can be I, Cl, Br, or F. For example, and without limitation, a 1,3-dihalopropane can be reacted with a magnesium-chelated malonate monoester to produce heptanedioic acid.

The invention also provides methods for production of hexanedioic acid by reaction of malonate and malonate-derived compounds with ethanedial. The formation of alkenoic acids by Knoevenagel reaction of malonate-diesters and aliphatic mono-aldehydes is known (see, for example, Rao et al., J Am Oil Chem Soc, 70:297-299 (1993); and, Zhang et al., Synth Comm, 40:3093-3100 (2010)). The present invention arises in part from the discovery that a dialdehyde can be reacted with a malonate-derived compound to produce the corresponding tetraester 1,3-diene-1,1,4,4-tetracarboxylate. For example, reaction of diethyl malonate with ethanedial yields tetraethyl buta-1,3-diene-1,1,4,4-tetracarboxylate; similarly, the reaction of malonate with ethanedial (glyoxal) yields hexa-2,4-dienedioic acid. Hydrolysis of the ester groups, when desired, proceeds through addition of acid to the reaction mixture. Decarboxylation to form the unsaturated dicarboxylic acid (i.e., hexa-2,4-dienedioic acid) is thermally induced, and subsequent hydrogenation of the unsaturated dicarboxylic acid yields hexanedioic acid. In some embodiments of the invention, diethyl malonate and ethanedial are condensed to yield the dialkylated bis-malonate ethyl ester; subsequent saponification to hydrolyze the ester groups followed by thermally-induced decarboxylation yields the unsaturated dicarboxylic acid. Hydrogenation of the unsaturated dicarboxylic acid yields hexanedioic acid. This process is summarized in the following reaction scheme, in which the alkyl groups (R₁ and R₂) are chosen from the non-limiting examples: H, CH₃, CH₃CH₂, CH₃(CH₂)₂, CH₃(CH₂)₃, (CH₃)₂CH₂, or (CH₃)₂CH₂CH₂.

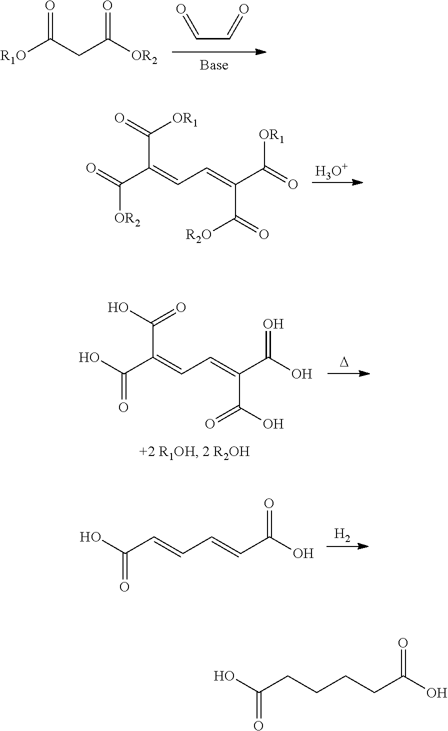

Those skilled in the art appreciate that Knoevenagel condensation of various dialdehydes and malonate-derived compound can be used according to the methods of the invention to synthesize saturated and unsaturated (with the alkenes at the 2 and n−2 positions) dicarboxylic acids. For example, and without limitation, propanedial can be used to synthesize heptanedicarboxylic acid, butane-1-4-dial is used to synthesize octanedicarboxylic acid, and pentane-1,5-dial can be used to synthesize nonanedicarboxylic acid.

In other embodiments of the invention, a malonate or a malonate-derived compound is used as a substrate in the chemical synthesis of heptanedioic acid. In addition to the dihaloalkane and dialdehyde routes to heptanedioic acid described above, the invention also provides methods for synthesis of heptanedioic acid through reaction of prop-2-enal (acrolein), which may or may not be derived from malonate as provided by the invention, and malonate. Michael addition of malonate to prop-2-enal yields a 2-(3-oxopropyl)malonic acid, and subsequent condensation to the aldehyde moiety with another molecule of malonate produces hex-1-ene-1,1,6,6-tetracarboxylic acid. Thermally induced decarboxylation followed by hydrogenation yields heptanedioic acid.

In another method of the invention, malonate is used as a substrate in the chemical synthesis of a lactam or lactone. In one embodiment, synthetic conversion of diethyl malonate to delta-valerolactam though the following steps; Diethyl malonate is produced by Fischer esterification of malonate with ethanol. Michael addition of diethyl malonate to acrylonitrile affords diethyl 2-(2-cyanoethyl) malonate. Hydrogenation of the nitrile moiety of diethyl 2-(2-cyanoethyl)malonate yields an amino acid that is lactamized under mild conditions to yield to ethyl 2-oxopiperidine-3-carboxylate. Saponification of the ester moiety of ethyl 2-oxopiperidine-3-carboxylate followed by heating results in decarboxylation to afford delta-valerolactam. This non-limiting example is illustrated here:

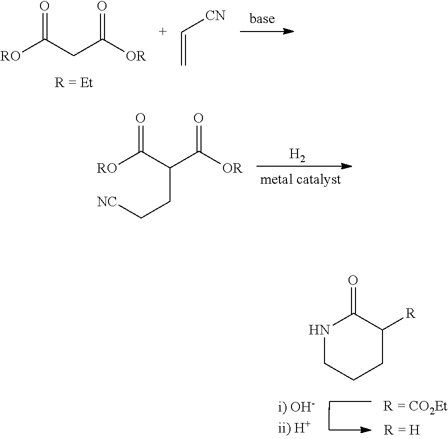

In another method of the invention, mono-ethyl malonate is used as a substrate in the chemical synthesis of ethyl acetate. Heating of the mono-ethyl malonate results in thermally induced decarboxylation to form ethyl acetate. In some embodiments, mono-ethyl malonate is used to synthesize ethyl acetate. One skilled in the art will recognize that there are a number of established methods for making mono-alkyl malonates from the malonic acid provided by the invention.

In another method of the invention, a mono-alkyl malonate (e.g. mono-methyl malonate, mono-ethyl malonate, mono-propyl malonate, mono-butyl malonate, mono-isobutyl malonate) is used as a substrate in the chemical synthesis of 3-hydroxypropionate or its related alkyl esters. Borane will chemoselectively reduce the free carboxylic acid moiety of the mono-alkyl malonate and produce the corresponding alkyl 3-hydroxypropionate. Alkyl 3-hydroxypropionates are themselves valuable products. The ester can also be hydrolyzed to yield 3-hydroxypropionate. 3-hydroxypropionate can in turn be dehydrated to acrylic acid, a valuable commodity chemical describe in more detail herein. In some embodiments, a mono-alkyl malonate is used to synthesize 3-hydroxypropionate. In some embodiments, the mono-alkyl malonate is mono-methyl malonate. In some embodiments, the mono-alkyl malonate is mono-ethyl malonate.

In another method of the invention, malonate is used to synthesize a dialkylated 1,3-dicarbonyl compound. In various embodiments, malonate is used as a substrate in the chemical synthesis of a compound selected from the group consisting of diethyl 2,2-dimethylmalonate, diethyl 2,2-diethylmalonate, diethyl 2,2-dipropylmalonate, diethyl 2,2-dibutylmalonate, dimethyl 2,2-dimethylmalonate, dimethyl 2,2-diethylmalonate, dimethyl 2,2-dipropylmalonate, and dimethyl 2,2-dibutylmalonate. Synthesis of dialkylated 1,3-dicarbonyl compounds can be formed in accordance with the invention from synthetically derived dialkyl malonates using alkyl halides (see Gatterman et al., “The practical methods of organic chemistry” 3rd ed, The Macmillan Company: New York, pgs. 189-191 (1916)).

In other embodiments of the invention, the malonic acid or malonate-derived compound provided by the invention is reacted with urea to form barbituric acid, the core structure of approximate fifty related pharmaceuticals currently on the market, including phenobarbital.

In some embodiments of the synthetic methods of the invention, a highly purified malonate preparation is used for the synthetic reaction and/or associated downstream applications; in other embodiments, a less purified malonate is used. In still other cases, a crude lysate or other relatively unpurified malonate can be used for the synthetic reaction and/or downstream applications. For most synthetic chemistry reactions, malonate purity greater than 90% w/w is typically desired. In cases where malonate is used for polymerization reactions even greater purity (i.e., 95% w/w or higher) may be desired. In other applications, however, one may employ a much less pure preparation of malonate. For example, malonate is useful as an animal foodstock, and for this purpose, one may employ the producing cells themselves or a crude lysate of them.

In another embodiment of the invention, malonate (produced by any means) is reacted with glutarate-semialdehyde (which can also be produced from malonate) to yield hept-2-enedioic acid. This is an additional example of a Knoevenagel condensation and can be conducted with the broad array of solvent and catalytic reagents associated with this reaction. Hept-2-enedioic acid is then reduced using catalytic hydrogenation to yield heptanedioic acid (pimelic acid). Pimelic acid has a wide variety of uses, including as a monomer in Nylon 5,7; as a fermentation media supplement used to enhance biotin biosynthesis; and as a plasticizer. Prior art methods for pimelic acid synthesis are costly and low yielding. The invention provides a new chemical synthesis for this important compound that may be derived from malonic acid as provided by the invention.

In other embodiments of the invention, vapor or liquid phase hydrogenation is used to convert dialkylmalonates into 1,3-propanediol. Due to the reactive nature of the number two carbon of malonate and malonate esters, polysubstituted derivatives of various compounds can be produced using the malonate provided by the invention. In some embodiments, neopentylglycol (2,2-dimethylpropane-1,3-diol), an important component of some polyesters, is produced by first generating 2,2-dimethyl-dialkyl malonate. This can compound can be produced using a variety of bases as catalysts and a methyl halide as the source of the methyl group. The subsequent hydrogenation of the ester groups results in the production neopentylglycol and the cognate alcohols from the ester groups. This represents a novel route to this important industrial compound. Furthermore, due to the highly reactive nature of the number two carbon of malonate and malonate derived compounds, other alkylhalides can be employed to result in new compounds with the potential for novel properties when incorporated into polymers.

The invention, having been described in detail, is illustrated by the following examples, which should not be construed as limiting the invention, given its diverse aspects, embodiments, and applications.

### EXAMPLES

### Example 1: Construction of Recombinant Nucleic Acids Encoding Wild-Type EHD3 and EHD3 Mutants E124S, E124A, E124A/E308V, and E124V Malonyl-CoA Hydrolases and Expression Vectors for Production of Malonate in E. coli

The present invention provides methods for producing malonate in an E. coli host, as well as E. coli host cells that produce malonate and express an EHD3 mutant enzyme, including but not limited to E124S, E124A, and E124V. This example describes the construction of protein coding sequences for EHD3 and the mutant EHD3 proteins useful in the invention, expression vectors containing those coding sequences, and host cells comprising those expression vectors. The nucleic acid encoding wild type S. cerevisiae EHD3 was amplified by PCR from Baker's yeast using primer pair A93/A94. Point mutation E124A was introduced using primer pairs A93/A96 and A95/A94, mutation E124V was introduced using primer pairs A93/A98 and A97/A94, and mutation E124S was introduced using primer pairs A93/A100 and A99/A94. The resulting nucleic acids were cloned into an E. coli expression vector containing the pSC101 origin of replication, a chloramphenicol resistance cassette, and a P_{LacO1} promoter using standard techniques. The resulting vectors were transformed into an E. coli DH10b host and plated on Luria-Bertani (LB) agar plates containing 50 μg/ml chloramphenicol (Cm⁵⁰) and 2% w/v glucose. Individual colonies were then inoculated into 3 ml LB media in 48-well plates; following 6 hours growth, plasmids were isolated and the EHD3 protein-coding region sequenced. When sequencing plasmids derived from clones of EHD3 (E124A), it was discovered that a second, point mutation, E308V, stabilized the EHD3 (124A) clone; the presence of an uncharged valine residue likely stabilizes protein folding. The EHD3 (E124A, E308V) strain was also used for malonate production (see Example 2). Primers A93-A100 are SEQ ID NO:13-20, respectively.

### Example 2: In Vivo Production of Malonate in E. coli Using EHD3 Mutants E124S and E124A/E308V

This example describes the host cells and culture conditions resulting in the in vivo production of malonate using a heterologous EHD3 malonyl-CoA hydrolase in an E. coli host cell. E. coli strain K12 was transformed with vectors containing wild-type EHD3, EHD3 (E124A), EHD2 (E124V), EHD3 (E124S), EHD3 (E124A, E308V) or an empty vector negative control. Transformants were streaked on LB agar plates (Cm⁵⁰, 2% glucose). Following overnight growth at 37° C., individual colonies were inoculated into 3 ml LB (Cm⁵⁰, 2% glucose) in a 48-well plate. Cultures were incubated on a plate shaker at 37° C. for 6 hours, at which point each culture was inoculated 1% v/v into M9 minimal medium supplemented with Cm⁵⁰ and a mixed carbon source (0.5% glycerol, 0.05% glucose, 0.2% lactose) in a 48-well plate. Cultures were incubated on a plate shaker at 30° C., and a 500 μL sample of the fermentation broth was removed for analysis after 48 hours incubation.

Samples were centrifuged (×6000 g, 1 min) and the supernatant analyzed for malonate quantification. Chemical standards were prepared in 20 μM of water. The separation of malonate was conducted on a Shimadzu Prominence XR UPLC connected to a refractive index detector and UV detector monitoring 210 nm. Product separation was performed on a Bio-Rad Aminex HPX-87h Fermentation Monitoring column. The UPLC was programmed to run isocratically using 5 mM H₂SO₄ as the eluant with a flow rate of 600 μL per minute. 10 μL were injected per sample, and the sample plate temperature was held at 4° C. Malonate standards began eluting at ˜19.8 minutes. Addition of the standard to samples containing malonate demonstrated a proportional increase in malonate peak area, confirming malonate production. Malonate concentrations (mg/l) were calculated by comparison to a standard curve prepared from authentic malonate.

For cultures harboring the empty vector control, no peak was observed at the same retention time as the malonate standard, and the integrated peak area was below the detection limit of the instrument (i.e., no malonate production was observed). Malonate production was observed in samples harboring wild-type EHD3, EHD3 (E124A), EHD3 (E124V), EHD3 (E124S), and EHD3 (E124A, E308V); malonate concentrations were (mean±std dev; n=3): Wild-type EHD3, 6.0±0.2 mg/l, EHD3 (E124A), 7.6±0.7 mg/l, EHD3 (E124V), 0.28±0.03 mg/l, EHD3 (E124S), 82.3±7.8 mg/l, and EHD3 (E124A/E308V), 8.35±2.5 mg/l. EHD3 (E124V) resulted in decreased production of malonate relative to the wild-type protein, likely due to both poor malonyl-CoA substrate binding and high promiscuous activity toward other, endogenous acyl-CoA molecules. Similarly, EHD3 (E124A) yielded only a minor increase in malonate production relative to wild-type EHD3. EHD3 mutant E124S resulted in a significant (p<0.05, t-test) increase in malonate production over wild-type EHD3, EHD3 (E124A), and EHD (E124V), demonstrating the importance of E124S in increasing malonyl-CoA substrate binding and malonate production.

### Example 3: Construction of Additional EHD3 E124 Mutants and Expression Vectors for In Vivo Production of Malonate in E. coli

Example 1 describes the construction of E. coli expression vectors for wild-type EHD3 and a subset of E124 mutations, specifically E124A, E124V, E124S, and E124A/E308V, and Example 2 describes their use to produce malonate in E. coli cells. This example describes construction of E. coli expression vectors for all EHD3 E124 point mutations, i.e., E124G, E124T, E124C, E124L, E124I, E124M, E124P, E124Y, E124W, E124D, E124N, E124Q, E124H, E124K, E124R, and E124F. These EHD3 mutants were constructed using an E. coli vector with pSC101 origin and P_{LacO1} origin of replication, as described in Example 1. The forward PCR primer comprises nucleic acid sequence (5′-aattttttactgatNNNtattctttgaattttcaaatagc-3′), where sequence “NNN” is the three nucleotides encoding the desired E124 amino acid point mutation; likewise, the reverse PCR primer comprises nucleic acid sequence (5′-ttcaaagaataaNNNatcagtaaaaaatttgatggacttg-3′), where sequence “NNN” is complementary to the three nucleotides encoding the desired E124 amino acid point mutation. The forward PCR primer was used in conjunction with PCR primer A94 (SEQ ID NO:14), and the reverse PCR primer was used in conjunction with PCR primer A93 (SEQ ID NO:13) to produce two overlapping EHD3 gene fragments containing the desired point mutation. Amplification of the two EHD3 gene fragments with primers A93 and A94 yielded the full length EHD3 gene containing the desired point mutation. The expression vectors were constructed using standard cloning protocols and transformed into E. coli DH10b and were subsequently isolated as described in Example 1 and verified by sequencing.

### Example 4: In Vivo Production of Malonate in Recombinant E. coli Using EHD3 E124 Mutants

This example describes fermentation of a set of E. coli host cells, each member of the set containing one of the 19 possible EHD3 E124 amino acid substitutions and determination of malonate levels produced. Specific E124 point mutations should theoretically improve malonyl-CoA binding and malonate production relative to wild-type; in particular, point mutations E124T, E124N, E124Q, E124H, E124K, and E124R should improve malonate production, theoretically due to introducing amino acids containing functional groups that improve interaction with the terminal carboxylic acid moiety of malonate. Point mutations E124S, E124Q, and E124K have side-chains that are located in the EHD3 binding pocket in positions that should theoretically best coordinate malonate binding. Malonate production is performed and quantified substantially as described in Example 2.

### Example 5: Construction of YciA Malonyl-CoA Hydrolase Expression Vectors for In Vivo Production of Malonate in E. coli

This example describes the use of E. coli YciA for the production of malonate in E. coli in accordance with the invention. Wild type E. coli acyl-CoA YciA is PCR amplified from the host genome using primers A120 (SEQ ID NO:21) and A121 (SEQ ID NO:22). The resulting nucleic acid is cloned behind the P_{LacO1} promoter on an E. coli expression plasmid containing a pSC101 origin of replication and an ampicillin resistant gene. The control vector comprises an empty vector without the yciA gene insertion behind the P_{LacO1} promoter. As described in Example 1, individual colonies are cultured, their plasmid isolated, and the coding region of the YciA gene insert sequenced.

### Example 6: In Vivo Production of Malonate in Recombinant E. coli Using Heterologous YciA

This example describes the host cells and culture conditions resulting in in vivo production of malonate using an expression vector encoding a heterologous YciA malonyl-CoA hydrolase as described in Example 5 in an E. coli host cell. Wild type E. coli strain K12 is transformed with vectors containing E. coli YciA; wild-type E. coli harboring an empty vector serves as a negative control. Malonate production is performed and quantified as described in Example 2.

### Example 7: Construction of Engineered EHD3 Malonyl-CoA Hydrolase Expression Vectors for In Vivo Production of Malonate in Yeast

The present invention also provides expression vectors, host cells, and methods for in vivo production of malonate in a yeast cell. Yeast cells can, in general, tolerate higher concentrations of organic acids in the fermentation broth and possess better-established industrial fermentation protocols than E. coli.

The yeast expression vectors described in this example were generated in part from use of the E. coli expression vectors described in Examples 1 and 3 as PCR templates for the EHD3 genes. The yeast expression vectors contain a 2-micron origin of replication, ura3 auxotrophic marker, and TEF promoter; the vectors also contain a puc origin of replication and an ampicillin or chloramphenicol resistance cassette for vector propagation in E. coli. The plasmids were transformed into either a S. cerevisiae BY4741 or BY4742 background, both derivatives of the S288C parental strain.

Additional mutations can be introduced into the EHD3 coding sequence to abrogate mitochondrial targeting. The basic amino acids R3, K7, K14, K18, and R22 in the EHD3 coding sequence are important for mitochondrial targeting, and mutation of any one or more of them to A or V decreases mitochondrial EHD3 expression and increases cytosolic EHD3 expression. Mutations are introduced by PCR amplification of an EHD3 template with forward and reverse primers containing the desired point mutation. Mutations are introduced using primers matching the EHD3 gene with the exception that the nucleotide sequence at the position of the desired amino acid point mutations is altered to “gyt” (where y is either a cysteine or thymine nucleotide in a mixed population of oligonucleotide PCR primers). The gene fragments are first cloned into an E. coli expression vector harboring a pSC101 origin of replication, p_{LacO1} promoter, and chloramphenicol resistance marker, and the vector sequences confirmed as described in Example 1; following isolation of the desired mutant, the EHD3 gene is amplified and cloned into a yeast expression vector.

### Example 8: In Vivo Production of Malonate in Recombinant Yeast Using Engineered EHD3 Malonyl-CoA Hydrolase

S. cerevisiae BY4742 host cells are transformed with a yeast expression vector prepared substantially as described in Example 7 harboring heterologous EHD3 or an empty vector negative control plasmid using standard protocols. Transformants are streaked on synthetic complete dropout medium (SD) agar plates lacking uracil and cultured at 30° C.; individual colonies are grown overnight in 3 mL SD media overnight at 30° C. and subsequently diluted 1% v/v into 3 ml of SD lacking uracil. Strains are cultured at 30° C. for 72 hours; 500 μl aliquots are sampled at 24, 48, and 72 hour timepoints for quantification of malonate production and OD₆₀₀. The TEF promoter is a constitutive promoter.

### Example 9: Increasing Malonate Biosynthesis in Engineered Yeast Through Expression of Heterologous Acetyl-CoA Synthetase

In addition to the methods, vectors, and host cells for expression of a heterologous malonyl-CoA hydrolase and in vivo production of malonate, as illustrated in Examples 1-8, the invention also provides methods and host cells for improved titer, yield, and/or productivity of malonate. In one aspect, malonate production is improved by increasing the biosynthesis of acetyl-CoA. This example describes heterologous expression of acetyl-CoA synthetase enzymes in a recombinant S. cerevisiae host comprising a malonyl-CoA hydrolase pathway and resulting improvement in malonate production. The five acetyl-CoA synthetase proteins illustrated are S. cerevisiae ACS1 and ACS2, E. coli AcsA, Salmonella enterica Acs, and Bacillus subtilis AcsA. All genes are PCR amplified from their respective hosts and cloned into a yeast expression vector harboring a 2-micron origin of replication, ura3 auxotrophic marker, and TEF promoter; the vectors also contain a puc origin of replication and ampicillin resistance cassette for vector propagation in E. coli.

### Example 10: Increasing Malonate Biosynthesis in Engineered Yeast Through Expression of Heterologous Pyruvate Dehydrogenase

While Example 9 describes increased acetyl-CoA biosynthesis through expression of heterologous acetyl-CoA synthetases, this example describes increased acetyl-CoA biosynthesis through expression of heterologous pyruvate dehydrogenase enzymes. In specific, S. cerevisiae pyruvate dehydrogenase enzymes PDA1, PDB1, LAT1, LPD1, and PDX1 are heterologously expressed in recombinant S. cerevisiae comprising a malonyl-CoA hydrolase pathway. The genes are all PCR amplified from the S. cerevisiae chromosome and cloned into a yeast expression vector harboring a 2-micron origin of replication, ura3 auxotrophic marker, and TEF promoter; the vectors also contain a puc origin of replication and ampicillin resistance cassette for vector propagation in E. coli.

### Example 11: Increasing Malonate Biosynthesis in E. coli and S. cerevisiae by Heterologous Expression of an Ethanol Catabolic Pathway

This example describes a third route to increase acetyl-CoA biosynthesis: heterologous expression of an ethanol catabolic pathway. An ethanol catabolic pathway comprises two or three enzymes. An alcohol dehydrogenase and an acetaldehyde dehydrogenase (acylating), or an alcohol dehydrogenase, acetaldehyde dehydrogenase (non-acylating), and an acetyl-CoA synthetase. The alcohol dehydrogenase enzymes S. cerevisiae ADH2, E. coli AdhP, and H. sapiens ADH1A, H. sapiens ADH1B, and H. sapiens ADH1C are combinatorially cloned with an acetaldehyde dehydrogenase (acylating) or aldehyde dehydrogenase and acetyl-CoA synthetase. The acetaldehyde dehydrogenase (acylating) enzymes E. coli MhpF, E. coli AdhE, Pseudomonas sp CF600 DmpF, and Pseudomonas putida TodL are also all cloned combinatorially. In ethanol catabolic pathways utilizing an acetaldehyde dehydrogenase (non-acylating) S. cerevisiae ALD2, ALD3, ALD4, ALD5, and ALD6 are used. Acetyl-CoA synthetase enzymes used are S. cerevisiae ACS1 and ACS2, and E. coli Acs. All genes are PCR amplified from genomic DNA.

For E. coli host cells, the ethanol catabolic pathway is expressed from a vector backbone harboring p15a origin of replication, ampicillin resistance marker, and P_{lacO1} promoter. All combinations of all two and three gene pathways are constructed as single operons. E. coli K12 is co-transformed with an EHD3 expression plasmid and an ethanol catabolic pathway plasmid and streaked on LB agar plates (Cm⁵⁰, Cb⁵⁰, 2% w/v glucose). Control strains harbor empty vector. Production cultures and analysis thereof are conducted as described in Example 2, with the notable exception of the addition of the second antibiotic require to maintain the second plasmid.

S. cerevisiae fermentations can be conducted using identical ethanol concentrations as the E. coli experiments. The yeast expression vectors harbor a 2-micron origin, ura3 auxotrophic marker, and CUP promoter, and all combinations of the ethanol catabolic pathways are constructed on this vector backbone; a plasmid absent an ethanol catabolic pathway serves as a negative control. An ethanol catabolic pathway plasmid is transformed into recombinant S. cerevisiae BY4742 comprising an engineered EHD3 malonyl-CoA hydrolase pathway on a yeast chromosome. All fermentations are conducted at 30° C. in SD media without uracil. Ethanol catabolic pathway expression is induced with 100 μM copper sulfate after 12 or 24 hours growth. 500 μl aliquots are sampled at 24, 48, and 72 hours for quantification of ethanol, acetaldehyde, acetate, and malonate concentrations; OD₆₀₀ measurements of cell density are also recorded at each timepoint. In addition to malonate titer, ethanol consumption is calculated.

### Example 12: Increasing Malonate Biosynthesis in S. cerevisiae by Heterologous Expression of an ATP Citrate Lyase

This example describes a fourth approach to increase acetyl-CoA biosynthesis and improve malonate production in recombinant S. cerevisiae. ATP citrate lyase (EC 2.3.3.8) catalyzes the formation of acetyl-CoA, oxaloacetate, and ADP in the cytosol from citrate. ATP citrate lyase enzymes from the oleaginous yeasts Candida curvata, Cryptococcus albidus, Lipomyces lipofer, Rhodospiridium toruloides, Rhodotorula glutanis, Trichosporon cutaneum, Yarrowia lipolytica, are PCR amplified from genomic DNA and cloned into a yeast expression vector behind a CUP promoter; the expression vector contains a 2-micron origin and leu2d auxotrophic marker. A plasmid absent an ATP citrate lyase enzyme serves as a negative control.

An ATP citrate lyase pathway plasmid is transformed into recombinant S. cerevisiae BY4742 comprising an engineered EHD3 malonyl-CoA hydrolase pathway on a yeast chromosome. All experiments are conducted at 30° C. in SD media without uracil. ATP citrate lyase pathway expression is induced with 100 μM copper sulfate after 12 or 24 hours growth. Some cultures are also supplemented with 0.5, 1, 2.5, or 5 g/l citrate to provide an additional demonstration of pathway activity. 500 μl aliquots are sampled at 24, 48, and 72 hours for quantification of citrate (where applicable) and malonate concentrations; OD₆₀₀ measurements of cell density are also recorded at each timepoint. In addition to malonate titer, citrate consumption can be calculated.

### Example 13: Increasing Malonate Biosynthesis in Recombinant Yeast Through Modification of Host Cell Fatty Acid Storage

This example describes a fifth approach to increase acetyl-CoA biosynthesis and improve malonate production in recombinant S. cerevisiae. Fatty acid biosynthesis pathways compete with malonate production for acetyl-CoA and malonyl-CoA, and altering host cell fatty acid anabolism can increase malonate production. The present invention provides host cells comprising genetic modifications of one or more nucleic acids encoding proteins affecting fatty acid storage and catabolism. In Saccharomyces cerevisiae, the proteins SNF2, IRA2, PRE9, PHO90, SPT21, POX1, ANT1, FOX3, EHD3, PAS1, PAS3, ARE1, ARE2, DGA1, LRO1, ACL1, MAE1, GLC3, GLG1, GLG2, PAT1, and PEX11 are knocked out individually and combinatorially and the resulting strains cultured for malonate production. All S. cerevisiae strains constructed comprise an engineered EHD3 malonyl-CoA hydrolase pathway for production of malonate. Fermentations are performed as described in Example 8, and malonate is quantified as described in Example 2.

### Example 14: Increasing Malonate Biosynthesis in Recombinant Yeast Through Increased Beta-Oxidase Activity

This example describes a sixth approach to increase acetyl-CoA biosynthesis and improve malonate production in recombinant S. cerevisiae. In addition to decreasing host cell fatty acid anabolism, increasing host cell fatty acid catabolism can increase malonate production. The present invention provides host cells modified for increased expression of PAT1 and/or PEX11. PAT1 and PEX11 are PCR amplified from genomic S. cerevisiae DNA and cloned into a yeast expression vector behind a CUP promoter; the expression vector contains a 2-micron origin and leu2d auxotrophic marker. A plasmid without a beta-peroxidase enzyme serves as a negative control.

A beta-oxidase pathway plasmid is transformed into recombinant S. cerevisiae BY4742 comprising an engineered EHD3 malonyl-CoA hydrolase pathway on a yeast chromosome. The engineered malonyl-CoA hydrolase is integrated onto the chromosome using standard recombination methods. The resulting strain serves as a base to test subsequent modifications and their impact on malonate production. All experiments are conducted at 30° C. in SD media without uracil. Pathway expression is induced with 100 μM copper sulfate after 12 or 24 hours growth. Some cultures are also supplemented with 0.5, 1, 2.5, or 5 g/l palmitic acid to provide an additional demonstration of pathway activity. 500 μl aliquots are sampled at 24, 48, and 72 hours for quantification of palmitic acid (where applicable) and malonate concentrations; OD₆₀₀ measurements of cell density are also recorded at each timepoint. In addition to malonate titer, palmitic acid consumption can be calculated.

### Example 15: Improving Malonate Biosynthesis in Engineered Yeast Through Increased Acetyl-CoA Carboxylase Activity

In addition to the methods, vectors, and host cells for expression of a heterologous malonyl-CoA hydrolase and in vivo production of malonate, the invention also provides methods and host cells for improved titer, yield, and/or productivity of malonate. In one aspect, malonate production is improved by increasing the biosynthesis of malonyl-CoA. Malonyl-CoA is the penultimate intermediate in the biosynthesis of malonate from acetyl-CoA, and in S. cerevisiae, this reaction is catalyzed by acetyl-CoA carboxylase (ACC1).

Malonyl-CoA biosynthesis is increased by overexpression of S. cerevisiae ACC1. Toward this end, the ACC gene is cloned using standard methods behind the CUP promoter on an S. cerevisiae expression plasmid containing a 2-micron origin of replication and ura3 auxotrophic marker. The control vector comprises an empty vector. S. cerevisiae host strains are engineered with chromosomal deletions of ACC1 and SNF1 protein kinase responsible for ACC1 phosphoregulation; chromosomal deletions are constructed both independently and in combination. Host cells harboring expression plasmids or control plasmids are grown as described in Example 8 and malonate production quantified as described in Example 2.

### Example 16: Improving Malonate Biosynthesis in Host Cells Through Supplementation of the Fermentation Broth with Cerulenin

In this example, malonate production in a recombinant host cell expressing an EHD3-derived malonyl-CoA hydrolase is improved by supplementation of the fermentation broth with cerulenin. The malonate production plasmid A4, comprising S. cerevisiae EHD3 (E124S) under control of a P_{LacO1} promoter, is transformed into an E. coli K12 host.

Individual colonies are inoculated into 3 ml LB medium (Cm⁵⁰, 2% w/v glucose) in 48-well plates and cultured for 6 hours at 37° C. on a plate shaker. Strains are then subcultured 1% v/v into 3 mL of M9 minimal medium (Cm⁵⁰, 0.5% w/v glycerol, 0.05% w/v glucose, 0.2% w/v lactose) and cultured at 30° C. on a plate shaker. Following 6 hours growth, one half of the cultures are supplemented with 10 mg/l cerulenin. After 48 hours growth, malonate concentration in the supernatant is measured as described in Example 2.

### Example 17: Improving Malonate Biosynthesis in Engineered Yeast Through Supplementation of Fermentation Broth with Carbon Dioxide

In this example, fermentation conditions are modified to increase the biosynthesis of malonyl-CoA. Enzymatic conversion of acetyl-CoA to malonyl-CoA by the enzyme acetyl-CoA carboxylase requires a stoichiometric amount of carbon dioxide, and supplementation of the growth media with carbon dioxide increases malonate production. Carbon dioxide is added to the growth media as either solid calcium carbonate or gaseous carbon dioxide.

Recombinant yeast cells harboring a malonyl-CoA biosynthetic pathway are grown in a defined minimal medium supplemented with between 0.1-10 g/l calcium carbonate. Control cultures are not supplemented with calcium carbon. Malonate production is quantified as described in Example 2 over the course of 48 hours growth.

### Example 18: Improving Malonate Biosynthesis in Engineered Yeast Through Decreased Malonate Catabolism

In this example, malonate production is increased by eliminating endogenous malonate catabolism in the host cell. S. cerevisiae contains multiple acyl-CoA synthetases, including FAA1, FAA2, FAA3, FAA4, LSC1, and LSC2; by deletion or modification of the nucleic acids on the host genome encoding these proteins, catabolism of malonate in the growth media can be decreased.

Malonyl-CoA knockout strains are constructed of each of the yeast acyl-CoA synthetases. The resulting strains are then cultured in a defined medium supplemented with 1-5 g/l sodium malonate. The malonate concentration in the fermentation broth is monitored over the course of 48 hours and quantified as described in Example 2. Strains with multiple knockouts can be constructed following similar procedures.

### Example 19: Improving Malonate Biosynthesis in Engineered Yeast Through Improved Malonate Secretion from the Host Cell

In this example, malonate production is improved by increasing secretion of malonate from the host cell. This is accomplished by overexpression of one or more of each of the S. cerevisiae pleiotropic resistant pumps, namely PDR5, PDR10, PDR11, PDR12, PDR15 and PDR18.

### Example 20: Improving Malonate Biosynthesis in Engineered Yeast Through Decreased Succinate Dehydrogenase Competitive Inhibition by Malonate

In this example, competitive inhibition of S. cerevisiae succinate dehydrogenase SDH1 is decreased, enabling higher titers of malonate to be achieved. First, the S. cerevisiae is genetically modified for deletion of the native, chromosomal copy of succinate dehydrogenase (SDH1) using standard methods. The resulting strain is grown anaerobically to facilitate growth in absence of SDH1 protein. The SDH1 deletion strain is subsequently transformed with a vector harboring a genetically modified SDH expression cassette containing an E300D, R331K, R331H, R442K, R442H mutation, or a combination of these mutations. These mutant SDH genes are cloned behind a constitutive TEF promoter on a yeast backbone harboring 2-micron origin and ura3 auxotrophic marker. Vectors are subsequently transformed into S. cerevisiae strain encoding malonyl-CoA hydrolase on the chromosome and the resulting strains grown in SD media lacking uracil. The transformed strains are cultured for malonate production as described in Example 8 and malonate is quantified as described in Example 2 to quantify the impact of these SDH mutations on malonate production.

### Example 21: MdcY Malonate Transcription Factor Biosensor Using Exogenously Added Malonate

Plasmid S14 was used to demonstrate biosensor response to exogenously added malonate in an E. coli host cell. S14 employs the malonate responsive transcription factor, MdcY (SEQ ID NO:3), and the MdcY-responsive promoter, P_{MdcL} (SEQ ID NO:6) derived from Acinetobacter calcoaceticus. This biosensor of the invention was constructed using an E. coli vector backbone with ampicillin resistance marker and ColE1 origin of replication; the tetA tetracycline resistance gene was placed under control of the P_{MdcL} promoter. Transformation of plasmid S14 into an E. coli host resulted in a strain expressing the tetA gene product following supplementation of the fermentation broth with malonate, and the strain exhibits a malonate-dependent increase in tetracycline resistance.

Nucleic acids encoding for MdcY and TetA gene products, P_{MdcL} promoter, and the E. coli vector backbone were synthetically produced; the biosensor vectors were than constructed by PCR amplification of the nucleic acids and subsequent cloning into the E. coli vector backbone. The plasmids were transformed into chemically competent E. coli DH10b and the resulting clones plated on LB agar plates containing 50 μg/ml carbenicillin (Cb⁵⁰). Individual colonies were grown overnight in 3 ml LB medium supplemented with antibiotic and the sequences of the purified plasmid were verified.

E. coli strain K12 was co-transformed with plasmids S14 and individual colonies isolated from LB agar plates (Cb⁵⁰). Colonies were grown in 25 ml LB broth (Cb⁵⁰) until reaching an optical density at 600 nm (OD₆₀₀) of approximately 0.50, at which point in time cell stocks were prepared and stored at −80° C.; cell stocks were 0.5 ml cell culture and 0.5 ml of a 50% v/v glycerol solution.

All biosensor demonstrations were performed with malonate. An aliquot of biosensor cell stock was thawed and used to inoculate 50 ml of LB medium (Cb⁵⁰) in a 250 ml, baffled Erlenmeyer flask. Cultures were incubated for 2 hours at 37° C.; subsequently, 0.6 ml biosensor culture was added to 48-well plates prepared with 2.3 ml LB medium (Cb⁵⁰) supplemented with tetracycline and malonate acid at the desired concentration (n=4). Plates were than grown at 30° C. on an orbital titer plate shaker. Following 12 hours incubation, 200 μl samples were taken for OD₆₀₀ measurement.

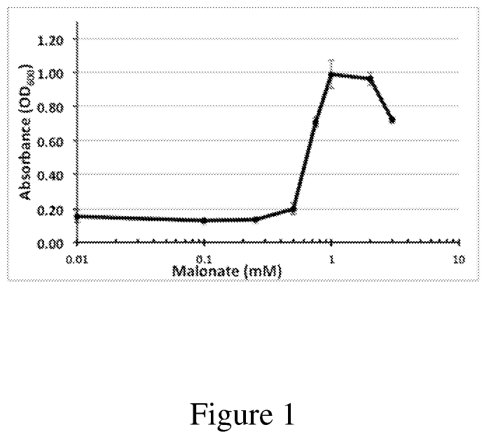

Biosensor cultures harboring S14 displayed a dose-dependent response for malonate (FIG. 1). The dynamic range (the maximum difference in OD₆₀₀ values between the fully induced samples and those samples absent malonate supplementation) was 1.2 OD₆₀₀ units, indicating the MdcY-P_{MdcL} based biosensor was highly responsive to exogenously added malonate. An increase in OD₆₀₀ was observed between 0.5-1 mM exogenously added malonate, providing a suitable range over which malonate can be quantified using this method.

### Example 22. MdcY Malonate Transcription Factor Biosensor to Detect Biologically Produced Malonate in Fermentation Broth

In this example, a malonate transcription factor biosensor was used to detect the production of malonate from a yeast strain engineered as described in other aspects of the invention.

Malonic acid was produced using a genetically engineered yeast strain as follows. S. cerevisiae BY4741 yeast cells harboring a vector for expression of malonyl-CoA hydrolase comprising a CYC1 terminator, an ampicillin resistance cassette, a PMB1 origin of replication, a CEN/ARS origin of replication, and a URA3 selection marker was used for fermentation. The F0PNG8-1 malonyl-CoA hydrolase (from Bacillus thuringiensis subsp. finitimus strain YBT-020; UniProt ID F0PNG8, with E91S mutation) and the F6AA82-2 malonyl-CoA hydrolase (from Pseudomonas fulva strain 12-X; UniProt ID F6AA82, with E95S and Q348A mutations) were each expressed from this plasmid under control of the TEF1 promoter. The culture medium described in Example 30 was used with 20 g/L glucose as a carbon source. Production was performed as follows. Two ml of culture medium in a 48-well plate was inoculated with 20 μl of a starter culture of the producer strain, in quadruplicate. The plate was covered with a breathable membrane, incubated on a plate shaker at 30° C., and sampled for HPLC and biosensor analysis of product accumulated after 142 h of growth. Cells and cell debris were removed from the culture media by centrifugation and filtered through 0.45 micron membrane prior to analysis by HPLC or biosensor.

E. coli cells harboring either plasmid pS14, encoding a tetracycline resistance gene (tetA) under expression control of the malonic acid-responsive P_{mdcL} promoter, or plasmid pS27, encoding a lacZ gene under expression control of the malonic acid-responsive P_{mdcL} promoter, were used as biosensor indicator strains. The vector pS27 was constructed in the same manner as described for pS14 in Example 21, with the lacZ gene, encoding a beta-galactosidase, inserted in place of the tetA gene. Biosensor strains were prepared as described in Example 21.

Yeast spent media obtained from 96-well production plates were added to 96-well plates containing 120 ul of biosensor cell culture. For the TetA (pS14) biosensor, 10 ul of tetracycline stock solution (to provide a range of 20-35 ug/ml) were added to each well. For both the TetA (pS14) and lacZ (pS27) biosensor, the remaining volume of each well was filled with LB medium (Cb⁵⁰) to a final volume of 600 ul and grown as previously described. Samples (200 ul) were collected to 96 well plates after 2 h for S27 cultures and after 5-8 h for S14 cultures, and OD600 was measured. An ortho-nitrophenyl-β-galactoside (ONPG) assay was performed on samples from S27 (beta-galactosidase reporter) biosensor plates as follows. Cells were diluted 1:4 in 25 ul lysis buffer and subsequently 90 ul of ONPG stock solution (10 mg/ml in deionized water) were added to each well. Contents of each well were completely mixed and left at 30° C. for 4-16 hours. Optical densities were measured at 420 nm.

A dose-dependent response to malonate was observed. Specific malonic acid concentrations were also measured by HPLC, as described in Example 2, and quantified by comparison to a standard curve. Linear regression analyses between the quantifiable output of the biosensor, OD420 (for pS27) or OD600 (for pS14) and specific malonic acid concentrations measured by HPLC were calculated to have coefficients of determination (R²) of 0.88492 plotting 37 OD420 samples and 0.89755 plotting 18 OD600 samples.

One skilled in the art will recognize that these very high coefficients of determination are indicative of the correlation between biosensor output and malonate concentration in the culture media. This aspect of the invention provides a tremendous advantage in both cost and time with regard to screening differential outputs in biological malonate production. Dilution of culture media used to challenge the biosensor can facilitate the extension of the dynamic response range of the sensor from zero to full solution saturation of malonate. The use of a plate based screen enables the screening of 96 samples in a few minutes in comparison to a time requirement of 2-20 minutes or more per sample for HPLC analysis. The savings in capital investment and solvent usage and disposal engendered by limiting or replacing HPLC altogether are also substantial.

### Example 23. Precipitation of Malonate from Fermentation Broth by Addition of Calcium Hydroxide, Calcium Carbonate, and Calcium Chloride

In this example, malonic acid was purified from the fermentation broth by precipitation with a divalent cation, specifically calcium. The purification methods were demonstrated using synthetic metabolites exogenously added to the fermentation broth. A yeast culture of S. cerevisiae BY4741 was grown in 0.5 L of synthetic complete media for 72 h at 30° C., 200 rpm. After 72 h, 25 ml aliquots of whole-cell fermentation broth were used to dissolve 0, 0.5, 1, 5, 10, 25, 50, 75, 100 g/l equivalents of malonic acid. Each 25 ml sample was divided into five-5 ml aliquots, and the pH of one aliquot of each concentration was adjusted to 5.5, 6.0, 6.5, 7.0, or 7.5. All samples were then centrifuged (×6000 rcf, 5 min, 25° C.), and the supernatants were transferred to separate tube. HPLC analysis showed malonic acid only in the supernatant fraction. Next, calcium chloride, a representative divalent cation, was employed for precipitating malonic acid from the clarified supernatant fraction by addition in equimolar equivalents to 0.5, 1, 5, 10, 25, 50, 75, 100 g/l of malonic acid of each sample at 25° C. The malonate concentration remaining in the supernatant and precipitate was then measured by HPLC as described in Example 2. At concentrations below 5 g/L, the extraction efficiency was 10% or less. It was also negligible once the pH was lowered to 5.5. However, at higher concentrations and pH values from 6-7.5 this method was quite effective at purifying malonate. The respective extraction efficiencies (percent isolated from fermentation broth) for this method at 100 g/L, 75 g/L, 50 g/l, 25 g/L, and 10 g/L are as follows: pH 7.5=89.8%, 88.5%, 83.3%, 70.1%, and 66.4%; pH 7.0=88.0%, 86.9%, 81.8%, 71.5%, and 63.9%; pH 6.5=80.5%, 79.0%, 75.8%, 65.0%, and 59.0%; pH 6.0=54.4%, 55.0%, 52.8%, 43.0%, 34.9%.

These results demonstrate that this method of the invention purified the malonic acid from the fermentation broth, separating it from both yeast cells and other dissolved chemicals.

### Example 24: Purification of Biologically Derived Malonate from Fermentation Broth by Reactive Extraction with Ethanol and Methanol

In this example, endogenously produced malonate from cultures of S. cerevisiae harboring a malonate biosynthesis pathway is purified from fermentation broth by reactive extraction. The host cells are first removed from 50 mL of fermentation broth by centrifugation (×6000 rcf, 5 min). Formation of the diethyl and dimethyl ester in the fermentation broth is performed using methods adapted from: Gatterman L. and Babsinian V S. “The practical methods of organic chemistry” 3rd ed, The Macmillan Company: New York, pg. 161-162 (1916). In brief, sodium chloride is added to the fermentation broth; subsequently, ethanol and sulfuric acid are added leading to formation of the diethyl malonate ester.

### Example 25: Reactive Extraction of Malonate from Fermentation Broth Using Tertiary Amines

Yeast fermentation broth is prepared as described in Example 23. Malonic acid is exogenously added to the fermentation broth to a final concentration of 50 g/l. The solution pH is adjusted to a value <4.0 by addition of an acid.

Three solutions of tertiary amines (TA) are prepared using 1-octanol as a diluent at 0.25, 0.5, and 0.75 mol-TA/kg 1-octanol. Tertiary amines used are triethylamine, tripropylamine, tributylamine, tripentylamine, trihexylamine, triheptylamine, trinonylamine, and tridecylamine.

An equal volume of organic solvent containing the amine and diluent are mixed with a fermentation broth containing malonate. The reaction is stirred at 1000 rpm, 25° C., for 2 hours; subsequently, the two phases are separated by centrifugation (×6000 rcf, 10 min). Malonate concentration in each phase can be measured by HPLC as described in Example 2.

### Example 26: Reactive Distillation of Malonate from Fermentation Broth Using Methanol and Ethanol

This example describes reactive distillation of malonate from fermentation broth using methanol and ethanol; a cationic exchange resin, Amberlyst-15, is used as the solid catalyst. The resin is dried in a vacuum oven for 6 hours at 70 degrees C. before use. The assembly consists of a glass column packed with ceramic attached to a collection container. Esterification takes place in a reactor connected to the bottom of the column. A condenser is placed at the top of the column for the condensation of low volatile vapors. The esterification reactor is fed with the fermentation broth and the ion-exchange resin, Amberlyst-15 (2% w/w), is added as a catalyst. Sufficient heat is applied to the reactor to vaporize the reaction mixture, and either methanol or ethanol is added to the reactor once the desired temperature is achieved. Samples are withdrawn from the drain valves of the esterification reactor and collection container. After completion of the reaction, volumes and masses of final reaction mixtures from the esterification reactor and collection containers are measured. Malonate consumption and product formation are measured as described in Example 2.

### Example 27: Synthetic Conversion of Malonate to Dimethyl Malonate and Diethyl Malonate

Biologically derived malonate is produced from recombinant S. cerevisiae in 1 L culture flasks as follows: strains are streaked on synthetic complete dropout medium (SD) agar plates lacking uracil and cultured at 30° C.; individual colonies are grown overnight in 3 mL SD media overnight at 30° C. and subsequently diluted 1% v/v into 500 ml of SD lacking uracil. Strains are cultured at 30° C. for 72 hours. The resulting fermentation broth is centrifuged (×6000 g, 10 min) and the supernatant separated from the cell pellet. Malonate is precipitated from the fermentation broth using calcium chloride (Refer to Example 23). The resulting calcium malonate is then converted to diethyl malonate by adding excess ethanol and equimolar sulfuric acid to catalyse a Fischer esterification.

### Example 28: Production of Acrylate from Malonate and Formaldehyde by Doebner Modification of the Knoevenagel Condensation

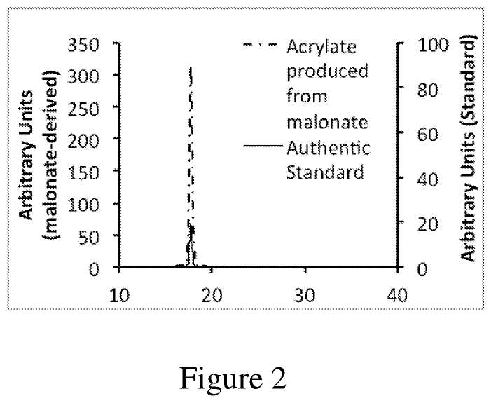

Acrylate was produced in accordance with the invention through the condensation of malonic acid with paraformaldehyde in pyridine. The reaction was conducted in a 3-necked round bottom flask with a magnetic stirrer. Fifteen ml pyridine and 15 ml toluene were added to the flask, and 10 g powdered malonic acid were added in 5 equal parts; subsequently, 1.1 equivalents (3.2 g) of paraformaldehyde was added to the reaction vessel over a thirty minute period. The mixture was stirred vigorously to promote solubilization of the components. The temperature of the reaction was started at 0° C. and then increased over the course of several hours to 50° C. until the formation of carbon dioxide, evidenced by the formation of bubbles, was observed. After 2 hours of heating at 50° C., the flask was allowed to return to room temperature and an aliquot of the reaction was diluted 100-fold in water and analyzed by HPLC using the method described in Example 2. The sample of malonate-derived acrylic acid co-eluted with an authentic standard at an elution time of approximately 17.5 minutes (see FIG. 2).

While the Doebner modification of the Knoevenagel reaction has been used to produce many compounds, its use in acrylate production is novel and important. Over a billion kilograms of acrylic acid are used annually to make wide a range of products from diapers to films and coatings. It is currently sourced largely from petroleum and production using the method described in this invention provides a partially or optionally fully renewable route to this commodity chemical, depending on the source of paraformaldehyde. In addition, malonic acid produced through the methods of this invention will be substantially less expensive than its petroleum derived counterpart. This facilitates production of sustainable or partially sustainable acrylic acid at a cost competitive with the incumbent petrochemical route.

### Example 29: Production of Pentanedioic Acid from Malonate and Formaldehyde

Biologically derived malonate is produced from recombinant S. cerevisiae in one L culture flasks as described in Example 8. The resulting fermentation broth is centrifuged (×6000 g, 10 min) and the supernatant separated from the cell pellet. Malonate is precipitated from the fermentation broth using calcium chloride and converted to diethyl malonate. The biologically derived diethyl malonate is then reacted with formaldehyde in pyridine and worked up to the desired acid according to literature methods (Hedge et al. (1961) JOC 26:3166-3170).

### Example 30: Bio-Catalytic Production of Malonate from Various Carbon Sources

In accordance with the invention, S. cerevisiae BY4741 yeast cells harboring a malonyl-CoA hydrolase expression vector comprising a CYC1 terminator, an ampicillin resistance gene, a PMB1 origin of replication, a CEN/ARS origin of replication, and a URA3 selection marker were grown in yeast fermentation media comprising 5 g/L ammonium sulfate, 1 g/L monopotassium phosphate, 0.5 g/L magnesium sulfate, 0.1 g/L sodium chloride, 0.1 g/L calcium chloride, 2 mg/L inositol, 0.5 mg/L boric acid, 0.4 mg/L calcium pantothenate, 0.4 mg/L niacin, 0.4 mg/L pyridoxine hydrochloride, 0.4 mg/L thiamine HCl, 0.4 mg/L zinc sulfate, 0.4 mg/L manganese sulfate, 0.2 mg/L p-aminobenzoic acid, 0.2 mg/L riboflavin, 0.2 mg/L sodium molybdate, 0.2 mg/L ferric chloride, 0.1 mg/L potassium iodide, 40 μg/L copper sulfate, 2 μg/L folic acid, 2 μg/L biotin, 10 mg/L adenine, 50 mg/L L-arginine HCl, 80 mg/L L-aspartic acid, 20 mg/L L-histidine HCl, 50 mg/L L-isoleucine, 100 mg/L L-leucine, 50 mg/L L-lysine HCl, 20 mg/L methionine, 50 mg/L L-phenylalanine, 100 mg/L L-threonine, 50 mg/L L-tryptophan, 50 mg/L L-tyrosine, and 140 mg/L L-valine (the base media). In different fermentations, each of the following was used as a sole carbon source: 20 g/L glucose, 2% v/v ethanol, or 2% v/v glycerol.

In this example, the F0PNG8-1 malonyl-CoA hydrolase (from Bacillus thuringiensis subsp. finitimus strain YBT-020; UniProt ID F0PNG8, with E91S mutation) was used under control of the TEF promoter. One and one-half ml of base media supplemented with 2% of the carbon source in a 48-well plate was inoculated with 50 μl of a saturated culture of the producer strain for culture in triplicate. The culture plate was covered with a breathable membrane, incubated on a plate shaker at 30° C., and sampled for HPLC analysis of product accumulation after 138 h of growth.

HPLC analysis of malonate accumulation was conducted as described in Example 2. The results were as follows: glucose as carbon source: 4.8 mM+/−0.2 mM (standard deviation) malonate; ethanol as carbon source: 7.5 mM+/−0.8 mM malonate; and glycerol as carbon source: 1.7 mM+/−0.1 mM malonate. These results show that the carbon sources tested were all suitable for use in production of malonate in accordance with the invention.

### Example 31: Construction and Expression of Recombinant Plasmid Vectors Encoding Various Malonyl-CoA Hydrolases, and their Use in the Production of Malonate in Yeast

Nucleic acids encoding various malonyl-CoA hydrolases provided by the invention were amplified by PCR from plasmids using the primers listed as follows: EHD3 (E124S) primers Y1-11_A13-R (SEQ ID NO:23)/Y1-11_A13-F (SEQ ID NO:24); B9IZZ9-1 primers YO012 (SEQ ID NO:25)/YO013 (SEQ ID NO:26); F0PNG8-1 primers YO014 (SEQ ID NO:27)/YO015 (SEQ ID NO:28); C3ALI3-1 primers YO018 (SEQ ID NO:29)/YO019 (SEQ ID NO:30); Q81DR3-1 primers YO020 (SEQ ID NO:31)/YO021 (SEQ ID NO:32); A4XS22-1 primers YO024 (SEQ ID NO:33)/YO025 (SEQ ID NO:34); E2XN63-1 primers YO026 (SEQ ID NO:35)/YO027 (SEQ ID NO:36); A5W8H3-1 primers YO028 (SEQ ID NO:37)/YO029 (SEQ ID NO:38); and F6AA82-1 primers YO030 (SEQ ID NO:39)/YO031 (SEQ ID NO:40). The purified PCR products were cloned downstream of the TEF1 promoter and upstream of the CYC1 terminator in a shuttle vector containing an ampicillin resistance cassette, a PMB1 origin of replication, a CEN/ARS origin of replication and a URA3 selection marker. The resulting plasmids were transformed into E. coli competent host cells and selected on LB agar plates containing Cb⁵⁰. Following overnight incubation at 37° C., individual colonies were inoculated in 2 ml of LB-Cb⁵⁰ in a 48-well plate and grown for 5 h at 37° C. on a shaker before the plasmids were isolated and confirmed by sequencing. Upon sequencing the construct containing protein F6AA82 (E95S), an unintended point mutation, Q348A, was found; this mutation was attributed to an error during PCR amplification. The resulting protein, F6AA82 (E95S/Q348A), is also referred to as F6AA82-2 herein. The Q348A point mutation was not shown to be necessary to obtain protein activity.

S. cerevisiae BY4741 cells were used as host for the vectors for expression of the various malonyl-CoA hydrolases. The plasmid vectors were individually introduced into the yeast host cells using standard procedures. Transformants were selected on agar plates of the media described in Example 30, containing 2% glucose as the carbon source.

The eight hydrolase-expressing S. cerevisiae strains were cultured as described in Example 22 and analyzed by HPLC as described in Example 2. The relative concentrations of malonate in the fermentation media were as follows (expressed as integrated area under the malonate peak; mean f S.D.; n=4): S. cerevisiae BY4741 (negative control) 48,865±9,345; EHD3 (E124S) 94,721±8,115; B9IZZ9 (E91S) 261,717±38,012; F0PNG8 (E91S) 216,654±31,145; F6AA82 (E95S/Q348A) 212,096±29,338; E2XN63 (E95S) 198,046±35,084; Q81DR3 (E91S) 193,665±37,898; Q63BK8 (E91S) 167,477±8,110; and A5W8H3 (E95S) 52,047±9,042. The identifiers are the Uniprot ID (http://www.uniprot.org/) followed by the mutation provided by the invention to result in malonate production. No malonate was detected in samples consisting of medium not inoculated with yeast cells.

In additional examples, C3ALI3 from Bacillus mycoides and A4XS22 from Pseudomonas medocina (strain ymp) containing E101S and E95S mutations, respectively, were utilized as malonyl-CoA hydrolases. Because the media conditions were varied slightly by buffering to pH 4.0, F0PNG8-1 and F6AA82-1 were included for comparison; all other fermentation, sampling, and analytical conditions were as described above. The results were as follows: C3ALI3 (E101S) 10±1 mM, A4XS22 (E95S) 7±1 mM, F0PNG8 (E91S) 11±2 mM, and F6AA82 (E95S/Q348A) 23±2 mM malonate. In the absence of a malonyl-CoA hydrolase protein, S. cerevisiae cells did not produce detectable concentrations of malonate.

This example demonstrates, in accordance with the invention, malonate can be produced in a yeast host cell expressing an enzyme containing an X₁ mutation conferring malonyl-CoA hydrolase activity. The E to S active site mutations common to all the mutant hydrolases used in this example can be utilized in other members of these enzyme classes to provide similar results.

### Example 32: Precipitation of Malonic Acid from Cells and Fermentation Broth Using a Monovalent Cation

In this example, malonic acid was purified from the fermentation broth by precipitation with a monovalent cation. The monovalent cation was sodium. The purification methods were demonstrated using synthetic malonic acid exogenously added to fermentation broth. A culture of S. cerevisiae BY4741 was grown up, prepared and seeded with malonic acids as described in example 23. Next, sodium chloride, a representative monovalent cation, was employed for precipitating malonic acid from the clarified supernatant fraction. Either 2 or 4 molar equivalents (as compared to malonate concentration) of sodium chloride was added to the supernatant fraction of each sample at 25° C. The malonate concentration remaining in the supernatant and precipitate was then measured by HPLC as described in Example 2. The concentration of malonate remaining in the media seeded with 100 g/L malonate and treated two molar equivalents of sodium chloride varied by pH as follows: pH 5.5=103%, pH 6.0=96%, pH 6.5=71%, pH 7.0=74%, pH 7.5=86%. The concentration of malonate remaining in the media seeded with 100 g/L malonate and treated four molar equivalents of sodium chloride varied by pH as follows: pH 5.5=92%, pH 6.0=86%, pH 6.5=66%, pH 7.0=81%, pH 7.5=86%.

### Example 33: Purification of Malonate from Cells and Fermentation Broth Using Diethyl Amine

In this example, malonic acid was purified from the fermentation broth by addition of diethyl amine. The purification method was demonstrated using commercially obtained malonic acid exogenously added to the fermentation broth. Yeast fermentation media was prepared and known quantities of malonic acid added as described in a previous example. Diethyl amine, a representative di-substituted amine, was employed for purifying malonic acid from the clarified supernatant fraction. Between 0.5 and 100 g/l of diethyl amine was added to the supernatant fraction of each sample at 25° C. The metabolite concentration remaining in the supernatant and precipitate was then measured as previously described via HPLC, as described in Example 2. The concentration of malonate remaining in the media seeded with 100 g/L malonate treated with 4 equivalents of diethylamine per equivalent of malonate varied by pH as follows: pH 5.5=100%, pH 6.0=86%, pH 6.5=65%, pH 7.0 67%, pH 7.5=57%.

### Example 34: Synthesis of Pimelic Acid

This example describes the synthesis of pimelic acid via the condensation of glutarate semialdehyde with malonate and catalytic hydrogenation. In a 250 ml round bottom flask, 10 grams of glutarate semialdehyde are dissolved in 40 ml of pyridine. Nine grams of malonic acid are added, and the mixture is heated, with stirring at 80° C. for 5 hours. The pyridine is removed using rotoevaporation, and the resulting material, containing the intermediate hept-2-en-1-7-dioic acid, is redissolved in hexanol. A Pd/C catalyst is added and the resulting mixture is stirred under a hydrogen atmosphere for 24 h. The catalyst is removed by filtration through Celite; the solvent is removed under reduced pressure; and the resulting pimelic acid is purified using by silica gel flash chromatography.

Pimelic acid is a key component of Nylon 5,7, which is used in fermentation to supplement biotin auxotrophy as well as in the production of several plastics. Prior art methods for pimelic acid synthesis are costly and low yielding. The invention provides a new chemical synthesis for this important compound that may be derived from malonic acid as provided by the invention.

### Example 35: Construction and Expression of Recombinant Vectors Encoding Additional Malonyl-CoA Hydrolases, and Production of Malonate in Yeast

In this example, E. coli malonyl-CoA:ACP transacylate FabD (SEQ ID NO:53) was mutated to contain one or more of the following amino acid changes at the indicated positions S92C, H201N, R117D, R117E, R117N, R117Y, R117G, R117H, Q11D, Q11E, Q11N, Q11Y, Q11G, Q11H, L93A, L93V, L93I, L93F, L93S, L93G and was employed as a malonyl-CoA hydrolase in S. cerevisiae. The nucleic acid encoding E. coli FabD was PCR amplified from E. coli strain K12 using primer F1 (5′-ATGACGCAATITGCATITGTGTTCCC-3′) and F2 (5′-TTAAAGCTCGAGCGCCGCT-3′). The amplified gene was then mutated using standard methods and inserted into a shuttle expression plasmid under the control of the TEF1 promoter and upstream of the CYC1 terminator. This vector contains an ampicillin resistance cassette, a PMB1 origin of replication, a CEN/ARS origin of replication, and a URA3 selection marker. The individual mutational combinations assayed are listed with the results below.

Individual colonies were inoculated into 1 ml of the media described in Example 30 containing 2% glucose as a carbon source. The cultures were incubated on a shaker at 30° C. for 24 h, and 20 μl of these cultures were used to inoculate production cultures of 2 ml of the same media. The production cultures were covered with a breathable membrane, incubated with shaking at 30° C. and sampled for HPLC analysis of product accumulation after 96 h and 168 h of growth.

No malonate was detected in samples consisting of medium not inoculated with yeast cells. Wild type yeast produced less than 0.1 mM malonate following 168 h fermentation. Expressing any of the four FabD variants produced malonate at levels higher then cells not expressing these proteins. Malonate accumulation after 96 and 168 h of fermentation using various engineered FabD malonyl-CoA-ACP transacylase enzymes expressed in S. cerevisiae were as follows: FabD S92C/L93V/R117H 96h=1.01 mM, 168h=2.49 mM; FabD L931/R117Y 96h=1.47 mM, 168h=2.48 mM; FabD L93S/R117G 96h=1.11 mM, 168h=2.89 mM; FabD L931/R117Y 96h=1.64 mM, 168h=3.47 mM.

### Example 36: Reactive Extraction of Malonic Acid from Water with Trialkylamines in 1-Octanol

In this example malonic acid was purified from water by reactive extraction with three trialkylamines; tripropylamine, trihexylamine, and trioctylamine. The purification method was demonstrated using authentic malonic acid added to distilled water. Malonic acid was added to water to a final concentration of 100 g/l; the pH of the solution was approximately 1.5.

250 ul of the aqueous malonic acid solution was mixed with 250 ul of an organic phase consisting of 25% v/v trialkylamine and 75% v/v 1-octanol. One sample was prepared without addition of the organic phase; this sample provided a measurement of the initial concentration malonic acid in each sample. Samples were mixed by inversion for 18 hours, centrifuged (×18,000 g) for 1 minute, and the aqueous phase sampled for analysis of malonic acid concentration by HPLC.

For HPLC analysis of malonic acid in the aqueous phase we employed a Shimadzu XR HPLC system equipped with a UV detector. 5 μl of each sample was injected into the system and separated with an Aminex HPX-87h fermentation-monitoring column (Bio-Rad, Hercules, CA). The mobile phase was de-ionized water (pH 1.95 with sulfuric acid), flow rate was 0.6 ml/min, oven temperature was 50C and, the UV detector monitored 210 nm. Samples containing were monitored for malonic acid elution at ˜10 minutes post injection.

33±2.6% of the malonic acid was extracted with tripropylamine, 73±4.4% of the malonic acid was extracted with trihexylamine, and 89±11.9% of the malonic acid was extracted with trioctylamine (n=3). Therefore, long chain length trialkylamines are preferred over short-chain length trialkylamines to increase extraction efficiency. Of the trialkylamines used in this example, trioctylamine is preferred to the shorter-chain length trihexylamine and tripropylamine.

### Example 37: Decreasing Aqueous Phase pH to Increase Reactive Extraction of Malonic Acid Using Trialkylamines

In this example malonic acid was purified from water at different pH values by reactive extraction with trioctylamine in 1-octanol. A stock solution of 100 g/l malonic acid was first prepared in water. The stock solution was then separated into working samples that were adjusted to the desired pH. Because addition of base diluted the malonic acid concentration at each pH value both malonic acid concentration before and after reactive extraction were taken. Calculation of the difference in malonate concentration between the pre- and post-extracted samples provided the percent yield at each pH value tested.

The reactive extraction was performed as follows. 250 ul of the aqueous malonic acid solution was mixed with 250 ul of an organic phase consisting of 25% v/v trioctylamine and 75% v/v 1-octanol. All samples were mixed by inversion for 18 hours, centrifuged (×18,000 g) for 1 minute, and the aqueous phase sampled for analysis of malonic acid concentration by HPLC as described in Example #36.

The extraction efficiencies at each pH were as follows; pH 1.5, 70%; pH 2.26, 57%; pH 2.93, 45%; pH 4.05, 30%; pH 4.62, 23%; pH 5.0, 15%; pH 5.5, 5%; pH 6.0, 0%; and pH 7.0, 3%.

Extraction efficiency decreased with increasing pH, and the highest extraction efficiency was achieved at pH 1.5. Above pH 6.0 extraction efficiencies were negligible. Thus, it is preferred that the pH of the fermentation broth be below 2.0 when extracting malonic acid using a trialkylamine.

### Example 38: Increasing Trialkylamine Concentration in an Organic Phase to Increase Reactive Extraction of Malonic Acid from an Aqueous Solution

In this example malonic acid was purified from water using a trialkylamine/1-octanol organic phase overlay containing different concentrations trioctylamine. The purification method was demonstrated using synthetic malonic acid added to distilled water to a final concentration of 100 g/l; the pH of the solution was approximately 1.5.

250 ul of the aqueous malonic acid solution was mixed with 250 ul of an organic phase consisting of the indicated amount of trialkylamine (expressed as the mol fraction relative to malonic acid in the aqueous phase) in 1-octanol. One sample was prepared without addition of the organic phase and was used to provide the measurement of the initial malonic acid concentration in the aqueous phase.

Samples were mixed by inversion for 18 hours, centrifuged (×18,000 g) for 1 minute, and the aqueous phase sampled for analysis of malonic acid concentration by HPLC as described in Example #36.

A linear relationship between extraction efficiency and trioctylamine:malonate mol fraction was observed; specifically the linear relationship existed between trioctylamine:malonic acid mol fractions of 0 to 1. Above 1 molar ratio, 100% of the malonic acid was extracted into the organic phase. Thus, the amount of trialkylamine in the organic phase must be equimolar to the amount of malonic acid in the aqueous phase (e.g., fermentation broth) to maximize extraction yield. Ideally a greater than equimolar amount of trialkylamine will be added to the organic phase to compensate for decreased extraction efficiencies due to other organic acids and anions in the fermentation broth.

### Example 39: Increasing Malonic Acid Reactive Extraction Yield from Aqueous Solutions at Different Ionic Strengths by Using Equimolar Amounts of Trioctylamine in 1-Octanol

In this example malonic acid was purified from water at different ionic strengths by reactive extraction with trioctylamine; by increasing the molar ration of trioctylamine in the organic phase to malonic acid in the aqueous phase to 1:1 we were able to improve the malonic acid extraction efficiency.

Malonic acid was added to water to a final concentration of 100 g/l. The ionic strength was adjusted to the indicated concentration by addition of sodium chloride. The final pH of all samples following addition of malonic acid and sodium chloride was approximately pH 1.5.

250 ul of the aqueous malonic acid solution was mixed with 250 ul of an organic phase consisting of trioctylamine in 1-octanol. The trioctylamine volumes were calculated such that the triocytylamine:malonic acid molar ratio was as indicated; the remainder of the volume was 1-octanol. One sample was prepared without addition of the organic phase; this sample provided a measurement of the initial malonic acid concentration. Samples were mixed by inversion for 18 hours at 18° C., centrifuged (×18,000 g) for 1 minute, and the aqueous phase sampled for analysis of malonic acid concentration by HPLC as described in Example 36.

Ionic strength (mM concentration) impacted the extraction of malonic acid using 0.59, 1.07, 1.61, and 2.14 molar equivalents of trioctylamine as follows; at an ionic strength of 0, 63.5%, 98.6%, 99.5%, and 99.4% of malonic acid was recovered respectively; at an ionic strength of 75, 60.3%, 91.3%, 93.7%, and 93.6% of malonic acid was recovered respectively; at an ionic strength of 150, 55.9%, 85.8%, 89.5%, and 89.1% of malonic acid was recovered respectively; at an ionic strength of 225, 52.4%, 83.5%, 86.0%, and 85.9% of malonic acid was recovered respectively; at an ionic strength of 300, 46.5%, 79.4%, 83.1%, and 82.6% of malonic acid was recovered respectively; at an ionic strength of 375, 44.3%, 76.7%, 79.9%, and 80.1% of malonic acid was recovered respectively; at an ionic strength of 450, 43.3%, 72.8%, 77.0%, and 78.1% of malonic acid was recovered respectively; at an ionic strength of 500, 43.1%, 72.2%, 75.1%, and 76.6% of malonic acid was recovered respectively.

Increasing the trioctylamine:malonic acid molar ratio to 1:1 increased the malonic acid extraction efficiency from the aqueous solution at all ionic concentrations tested. No meaningful improvements in malonic acid extraction efficiency were obtained by increasing the trioctylamine:malonic acid molar ratio above 1:1. When extracting malonic acid from aqueous solutions it is preferable to use a trioctylamine:malonic acid molar ratio of at least 1:1, and if there are no other organic acids a molar ratio of exactly 1:1 is preferred. If contaminating organic acids are present, a molar ratio higher than 1:1 may be preferred in order to improve the malonic acid extraction efficiencies.

### Example 40: Increasing Malonic Acid Back Extraction Yields from Trialkylamine/1-Octanol Organic Phase by Increasing Ionic Strength of Aqueous Phase

In this example we demonstrate methods to back extract malonic acid from an organic phase consisting of a trioctylamine in 1-octanol and into an aqueous phase.

Malonic acid was added to water to a final concentration of 100 g/l and extracted into an organic phase consisting of trioctylamine (used at a 1:1 molar ratio to malonic acid) dissolved in 1-octanol. 10 ml of aqueous solution was extracted into 10 ml of organic phase at 18° C. for 18 hours. The organic phase was then separated by centrifugation (×4000 g, 5 min). The aqueous phase was also sampled to quantify the non-extracted malonic acid concentration.

250 ul organic phase were then mixed with 250 ul aqueous solutions at the given ionic strength sodium chloride or sodium hydroxide. Samples were mixed by inversion at 18° C. for 18 hours. The samples were than centrifuged (×18,000 g, 1 min) and the aqueous phase sampled for analysis of malonic acid concentration by HPLC as described for Example 36.

The data presented below was normalized to the malonic acid concentration that was back-extracted into distilled water. Addition of either 0.5 molar sodium chloride or sodium hydroxide increased the yields of the back extraction reaction over 25-fold. Addition of sodium chloride above 1.5 M decreased back extraction yields; however, with sodium hydroxide back extraction yields continued to improve with further addition of sodium hydroxide. Back extraction yields would be further increased by running back extraction reactions at an elevated temperature (i.e. above 18° C.). Millimolar concentration of NaCl and fold improvement: 0 mM, 1; 0.5 mM, 27.3; 1 mM, 43.1; 1.5 mM, 74.5; 2 mM, 63.4; 2.5 mM, 66.5; 3 mM, 69.4; 3.5 mM, 63.7; 4 mM, 57.8; 4.5 mM, 53.3; and 5 mM, 45.2. Millimolar concentrations of NaOH and fold improvement: 0 mM, 1; 0.5 mM, 33.6; 1 mM, 46.5; 1.5 mM, 57.5; 2 mM, 52.7; 2.5 mM, 56.4; 3 mM, 62.6; 3.5 mM, 58.2; 4 mM, 68.5; 4.5 mM, 67; 5 mM, 74.2.

### Example 41: Purification of Malonic Acid from Aqueous Solution by Esterification and Subsequent Phase Separation

In this example we demonstrated esterification of malonic acid and ethanol to form diethyl malonate and subsequent phase separation of the diethyl malonate into a hexane organic phase.

500 μl aqueous solutions containing malonic acid at 100 g/l concentration, ethanol at the indicated concentration, and sulfuric acid at the indicated concentration were prepared. Malonic acid and ethanol were the substrates for forming diethyl malonate; sulfuric acid was added as the catalyst. An organic overlay of 250 μl hexane was added to each of the samples, they were mixed by inversion for 18 hours at 25° C. and atmospheric pressure, centrifuged (×18,000 g) for 1 minute, and the aqueous phase sampled for analysis of malonic acid concentration by HPLC as described in Example 36.

Consumption of malonic acid was measured by the decrease in malonic acid concentration in the aqueous phase. The baseline malonic acid concentration (i.e. 100% unreacted malonic acid) in the aqueous phase was established from a sample containing 0% v/v ethanol and 0% v/v sulfuric acid.

The addition of both ethanol and sulfuric acid to the reaction mixture was necessary to catalyze malonic acid esterification. The most preferable reaction conditions, as measured by percent consumption of malonic acid, were achieved at high ethanol and sulfuric acid concentrations. Namely, greater than 40% v/v ethanol (7.14 molar ratio to malonic acid) and greater than 10% v/v sulfuric acid (1.94 molar ratio to malonic acid) were necessary to catalyze near complete consumption of malonic acid. The results of this demonstration are as follows: esterification reactions were conducted with 5, 10, 20, 30, 40, and 50% (V/V) ethanol. The malonic acid remaining in the aqueous phase was as follows: with 50% (V/V) sulfuric acid 78.9%, 61.6%, 43.2%, 43.2%, 28.2%, and 16.6%; 10% (V/V) sulfuric acid 83.5%, 70.3%, 49.5%, 28.6%, 16.6%, and 8.5%; 5% (V/V) sulfuric acid 71.8%, 53.2%, 28.1%, 11.8%, 4.4%, and 0.6%, respectively. The no acid control showed 95.1%, 93.2%, 85.8%, 69.1%, 63.0%, and 87.3% malonate remaining in the respective solutions with ethanol. The addition of acid with no ethanol present resulting in the following concentrations of malonic acid remaining in the aqueous phase: 96.1%, 92.7%, and 84.4% for 5, 10, and 20% (V/V) sulfuric acid, respectively.

### Example 42: Improving Malonate Biosynthesis in Engineered Yeast Through Increased Acetyl-CoA Carboxylase Activity

In addition to the methods, vectors, and host cells for expression of a heterologous malonyl-CoA hydrolase and in vivo production of malonate, the invention also provides methods and host cells for improved titer, yield, and/or productivity of malonate. In one aspect, malonate production is improved by increasing the biosynthesis of malonyl-CoA. Malonyl-CoA is the penultimate intermediate in the biosynthesis of malonate from acetyl-CoA, and in S. cerevisiae, this reaction is catalyzed by acetyl-CoA carboxylase (ACC1).

In this example, the ACC from the yeast Yarrowia lipolytica CLIB122 (NCBI Reference Sequence: XP_501721) was back-translated to a DNA sequence using standard codon optimization tables for expression in S. cerevisiae. The resulting in the Y1ACC DNA sequence is included below as SEQ ID NO:11.

DNA encoding 50 nucleotide of upstream S. cerevisiae FAA1 homolog followed by 300 base pairs of the S. cerevisae TEF1 promoter sequence followed by the Y1ACC sequence (SEQ ID NO:11) and finally 50 nucleotides of downstream S. cerevisiae FAA1 were synthesized as a single piece of DNA from oligonucleotides using standard protocols and amplified by PCR. The resulting, flanked, Y1ACC PCR product and a linear DNA construct containing the selectable URA3 gene and flanking FAA1 homologous sequences, to direct recombination into the FAA1 site on the chromosome, were co-transformed into BY4741 containing 2 copies of malonyl-CoA hydrolase F6AA82-2 provided by the invention (strain LYM004), using a standard lithium acetate transformation procedure, and selected for by plating on Synthetic Defined medium containing 2% glucose (SD) and lacking uracil (−Ura) plates. Clones and corresponding genomic DNA were isolated and verified by PCR to contain the Y1ACC integration into FAA1.

For in vivo malonate production, one individual colony for each transformant picked was inoculated in a 50 μl aliquot of SD-Ura medium in a 96-well plate. LYM004 was grown for comparison of yeast lacking expression of a heterologous Y1ACC. The plate was incubated on a shaker at 30° C. for approximately 4 h and 25 μl of these pre-cultures were used to inoculate fermentation plates. In this example, 2 ml of SD medium in 48-well plates was used for the fermentation. The fermentation plates were covered with a breathable membrane, incubated on a plate shaker at 30° C. and sampled for HPLC analysis of product accumulation after 72 and 120h of fermentation. HPLC analysis of malonate accumulation in the fermentation broth was conducted as described in example 36.

No malonate was detected in samples consisting of medium only. LYM004 expressing Y1ACC produced 1.94 and 2.43-fold more malonate at 72 and 120h, respectively, than LYM004 alone. The highest concentration of malonate measured in the fermentation media were observed in samples collected after 120 h of fermentation.

### Example 43: Production of Malonate in S. cerevisiae Using Engineered F6AA82 Malonyl-CoA Hydrolase

In this example all twenty proteogenic amino acid point mutations were introduced at position E95 in protein F6AA82 (Q348A). Amino acid position 95 was believed to interact with the terminus of malonyl-CoA, and mutation of E95 would introduce malonyl-CoA hydrolase activity into the protein.

F6AA82 (Q348A) mutants at position 95 (i.e., all amino acids) were constructed using standard methods and cloned into a yeast plasmid containing a uracil auxotrophic marker, CEN/ARS origin of replication, TEF promoter and CYC terminator. The F6AA82 mutants were cloned behind the TEF promoter and directly upstream of the CYC terminator. All plasmids contained an ampicillin resistance marker and ColE1 origin for propagation of the plasmid in E. coli.

Plasmids were transformed into S. cerevisiae using a lithium acetate procedure and transformants were selected on SD-Uracil agar plates at 30° C. Twelve colonies of each mutant were inoculated into pre-cultures of 500 μl SD-Ura medium in a 96-well plate and were incubated ˜16h with shaking at 30° C. A 5 μl aliquot of these pre-cultures was used to inoculate 96-well production plates containing 500 μl RD4-Ura (1×YNB, 3×SC supplement, 2% glucose, 75 mM succinic acid buffer pH 4.0, uracil dropout) media. The production plates were incubated at 30° C. with shaking for 3 days before sampling the fermentation broth. The samples were clarified by centrifugation and filtered on a 0.45 μm membrane prior to HPLC analysis. HPLC analysis of malonate accumulation in the fermentation broth was conducted as described in example 36.

The production from each F6AA82 mutant (containing mutation Q348A in addition to the described E95 mutations), under these conditions, was as follows: F6AA82 (E95N) 8.03±0.14 mM, F6AA82 (E95S) 4.18±0.61 mM, F6AA82 (E95Y) 3.87±0.30 mM, F6AA82 (E95A) 2.33±0.50 mM, F6AA82 (E95K) 1.65±0.23 mM, F6AA82 (E95T) 1.16±0.62 mM, F6AA82 (E95D) 0.75±0.27 mM, F6AA82 (E95F) 0.17±0.13 mM, F6AA82 (E95V) 0.11±0.32 mM, F6AA82 (E95L) 0.11±0.12 mM, F6AA82 (E95G) 0.00±0.01 mM, F6AA82 (E95P) −0.31±0.78 mM, F6AA82 (E95R) −0.32±0.69 mM, F6AA82 (E95) −0.32±0.40 mM, F6AA82 (E95W) −0.34±0.73 mM, F6AA82 (E95Q) −0.37±0.74 mM, F6AA82 (E95H) −0.53±0.70 mM, F6AA82 (E95C) −0.57±0.69 mM, F6AA82 (E951) −0.64±0.81 mM, and F6AA82 (E95M) −0.77±0.79 mM. Negative concentrations of malonate were due to the production titers falling below the detectable level of the HPLC and indicate the absence of malonate production.

F6AA82 (Q348A) proteins containing mutations E95S, E95Y, E95T, E95N, E96K, E95V, and E95D produced significantly (t-test, p<0.05) more malonate than the F6AA82 (Q348A) protein. F6AA82 proteins containing these mutations are suitable for the hydrolysis of malonyl-CoA and production of malonate. F6AA82 mutations E95S and E95N are preferred for the hydrolysis of malonyl-CoA and production malonate.

E95Y, E95T, E95K, E95V, and E95D produced significantly (t-test, p<0.05) more malonate than the wild type F6AA82 protein. F6AA82 proteins containing these mutations are suitable for the hydrolysis of malonyl-CoA and production of malonate.

### Example 44: Impact of Promoter Selection on the Production of Malonate in S. cerevisiae

In this example, an expression plasmid backbone comprising a CEN/ARS origin of replication, a PMB1 origin of replication, an ampicillin resistance marker, an URA3 marker and an hph marker was amplified from plasmid Y20. The malonyl-CoA hydrolase F6AA82-2 flanked by the CYC1 terminator was amplified by PCR from plasmid Y1-F6AA82-2 and assembled with the backbone using standard techniques. The resulting plasmid, pPLIB0-Q9I, was used as a backbone for cloning of the different promoters selected.

Ninety-six reference genes were selected from S. cerevisiae strain BY4741. Promoter sequences to these genes were generated by PCR amplifying, using S. cerevisiae strain BY4741 genomic DNA as template, nucleic acid fragments corresponding to approximately 750 base pairs immediately upstream of their open reading frame, including the start codon of the gene of interest. Using standard cloning techniques, these promoter sequences were cloned immediately upstream of F6AA82-2 (Start codon at 3739 in SEQ ID NO:12) in pPLIB0-Q9I. The resulting plasmids were propagated in E. coli and the presence of the desired promoter was verified by sequencing.

The plasmids described above were used to transform S. cerevisiae BY4741 using standard lithium acetate procedures. Transformants were selected on agar plates of CSM medium without uracil at 30° C. One transformant harboring each plasmid was inoculated in a pre-culture plate consisting of 300 ul of RD-Ura medium (2×YNB, 3×SC-U, 2% glucose) in a 96-well plate. The pre-culture plate was incubated at 30° C. approximately 20 h with shaking, and 30 μl of these pre-cultures was used to inoculate production plates consisting of 1.8 ml of RD4-Ura medium (2×YNB, 3×SC-U, 2% glucose, 75 mM succinate buffer pH 4.0) in 48-well culture plates (1 production well for each pre-culture). The plates were incubated at 30° C. with shaking for 144h before sampling the fermentation broth. The samples were clarified by centrifugation and filtered on a 0.45 μm membrane prior to HPLC analysis.

HPLC analysis of malonate accumulation in the fermentation broth was conducted as described in example 36. Millimolar malonate concentration in the fermentation broth after 144h of fermentation with different promoters driving expression of F6AA82-2 are as follows: HSP150 8.8; PGK1 8.6; PHO5 8.5; SCT1 7.8; PRB1 7.6; TPI1 7.1; ACH1 7; HXK2 6.9; ACO1 6.9; JEN1 6.9; MDH2 6.8; POX1 6.6; CIT1 6.6; ALD4 6.6; ADH1 6.5; TDH3 6.4; ADH2 6.4; SDH1 6.4; TDH1 6.1; MLS1 6.1; RPN6 6; GLK1 5.9; POT1 5.8; HSP26 5.8; FBA1 5.7; LPD1 5.7; CYC1 5.5; COX5a 5.5; TEF1 5.5; SHH4 5.5; GND2 5.5; TPS1 5.5; MDH1 5.4; PDC1 5.4; HXK1 5.3; TDH2 5.3; IDH2 5.3; DDR2 5.2; SLT2 5.2; ENO2 5.1; COX6 5; CHO1 4.9; PHO3 4.9; PFK1 4.9; ACS1 4.9; GUT2 4.8; PHM7 4.8; CIT2 4.7; ACS2 4.7; ALD2 4.5; IDH1 4.5; IDP2 4.5; FBP1 4.3; PHO12 4.3; PDC5 4.2; PFY1 4.1; GDH1 4.1; PEX13 4.1; ICT1 4.1; YSA1 4; KGD2 3.9; GIM5 3.9; GAP1 3.9; DBP5 3.8; STE5 3.7; BIO2 3.7; PDC6 3.6; HXT5 3.6; REG1 3.6; TPS3 3.5; BIO5 3.3; PHO8 3.3; IRC7 3.2; GPM1 3.2; ALA1 3.1; KGD1 3.1; MIG1 3.1; YKT6 2.9; SNO4 2.8; ARA1 2.8; PDR10 2.7; YBR139W 2.3; ERR2 2.2; CRC1 1.9; TSL1 1.3; ENO1 1.2; PFK2 1.1; RPL6A 1.1 As many of these promoters are unnamed, the letter number code in front of each number indicates the gene immediately downstream of the cloned promoter, in S. cerevisiae strain BY4741.

This example demonstrates that a wide variety of promoters can be used to control expression of a malonyl-CoA hydrolase to produce malonate in vivo in yeast. Furthermore, 40 different promoters resulted in titers of at least 5 mM malonate, and 7 promoters resulted in production of at least 7 mM in this experiment.

### Example 45: Integration of a Malonyl-CoA Hydrolase Encoding Gene to the S. cerevisiae Genome for the Production of Malonate

In this example, S. cerevisiae strain BY4741 is used as a host strain, for the genomic integration of the malonyl-CoA hydrolase afforded by the invention. Three sites on the S. cerevisiae genome were chosen for integration of the synthetic nucleic acid constructs in this strain and the three linear nucleic acids cassettes to be integrated each carried unique homology sites to target integration and a unique selectable marker encoded upstream of the malonyl-CoA hydrolase. In all three cases nucleic acid encoding the malonyl-CoA hydrolase F6AA82 (E95S/Q348A) flanked by the TEF1 promoter and the CYC1 terminator was utilized as the expression portion of the cassette.

In two cases the solo long terminal repeats YPRCdelta15 and YORWdelta22, non-coding DNA of the chromosome, were targeted for integration. In the third case, a malonyl-CoA hydrolase cassette provided by the invention (F6AA82 (E95S/Q348A)) was integrated in place of the BUD21 gene. The sequences of these linear insertion cassettes are included below as SEQ ID NO:41, SEQ ID NO:42, and SEQ ID NO:43. The unique integration sites and selectable markers are YPRCdelta15 (HIS3), YORWdelta22 (LEU2), and BUD21 (URA3), respectively.

These nucleic acid cassettes were constructed and transformed into S. cerevisiae BY4741 using standard protocols. Transformants were selected on agar plates made of SC medium with appropriate amino acid dropouts at 30° C. Several transformants for each construction were re-streaked on selective agar plates. Transformants were then screened and verified by production of malonate and by PCR The following strains resulted from these integrations: LYM002 contains 1 copy of F6AA82 (E95S/Q348A) integrated at YPRCdelta15 (HIS3); LYM004 carries 2 copies of F6AA82 (E95S/Q348A), one at YPRCdelta15 (HIS3) the second at YORWdelta22 (LEU2); LYM007 is derived from LYM004 and has a third copy of F6AA82 (E95S/Q348A) integrated at the BUD21 locus.

For fermentation and malonate production, S. cerevisiae BY4741 carrying plasmid Y1-F6AA82 (E95S/Q348A) served as a positive control. Pre-cultures of 500 μl of RD4 medium in a 96-well plate were incubated 16-20h with shaking at 30° C. A 5 μl aliquot of these pre-cultures was used to inoculate 96-well production plates containing 500 μl of RD4 medium (1×YNB, 3×SC supplement, 2% glucose, 75 mM succinic acid buffer pH 4.0). The production plates were incubated at 30° C. with shaking for 120h before sampling of the fermentation broth. The samples were prepared and analyzed by HPLC as described in Example 36.

A standard curve established with an authentic standard was used to determine malonate concentration in the fermentation broth. The malonic acid production levels from these fermentations were as follows S. cerevisiae BY4741+Y1-F6AA82 (E95S/Q348A), 2.6±0.4 mM; LYM002, 3.2±0.2 mM; LYM004, 4.6±0.2 mM; LYM007, 7.7±0.3 mM.

This example illustrates the benefits of genomic integration of a malonyl-CoA hydrolase encoding gene provided by the invention in that a single integrated copy of the gene results in higher malonic acid titers than the same gene expressed from a plasmid. This example also serves to demonstrate modulation of malonic acid by the invention via the number of malonyl-CoA hydrolase encoding genes are present in the cell.

### Example 46: Expression of Acetyl-CoA Synthetases for Increased Production of Malonate from an Engineered Host Cell

Acetyl-CoA synthetases (ACSs) are common to many organisms. These enzymes produce acetyl-CoA using acetate and adenosine triphosphate (ATP) as substrates. In accordance with this invention, these enzymes can be used to convert acetate from any source, including endogenous metabolism, feedstock hydrolysis, or by feeding, into acetyl-CoA, the penultimate precursor in malonyl-CoA biosynthesis. By increasing acetyl-CoA levels, one can increase malonyl-CoA, and hence, malonate, in host cells provided by the invention.

An illustrative ACS suitable for this purpose can be obtained from the bacteria Salmonella enterica. To express the S. enterica ACS (SeACS) in S. cerevisiae, the amino acid sequence was back-translated to a DNA sequence using codon optimization tables, and one residue determined to be involved in decreasing enzymatic activity was mutated (L641P). The resulting codon optimized DNA sequence provided by the invention is SEQ ID NO:44.

Nucleic acids encoding the SeACS1 (L641P) were synthesized de novo from oligonucleotides and inserted into a yeast plasmid, Y1, under the control of the TEF1 promoter from S. cerevisiae using standard protocols. This plasmid also contains a CEN/ARS origin and a gene for conferring uracil prototrophy. Clones of LYM004 containing this plasmid were selected by plating on SD-uracil plates and verified to contain the SeACS1(L641P) gene by sequencing.

LYM004 was used as host for the expression of the SeACS1(L641P) acetyl-CoA synthetase. For in vivo malonate production, one individual colony for each transformant picked was inoculated in a 50 μl aliquot of SD-Ura medium in a 96-well plate. LYM004 was grown for comparison of yeast lacking expression of a heterologous SeACS1(L641P), but containing a Y1 empty vector. The plate was incubated on a shaker at 30° C. for approximately 48h, and 50 μl of these pre-cultures were used to inoculate fermentation plates. In this example, 500 uL of RD4U medium in 96-well plates were used for the fermentation. The fermentation plates were covered with a breathable membrane, incubated on a plate shaker at 30° C., and sampled for HPLC analysis of product accumulation after 120h of fermentation. HPLC analysis was conducted as described in Example 36.

No malonate was detected in samples consisting of medium not inoculated with yeast cells. Under the conditions tested, LYM004 expressing SeACS1 produced 1.86-fold more (5.5±0.62 mM) malonate at 120h than LYM004 with an empty Y1 control alone, thus illustrating the improvements in malonate production provided by this embodiment of the invention.

### Example 47: Utilization of Different Carbon Sources for Production of Malonate Using a Modified Strain of Pichia kudriavzevii

Strain LPK3003 was derived from Pichia kudriavzevii strain Y-134 (obtained from the USDA Agricultural Research Services, Peoria, IL) by genomic integration of a nucleic acid cassette encoding the hph hygromycin phosphotransferase (conferring resistance to hygromycin B) driven by the PkTEF1 promoter, and the F6AA82 (E95S/Q348A) malonyl-CoA hydrolase driven by the PkTDH1 promoter.

Seed cultures of LPK3003 were grown for 16-20h in YPD medium with shaking at 30° C. This seed culture was used to inoculate 1.1×YNB, which was then aliquoted at 450 μl per well in a 96-well plate. 50 μl of solutions of various carbon sources (glucose, sucrose, ethanol, glycerol or acetate from sodium acetate) were added to each well (in triplicate) to a final concentration of 2% (w/v). The plate was incubated at 30° C. with shaking for 115 h and the fermentation broth was sampled. Samples were prepared and analyzed by HPLC as described in Example 36.

In this example, there was little or no growth observed for sucrose or acetate carbon sources, and malonate accumulation was negligible in these samples. As described above, host cells can be modified to confer or enhance sucrose and/or acetate catabolism by introducing a sucrose invertase and/or acetyl-CoA synthase (ACS), respectively. The other carbon sources tested in this example resulted in malonate accumulation (average replicates f S.D.): 4.6±0.5 mM from glucose, 5.38±0.05 mM from glycerol and 3.7±0.3 mM from ethanol.

These results demonstrate that a variety of carbon sources can be used to produce malonate from an engineered Pichia kudriavzevii strain expressing a malonate-CoA hydrolase. Notably, glycerol provided the highest titers in this example.

### Example 48: Bio-Reactor Based Production of Malonate

In this example, yeast strain LYM004 (see Example 45 for construction details) was grown in fed-batch control in a 0.5 L bioreactor. A single colony of LYM004 was isolated from a SC plate and cultured in 5 mL of RD4 media (see Example 43 for recipe). The culture was maintained at 30° C. overnight, shaking at 200 rpm. The 4 mL of culture was used to inoculate 50 mL of fresh RD4 media in a 250 mL non-baffled flask and grown overnight at 30° C., 200 rpm. The time zero OD 600 nm absorbance was 0.304. After overnight growth (16h), this culture was used to inoculate 1 L of RD4 media. This culture was split into 2 separate 500 mL aliquots and added to two separate bioreactors. Both fermentations were maintained at 30° C., with a single impeller run at 400 rpm, and sparge rate of 1 vessel volumes per minute (VVM) using compressed air. The cultures were grown overnight (21h) to allow for glucose consumption prior to starting the fed-batch phase. The feed (recipe below) was delivered for 2s, every 980s. 0.5 mL samples were taken daily and analyzed for production of malonic acid. After 4 days, the cultures had accumulated 34 mM of malonic acid and reached an OD 600 nm of 16.2. After 9 days, the cultures had accumulated 116 mM of malonic acid and reached an OD 600 nm of 52.1.

The batch feed media consisted of 17 g/L Difco YNB; 50 g/L ammonium sulfate; 49.8 g/L Synthetic Complete (SC) supplement lacking histidine, methionine, and leucine; 2.57 g/L methionine; 8.85 g/L succinic acid, and 20 g/L glucose; the pH o was adjusted to 4.0.

This protocol was repeated a strain, LYM007, that expressed 3 copies of a malonyl-CoA hydrolase (see Example 45). In this example, a single impeller was run at 700 rpm and the composition of the feed was as follows: 68 g/L YNB (Sigma), 16.6 g/L SC-his-met-leu (Sunrise Science), 1.284 g/L methionine, 75 mM succinate buffer (pH 4.0), 400 g/L glucose. The feed rate used was cycle 5s on every 980s. 0.2 mL samples were taken daily and analyzed for production of malonic acid, acetic acid, succinic acid, pyruvic acid via HPLC. Growth was monitored by measuring optical density at 600 nm (0D600). The OD600 and malonate concentration from each time point was as follows: 18h, OD=6.9, 3.2 mM; 45 h, OD=13.8, 14.0 mM; 69h, OD=16.6, 24.2 mM; 88.5h OD=24.4, 31.1 mM; 111.8h, OD=36.9, 47.8 mM.

In a comparative example, LPK3003 (see Example 47) was grown in fed-batch control in a 0.5 L fermenter. A single colony of LPK3003 was isolated from a YPD plate and cultured in 5 mL of YPD media. The culture was grown at 30° C. overnight, shaking at 200 rpm. The 5 mL of culture was used to inoculate 50 mL of fresh minimal media, containing 6.8 g/L YNB (Difco) and 2 g/L glucose and grown overnight at 30° C., 200 rpm. This culture was then used to inoculate 500 mL of 6.8 g/L YNB (Difco) media, 2 g/L glucose and grown in a 1 liter fermenter. Temperature was maintained at 30° C., a single impeller was run at 700 rpm, and sparge rate was set to 1 vessel volumes per minute (VVM) using compressed air. The cultures were grown overnight for 21h to allow for glucose consumption prior to starting the fed-batch phase. A feed, containing 68 g/L YNB and 400 g/L of glucose was initiated by setting the fermenter feed to cycle on every 980s for 5s. 200 uL samples were taken daily and analyzed for growth (0D600) and production of malonic acid (as described in Example 36). Foaming was controlled by addition of 200 uL of antifoam at every sampling interval. pH was measured and adjusted to 4.5 with 10N NaOH at every sampling interval. Production of malonic acid continued beyond 111 h, accumulating 36.4 mM of malonic acid. Importantly, this is the first demonstration of production without addition of amino acids, uracil, or adenine. The 0D600, malonate concentration, and pH from each time point was as follows: 18h, OD=2.1, 0.7 mM, pH=2.3; 45h, OD=12.2, 8.8 mM, pH=2.0; 69h, OD=12.8, 18.1 mM, pH=2.0; 88.5h, OD=27.6, 22.9 mM, pH=2.7; 111.8h, OD=40.1, 36.4 mM, pH=2.4; 194h, OD=60.6, 94 mM, pH=3.3.

While the titer of malonate was higher from LYM007, the conditions under which LPK3003 can be grown (minimal media, low pH) and the fact that it contains only a single copy of F6AA82-2 malonyl-CoA hydrolase make this a superior strain for additional engineering efforts.

SEQUENCE LISTING

```
SEQ ID NO: 1.
Wild type Saccharomyces cerevisiae EHD3 3-hydroxypropionyl-CoA
hydrolase amino acid sequence.
      1- MLRNTLKCAQ LSSKYGFKTT TRTFMTTQPQ LNVTDAPPVL
    41- FTVQDTARVI TLNRPKKLNA LNAEMSESMF KTLNEYAKSD
    81- TTNLVILKSS NRPRSFCAGG DVATVAIFNF NKEFAKSIKF
  121- FTDEYSLNFQ IATYLKPIVT FMDGITMGGG VGLSIHTPFR
  161- IATENTKWAM PEMDIGFFPD VGSTFALPRI VTLANSNSQM
  201- ALYLCLTGEV VTGADAYMLG LASHYVSSEN LDALQKRLGE
  241- ISPPFNNDPQ SAYFFGMVNE SIDEFVSPLP KDYVFKYSNE
  281- KLNVIEACFN LSKNGTIEDI MNNLRQYEGS AEGKAFAQEI
  321- KTKLLTKSPS SLQIALRLVQ ENSRDHIESA IKRDLYTAAN
  361- MCMNQDSLVE FSEATKHKLI DKQRVPYPWT KKEQLFVSQL
  401- TSITSPKPSL PMSLLRNTSN VTWTQYPYHS KYQLPTEQEI
  441- AAYIEKRTND DTGAKVTERE VLNHFANVIP SRRGKLGIQS
  481- LCKIVCERKC EEVNDGLRWK -500
SEQ ID NO: 2.
Wild type Haemophilus influenzae nucleic acid sequence
encoding YciA acyl-CoA hydrolase.
      1- ATGTTTTACA CTGAAACTTA TGATGTGATT GTGATCGGTG
    41- GTGGTCATGC GGGTACAGAA GCCGCACTTG CACCAGCTCG
    81- TATGGGATTT AAAACCCTTT TATTAACACA TAATGTAGAT
  121- ACTTTAGGGC AAATGTCTTG TAACCCTGCA ATTGGTGGGA
  161- TCGGTAAAGG TCATTTAGTA AAAGAAGTAG ATGCAATGGG
  201- CGGTTTAATG GCGCATGCTG CAGATAAAGC AGGGATCCAA
  241- TTTCGTACTT TAAATAGCAG TAAAGGCCCA GCAGTGCGTG
  281- CTACTCGAGC TCAAGCTGAC AGAGTTCTAT ATCGTCAAGC
  321- TGTTCGTACT GCATTAGAAA ATCAACCTAA TTTAGATATT
  361- TTCCAACAAG AAGCGACCGA TATTCTGATT AAGCAAGATC
  401- GAGTTACAGG CGTTAGCACA AAAATGGGAT TAACTTTTCG
  441- TGCTAAATCA GTGGTATTAA CTGCGGGTAC TTTCTTAGCT
  481- GGTAAAATTC ATATTGGTTT GGAAAATTAT GAAGGTGGCC
  521- GTGCAGGGGA TCCTGCTTCT GTAAATCTTT CACATCGATT
  561- AAGAGATCTC GGATTACGTG TAGATCGCCT TAAAACAGGT
  601- ACACCGCCGC GTATTGATGC ACGTACGATC AATTTTGATA
  641- TTTTAGCTAA ACAACACGGT GATGCTGTTT TACCTGTGTT
  681- TTCTTTTATG GGATCAGTTG ATGATCACCC TCAACAAATT
  721- CCTTGTTATA TAACTCATAC CAATGAACAA ACCCATGAAG
  761- TGATCCGTAA TAACTTGGAT CGCAGTCCAA TGTATACTGG
  801- TGTGATTGAA GGGATCGGTC CACGTTATTG CCCATCCATT
  841- GAAGATAAAG TGATGCGTTT CTCGGATCGT AATTCACATC
  881- AAATTTATTT AGAACCAGAA GGCTTAACCA GTAATGAAGT
  921- GTATCCAAAC GGGATCTCTA CCAGTTTACC GTTTGACGTG
  961- CAAATGGGCA TTGTGAATTC TATGAAAGGT TTAGAAAACG
      -1000
SEQ ID NO: 3
Acinetobacter calcoaceticus MdcY malonate-binding
transcription factor amino acid sequence.
      1- MNSIAELPLS IQISKKLEDD IIYGFYLPGT KLDEQELCER
    41- YGASRTPIRE ALKLLAAEGL VEIRPRRGAI IPTINPLTLC
    81- EMFEVMAELE AMCGRLAARR IQPEEKLELQ RLHQLCQDYL
  121- NQNDSENYYE ANRLFHFAIY QASHNAFLIE QACTLHKRLH
  161- PYRRLQLRVN NRMNNSFTEH NEILEAIFAG NEQQAEALLK
  201- AHVVIQGQKF TDFISTIESL QPKS -224
SEQ ID NO: 4.
Rhizobium leguminosarum MatR malonate-binding
transcription factor amino acid sequence.
      1- MRKVKRMSEN VGRWLRDEIE NSILSNEFSP GERLDETVLA
    41- TRFGVSRTPV REALMOLDAI GLIEIRPRRG AIVIDPGPHR
    81- VYEMFEVMAE LEGLAGSLAA RRLDKTSREA ITATHGRCEK
  121- SAAAGDSDAY YYDNEEFHKA IYAAGRSDFL EEQCLQLHRR
  161- LRPDRRLQLR VRNRLSTSFL EHCAIVDAIF AGDGDEARRL
  201- LRGHVGIQGE RFSDLVASMA AR -222
SEQ ID NO: 5.
Klebsiella pneumoniae MauR malonate-binding
transcription factor amino acid sequence.
      1- MKDDINQEIT FRKLSVFMMF MAKGNIARTA EAMKLSSVSV
    41- HRALHTLEEG VGCPLFVHKG RNLLPLQAAW TLLEYCQDVI
    81- SLMNRGLEAT RKVAGVGQGR LRIGTLYSLT LETVPRIIMG
  121- MKLRRPELEL DLTMGSNQML LDMLEDDALD AILIATNEGE
  161- FNNTAFDVVP LFEDDIFLAA PATERLDASR LADLRDYADR
  201- KFVSLAEGFA TYAGFREAFH IAGFEPEIVT RVNDIFSMIS
  241- LVQAGVGFAL LPGRMKKVYE KDVQLLKLAE PYQMROLISI
  281- VYSHHRERDA DLLALAAEGR MYARSINR -308
SEQ ID NO: 6. MdcY regulated PMdeL promoter from
Acinetobacter calcoaceticus.
      1- AAAAAAATTG TATACAATTT ATGTTTATTT GAGTACAAAG
    41- CATTGTACAC TGAATACAGA TAGGCTATAA CTATACC -77
SEQ ID NO: 7.
EHD3 EC 3.1.2.4 malonyl-CoA hydrolase
consensus sequence.
      1- −−−−−−−−−− −−−−−−MOBU UOB+AQ+UBB +−−−−+−−−−
    41- +GFOBBOB−− −−+−−−−−+B UOUUU−−−− −−−−−−−−−−
    81- −−−−−−−−−− AQON+++UUU VOFUOQJUAR OOULNRPBKL
  121- NALNUJMUJU OFBULNEYUK SJUUNOOOOB SUNQPRUOCA
  161- GGDVAUOAO+ NOJBBF−−BB UOJFFBUX₁YS ONFQOATYOK
  201- POOOOMJGIT MGGGVGOUOH UPFROATENT BWAMPEMDIG
  241- FFPDVGUUFA OPBOOUOANU BUQOAOYLCO TGJOOUGJJA
  281- YOOGOASHYO UBJNOJJLJB RLGEOBPUJ+ OJ+−+++UQU
  321- JJFFJOONJU OJEFUUP−OP BJYBFBYUNJ BLJVIJBCFJ
  361- OUBOUUOBJO OBBLJJO−++ −YJGUJJABJ FABJOBJBLO
  401- UKSPUSOQOA OBOOBJNUBJ BOJUAOBBDL OTAUNMCON−
  441- +++QJUOOEF UJAUBBKLOJ KQBOPYPWBB B+JJOUOUQO
  481- UUOOUPBPUO POULOBNUUN OTWBJYPBBO BYQLPUJUJO
  521- BQYOBBBJNB N−−−+G−++O BOUBBJOOBB FUNONJUBBJ
  561- KOGOJUOOBO OOJBBCUJJJ A+GGOBWB++ −+−−−−−− -598
SEQ ID NO: 8.
Bacillus EC 3.1.2.4 malonyl-CoA hydrolase consensus sequence.
      1- MTEBVLFSOU JNGVAUITLN RPKALNSLSY JMLQPIGQKL
    41- KEWEBJJBIA OIVLKGAGUK GFCAGGDIKT LYEARSNEOA
    81- LQBAEBFFJE X₁YJIDTYOYQ YBKPIIACLD GIVMGGGVGL
  121- TNGABYRIVT JBTKWAMPEM NIGFFPDVGA AYFLNBAPGY
  161- UGBYVALUAU OLKAUDVLFI NAADYFMUUJ ULPBFLUJOJ
  201- UONWBBJJJV BUBLKJOOBU FAUUUUOJUJ LUUOOEJONU
  241- HFAFJUOEJI IBSLEBJQUU FAOBUBJULL SKSPOSLKVT
  281- LKQFOJGBJK SOEJCFATDL OLAKNFMRHJ DFFEGVRSOV
  321- ODKDQNPNYK YBQOUDVUJJ JVNBFFNLLN A -351
SEQ ID NO: 9.
Pseudomonas EC 3.1.2.4 malonyl-CoA hydrolase consensus
sequence.
      1- MNOBFEJBUU OBGARIGOAU LDAJBULNAL ULPMIJOLGJ
    41- BOBAWABJPG OOCVOLRGNG AKAFCAGGJV BBLOJACBJB
    81- PGJOPPLAAB FFAJX₁YRLJB BOHUYPKPOO CWGHGBVOGG
  121- GMGLOQGAUO RIVTPUURLA MPEOUIGLYP DVGASWFLUR
  161- OPGBLGLFOG LUGABONABD AODLJLADRF OBJBQQJJLO
  201- JJLOQONWQE QUJOQLBSLO BAJJBBABJJ OPJAQOLPRR
  241- QBODJOLDOA JOAUAWBAOJ AOBJBJDPLO ABAABBOBJG
  281- CPOUABOVWJ QOBRARBLSL AJOFBMEYUO SLNCCRHPJF
  321- UEGVRARLOD BDBQPBWBWP JOAQOPJAOO JAHFJBOWJG
  361- BBPOAJOU++
SEQ ID NO: 10. General bacterial EC 3.1.2.4 malonyl-CoA
hydrolase consensus sequence.
      1- −−−−−−−−−− +−−−−−−−−− −−−+−−−−−− M+M−−TEHOO
    41- FUOSENGOAS IULNRPBALN SLUYDMOQPO GQBOBEWENJ
    81- EROALOOLB− GAGTBGFCAG GJOBUOYJAR SNEPG+ALQH
  121- AERFFEJX₁YE OJTYOYQYKK POOACLDGIO MGGGVGLTNG
  161- AKYBOOTERU BWAMPEMNIG FFPDVGAAYF LNBA−−−−−−
  201- PGYLGRYOAL UASIOKASDV OFONAAJYFM TSJSLPAFOT
  241- EOESONWHKE DJOHTHLLKE +−+VORTFAT APNLJSEOAP
  281- −−−−SLEEON SHFAF−−−DT OEEIW+AOHS OE−−KJQSSF
  321- ALKTKETOLS KUPOULBOTL KQFIDGRDKU OEJCFATJLV
  361- OAKNFMBB−− −−EJFFEGOB SVOODBJQNP NYBYKQOSDO
  401- SJED−−−−−− −−−−−−−−−O NRFFNLONAG +H−−PLADL+
  441- −−−−−−++−− −−−−−−−−−− −−−−−−−−−− −−−−−−−−−−
  481- −−−−−−−−−− −−−−−−−−−− −−−−− -505
SEQ ID NO: 11. YIACC codon optimized DNA sequence
      1 ATGAGATTGC AGTTGAGAAC CTTGACTAGA AGGTTTTTCT CTATGGCTTC TGGTTCTTCT
    61 ACTCCAGATG TTGCTCCATT GGTTGATCCA AATATTCATA AGGGTTTGGC CTCTCACTTT
  121 TTCGGTTTGA ACTCTGTTCA TACTGCTAAG CCATCTAAGG TCAAAGAATT TGTTGCTTCT
  181 CATGGTGGTC ATACCGTTAT CAACAAAGTT TTGATTGCCA ACAACGGTAT TGCTGCCGTA
  241 AAAGAAATTA GGTCTGTTAG AAAATGGGCC TACGAAACTT TTGGTGACGA AAGAGCTATT
  301 TCCTTCACTG TTATGGCTAC ACCTGAAGAT TTGGCTGCTA ATGCTGATTA CATTAGAATG
  361 GCCGATCAAT ACGTTGAAGT TCCAGGTGGT ACAAACAACA ACAATTACGC TAACGTTGAA
  421 TTGATCGTTG ATGTCGCTGA AAGATTTGGT GTTGATGCTG TTTGGGCTGG TTGGGGTCAT
  481 GCTTCTGAAA ATCCATTATT GCCAGAATCT TTGGCTGCTT CTCCAAGAAA GATCGTTTTT
  541 ATTGGTCCAC CAGGTGCTGC TATGAGATCA TTGGGTGATA AGATTTCTTC TACCATCGTT
  601 GCTCAACATG CTAAGGTTCC ATGTATTCCA TGGTCTGGTA CTGGTGTTGA TGAAGTTGTT
  661 GTTGATAAGT CCACCAACTT GGTTTCCGTT TCTGAAGAAG TTTACACTAA GGGTTGTACT
  721 ACTGGTCCAA AACAAGGTTT GGAAAAGGCT AAGCAAATTG GTTTCCCAGT TATGATTAAG
  781 GCTTCTGAAG GTGGTGGTGG TAAAGGTATT AGAAAGGTTG AAAGGGAAGA AGATTTCGAA
  841 GCTGCTTACC ATCAAGTTGA AGGTGAAATT CCAGGTTCCC CAATTTTCAT TATGCAATTG
  901 GCTGGTAACG CCAGACATTT GGAAGTTCAA TTATTGGCTG ATCAGTACGG CAACAACATT
  961 TCTTTGTTTG GTAGAGATTG CTCCGTTCAA AGAAGGCATC AAAAGATTAT TGAAGAAGCC
1021 CCAGTTACTG TTGCTGGTCA ACAAACTTTT ACTGCTATGG AAAAAGCTGC CGTCAGATTG
1081 GGTAAATTGG TTGGTTATGT TTCTGCCGGT ACTGTCGAAT ACTTGTACTC TCATGAAGAT
1141 GACAAGTTCT ACTTCTTGGA ATTGAACCCA AGATTGCAAG TTGAACATCC AACTACTGAA
1201 ATGGTTACCG GTGTTAATTT GCCAGCTGCT CAATTGCAAA TTGCTATGGG TATTCCATTG
1261 GACAGAATCA AGGATATTAG GTTGTTCTAC GGTGTTAACC CACATACCAC TACTCCAATT
1321 GATTTCGATT TCTCTGGTGA AGATGCTGAC AAAACTCAAA GAAGGCCAGT TCCAAGAGGT
1381 CATACAACTG CTTGTAGAAT TACTTCTGAA GATCCAGGTG AAGGTTTTAA ACCATCTGGT
1441 GGTACTATGC ACGAATTGAA CTTTAGGTCC TCTTCTAATG TTTGGGGTTA CTTCTCTGTT
1501 GGTAATCAAG GTGGTATCCA CTCTTTCTCT GATTCTCAAT TCGGTCATAT TTTCGCCTTT
1561 GGTGAAAACA GATCCGCCTC AAGAAAACAT ATGGTTGTTG CCTTGAAAGA ATTGTCCATC
1621 AGAGGTGATT TCAGAACCAC TGTTGAATAC TTGATCAAAT TATTGGAAAC CCCAGACTTC
1681 GAGGATAACA CTATTACTAC TGGTTGGTTG GACGAGTTGA TCTCTAACAA ATTGACTGCT
1741 GAAAGACCAG ATTCCTTCTT GGCTGTTGTT TGTGGTGCTG CTACAAAAGC TCATAGAGCT
1801 TCAGAAGATT CTATCGCTAC TTACATGGCT TCTTTGGAAA AAGGTCAAGT TCCAGCCAGA
1861 GACATTTTGA AAACTTTGTT CCCAGTTGAC TTCATCTACG AAGGTCAAAG ATACAAGTTT
1921 ACCGCTACCA GATCATCCGA AGATTCTTAC ACTTTGTTCA TCAACGGTTC CAGATGCGAT
1981 ATTGGTGTTA GACCATTGTC TGATGGTGGT ATTTTGTGTT TGGTTGGTGG TAGATCCCAT
2041 AACGTTTATT GGAAAGAAGA AGTTGGCGCT ACCAGATTGT CTGTTGATTC TAAAACTTGC
2101 TTGTTGGAAG TCGAAAACGA TCCAACTCAA TTGAGATCAC CATCTCCAGG TAAGTTGGTT
2161 AAGTTCTTGG TTGAAAACGG TGATCACGTT AGAGCTAATC AACCATACGC TGAAATCGAA
2221 GTCATGAAGA TGTACATGAC TTTGACCGCT CAAGAAGATG GTATCGTTCA ATTGATGAAG
2281 CAACCAGGTT CTACTATTGA AGCCGGTGAT ATTTTGGGTA TTTTGGCTTT GGATGATCCA
2341 TCCAAGGTCA AACATGCTAA ACCATTTGAA GGTCAGTTGC CAGAATTGGG TCCACCAACA
2401 TTGTCTGGTA ACAAACCACA TCAAAGATAC GAACATTGCC AAAACGTCTT GCACAACATT
2461 TTGTTGGGTT TCGATAACCA GGTTGTCATG AAGTCTACAT TGCAAGAAAT GGTCGGTTTG
2521 TTGAGAAATC CAGAATTGCC ATACTTGCAA TGGGCTCATC AAGTTTCTTC ATTGCATACA
2581 AGAATGTCCG CTAAGTTGGA TGCTACTTTG GCTGGTTTGA TTGATAAGGC TAAACAAAGG
2641 GGTGGTGAAT TTCCAGCTAA GCAATTATTG AGAGCCTTGG AAAAAGAAGC TTCATCTGGC
2701 GAAGTTGATG CTTTGTTTCA ACAAACATTG GCCCCTTTGT TTGATTTGGC TAGAGAATAT
2761 CAAGATGGTT TGGCCATCCA TGAATTGCAA GTTGCTGCTG GTTTGTTGCA AGCTTATTAT
2821 GATTCTGAAG CTAGATTCTG CGGTCCAAAC GTTAGAGATG AAGATGTTAT CTTGAAGTTG
2881 AGGGAAGAAA ACAGGGACTC TTTGAGAAAA GTTGTTATGG CCCAATTGTC CCATTCAAGA
2941 GTTGGTGCTA AAAACAACTT GGTTTTGGCT TTGTTGGATG AGTACAAGGT TGCTGATCAA
3001 GCTGGTACTG ATTCTCCAGC TTCTAATGTT CATGTTGCTA AATACTTGAG GCCAGTCTTG
3061 AGAAAGATTG TCGAATTGGA ATCAAGAGCT TCCGCTAAGG TTTCTTTGAA GGCTAGAGAA
3121 ATTTTGATCC AATGCGCTTT GCCATCCTTG AAAGAAAGAA CTGATCAATT GGAACACATC
3181 TTGAGATCCT CTGTTGTTGA ATCAAGATAC GGTGAAGTTG GTTTGGAACA TAGAACTCCA
3241 AGAGCTGACA TCTTGAAAGA AGTTGTTGAC TCCAAGTACA TCGTGTTCGA TGTTTTGGCT
3301 CAATTCTTCG CTCATGATGA TCCATGGATA GTTTTGGCTG CTTTGGAGTT GTATATTAGA
3361 AGGGCTTGTA AGGCCTACTC CATTTTGGAT ATTAACTACC ACCAAGACTC TGATTTGCCA
3421 CCAGTTATTT CTTGGAGATT CAGATTGCCA ACTATGTCAT CTGCCTTGTA CAACTCTGTT
3481 GTTTCTTCTG GTTCTAAGAC TCCAACTTCT CCATCTGTTT CAAGAGCTGA TTCTGTTTCC
3541 GATTTCTCTT ACACCGTTGA AAGAGATTCT GCTCCAGCTA GAACTGGTGC TATAGTTGCT
3601 GTTCCACATT TGGATGATTT GGAAGATGCT TTGACCAGAG TCTTGGAAAA TTTGCCAAAA
3661 AGAGGTGCTG GTTTGGCTAT TTCTGTTGGT GCTTCTAACA AATCAGCTGC TGCTTCTGCT
3721 AGAGATGCTG CTGCTGCAGC TGCTTCTTCT GTTGATACTG GTTTGTCTAA CATCTGCAAC
3781 GTTATGATCG GTAGAGTTGA TGAATCCGAT GATGATGATA CCTTGATCGC CAGAATTTCC
3841 CAAGTTATCG AAGATTTCAA AGAGGACTTC GAAGCTTGCT CCTTGAGAAG AATTACTTTC
3901 TCATTCGGTA ACTCCAGAGG TACTTACCCA AAGTACTTTA CTTTTAGAGG TCCAGCCTAT
3961 GAAGAAGATC CAACCATTAG ACATATTGAA CCAGCTTTGG CCTTTCAATT GGAATTGGCT
4021 AGATTGTCTA ACTTCGACAT CAAGCCAGTT CATACCGATA ACAGAAACAT CCATGTTTAC
4081 GAAGCTACTG GTAAGAATGC TGCTTCCGAT AAGAGATTTT TCACCAGAGG TATAGTTAGA
4141 CCAGGTAGAT TGAGAGAAAA CATCCCAACA TCCGAGTACT TGATTTCTGA AGCTGATAGA
4201 TTGATGTCCG ATATTTTGGA TGCCTTGGAA GTTATTGGTA CTACCAACTC TGATTTGAAC
4261 CACATCTTCA TTAACTTCTC CGCTGTTTTT GCTTTGAAGC CAGAAGAAGT TGAAGCTGCT
4321 TTTGGTGGTT TTTTGGAAAG ATTCGGTAGA AGATTGTGGA GATTGAGAGT TACTGGTGCC
4381 GAAATTAGAA TGATGGTTTC TGATCCAGAA ACTGGTTCTG CTTTTCCATT GAGAGCTATG
4441 ATCAACAACG TTTCCGGTTA CGTTGTCCAA TCTGAATTAT ACGCTGAAGC CAAGAATGAT
4501 AAGGGTCAAT GGATCTTTAA GTCCTTGGGT AAACCAGGTT CAATGCATAT GAGATCCATT
4561 AACACTCCAT ACCCTACCAA AGAATGGTTG CAACCTAAAA GATACAAGGC CCATTTGATG
4621 GGTACTACCT ACTGTTATGA TTTCCCAGAG TTGTTCAGAC AGTCCATTGA ATCTGATTGG
4681 AAAAAGTACG ATGGTAAGGC TCCAGATGAT TTGATGACTT GCAACGAATT GATCTTGGAC
4741 GAAGATTCTG GTGAATTGCA AGAAGTTAAT AGAGAACCAG GTGCTAACAA CGTTGGTATG
4801 GTTGCTTGGA AATTTGAAGC TAAGACTCCA GAATATCCAA GGGGTAGAAG TTTTATCGTT
4861 GTTGCCAACG ATATTACCTT CCAGATTGGT TCTTTTGGTC CAGCTGAAGA TCAATTCTTC
4921 TTCAAGGTTA CTGAATTGGC CAGAAAGTTG GGTATTCCAA GAATCTACTT GTCTGCTAAT
4981 TCCGGTGCTA GAATTGGTAT TGCTGATGAA TTGGTCGGTA AGTACAAAGT TGCTTGGAAT
5041 GACGAAACTG ATCCATCTAA GGGTTTCAAG TACTTGTACT TCACTCCAGA ATCATTGGCT
5101 ACCTTGAAAC CAGATACTGT TGTTACCACC GAAATTGAAG AAGAAGGTCC AAACGGCGTT
5161 GAAAAGAGAC ATGTTATTGA TTACATCGTC GGTGAAAAGG ATGGTTTGGG TGTTGAATGT
5221 TTGAGAGGTT CTGGTTTAAT TGCTGGTGCT ACTTCAAGAG CTTACAAGGA TATTTTCACC
5281 TTGACCTTGG TTACCTGTAG ATCAGTTGGT ATTGGTGCTT ACTTGGTTAG ATTGGGTCAA
5341 AGAGCCATTC AAATTGAAGG TCAGCCAATT ATCTTGACTG GTGCTCCAGC TATTAACAAG
5401 TTGTTGGGTA GAGAAGTCTA CTCCTCTAAC TTGCAATTGG GTGGTACTCA AATCATGTAC
5461 AACAACGGTG TTTCTCATTT GACCGCTAGA GATGATTTGA ACGGTGTTCA TAAGATCATG
5521 CAGTGGTTGT CTTATATTCC AGCTTCAAGA GGTTTGCCAG TTCCAGTTTT GCCACATAAG
5581 ACTGATGTTT GGGATAGAGA TGTTACCTTC CAACCAGTTA GAGGTGAACA ATATGATGTC
5641 AGATGGTTGA TTTCTGGTAG GACTTTGGAA GATGGTGCTT TTGAATCTGG TTTGTTCGAT
5701 AAGGACTCTT TCCAAGAAAC TTTATCTGGT TGGGCTAAGG GTGTTGTTGT TGGTAGAGCT
5761 AGATTGGGTG GTATTCCATT TGGTGTTATT GGTGTTGAAA CTGCCACTGT TGATAACACT
5821 ACTCCAGCTG ATCCAGCTAA TCCAGATTCT ATTGAAATGT CTACTTCCGA AGCTGGTCAA
5881 GTTTGGTATC CAAATTCTGC TTTCAAGACC TCCCAAGCCA TTAACGATTT TAATCATGGT
5941 GAAGCCTTGC CATTGATGAT TTTGGCTAAT TGGAGAGGTT TTTCCGGTGG TCAAAGAGAT
6001 ATGTACAACG AAGTTTTGAA GTACGGCTCC TTTATTGTCG ATGCTTTGGT TGATTACAAG
6061 CAGCCAATTA TGGTTTACAT TCCACCAACT GGTGAATTGA GAGGTGGTTC TTGGGTTGTT
6121 GTTGACCCAA CTATTAACTC CGATATGATG GAAATGTACG CCGATGTTGA AAGTAGAGGT
6181 GGTGTTTTGG AACCAGAAGG TATGGTTGGT ATTAAGTACA GAAGAGACAA GTTGTTAGAT
6241 ACCATGGCCA GATTAGATCC AGAGTACTCT TCCTTGAAAA AACAATTGGA AGAGTCCCCA
6301 GACTCCGAAG AATTGAAAGT TAAGTTGTCC GTCAGGGAAA AGTCTTTGAT GCCAATCTAC
6361 CAACAAATCT CCGTTCAATT TGCTGACTTG CATGATAGAG CTGGTAGAAT GGAAGCTAAA
6421 GGTGTTATTA GAGAAGCCTT GGTTTGGAAG GATGCTAGAA GATTTTTCTT TTGGAGGATC
6481 AGAAGAAGGT TGGTCGAGGA ATATTTGATC ACCAAGATCA ACTCCATTTT GCCATCTTGT
6541 ACCAGATTGG AATGTTTGGC TAGAATCAAA TCTTGGAAGC CAGCTACTTT GGATCAAGGT
6601 TCTGATAGAG GTGTTGCTGA ATGGTTTGAC GAAAATTCTG ATGCTGTTTC TGCCAGGTTG
6661 TCTGAATTGA AAAAAGATGC TTCTGCTCAG TCCTTCGCTT CCCAATTGAG AAAAGATAGA
6721 CAAGGTACAT TGCAGGGTAT GAAGCAAGCT TTGGCTTCTT TGTCTGAAGC TGAAAGAGCT
6781 GAATTATTGA AGGGCTTGTA A
SEQ ID NO: 12. Promoter study base plasmid
      1 tcgcgcgttt cggtgatgac ggtgaaaacc tctgacacat gcagctcccg gagacggtca
    61 cagcttgtct gtaagcggat gccgggagca gacaagcccg tcagggcgcg tcagcgggtg
  121 ttggcgggtg tcggggctgg cttaactatg cggcatcaga gcagattgta ctgagagtgc
  181 accacgcttt tcaattcaat tcatcatttt ttttttattc ttttttttga tttcggtttc
  241 tttgaaattt ttttgattcg gtaatctccg aacagaagga agaacgaagg aaggagcaca
  301 gacttagatt ggtatatata cgcatatgta gtgttgaaga aacatgaaat tgcccagtat
  361 tcttaaccca actgcacaga acaaaaacct gcaggaaacg aagataaatc atgtcgaaag
  421 ctacatataa ggaacgtgct gctactcatc ctagtcctgt tgctgccaag ctatttaata
  481 tcatgcacga aaagcaaaca aacttgtgtg cttcattgga tgttcgtacc accaaggaat
  541 tactggagtt agttgaagca ttaggtccca aaatttgttt actaaaaaca catgtggata
  601 tcttgactga tttttccatg gagggcacag ttaagccgct aaaggcatta tccgccaagt
  661 acaatttttt actcttcgaa gacagaaaat ttgctgacat tggtaataca gtcaaattgc
  721 agtactctgc gggtgtatac agaatagcag aatgggcaga cattacgaat gcacacggtg
  781 tggtgggccc aggtattgtt agcggtttga agcaggcggc agaagaagta acaaaggaac
  841 ctagaggcct tttgatgtta gcagaattgt catgcaaggg ctccctatct actggagaat
  901 atactaaggg tactgttgac attgcgaaga gcgacaaaga ttttgttatc ggctttattg
  961 ctcaaagaga catgggtgga agagatgaag gttacgattg gttgattatg acacccggtg
1021 tgggtttaga tgacaaggga gacgcattgg gtcaacagta tagaaccgtg gatgatgtgg
1081 tctctacagg atctgacatt attattgttg gaagaggact atttgcaaag ggaagggatg
1141 ctaaggtaga gggtgaacgt tacagaaaag caggctggga agcatatttg agaagatgcg
1201 gccagcaaaa ctaaaaaact gtattataag taaatgcatg tatactaaac tcacaaatta
1261 gagcttcaat ttaattatat cagttattac cctgcggtgt gaaataccgc acagatgcgt
1321 aaggagaaaa taccgcatca ggaaattgta aacgttaata ttttgttaaa attcgcgtta
1381 aatttttgtt aaatcagctc attttttaac caataggccg aaatcggcaa aatcccttat
1441 aaatcaaaag aatagaccga gatagggttg agtgttgttc cagtttggaa caagagtcca
1501 ctattaaaga acgtggactc caacgtcaaa gggcgaaaaa ccgtctatca gggcgatggc
1561 ccactacgtg aaccatcacc ctaatcaagt tttttggggt cgaggtgccg taaagcacta
1621 aatcggaacc ctaaagggag cccccgattt agagcttgac ggggaaagcc ggcgaacgtg
1681 gcgagaaagg aagggaagaa agcgaaagga gcgggcgcta gggcgctggc aagtgtagcg
1741 gtcacgctgc gcgtaaccac cacacccgcc gcgcttaatg cgccgctaca gggcgcgtcg
1801 cgccattcgc cattcaggct gcgcaactgt tgggaagggc gatcggtgcg ggcctcttcg
1861 ctattacgcc agctggcgaa ggggggatgt gctgcaaggc gattaagttg ggtaacgcca
1921 gggttttccc agtcacgacg ttgtaaaacg acggccagtg aattgtaata cgactcacta
1981 tagggcgaat tggagctcca ccgcggtggc ggccgcatag gccactagtg gatctgatat
2041 catcgatgaa ttcgagctcg ttttcgacac tggatggcgg cgttagtatc gaatcgacag
2101 cagtatagcg accagcattc acatacgatt gacgcatgat attactttct gcgcacttaa
2161 cttcgcatct gggcagatga tgtcgaggcg aaaaaaaata taaatcacgc taacatttga
2221 ttaaaataga acaactacaa tataaaaaaa ctatacaaat gacaagttct tgaaaacaag
2281 aatcttttta ttgtcagtac tgattattcc tttgccctcg gacgagtgct ggggcgtcgg
2341 tttccactat cggcgagtac ttctacacag ccatcggtcc agacggccgc gcttctgcgg
2401 gcgatttgtg tacgcccgac agtcccggct ccggatcgga cgattgcgtc gcatcgaccc
2461 tgcgcccaag ctgcatcatc gaaattgccg tcaaccaagc tctgatagag ttggtcaaga
2521 ccaatgcgga gcatatacgc ccggagccgc ggcgatcctg caagctccgg atgcctccgc
2581 tcgaagtagc gcgtctgctg ctccatacaa gccaaccacg gcctccagaa gaagatgttg
2641 gcgacctcgt attgggaatc cccgaacatc gcctcgctcc agtcaatgac cgctgttatg
2701 cggccattgt ccgtcaggac attgttggag ccgaaatccg cgtgcacgag gtgccggact
2761 tcggggcagt cctcggccca aagcatcage tcatcgagag cctgcgcgac ggacgcactg
2821 acggtgtcgt ccatcacagt ttgccagtga tacacatggg gatcagcaat cgcgcatatg
2881 aaatcacgcc atgtagtgta ttgaccgatt ccttgcggtc cgaatgggcc gaacccgctc
2941 gtctggctaa gatcggccgc agcgatcgca tccatggcct ccgcgaccgg ctgcagaaca
3001 gcgggcagtt cggtttcagg caggtcttgc aacgtgacac cctgtgcacg gcgggagatg
3061 caataggtca ggctctcgct gaattcccca atgtcaagca cttccggaat cgggagcgcg
3121 gccgatgcaa agtgccgata aacataacga tctttgtaga aaccatcggc gcagctattt
3181 acccgcagga catatccacg ccctcctaca tcgaagctga aagcacgaga ttcttcgccc
3241 tccgagagct gcatcaggtc ggagacgctg tcgaactttt cgatcagaaa cttctcgaca
3301 gacgtcgcgg tgagttcagg ctttttaccc atggttgttt atgttcggat gtgatgtgag
3361 aactgtatcc tagcaagatt ttaaaaggaa gtatatgaaa gaagaacctc agtggcaaat
3421 cctaaccttt tatatttctc tacaggggcg cggcgtgggg acaattcaac gcgtctgtga
3481 ggggagcgtt tccctgctcg caggtctgca gcgaggagcc gtaatttttg cttcgcgccg
3541 tgcggccatc aaaatgtatg gatgcaaatg attatacatg gggatgtatg ggctaaatgt
3601 acgggcgaca gtcacatcat gcccctgagc tgcgcacgtc aagactgtca aggagggtat
3661 tctgggcctc catgtcgctg gccgggtgac ccggcgggga cGaggcaagc taaacagatc
3721 tCCCGGGtaG TTTaaacaAT GAATGTCACC TTTGAAGAAA GAGCCAGTTT ACACGGTTAC
3781 AGAATCGGTA TCGCATCCTT AGACGCCCCA GCATCCTTGA ACGCTTTGTC CTTACCAATG
3841 ATTGATGCTT TACAAGACAG ATTGAGAGCT TGGGCAGAAG ATGCAGACAT AGCCTGTGTC
3901 TTGTTACGTG GTAATGGTTC TAAAGCATTT TGCGCAGGTG GTGACGTTGT CCAATTAGCT
3961 AAGAAATGTT TGGCCTCCCC AGGTGAAGCC CCTGAATTGG CTGAAAGATT TTTCGCTAGA
4021 AGTTACAGAT TAGATCATTA TTTGCACACA TACCCAAAGC CTTTGATATG CTGGGCTCAT
4081 GGTCACGTTT TAGGTGGTGG TATGGGTTTG TTACAAGGTG CTGGTATTAG AATAGTCACC
4141 CCATCTTCAA GATTGGCAAT GCCTGAAATC TCTATTGGTT TATTTCCAGA TGTTGGTGGT
4201 TCCCATTTCT TAAGTAGATT GCCTGGTAAA TTGGGTTTAT TTTTCGGTTT AACCGCTTCA
4261 CCATTGAATG CCAGAGATGC TTTGGACTTA AACTTGGCTG ATAGATTCTT GTTAGATACT
4321 CAACAAGACG CATTGATCGA TGGTTTGATC CAATTGAACT GGAGAGAACA ACCAGATTTG
4381 CAATTGCATT CCTTGTTAAA GGCTTTAGAA CAACAAGCAA GAAGTGAATT ACCAGCTGCA
4441 CAATGGTTGC CTAGAAGAGA AAGATTAGAC GCCTTGTTAG ATCAAGCTAC TTTACCATTG
4501 TCTTGGCAAG CCTTAGCTTC ATTGGAAAAC GATGAAGACG CATTGTTAGC CAAAGCCGCT
4561 AAGACTATGT TGGGTGGTTC ACCTTTAACA GGTCATTTGG TCTGGGGTCA AATTAGAAGA
4621 GCTAGACACT TATCTTTGGC ACAAGTATTT CAAATGGAAT ACGGCATGTC ATTGAATTGT
4681 TGCAGACATC CAGAATTTGC AGAAGGTGTA AGAGCCAGAT TAATCGATAA AGACCACGCT
4741 CCACATTGGC ACTGGCCTGA CGTAAACCAA GTTCCTGAAG CCGTTATTGC AGCCCATTTT
4801 GCACCATTAG ATGACCACCC TTTAGCTGAT TTGGCATAAg tttAAACtca tgtaattagt
4861 tatgtcacgc ttacattcac gccctccccc cacatccgct ctaaccgaaa aggaaggagt
4921 tagacaacct gaagtctagg tccctattta tttttttata gttatgttag tattaagaac
4981 gttatttata tttcaaattt ttcttttttt tctgtacaga cgcgtgtacg catgtaacat
5041 tatactgaaa accttgcttg agaaggtttt gggacgctcg aaggctttaa tttgcggcct
5101 ctagcagctt ttgttccctt tagtgagggt taattTcgag cttggcgtaa tcatggtcat
5161 agctgtttcc tgtgtgaaat tgttatccgc tcacaattcc acacaacata ggagccggaa
5221 gcataaagtg taaagcctgg ggtgcctaat gagtgaggta actcacatta attgcgttgc
5281 gctcactgcc cgctttccag tcgggaaacc tgtcgtgcca gctgcattaa tgaatcggcc
5341 aacgcgcggg gagaggcggt ttgcgtattg ggcgctcttc cgcttcctcg ctcactgact
5401 cgctgcgctc ggtcgttcgg ctgcggcgag cggtatcagc tcactcaaag gcggtaatac
5461 ggttatccac agaatcaggg gataacgcag gaaagaacat gtgagcaaaa ggccagcaaa
5521 aggccaggaa ccgtaaaaag gccgcgttgc tggcgttttt ccataggctc ggcccccctg
5581 acgagcatca caaaaatcga cgctcaagtc agaggtggcg aaacccgaca ggactataaa
5641 gataccaggc gttcccccct ggaagctccc tcgtgcgctc tcctgttccg accctgccgc
5701 ttaccggata cctgtccgcc tttctccctt cgggaagcgt ggcgctttct caatgctcac
5761 gctgtaggta tctcagttcg gtgtaggtcg ttcgctccaa gctgggctgt gtgcacgaac
5821 cccccgttca gcccgaccgc tgcgccttat ccggtaacta tcgtcttgag tccaacccgg
5881 taagacacga cttatcgcca ctggcagcag ccactggtaa caggattagc agagcgaggt
5941 atgtaggcgg tgctacagag ttcttgaagt ggtggcctaa ctacggctac actagaagga
6001 cagtatttgg tatctgcgct ctgctgaagc cagttacctt cggaaaaaga gttggtagct
6061 cttgatccgg caaacaaacc accgctggta gcggtggttt ttttgtttgc aagcagcaga
6121 ttacgcgcag aaaaaaagga tctcaagaag atcctttgat cttttctacg gggtctgacg
6181 ctcagtggaa cgaaaactca cgttaaggga ttttggtcat gagattatca aaaaggatct
6241 tcacctagat ccttttaaat taaaaatgaa gttttaaatc aatctaaagt atatatgagt
6301 aaacttggtc tgacagttac caatgcttaa tcagtgaggc acctatctca gcgatctgtc
6361 tatttcgttc atccatagtt gcctgactgc ccgtcgtgta gataactacg atacgggagg
6421 gcttaccatc tggccccagt gctgcaatga taccgcgaga cccacgctca ccggctccag
6481 atttatcagc aataaaccag ccagccggaa gggccgagcg cagaagtggt cctgcaactt
6541 tatccgcctc catccagtct attaattgtt gccgggaagc tagagtaagt agttcgccag
6601 ttaatagttt gcgcaacgtt gttgccattg ctacaggcat cgtggtgtca cgctcgtcgt
6661 ttggtatggc ttcattcagc tccggttccc aacgatcaag gcgagttaca tgatccccca
6721 tgttgtgaaa aaaagcggtt agctccttcg gtcctccgat cgttgtcaga agtaagttgg
6781 ccgcagtgtt atcactcatg gttatggcag cactgcataa ttctcttact gtcatgccat
6841 ccgtaagatg cttttctgtg actggtgagt actcaaccaa gtcattctga gaatagtgta
6901 tgcggcgacc gagttgctct tgcccggcgt caatacggga taataccgcg ccacatagca
6961 gaactttaaa agtgctcatc attggaaaac gttcttcggg gcgaaaactc tcaaggatct
7021 taccgctgtt gagatccagt tcgatgtaac ccactcgtgc acccaactga tottcagcat
7081 cttttacttt caccagcgtt tctgggtgag caaaaacagg aaggcaaaat gccgcaaaaa
7141 agggaataag ggcgacacgg aaatgttgaa tactcatact cttccttttt caatattatt
7201 gaagcattta tcagggttat tgtctcatga gcggatacat atttgaatgt atttagaaaa
7261 ataaacaaat aggggttccg cgcacatttc cccgaaaagt gccacctggg tccttttcat
7321 cacgtgctat aaaaataatt ataatttaaa ttttttaata taaatatata aattaaaaat
7381 agaaagtaaa aaaagaaatt aaagaaaaaa tagtttttgt tttccgaaga tgtaaaagac
7441 tctaggggga tcgccaacaa atactacctt ttatcttgct cttcctgctc tcaggtatta
7501 atgccgaatt gtttcatctt gtctgtgtag aagaccacac acgaaaatcc tgtgatttta
7561 cattttactt atcgttaatc gaatgtatat ctatttaatc tgcttttctt gtctaataaa
7621 tatatatgta aagtacgctt tttgttgaaa ttttttaaac ctttgtttat ttttttttct
7681 tcattccgta actcttctac cttctttatt tactttctaa aatccaaata caaaacataa
7741 aaataaataa acacagagta aattcccaaa ttattccatc attaaaagat acgaggcgcg
7801 tgtaagttac aggcaagcga tccgtcctaa gaaaccatta ttatcatgac attaacctat
7861 aaaaataggc gtatcacgag gccctttcgt c //
SEQ ID NO: 13. Primer A93
CCAATATATAATAAAATATGGAGGAATGCGATGCTCAGAAATACGCTAAAATGTGCCCAA
SEQ ID NO: 14. Primer A94
TGCCTGGAGATCCTTACTCGAGTTGGATCCTTATTTCCATCTTAAGCCATCGTTAACTTC
SEQ ID NO: 15. Primer A95
TTTTACTGATGCGTATTCTTTGAATTTTCAAATAGCA
SEQ ID NO: 16. Primer A96
TCAAAGAATACGCATCAGTAAAAAATTTGATGGA
SEQ ID NO: 17. Primer A97
TTTTACTGATGTTTATTCTTTGAATTTTCAAATAGCAACTT
SEQ ID NO: 18. Primer A98
TCAAAGAATAAACATCAGTAAAAAATTTGATGGACTTGG
SEQ ID NO: 19. Primer A99
TTTTACTGATTCGTATTCTTTGAATTTTCAAATAGCAAC
SEQ ID NO: 20. Primer A100
TCAAAGAATACGAATCAGTAAAAAATTTGATGGACT
SEQ ID NO: 21. Primer A120
CCAATATATAATAAAATATGGAGGAATGCGATGTCTACAACACATAACGTCCCTC
SEQ ID NO: 22. Primer A121
TGCCTGGAGATCCTTACTCGAGTTGGATCCTTACTCAACAGGTAAGGCGCGAG
SEQ ID NO: 23. Primer Y1-11_A13-R
CTAATTACATGACTCGAGGTCGACGGTATCGTTATTTCCATCTTAAGCCATCGTTAACTTC
SEQ ID NO: 24. Primer Y1-11_A13-F
CATTAGAAAGAAAGCATAGCAATCTAATCTAAGTTTAAAACAATGACTACTCAACCCCAGCTAAA
TG
SEQ ID NO: 25. Primer YO012
GAAAGCATAGCAATCTAATCTAAGTTTAAAACAATGACCGAACAAGTCTTATTCTCAGTA
SEQ ID NO: 26. Primer YO013
CTAATTACATGACTCGAGGTCGACGGTATCGTTAAGCGTTCAACAAATTGAAAAATCTG
SEQ ID NO: 27. Primer YO014
GAAAGCATAGCAATCTAATCTAAGTTTAAAACAATGACCGAACATGTATTATTCTCAG
SEQ ID NO: 28. Primer YO015
CTAATTACATGACTCGAGGTCGACGGTATCGTTAAGCGTTTAACAAATTGAAAAATC
SEQ ID NO: 29. Primer YO018
GAAAGCATAGCAATCTAATCTAAGTTTAAAACAATGAGAAGATACATCAGAGGTGGT
SEQ ID NO: 30. Primer YO019
CTAATTACATGACTCGAGGTCGACGGTATCGTTATGCAGCGTTCAACAAATTGAAAA
SEQ ID NO: 31. Primer YO020
GAAAGCATAGCAATCTAATCTAAGTTTAAAACAATGACCGAACAAGTCTTATTCTCAG
SEQ ID NO: 32. Primer YO021
CTAATTACATGACTCGAGGTCGACGGTATCGTTAAGCGTTCAACAAATTGAAAAATCT
SEQ ID NO: 33. Primer YO024
GAAAGCATAGCAATCTAATCTAAGTTTAAAACAATGAACTTACAATTTGAAGAAAGACCA
SEQ ID NO: 34. Primer YO025
CTAATTACATGACTCGAGGTCGACGGTATCGTTACAAATCAGCTAAAGGGTGTTCAC
SEQ ID NO: 35. Primer YO026
GAAAGCATAGCAATCTAATCTAAGTTTAAAACAATGAACTTACACTTTGAAGAATTGAC
SEQ ID NO: 36. Primer YO027
CTAATTACATGACTCGAGGTCGACGGTATCGTTAGTAGTCAGACAAATCTGCTAAAG
SEQ ID NO: 37. Primer YO028
GAAAGCATAGCAATCTAATCTAAGTTTAAAACAATGACAATCCACTGTGAAGTATTAAC
SEQ ID NO: 38. Primer YO029
CTAATTACATGACTCGAGGTCGACGGTATCGTTAACCAACGTCAGCCAAAGGGTG
SEQ ID NO: 39. Primer YO030
GAAAGCATAGCAATCTAATCTAAGTTTAAAACAATGAATGTCACCTTTGAAGAAAGAG
SEQ ID NO: 40. Primer YO031
CTAATTACATGACTCGAGGTCGACGGTATCGTTATGCCAAATCAGCTAAAGGGTG
SEQ ID NO: 41.
Linear recombination fragment for genomic integration of
F6AA82-2 in YPRCdelta15 site of S. cerevisiae strain BY4741
      1 CGAAACCCTA TGCTCTGTTG TTCGGATTTG AAATTTTAAA ACTACATTAA TGTGTTAGTT
    61 TTTCTTTCTT TCTTTCTTTG TCTTGACGTG ATTTGGACTT CTGTCTTGCA TTCGCGTCCA
  121 TTCATCTGAC CCAATATTCC TTTTGGTTTT GTTATCCTTA TAAAAAGAAA GGAAGCTTCT
  181 TAGAGGGAAA AAAATGATGA AGAGTAATGC CAAAATATAA ATAAATAAAT AAATATGAAA
  241 ATCATTTTCT ATTTTTAATA GAATAAGAAG AGCATCTTAA GATTACAATT TCAAGAAATA
  301 GTTTACACAG TATATCCAAT AACTCCAATA AACTACTTTC CTATACAAAT TTCTATGGTG
  361 GGATTAATAG TAAAACTTCT GTACTTCTCT AATTCACCAA GAAATTAAGG TAAACATCTG
  421 GTAAGCACTA TCCAGCTTTT TGCTATTACA CATATGGCTT TTCTGCAATC ATTTCTTCCC
  481 ATTTTGTCTC AAGCCGTTAG TCTTGAAACC ACAGGCGGAG TAGAGTTACT TGATGCGGTA
  541 TTTTACATGC CTTTTTTCAC TGCAAAAAAA ATGAAATACA TATTTACACG ATTTGCAGGA
  601 CAGTTTACGA TAGTGAGTAT GCAGAATAGT TAACACCTTT GTTTTATCCT TTTGTGTCTT
  661 AATTATATGA TATAAAGGCG CCTGGCAAAT TCCCGTTTTA AGAGCTTGGT GAGCGCTAGG
  721 AGTCACTGCC AGGTATCGTT TGAACACGGC ATTAGTCAGG GAAGTCATAA CACAGTCCTT
  781 TCCCGCAATT TTCTTTTTCT ATTACTCTTG GCCTCCTCTA GTACACTCTA TATTTTTTTA
  841 TGCCTCGGTA ATGATTTTCA TTTTTTTTTT TCCCCTAGCG GATGACTCTT TTTTTTTCTT
  901 AGCGATTGGC ATTATCACAT AATGAATTAT ACATTATATA AAGTAATGTG ATTTCTTCGA
  961 AGAATATACT AAAAAATGAG CAGGCAAGAT AAACGAAGGC AAAGATGACA GAGCAGAAAG
1021 CCCTAGTAAA GCGTATTACA AATGAAACCA AGATTCAGAT TGCGATCTCT TTAAAGGGTG
1081 GTCCCCTAGC GATAGAGCAC TCGATCTTCC CAGAAAAAGA GGCAGAAGCA GTAGCAGAAC
1141 AGGCCACACA ATCGCAAGTG ATTAACGTCC ACACAGGTAT AGGGTTTCTG GACCATATGA
1201 TACATGCTCT GGCCAAGCAT TCCGGCTGGT CGCTAATCGT TGAGTGCATT GGTGACTTAC
1261 ACATAGACGA CCATCACACC ACTGAAGACT GCGGGATTGC TCTCGGTCAA GCTTTTAAAG
1321 AGGCCCTACT GGCGCGTGGA GTAAAAAGGT TTGGATCAGG ATTTGCGCCT TTGGATGAGG
1381 CACTTTCCAG AGCGGTGGTA GATCTTTCGA ACAGGCCGTA CGCAGTTGTC GAACTTGGTT
1441 TGCAAAGGGA GAAAGTAGGA GATCTCTCTT GCGAGATGAT CCCGCATTTT CTTGAAAGCT
1501 TTGCAGAGGC TAGCAGAATT ACCCTCCACG TTGATTGTCT GCGAGGCAAG AATGATCATC
1561 ACCGTAGTGA GAGTGCGTTC AAGGCTCTTG CGGTTGCCAT AAGAGAAGCC ACCTCGCCCA
1621 ATGGTACCAA CGATGTTCCC TCCACCAAAG GTGTTCTTAT GTAGTGACAC CGATTATTTA
1681 AAGCTGCAGC ATACGATATA TATACATGTG TATATATGTA TACCTATGAA TGTCAGTAAG
1741 TATGTATACG AACAGTATGA TACTGAAGAT GACAAGGTAA TGCATCATTC TATACGTGTC
1801 ATTCTGAACG AGGCGCGCTT TCCTTTTTTC TTTTTGCTTT TTCTTTTTTT TTCTCTTGAA
1861 CTCGACGGAT CTATGCGGTG TGAAATACCG CACAGATGCG TAAGGAGAAA ATACCGCATC
1921 AGGAAATTGT AAACGTTAAT ATTTTGTTAA AATTCGCGTT AAATTTTTGT TAAATCAGCT
1981 CATTTTTTAA CCAATAGGCC GATAGCTTCA AAATGTTTCT ACTCCTTTTT TACTCTTCCA
2041 GATTTTCTCG GACTCCGCGC ATCGCCGTAC CACTTCAAAA CACCCAAGCA CAGCATACTA
2101 AATTTCCCCT CTTTCTTCCT CTAGGGTGTC GTTAATTACC CGTACTAAAG GTTTGGAAAA
2161 GAAAAAAGAG ACCGCCTCGT TTCTTTTTCT TCGTCGAAAA AGGCAATAAA AATTTTTATC
2221 ACGTTTCTTT TTCTTGAAAA TTTTTTTTTT GATTTTTTTC TCTTTCGATG ACCTCCCATT
2281 GATATTTAAG TTAATAAACG GTCTTCAATT TCTCAAGTTT CAGTTTCATT TTTCTTGTTC
2341 TATTACAACT TTTTTTACTT CTTGCTCATT AGAAAGAAAG CATAGCAATC TAATCTAAGT
2401 TTAAAACAAT GAATGTCACC TTTGAAGAAA GAGCCAGTTT ACACGGTTAC AGAATCGGTA
2461 TCGCATCCTT AGACGCCCCA GCATCCTTGA ACGCTTTGTC CTTACCAATG ATTGATGCTT
2521 TACAAGACAG ATTGAGAGCT TGGGCAGAAG ATGCAGACAT AGCCTGTGTC TTGTTACGTG
2581 GTAATGGTTC TAAAGCATTT TGCGCAGGTG GTGACGTTGT CCAATTAGCT AAGAAATGTT
2641 TGGCCTCCCC AGGTGAAGCC CCTGAATTGG CTGAAAGATT TTTCGCTAGA AGTTACAGAT
2701 TAGATCATTA TTTGCACACA TACCCAAAGC CTTTGATATG CTGGGCTCAT GGTCACGTTT
2761 TAGGTGGTGG TATGGGTTTG TTACAAGGTG CTGGTATTAG AATAGTCACC CCATCTTCAA
2821 GATTGGCAAT GCCTGAAATC TCTATTGGTT TATTTCCAGA TGTTGGTGGT TCCCATTTCT
2881 TAAGTAGATT GCCTGGTAAA TTGGGTTTAT TTTTCGGTTT AACCGCTTCA CCATTGAATG
2941 CCAGAGATGC TTTGGACTTA AACTTGGCTG ATAGATTCTT GTTAGATACT CAACAAGACG
3001 CATTGATCGA TGGTTTGATC CAATTGAACT GGAGAGAACA ACCAGATTTG CAATTGCATT
3061 CCTTGTTAAA GGCTTTAGAA CAACAAGCAA GAAGTGAATT ACCAGCTGCA CAATGGTTGC
3121 CTAGAAGAGA AAGATTAGAC GCCTTGTTAG ATCAAGCTAC TTTACCATTG TCTTGGCAAG
3181 CCTTAGCTTC ATTGGAAAAC GATGAAGACG CATTGTTAGC CAAAGCCGCT AAGACTATGT
3241 TGGGTGGTTC ACCTTTAACA GGTCATTTGG TCTGGGGTCA AATTAGAAGA GCTAGACACT
3301 TATCTTTGGC ACAAGTATTT CAAATGGAAT ACGGCATGTC ATTGAATTGT TGCAGACATC
3361 CAGAATTTGC AGAAGGTGTA AGAGCCAGAT TAATCGATAA AGACCACGCT CCACATTGGC
3421 ACTGGCCTGA CGTAAACCAA GTTCCTGAAG CCGTTATTGC AGCCCATTTT GCACCATTAG
3481 ATGACCACCC TTTAGCTGAT TTGGCATAAC GATACCGTCG ACCTCGAGTC ATGTAATTAG
3541 TTATGTCACG CTTACATTCA CGCCCTCCCC CCACATCCGC TCTAACCGAA AAGGAAGGAG
3601 TTAGACAACC TGAAGTCTAG GTCCCTATTT ATTTTTTTAT AGTTATGTTA GTATTAAGAA
3661 CGTTATTTAT ATTTCAAATT TTTCTTTTTT TTCTGTACAG ACGCGTGTAC GCATGTAACA
3721 TTATACTGAA AACCTTGCTT GAGAAGGTTT TGGGACGCTC GAAGGCTTTA ATTTGCGGCC
3781 ATAAAGCAGC CGCTACCAAA CAGACAAGAT TCAGTATGTA AGGTAAATAC CTTTTTGCAC
3841 AGTTAAACTA CCCAAACTTA TTAAAGCTTG ATAAATTACT GAAATTCCAC CTTTCAGTTA
3901 GATTCAGGCC TCATATAGAT TAGATATAGG GTACGTAACA TTCTGTCAAC CAAGTTGTTG
3961 GAATGAAAGT CTAAAATGTC ATCTATTCGG TAGCACTCAT GTTACTAGTA TACTGTCACA
4021 TGCGGTGTAA CGTGGGGACA TAAAACAGAC ATCAAATATA ATGGAAGCTG AAATGCAAAG
4081 ATCGATAATG TAATAGGAAT GAAACATATA AAACGAAAGG AGAAGTAATG GTAATATTAG
4141 TATGTAGAAA TACCGATTCA ATTTTGGGGA TTCTTATATT CTCGAGAGAA TTTCTAGTAT
4201 AATCTGTATA CATAATATTA TAGGCTTTAC CAACAATGGA ATTTCGACAA TTATCATATT
4261 ATTCACCAAT TAATCACAAG TTGGTAATGA GTTTGATAAC AAGTTACTTT CTTAACAACG
4321 TTAGTATCGT CAAAACACTC GGTTTTACTC GAGCTTGTAG CACAATAATA CCGTGTAGAG
4381 TTCTGTATTG TTCTTCTTAG TGCTTGTATA TGCTCATCCC GACCTTCCAT T
SEQ ID NO: 42. Linear recombination fragment for genomic
integration of F6AA82-2 in
YORWdelta22 site of S. cerevisiae strain BY4741
      1 ACCGGAGCTT GGATATGATA AACGAAATAT TCTTGAATCG TGAGATCGCC TGTTTTCAAA
    61 ACCGTTGGAG GCAGAAACAA TTTTGTCACA AGATGGGCAT TCTACCCCAT CCTTGCTGTA
  121 TTATTGTAGT CTCGCTTTCT TTTATGCTGG ACAAATGAGA CTACTGCACA TTTTTATACG
  181 TTCTTGGTTT TTTTTAAAGG TGTGGTTTCG GCATTATCCT GCCGCACGTT TCTTGGATAA
  241 TTCATCCTGA TTCTCTATTT TAAACGCTTC AGCCTATCAG GATTTGGTTT TGATACATAC
  301 TGCAAGAGTG TATCTCGGGA ACAGTCATTT ATTCCGCAAC AAACTTAATT GCGGAACGCG
  361 TTAGGCGATT TCTAGCATAT ATCAAATACC GTTCGCGATT TCTTCTGGGT TCGTCTCTTT
  421 TCTTTTAAAT ACTTATTAAC GTACTCAAAC AACTACACTT CGTTGTATCT CAGAATGAGA
  481 TCCCTCAGTA TGACAATACA TCATTCTAAA CGTTCGTAAA ACACATATGA AACAACTTTA
  541 TAACAAAGCG AACAAAATGG GCAACATGAG ATGAAACTCC GCGTCCCTTA GCTGAACTAC
  601 CCAAACGTAC GAATGCCTGA ACAATTAGTT TAGATCCGAG ATTCCGCGCT TCCATCATTT
  661 AGTATAATCC ATATTTTATA TAATATATAG GATAAGTAAC AGCCCGCGAA TCGAGGAGAA
  721 CTTCTAGTAT ATCCACATAC CTAATATTAT TGCCTTATTA AAAATGGAAT CCCAACAATT
  781 ACATCAAAAT CCACATTCTC TTCAAAATCA ATTGTCCTGT ACTTCCTTGT TCATGTGTGT
  841 TCAAAAACGT TATATTTATA GGATAATTAT ACTCTATTTC TCAACAAGTA ATTGGTTGTT
  901 TGGCCGAGCG GTCTAAGGCG CCTGATTCAA GAAATATCTT GACCGCAGTT AACTGTGGGA
  961 ATACTCAGGT ATCGTAAGAT GCAAGAGTTC GAATCTCTTA GCAACCATTA TTTTTTTCCT
1021 CAACATAACG AGAACACACA GGGGCGCTAT CGCACAGAAT CAAATTCGAT GACTGGAAAT
1081 TTTTTGTTAA TTTCAGAGGT CGCCTGACGC ATATACCTTT TTCAACTGAA AAATTGGGAG
1141 AAAAAGGAAA GGTGAGAGGC CGGAACCGGC TTTTCATATA GAATAGAGAA GCGTTCATGA
1201 CTAAATGCTT GCATCACAAT ACTTGAAGTT GACAATATTA TTTAAGGACC TATTGTTTTT
1261 TCCAATAGGT GGTTAGCAAT CGTCTTACTT TCTAACTTTT CTTACCTTTT ACATTTCAGC
1321 AATATATATA TATATTTCAA GGATATACCA TTCTAATGTC TGCCCCTATG TCTGCCCCTA
1381 AGAAGATCGT CGTTTTGCCA GGTGACCACG TTGGTCAAGA AATCACAGCC GAAGCCATTA
1441 AGGTTCTTAA AGCTATTTCT GATGTTCGTT CCAATGTCAA GTTCGATTTC GAAAATCATT
1501 TAATTGGTGG TGCTGCTATC GATGCTACAG GTGTCCCACT TCCAGATGAG GCGCTGGAAG
1561 CCTCCAAGAA GGTTGATGCC GTTTTGTTAG GTGCTGTGGC TGGTCCTAAA TGGGGTACCG
1621 GTAGTGTTAG ACCTGAACAA GGTTTACTAA AAATCCGTAA AGAACTTCAA TTGTACGCCA
1681 ACTTAAGACC ATGTAACTTT GCATCCGACT CTCTTTTAGA CTTATCTCCA ATCAAGCCAC
1741 AATTTGCTAA AGGTACTGAC TTCGTTGTTG TCAGAGAATT AGTGGGAGGT ATTTACTTTG
1801 GTAAGAGAAA GGAAGACGAT GGTGATGGTG TCGCTTGGGA TAGTGAACAA TACACCGTTC
1861 CAGAAGTGCA AAGAATCACA AGAATGGCCG CTTTCATGGC CCTACAACAT GAGCCACCAT
1921 TGCCTATTTG GTCCTTGGAT AAAGCTAATC TTTTGGCCTC TTCAAGATTA TGGAGAAAAA
1981 CTGTGGAGGA AACCATCAAG AACGAATTCC CTACATTGAA GGTTCAACAT CAATTGATTG
2041 ATTCTGCCGC CATGATCCTA GTTAAGAACC CAACCCACCT AAATGGTATT ATAATCACCA
2101 GCAACATGTT TGGTGATATC ATCTCCGATG AAGCCTCCGT TATCCCAGGT TCCTTGGGTT
2161 TGTTGCCATC TGCGTCCTTG GCCTCTTTGC CAGACAAGAA CACCGCATTT GGTTTGTACG
2221 AACCATGCCA CGGTTCTGCT CCAGATTTGC CAAAGAATAA GGTTGACCCT ATCGCCACTA
2281 TCTTGTCTGC TGCAATGATG TTGAAATTGT CATTGAACTT GCCTGAAGAA GGTAAGGCCA
2341 TTGAAGATGC AGTTAAAAAG GTTTTGGATG CAGGTATCAG AACTGGTGAT TTAGGTGGTT
2401 CCAACAGTAC CACCGAAGTC GGTGATGCTG TCGCCGAAGA AGTTAAGAAA ATCCTTGCTT
2461 AAAAAGATTC TCTTTTTTTA TGATATTTGT ACATAAACTT TATAAATGAA ATTCATAATA
2521 GAAACGACAC GAAATTACAA AATGGAATAT GTTCATAGGG TAGACGAAAC TATATACGCA
2581 ATCTACATAC ATTTATCAAG AAGGAGAAAA AGGAGGATAG TAAAGGAATA CAGGTAAGCA
2641 AATTGATACT AATGGCTCAA CGTGATAAGG AAAAAGAATT GCACTTTAAC ATTAATATTG
2701 ACAAGGAGGA GGGCACCACA CAAAAAGTTA GGTGTAACAG AAAATCATGA AACTACGATT
2761 CCTAATTTGA TATTGGAGGA TTTTCTCTAA AAAAAAAAAA ATACAACAAA TAAAAAACAC
2821 TCAATGACCT GACCATTTGA TGGAGTTTAA GTCAATACCT TCTTGAAGCA TTTCCCATAA
2881 TGGTGAAAGT TCCCTCAAGA ATTTTACTCT GTCAGAAACG GCCTATAGCT TCAAAATGTT
2941 TCTACTCCTT TTTTACTCTT CCAGATTTTC TCGGACTCCG CGCATCGCCG TACCACTTCA
3001 AAACACCCAA GCACAGCATA CTAAATTTCC CCTCTTTCTT CCTCTAGGGT GTCGTTAATT
3061 ACCCGTACTA AAGGTTTGGA AAAGAAAAAA GAGACCGCCT CGTTTCTTTT TCTTCGTCGA
3121 AAAAGGCAAT AAAAATTTTT ATCACGTTTC TTTTTCTTGA AAATTTTTTT TTTGATTTTT
3181 TTCTCTTTCG ATGACCTCCC ATTGATATTT AAGTTAATAA ACGGTCTTCA ATTTCTCAAG
3241 TTTCAGTTTC ATTTTTCTTG TTCTATTACA ACTTTTTTTA CTTCTTGCTC ATTAGAAAGA
3301 AAGCATAGCA ATCTAATCTA AGTTTAAAAC AATGAATGTC ACCTTTGAAG AAAGAGCCAG
3361 TTTACACGGT TACAGAATCG GTATCGCATC CTTAGACGCC CCAGCATCCT TGAACGCTTT
3421 GTCCTTACCA ATGATTGATG CTTTACAAGA CAGATTGAGA GCTTGGGCAG AAGATGCAGA
3481 CATAGCCTGT GTCTTGTTAC GTGGTAATGG TTCTAAAGCA TTTTGCGCAG GTGGTGACGT
3541 TGTCCAATTA GCTAAGAAAT GTTTGGCCTC CCCAGGTGAA GCCCCTGAAT TGGCTGAAAG
3601 ATTTTTCGCT AGAAGTTACA GATTAGATCA TTATTTGCAC ACATACCCAA AGCCTTTGAT
3661 ATGCTGGGCT CATGGTCACG TTTTAGGTGG TGGTATGGGT TTGTTACAAG GTGCTGGTAT
3721 TAGAATAGTC ACCCCATCTT CAAGATTGGC AATGCCTGAA ATCTCTATTG GTTTATTTCC
3781 AGATGTTGGT GGTTCCCATT TCTTAAGTAG ATTGCCTGGT AAATTGGGTT TATTTTTCGG
3841 TTTAACCGCT TCACCATTGA ATGCCAGAGA TGCTTTGGAC TTAAACTTGG CTGATAGATT
3901 CTTGTTAGAT ACTCAACAAG ACGCATTGAT CGATGGTTTG ATCCAATTGA ACTGGAGAGA
3961 ACAACCAGAT TTGCAATTGC ATTCCTTGTT AAAGGCTTTA GAACAACAAG CAAGAAGTGA
4021 ATTACCAGCT GCACAATGGT TGCCTAGAAG AGAAAGATTA GACGCCTTGT TAGATCAAGC
4081 TACTTTACCA TTGTCTTGGC AAGCCTTAGC TTCATTGGAA AACGATGAAG ACGCATTGTT
4141 AGCCAAAGCC GCTAAGACTA TGTTGGGTGG TTCACCTTTA ACAGGTCATT TGGTCTGGGG
4201 TCAAATTAGA AGAGCTAGAC ACTTATCTTT GGCACAAGTA TTTCAAATGG AATACGGCAT
4261 GTCATTGAAT TGTTGCAGAC ATCCAGAATT TGCAGAAGGT GTAAGAGCCA GATTAATCGA
4321 TAAAGACCAC GCTCCACATT GGCACTGGCC TGACGTAAAC CAAGTTCCTG AAGCCGTTAT
4381 TGCAGCCCAT TTTGCACCAT TAGATGACCA CCCTTTAGCT GATTTGGCAT AACGATACCG
4441 TCGACCTCGA GTCATGTAAT TAGTTATGTC ACGCTTACAT TCACGCCCTC CCCCCACATC
4501 CGCTCTAACC GAAAAGGAAG GAGTTAGACA ACCTGAAGTC TAGGTCCCTA TTTATTTTTT
4561 TATAGTTATG TTAGTATTAA GAACGTTATT TATATTTCAA ATTTTTCTTT TTTTTCTGTA
4621 CAGACGCGTG TACGCATGTA ACATTATACT GAAAACCTTG CTTGAGAAGG TTTTGGGACG
4681 CTCGAAGGCT TTAATTTGCG GCCGGACCAA CTATCATCCG CTAATTACTG ACATTACCAA
4741 ATGAGATCTG TGAATGGGCA AGATAAAAAA CAAAAATTGA AATGTTTGAC GTTATGTAAA
4801 ACTATTAATT CCTTCGCTTT CGGCGGTCAC AGAATTTGCG TGTAGCTGAC TCTTGTTCAA
4861 TCAATATCAT TTGTTACTTT ATTTGAAAGT CTGTATTACT GCGCCTATTG TCATCCGTAC
4921 CAAAGAACGT CAAAAAGAAA CAAGATAATT TTTGTGCTTA CACCATTTAT AGATCACTGA
4981 GCCCAGAATA TCGCTGGAGC TCAGTGTAAG TGGCATGAAC ACAACTCTGA CTGATCGCAC
5041 ATATTGCCGT TATCATAAAT ACTAGTTGTA CTTGTCAATG CGACGAATGG CATCATGCCT
5101 ATTATTACGT TCCTCTTTTT CCGTTTCATG TTTCCAGAAT GCTATTGAAT CTAACACTTC
5161 AATTATAAAA AAGAATAAAT CCGCAATAAT TTTAGGCTAA TTGTTGTACT GTCAAGCGAA
5221 CCTAATGGTT AAAATTCAGA GGAACCTTCG ACGTAGTCTG ATCGCTACTT CTATATCTTA
5281 TGTTCCCAGT CAATCAAAAG TTGATACTAT AATAGCTGCC ATTTATACCT GTTAGTTATG
5341 GCGATCGTTT ATCACG
SEQ ID NO: 43. Linear recombination fragment for genomic
integration of F6AA82-2 in BUD21 gene
of S. cerevisiae strain BY4741
      1 AACTCAATCG TGCTAAGCGA CAAACTGAAA AGGTATATCA ACCAGAGATG AAATCGGAAA
    61 TTAGCGGGGA TAGCACTCTG AAACGTGTTC AAGTCACCTT CCACGGGAAT GGTCGGGTAA
  121 ATAAAAAGAA AAAGAAGGTT CCTCCACGGA AAGATGGTAT CAAGTTCCGT TGATTGAAAA
  181 TTTTTCTTTT CAAGCGATGA GGTTAGCGAA GTTGTTCGAA CAAGCCCAAA TATGTTTAGA
  241 AGGCAGTACG AATTGATACC CTGAACTGTT ACTACATACC ACAGCTTTTC AATTCAATTC
  301 ATCATTTTTT TTTTATTCTT TTTTTTGATT TCGGTTTCTT TGAAATTTTT TTGATTCGGT
  361 AATCTCCGAA CAGAAGGAAG AACGAAGGAA GGAGCACAGA CTTAGATTGG TATATATACG
  421 CATATGTAGT GTTGAAGAAA CATGAAATTG CCCAGTATTC TTAACCCAAC TGCACAGAAC
  481 AAAAACCTGC AGGAAACGAA GATAAATCAT GTCGAAAGCT ACATATAAGG AACGTGCTGC
  541 TACTCATCCT AGTCCTGTTG CTGCCAAGCT ATTTAATATC ATGCACGAAA AGCAAACAAA
  601 CTTGTGTGCT TCATTGGATG TTCGTACCAC CAAGGAATTA CTGGAGTTAG TTGAAGCATT
  661 AGGTCCCAAA ATTTGTTTAC TAAAAACACA TGTGGATATC TTGACTGATT TTTCCATGGA
  721 GGGCACAGTT AAGCCGCTAA AGGCATTATC CGCCAAGTAC AATTTTTTAC TCTTCGAAGA
  781 CAGAAAATTT GCTGACATTG GTAATACAGT CAAATTGCAG TACTCTGCGG GTGTATACAG
  841 AATAGCAGAA TGGGCAGACA TTACGAATGC ACACGGTGTG GTGGGCCCAG GTATTGTTAG
  901 CGGTTTGAAG CAGGCGGCAG AAGAAGTAAC AAAGGAACCT AGAGGCCTTT TGATGTTAGC
  961 AGAATTGTCA TGCAAGGGCT CCCTATCTAC TGGAGAATAT ACTAAGGGTA CTGTTGACAT
1021 TGCGAAGAGC GACAAAGATT TTGTTATCGG CTTTATTGCT CAAAGAGACA TGGGTGGAAG
1081 AGATGAAGGT TACGATTGGT TGATTATGAC ACCCGGTGTG GGTTTAGATG ACAAGGGAGA
1141 CGCATTGGGT CAACAGTATA GAACCGTGGA TGATGTGGTC TCTACAGGAT CTGACATTAT
1201 TATTGTTGGA AGAGGACTAT TTGCAAAGGG AAGGGATGCT AAGGTAGAGG GTGAACGTTA
1261 CAGAAAAGCA GGCTGGGAAG CATATTTGAG AAGATGCGGC CAGCAAAACT AAAAAACTGT
1321 ATTATAAGTA AATGCATGTA TACTAAACTC ACAAATTAGA GCTTCAATTT AATTATATCA
1381 GTTATTACCC TATGCGGTGT GAAATACCGC ACAGATGCGT AAGGAGAAAA TACCGCATCA
1441 GGATAGCTTC AAAATGTTTC TACTCCTTTT TTACTCTTCC AGATTTTCTC GGACTCCGCG
1501 CATCGCCGTA CCACTTCAAA ACACCCAAGC ACAGCATACT AAATTTCCCC TCTTTCTTCC
1561 TCTAGGGTGT CGTTAATTAC CCGTACTAAA GGTTTGGAAA AGAAAAAAGA GACCGCCTCG
1621 TTTCTTTTTC TTCGTCGAAA AAGGCAATAA AAATTTTTAT CACGTTTCTT TTTCTTGAAA
1681 ATTTTTTTTT TGATTTTTTT CTCTTTCGAT GACCTCCCAT TGATATTTAA GTTAATAAAC
1741 GGTCTTCAAT TTCTCAAGTT TCAGTTTCAT TTTTCTTGTT CTATTACAAC TTTTTTTACT
1801 TCTTGCTCAT TAGAAAGAAA GCATAGCAAT CTAATCTAAG TTTAAAACAA TGAATGTCAC
1861 CTTTGAAGAA AGAGCCAGTT TACACGGTTA CAGAATCGGT ATCGCATCCT TAGACGCCCC
1921 AGCATCCTTG AACGCTTTGT CCTTACCAAT GATTGATGCT TTACAAGACA GATTGAGAGC
1981 TTGGGCAGAA GATGCAGACA TAGCCTGTGT CTTGTTACGT GGTAATGGTT CTAAAGCATT
2041 TTGCGCAGGT GGTGACGTTG TCCAATTAGC TAAGAAATGT TTGGCCTCCC CAGGTGAAGC
2101 CCCTGAATTG GCTGAAAGAT TTTTCGCTAG AAGTTACAGA TTAGATCATT ATTTGCACAC
2161 ATACCCAAAG CCTTTGATAT GCTGGGCTCA TGGTCACGTT TTAGGTGGTG GTATGGGTTT
2221 GTTACAAGGT GCTGGTATTA GAATAGTCAC CCCATCTTCA AGATTGGCAA TGCCTGAAAT
2281 CTCTATTGGT TTATTTCCAG ATGTTGGTGG TTCCCATTTC TTAAGTAGAT TGCCTGGTAA
2341 ATTGGGTTTA TTTTTCGGTT TAACCGCTTC ACCATTGAAT GCCAGAGATG CTTTGGACTT
2401 AAACTTGGCT GATAGATTCT TGTTAGATAC TCAACAAGAC GCATTGATCG ATGGTTTGAT
2461 CCAATTGAAC TGGAGAGAAC AACCAGATTT GCAATTGCAT TCCTTGTTAA AGGCTTTAGA
2521 ACAACAAGCA AGAAGTGAAT TACCAGCTGC ACAATGGTTG CCTAGAAGAG AAAGATTAGA
2581 CGCCTTGTTA GATCAAGCTA CTTTACCATT GTCTTGGCAA GCCTTAGCTT CATTGGAAAA
2641 CGATGAAGAC GCATTGTTAG CCAAAGCCGC TAAGACTATG TTGGGTGGTT CACCTTTAAC
2701 AGGTCATTTG GTCTGGGGTC AAATTAGAAG AGCTAGACAC TTATCTTTGG CACAAGTATT
2761 TCAAATGGAA TACGGCATGT CATTGAATTG TTGCAGACAT CCAGAATTTG CAGAAGGTGT
2821 AAGAGCCAGA TTAATCGATA AAGACCACGC TCCACATTGG CACTGGCCTG ACGTAAACCA
2881 AGTTCCTGAA GCCGTTATTG CAGCCCATTT TGCACCATTA GATGACCACC CTTTAGCTGA
2941 TTTGGCATAA CGATACCGTC GACCTCGAGT CATGTAATTA GTTATGTCAC GCTTACATTC
3001 ACGCCCTCCC CCCACATCCG CTCTAACCGA AAAGGAAGGA GTTAGACAAC CTGAAGTCTA
3061 GGTCCCTATT TATTTTTTTA TAGTTATGTT AGTATTAAGA ACGTTATTTA TATTTCAAAT
3121 TTTTCTTTTT TTTCTGTACA GACGCGTGTA CGCATGTAAC ATTATACTGA AAACCTTGCT
3181 TGAGAAGGTT TTGGGACGCT CGAAGGCTTT AATTTGCGGC CGTACAATCA CAAAATTAAT
3241 CATAATGTTC ATTTATCTAT AATCAAACAA TGAGAATATA TACTGAAGTT TGGTAGGTTA
3301 TTCTTCAGAG ATGCAGTACG ATATCACCAA CGGTATGCAG ACACCTATCA AAACATAAAC
3361 GGATTCATCA TGAGGTAAAA CGACTTCCTG TTCAACGCTG AGATCGTGAT CATGAGAATC
3421 TCCACCAACA AATGCTGTGA AAGACGCATA TAGTAAACTG CCACCACTCA TAAGCAGAAG
3481 ATTTCCACTT AT
SEQ ID NO: 44.
SeACS(L641P) DNA sequence codon optimized for expression in
S. cerevisiae
      1 ATGTCCCAAA CTCATAAGCA CGCTATTCCA GCTAATATTG CTGATAGATG CTTGATCAAC
    61 CCAGAACAAT ACGAAACTAA GTACAAGCAA TCCATCAACG ATCCAGATAC TTTTTGGGGT
  121 GAACAAGGTA AGATTTTGGA TTGGATTACC CCATACCAAA AGGTCAAGAA TACTTCTTTT
  181 GCTCCAGGTA ACGTTTCCAT CAAATGGTAT GAAGATGGTA CTTTGAACTT GGCTGCTAAC
  241 TGTTTGGATA GACACTTGCA AGAAAACGGT GATAGAACCG CTATTATTTG GGAAGGTGAT
  301 GATGCCTCTC AATCCAAACA TATCTCTTAC AGAGAATTGC ACAGAGATGT CTGTAGATTC
  361 GCTAACACTT TGTTGGATTT GGGTATTAAG AAGGGTGACG TTGTTGCTAT CTATATGCCA
  421 ATGGTTCCTG AAGCTGCTGT TGCTATGTTG GCTTGTGCTA GAATTGGTGC TGTTCATTCT
  481 GTTATTTTCG GTGGTTTTTC ACCAGAAGCT GTTGCCGGTA GAATTATCGA TTCTTCATCC
  541 AGATTGGTTA TCACCGCTGA TGAAGGTGTT AGAGCTGGTA GATCAATTCC ATTGAAAAAG
  601 AACGTTGATG ACGCCTTGAA GAACCCAAAT GTTACTTCTG TTGAACACGT CATCGTTTTG
  661 AAGAGAACCG ATTCTGATAT TGACTGGCAA GAAGGTAGAG ATTTGTGGTG GAGAGATTTG
  721 ATTGAAAAGG CTTCTCCAGA ACATCAACCA GAAGCAATGA ACGCTGAAGA TCCTTTGTTT
  781 ATCTTGTACA CTTCTGGTTC TACTGGTAAG CCAAAAGGTG TTTTACACAC TACTGGTGGT
  841 TATTTGGTTT ACGCTGCTAC TACTTTCAAG TACGTTTTCG ATTATCATCC AGGTGACATC
  901 TATTGGTGTA CTGCTGATGT TGGTTGGGTT ACTGGTCATT CTTATTTGTT GTATGGTCCA
  961 TTAGCTTGTG GTGCTACTAC CTTGATGTTC GAAGGTGTTC CAAATTGGCC AACTCCAGCT
1021 AGAATGTGTC AAGTTGTTGA CAAACACCAA GTCAACATCT TGTATACTGC TCCAACTGCT
1081 ATTAGAGCTT TGATGGCTGA AGGTGATAAG GCTATTGAAG GTACTGATAG ATCCTCCTTG
1141 AGAATCTTGG GTTCTGTTGG TGAACCTATT AACCCTGAAG CTTGGGAATG GTACTGGAAG
1201 AAAATTGGTA AAGAAAAGTG CCCAGTTGTT GATACTTGGT GGCAAACTGA AACAGGTGGT
1261 TTTATGATTA CTCCATTGCC AGGTGCTATT GAATTGAAAG CTGGTTCTGC TACTAGACCA
1321 TTTTTTGGTG TTCAACCAGC CTTGGTTGAT AATGAAGGTC ATCCACAAGA AGGTGCTACT
1381 GAAGGTAATT TGGTTATTAC TGATTCTTGG CCAGGTCAAG CTAGAACTTT GTTTGGTGAT
1441 CACGAAAGAT TCGAACAAAC CTACTTTTCC ACCTTCAAGA ACATGTACTT TTCTGGTGAT
1501 GGTGCTAGAA GAGATGAAGA TGGTTACTAT TGGATTACCG GTAGAGTTGA TGATGTCTTG
1561 AATGTTTCTG GTCACAGATT GGGTACAGCC GAAATTGAAT CTGCTTTGGT TGCTCATCCA
1621 AAGATTGCTG AAGCAGCAGT TGTTGGTATT CCACATGCTA TTAAGGGTCA AGCAATCTAC
1681 GCTTACGTTA CCTTGAATCA TGGTGAAGAA CCATCCCCAG AATTATACGC TGAAGTTAGA
1741 AACTGGGTCA GAAAAGAAAT TGGTCCATTG GCTACTCCAG ATGTTTTACA TTGGACAGAT
1801 TCTTTGCCAA AGACCAGATC AGGTAAGATC ATGAGAAGAA TCTTGAGAAA AATTGCTGCC
1861 GGTGATACTT CTAACTTGGG TGATACCTCT ACTTTGGCTG ATCCAGGTGT TGTTGAAAAG
1921 CCATTAGAAG AAAAGCAAGC CATTGCCATG CCATCTTAA
SEQ ID NO: 45. UniProt ID A4XS22 derived Malonyl-CoA hydrolase from
Pseudomonas mendocina (strain ymp)
      1 MNLQFEERPSLHGYRIGIASLDAEKSLNALTLPMIQALDARLQAWAEDPTIACVMLRGNG
    61 PKAFCAGGDVVQLVQQCREHPGEVPPLARRFFADX₁YRLDHRIHSYPKPFICWAHGHVLGG
  121 GMGLMQGAGVRIVTPSSRLGMPEINIGLYPDVGGSWFLARLPGRLGLFLGLTAASINARD
  181 ALDLNLADRFLRDDQQDALLEGLVQLNWREQPAAQLHSLLRALENEARGELPAAQWLPRR
  241 ERIDELLDVADLPAAVQAISALQQDDDALLARAAKTLAHGCPLTAHLVWQQIRRARHLSL
  301 AEVFRMEYAMSLNCCRHPDFPEGVRARLIDKDQTPHWHWPDVAAIPEAVIEAHFAPAWEG
  361 EHPLADL
SEQ ID NO: 46. UniProt ID B9IZZ9 derived Malonyl-CoA hydrolase
from Bacillus cereus (strain Q1)
      1 MTEHVLFSVSENGVASITLNRPKALNSLSYDMLQPIGQKLKEWENDERIALIVLKGAGTK
    61 GFCAGGDIKTLYEARSNEAALQHAERFFEEX₁YEIDTYIYQYKKPIIACLDGIVMGGGVGL
  121 TNGAKYRIVTERTKWAMPEMNIGFFPDVGAAYFLNKAPGYAGRYVALTASILKASDVLFI
  181 NAADYFIASDSLPNFLTELESVNWSKEDDVHTHLKEVIRTFATAPTLESELAPSLEEINS
  241 HFAFDTIEEIIHSLEKDQSSFSLKAKETLLSKSPISLKVTLKQFIDGQNKSVEECFATDL
  301 VLAKNFMRHEDFFEGVRSVVVDKDQNPNYKYKQLSDVSEEDVNRFFNLLNA
SEQ ID NO: 47. UniProt ID C3ALI3 derived Malonyl-CoA hydrolase
from Bacillus mycoides Rock1-4
      1 MRRYIRGGFKMTENVLFSIHENGVASITLNRPKALNSLSYDMLHPIGQKLKEWEKDDRIA
    61 VVILKGAGTKGFCAGGDIKTLYEARSNEVALQHAEHFFEEX₁YEIDTYIYHYPKPIIACLD
  121 GIVMGGGVGLTNGAKYRIVTDRTKWAMPEMNIGFFPDVGAAYFLNKAPGQTGRYVALTAS
  181 VLKAADVLYIKAADHYMPSETLPTFLDAIEKVNWHDQNIHITLKELINKYETAPSVESEL
  241 VSLLEEIDQHFSFHTVEDIIHSLDNADGSFASKTKETLLSKSPFSLKVTLKQLIDGKEKS
  301 IEECFATDLVLAKNFMRHEDFFEGVRSVVVDKDQNPKYKYKQLSDVSDEDVNRFFNLLNA
SEQ ID NO: 48. UniProt ID E2XN63 derived Malonyl-CoA hydrolase from
Pseudomonas fluorescens WH6
      1 MNLHFEELTGSDGARIGIASLDAEKSLNALSLPMILALGDRLDAWAKDPNIVCVLLRGNG
    61 PKAFCAGGEVRSLALACREQPGEVPALAAQFFAAX₁YRLDYRLHTFPKPLICWGHGYVLGG
  121 GMGLLQSAAVRIVTPSSRLAMPEISIGLYPDVGASWFLSRLPGKLGLFLGLTGAHVNGRD
  181 ALDLGLADRFLRDDQQDELIEGLLQLNWQEQTAMQLNSLFKALAQEAVDQLPEAQWLPRR
  241 AQIDEWLDVGDVRSAWRALSQLRDHADPLFSRAGKTLSEGCPLTAHLVWEQIQRARHLSL
  301 AQVFQMEYTLSLNCCRHPEFSEGVRARLIDKDQTPRWHWPDVHTLPDAVVQAHFNKAWEG
  361 RHPLADLSDY
SEQ ID NO: 49. UniProt ID FOPNG8 derived Malonyl-CoA hydrolase
from Bacillus thuringiensis subsp. finitimus (strain YBT-020)
    1 MTEHVLFSVSENGVASITLNRPKALNSLSYDMLQPIGQKLKEWEHDERIALIVLKGAGTK
    61 GFCAGGDIKTLYEARSNEAALQHAERFFEEX₁YEIDTYIYQYKKPIIACLDGIVMGGGVGL
  121 TNGAKYRIVTERTKWAMPEMNIGFFPDVGAAYFLNKAPGYAGRYVALTASILKAADVLFI
  181 NAADYFIASDSLPNFLTELESVNWPKKDDVHTHLKEVIRTFATAPTLESELAPSLEEINS
  241 HFAFDTIEEIIHSLEKDQSSFALKAKETLLSKSPISLKVTLKQFIDGQNKSVEECFATDL
  301 VLAKNFMRHEDFFEGVRSVVVDKDQNPDYKYKQLSDVSEEDVNRFFNLLNA
SEQ ID NO: 50. UniProt ID F6AA82 derived Malonyl-CoA hydrolase from
Pseudomonas fulva (strain 12-X)
      1 MNVTFEERASLHGYRIGIASLDAPASLNALSLPMIDALQDRLRAWAEDADIACVLLRGNG
    61 SKAFCAGGDVVQLAKKCLASPGEAPELAERFFARX₁YRLDHYLHTYPKPLICWAHGHVLGG
  121 GMGLLQGAGIRIVTPSSRLAMPEISIGLFPDVGGSHFLSRLPGKLGLFFGLTASPLNARD
  181 ALDLNLADRFLLDTQQDALIDGLIQLNWREQPDLQLHSLLKALEQQARSELPAAQWLPRR
  241 ERLDALLDQATLPLSWQALASLENDEDALLAKAAKTMLGGSPLTGHLVWGQIRRARHLSL
  301 AQVFQMEYGMSLNCCRHPEFAEGVRARLIDKDHAPHWHWPDVNQVPEQVIAAHFAPLDDH
  361 PLADLA
SEQ ID NO: 51. UniProt ID Q63BK8 derived Malonyl-CoA
hydrolase from Bacillus cereus (strain ZK/E33L)
      1 MTEHVLFSVSENGVASITLNRPKALNSLSYEMLQPIGKKLKEWENDEQIALIVLKGAGTK
    61 GFCAGGDIKTLYEARSNEIALQHAERFFEEX₁YEIDTYIYQYKKPIIACLDGIVMGGGVGL
  121 TNGAKYRIVTERTKWAMPEMNIGFFPDVGAAYFLNKAPGFAGRYVALTASILKASDVLFI
  181 NAADYFMTSDSLPKFLTELESVNWHKGDDVHIHLKEVIRTFATTSNLESELAPLLEEINA
  241 HFAFDTIEEIIHSLEKDQSSFALKTKKTLLSKSPISLKVTLKQFIDGHDKSVEECFATDL
  301 VLAKNFMRHEDFFEGVRSVVVDKDQNPNYKYKQLSDVSDEDVNRFFNLLNA
SEQ ID NO: 52. UniProt ID Q81DR3 derived Malonyl-CoA
hydrolase from Bacillus cereus (strain ATCC 14579/DSM 31)
      1 MTEQVLFSVSENGVATITLNRPKALNSLSYDMLQPIGQKLKEWEHDERIALIVLKGAGTK
    61 GFCAGGDIKTLYEARSNEVALQHAERFFEEX₁YEIDTYIYQYTKPIIACLDGIVMGGGVGL
  121 TNGAKYRIVTERTKWAMPEMNIGFFPDVGAAYFLNKAPGYTGRFVALTASILKASDVLFI
  181 NAADYFMTSDSLPEFLTELESVNWHKEDDVHTNLKEVIRTFATAPNLESELAPSLEVINS
  241 HFAFDTIEEIIHSLEKDESSFALKTKEILLSKSPISLKVTLKQFIDGQDKSVEECFATDL
  301 ILAKNFMRHEDFFEGVRSVVVDKDQNPNYKYKQLSDVSEEDVNRFFNLLNA
SEQ ID NO: 53. Wild-type FabD from E. coli
      1 MTQFAFVFPG QGSQTVGMLA DMAASYPIVE ETFAEASAAL GYDLWALTQQ GPAEELNKTW
    61 QTQPALLTAS VALYRVWQQQ GGKAPAMMAG HSLGEYSALV CAGVIDFADA VRLVEMRGKF
  121 MQEAVPEGTG AMAAIIGLDD ASIAKACEEA AEGQVVSPVN FNSPGQVVIA GHKEAVERAG
  181 AACKAAGAKR ALPLPVSVPS HCALMKPAAD KLAVELAKIT FNAPTVPVVN NVDVKCETNG
  241 DAIRDALVRQ LYNPVQWTKS VEYMAAQGVE HLYEVGPGKV LTGLTKRIVD TLTASALNEP
  301 SAMAAALEL
```

1-15. (canceled)

16. A malonyl-CoA hydrolase having at least 90%, 95%, or more sequence identity when aligned relative to SEQ ID NO: 53 and containing one or more of the following amino acid mutations at the aligned positions: S92C, H201N, R117D, R117E, R117N, R117Y, R117G, R117H, Q11D, QUE, Q11N, Q11Y, Q11G, Q111H, L93A, L93V, L93L, L93F, L93S, L93G.

17. A recombinant host microorganism comprising the malonyl-CoA hydrolase of claim 16.

18. A method of producing malonate comprising culturing the recombinant host microorganism of claim 17 under conditions that result in production of malonate.

19. A method of producing malonate comprising culturing a recombinant host microorganism comprising the malonyl-CoA hydrolase of claim 16 under conditions that result in production of malonate.

20. A recombinant host microorganism that comprises a heterologous malonyl-CoA hydrolase selected from the group consisting of:
a. SEQ ID No 53, wherein the serine residue at position 92 is mutated to a cysteine residue;
b. SEQ ID No 53, wherein the arginine residue at position 117 is mutated to an aspartic acid, glutamic acid, asparagine, tyrosine, glycine, or histidine residue;
c. SEQ ID No 53, wherein the glutamine residue at position 11 is mutated to an aspartic acid, glutamic acid, asparagine, tyrosine, glycine, or histidine residue;
d. SEQ ID No 53, wherein the leucine residue at position 93 is mutated to an alanine, valine, isoleucine, phenylalanine, serine, or glycine residue; and
e. SEQ ID No 53, wherein any combination of more than one residue listed in 2a-2d are mutated to the combinations of residues listed for each position.

21. The recombinant host microorganism of claim 20, that comprises a malonyl-CoA hydrolase from Escherichia coli (SEQ ID No 53) wherein the serine residue at position 92 is mutated to a cysteine residue, the leucine residue at position 93 is mutated to a valine residue, and the arginine residue at position 117 is mutated to a histidine residue.

22. The recombinant host microorganism of claim 20, that comprises a malonyl-CoA hydrolase from Escherichia coli (SEQ ID No 53) wherein the leucine residue at position 93 is mutated to an isoleucine residue, and the arginine residue at position 117 is mutated to a tyrosine residue.

23. The recombinant host microorganism of claim 20, that comprises a malonyl-CoA hydrolase from Escherichia coli (SEQ ID No 53) wherein the leucine residue at position 93 is mutated to a serine residue, and the arginine residue at position 117 is mutated to a glycine residue.

24. The recombinant host microorganism of claim 20, that comprises a malonyl-CoA hydrolase from Escherichia coli (SEQ ID No 53) wherein the leucine residue at position 93 is mutated to a isoleucine residue, and the arginine residue at position 117 is mutated to a tyrosine residue.

25. The recombinant host microorganism of claim 20, that comprises a malonyl-CoA hydrolase from Escherichia coli (SEQ ID No 53) wherein the serine residue at position 92 is mutated to a cysteine residue, the leucine residue at position 93 is mutated to a phenylalanine residue, and the arginine residue at position 117 is mutated to a glycine residue.

26. The recombinant host microorganism of claim 20, that comprises a malonyl-CoA hydrolase from Escherichia coli (SEQ ID No 53) wherein the serine residue at position 92 is mutated to a cysteine residue, the glutamine residue at position 11 is mutated to an asparagine residue, and the arginine residue at position 117 is mutated to an aspartic acid residue.


SEQUENCE LISTING (53 sequences)

SEQ ID NO: 1 - protein, 500 aa, Saccharomyces cerevisiae
MLRNTLKCAQLSSKYGFKTTTRTFMTTQPQLNVTDAPPVLFTVQDTARVITLNRPKKLNALNAEMSESMFKTLNEYAKSDTTNLVILKSSNRPRSFCAGGDVATVAIFNFNKEFAKSIKFFTDEYSLNFQIATYLKPIVTFMDGITMGGGVGLSIHTPFRIATENTKWAMPEMDIGFFPDVGSTFALPRIVTLANSNSQMALYLCLTGEVVTGADAYMLGLASHYVSSENLDALQKRLGEISPPFNNDPQSAYFFGMVNESIDEFVSPLPKDYVFKYSNEKLNVIEACFNLSKNGTIEDIMNNLRQYEGSAEGKAFAQEIKTKLLTKSPSSLQIALRLVQENSRDHIESAIKRDLYTAANMCMNQDSLVEFSEATKHKLIDKQRVPYPWTKKEQLFVSQLTSITSPKPSLPMSLLRNTSNVTWTQYPYHSKYQLPTEQEIAAYIEKRTNDDTGAKVTEREVLNHFANVIPSRRGKLGIQSLCKIVCERKCEEVNDGLRWK

SEQ ID NO: 2 - DNA, 1000 bp, Haemophilus influenzae
  source 1..1000: mol_type = genomic DNA
atgttttacactgaaacttatgatgtgattgtgatcggtggtggtcatgcgggtacagaagccgcacttgcaccagctcgtatgggatttaaaacccttttattaacacataatgtagatactttagggcaaatgtcttgtaaccctgcaattggtgggatcggtaaaggtcatttagtaaaagaagtagatgcaatgggcggtttaatggcgcatgctgcagataaagcagggatccaatttcgtactttaaatagcagtaaaggcccagcagtgcgtgctactcgagctcaagctgacagagttctatatcgtcaagctgttcgtactgcattagaaaatcaacctaatttagatattttccaacaagaagcgaccgatattctgattaagcaagatcgagttacaggcgttagcacaaaaatgggattaacttttcgtgctaaatcagtggtattaactgcgggtactttcttagctggtaaaattcatattggtttggaaaattatgaaggtggccgtgcaggggatcctgcttctgtaaatctttcacatcgattaagagatctcggattacgtgtagatcgccttaaaacaggtacaccgccgcgtattgatgcacgtacgatcaattttgatattttagctaaacaacacggtgatgctgttttacctgtgttttcttttatgggatcagttgatgatcaccctcaacaaattccttgttatataactcataccaatgaacaaacccatgaagtgatccgtaataacttggatcgcagtccaatgtatactggtgtgattgaagggatcggtccacgttattgcccatccattgaagataaagtgatgcgtttctcggatcgtaattcacatcaaatttatttagaaccagaaggcttaaccagtaatgaagtgtatccaaacgggatctctaccagtttaccgtttgacgtgcaaatgggcattgtgaattctatgaaaggtttagaaaacg

SEQ ID NO: 3 - protein, 224 aa, Acinetobacter calcoaceticus
MNSIAELPLSIQISKKLEDDIIYGFYLPGTKLDEQELCERYGASRTPIREALKLLAAEGLVEIRPRRGAIIPTINPLTLCEMFEVMAELEAMCGRLAARRIQPEEKLELQRLHQLCQDYLNQNDSENYYEANRLFHFAIYQASHNAFLIEQACTLHKRLHPYRRLQLRVNNRMNNSFTEHNEILEAIFAGNEQQAEALLKAHVVIQGQKFTDFISTIESLQPKS

SEQ ID NO: 4 - protein, 222 aa, Rhizobium leguminosarum
MRKVKRMSENVGRWLRDEIENSILSNEFSPGERLDETVLATRFGVSRTPVREALMQLDAIGLIEIRPRRGAIVIDPGPHRVYEMFEVMAELEGLAGSLAARRLDKTSREAITATHGRCEKSAAAGDSDAYYYDNEEFHKAIYAAGRSDFLEEQCLQLHRRLRPDRRLQLRVRNRLSTSFLEHCAIVDAIFAGDGDEARRLLRGHVGIQGERFSDLVASMAAR

SEQ ID NO: 5 - protein, 308 aa, Klebsiella pneumoniae
MKDDINQEITFRKLSVFMMFMAKGNIARTAEAMKLSSVSVHRALHTLEEGVGCPLFVHKGRNLLPLQAAWTLLEYCQDVISLMNRGLEATRKVAGVGQGRLRIGTLYSLTLETVPRIIMGMKLRRPELELDLTMGSNQMLLDMLEDDALDAILIATNEGEFNNTAFDVVPLFEDDIFLAAPATERLDASRLADLRDYADRKFVSLAEGFATYAGFREAFHIAGFEPEIVTRVNDIFSMISLVQAGVGFALLPGRMKKVYEKDVQLLKLAEPYQMRQLISIVYSHHRERDADLLALAAEGRMYARSINR

SEQ ID NO: 6 - DNA, 77 bp, Acinetobacter calcoaceticus
  source 1..77: mol_type = genomic DNA
aaaaaaattgcattgtacactatacaattttgaatacagaatgtttattttaggctataagagtacaaagctatacc

SEQ ID NO: 7 - protein, 598 aa, synthetic construct
  VARIANT 1..16: note = Xaa can be any naturally occurring amino acid or absent
  VARIANT 18: note = Ile, Leu or Val
  VARIANT 19: note = Arg, Lys or His
  VARIANT 20..21: note = Ser or Thr
  VARIANT 22: note = Ile, Leu or Val
  VARIANT 23: note = Arg, Lys or His
  VARIANT 24: note = Xaa can be any naturally occurring amino acid
  VARIANT 27: note = Xaa can be any naturally occurring amino acid
  VARIANT 28: note = Ser or Thr
  VARIANT 29..30: note = Arg, Lys or His
  VARIANT 31: note = Xaa can be any naturally occurring amino acid
  VARIANT 32..35: note = Xaa can be any naturally occurring amino acid or absent
  VARIANT 36: note = Xaa can be any naturally occurring amino acid
  VARIANT 37..40: note = Xaa can be any naturally occurring amino acid or absent
  VARIANT 41: note = Xaa can be any naturally occurring amino acid
  VARIANT 44: note = Ile, Leu or Val
  VARIANT 45..46: note = Arg, Lys or His
  VARIANT 47: note = Ile, Leu or Val
  VARIANT 48: note = Arg, Lys or His
  VARIANT 49..52: note = Xaa can be any naturally occurring amino acid or absent
  VARIANT 53: note = Xaa can be any naturally occurring amino acid
  VARIANT 54..58: note = Xaa can be any naturally occurring amino acid or absent
  VARIANT 59: note = Xaa can be any naturally occurring amino acid
  VARIANT 60: note = Arg, Lys or His
  VARIANT 61: note = Ser or Thr
  VARIANT 62: note = Ile, Leu or Val
  VARIANT 63..65: note = Ser or Thr
  VARIANT 66..90: note = Xaa can be any naturally occurring amino acid or absent
  VARIANT 93: note = Ile, Leu or Val
  VARIANT 95..97: note = Xaa can be any naturally occurring amino acid
  VARIANT 98..100: note = Ser or Thr
  VARIANT 102: note = Ile, Leu or Val
  VARIANT 104: note = Ser or Thr
  VARIANT 105: note = Ile, Leu or Val
  VARIANT 107: note = Asp or Glu
  VARIANT 108: note = Ser or Thr
  VARIANT 111..112: note = Ile, Leu or Val
  VARIANT 113: note = Ser or Thr
  VARIANT 118: note = Arg, Lys or His
  VARIANT 125: note = Ser or Thr
  VARIANT 126: note = Asp or Glu
  VARIANT 128: note = Ser or Thr
  VARIANT 129: note = Asp or Glu
  VARIANT 130: note = Ser or Thr
  VARIANT 131: note = Ile, Leu or Val
  VARIANT 133: note = Arg, Lys or His
  VARIANT 134: note = Ser or Thr
  VARIANT 139: note = Ser or Thr
  VARIANT 142: note = Asp or Glu
  VARIANT 143..144: note = Ser or Thr
  VARIANT 146..149: note = Ile, Leu or Val
  VARIANT 150: note = Arg, Lys or His
  VARIANT 152: note = Ser or Thr
  VARIANT 157: note = Ser or Thr
  VARIANT 158: note = Ile, Leu or Val
  VARIANT 166: note = Ser or Thr
  VARIANT 167: note = Ile, Leu or Val
  VARIANT 169: note = Ile, Leu or Val
  VARIANT 170: note = Xaa can be any naturally occurring amino acid
  VARIANT 172: note = Ile, Leu or Val
  VARIANT 173: note = Asp or Glu
  VARIANT 174..175: note = Arg, Lys or His
  VARIANT 177..178: note = Xaa can be any naturally occurring amino acid or absent
  VARIANT 179..180: note = Arg, Lys or His
  VARIANT 181: note = Ser or Thr
  VARIANT 182: note = Ile, Leu or Val
  VARIANT 183: note = Asp or Glu
  VARIANT 186: note = Arg, Lys or His
  VARIANT 187: note = Ser or Thr
  VARIANT 188: note = Ala, Asp, Arg, His, Lys, Ser, Thr, Asn, Gln, Tyr
  VARIANT 191: note = Ile, Leu or Val
  VARIANT 195: note = Ile, Leu or Val
  VARIANT 199: note = Ile, Leu or Val
  VARIANT 202..205: note = Ile, Leu or Val
  VARIANT 207: note = Asp or Glu
  VARIANT 217: note = Ile, Leu or Val
  VARIANT 218: note = Ser or Thr
  VARIANT 219: note = Ile, Leu or Val
  VARIANT 221: note = Ser or Thr
  VARIANT 225: note = Ile, Leu or Val
  VARIANT 231: note = Arg, Lys or His
  VARIANT 247..248: note = Ser or Thr
  VARIANT 251: note = Ile, Leu or Val
  VARIANT 253: note = Arg, Lys or His
  VARIANT 254..255: note = Ile, Leu or Val
  VARIANT 256: note = Ser or Thr
  VARIANT 257: note = Ile, Leu or Val
  VARIANT 260: note = Ser or Thr
  VARIANT 261: note = Arg, Lys or His
  VARIANT 262: note = Ser or Thr
  VARIANT 264: note = Ile, Leu or Val
  VARIANT 266: note = Ile, Leu or Val
  VARIANT 270: note = Ile, Leu or Val
  VARIANT 273: note = Asp or Glu
  VARIANT 274..275: note = Ile, Leu or Val
  VARIANT 276: note = Ser or Thr
  VARIANT 278..279: note = Asp or Glu
  VARIANT 282..283: note = Ile, Leu or Val
  VARIANT 285: note = Ile, Leu or Val
  VARIANT 290: note = Ile, Leu or Val
  VARIANT 291: note = Ser or Thr
  VARIANT 292: note = Arg, Lys or His
  VARIANT 293: note = Asp or Glu
  VARIANT 295: note = Ile, Leu or Val
  VARIANT 296..297: note = Asp or Glu
  VARIANT 299: note = Asp or Glu
  VARIANT 300: note = Arg, Lys or His
  VARIANT 305: note = Ile, Leu or Val
  VARIANT 306: note = Arg, Lys or His
  VARIANT 308: note = Ser or Thr
  VARIANT 309: note = Asp or Glu
  VARIANT 310: note = Xaa can be any naturally occurring amino acid
  VARIANT 311: note = Ile, Leu or Val
  VARIANT 312: note = Asp or Glu
  VARIANT 313: note = Xaa can be any naturally occurring amino acid
  VARIANT 314: note = Xaa can be any naturally occurring amino acid or absent
  VARIANT 315..317: note = Xaa can be any naturally occurring amino acid
  VARIANT 318: note = Ser or Thr
  VARIANT 320: note = Ser or Thr
  VARIANT 321..322: note = Asp or Glu
  VARIANT 325: note = Asp or Glu
  VARIANT 326..327: note = Ile, Leu or Val
  VARIANT 329: note = Asp or Glu
  VARIANT 330: note = Ser or Thr
  VARIANT 331: note = Ile, Leu or Val
  VARIANT 332: note = Asp or Glu
  VARIANT 335..336: note = Ser or Thr
  VARIANT 338: note = Xaa can be any naturally occurring amino acid or absent
  VARIANT 339: note = Ile, Leu or Val
  VARIANT 341: note = Arg, Lys or His
  VARIANT 342: note = Asp or Glu
  VARIANT 344: note = Arg, Lys or His
  VARIANT 346: note = Arg, Lys or His
  VARIANT 348: note = Ser or Thr
  VARIANT 350: note = Asp or Glu
  VARIANT 351: note = Arg, Lys or His
  VARIANT 353: note = Asp or Glu
  VARIANT 356: note = Asp or Glu
  VARIANT 357: note = Arg, Lys or His
  VARIANT 360: note = Asp or Glu
  VARIANT 361: note = Ile, Leu or Val
  VARIANT 362: note = Ser or Thr
  VARIANT 363: note = Arg, Lys or His
  VARIANT 364: note = Ile, Leu or Val
  VARIANT 365..366: note = Ser or Thr
  VARIANT 367: note = Ile, Leu or Val
  VARIANT 368: note = Arg, Lys or His
  VARIANT 369: note = Asp or Glu
  VARIANT 370..371: note = Ile, Leu or Val
  VARIANT 372..373: note = Arg, Lys or His
  VARIANT 375..376: note = Asp or Glu
  VARIANT 377: note = Ile, Leu or Val
  VARIANT 378: note = Xaa can be any naturally occurring amino acid or absent
  VARIANT 379..380: note = Xaa can be any naturally occurring amino acid
  VARIANT 381: note = Xaa can be any naturally occurring amino acid or absent
  VARIANT 383: note = Asp or Glu
  VARIANT 385: note = Ser or Thr
  VARIANT 386..387: note = Asp or Glu
  VARIANT 389: note = Arg, Lys or His
  VARIANT 390: note = Asp or Glu
  VARIANT 393: note = Arg, Lys or His
  VARIANT 394: note = Asp or Glu
  VARIANT 395: note = Ile, Leu or Val
  VARIANT 396: note = Arg, Lys or His
  VARIANT 397: note = Asp or Glu
  VARIANT 398: note = Arg, Lys or His
  VARIANT 400: note = Ile, Leu or Val
  VARIANT 401: note = Ser or Thr
  VARIANT 405: note = Ser or Thr
  VARIANT 407: note = Ile, Leu or Val
  VARIANT 409: note = Ile, Leu or Val
  VARIANT 411: note = Ile, Leu or Val
  VARIANT 412: note = Arg, Lys or His
  VARIANT 413..414: note = Ile, Leu or Val
  VARIANT 415: note = Arg, Lys or His
  VARIANT 416: note = Asp or Glu
  VARIANT 418: note = Ser or Thr
  VARIANT 419: note = Arg, Lys or His
  VARIANT 420: note = Asp or Glu
  VARIANT 421: note = Arg, Lys or His
  VARIANT 422: note = Ile, Leu or Val
  VARIANT 423: note = Asp or Glu
  VARIANT 424: note = Ser or Thr
  VARIANT 426: note = Ile, Leu or Val
  VARIANT 427..428: note = Arg, Lys or His
  VARIANT 431: note = Ile, Leu or Val
  VARIANT 434: note = Ser or Thr
  VARIANT 438: note = Ile, Leu or Val
  VARIANT 440: note = Xaa can be any naturally occurring amino acid or absent
  VARIANT 441..443: note = Xaa can be any naturally occurring amino acid
  VARIANT 445: note = Asp or Glu
  VARIANT 446: note = Ser or Thr
  VARIANT 447..448: note = Ile, Leu or Val
  VARIANT 451: note = Ser or Thr
  VARIANT 452: note = Asp or Glu
  VARIANT 454: note = Ser or Thr
  VARIANT 455..456: note = Arg, Lys or His
  VARIANT 459: note = Ile, Leu or Val
  VARIANT 460: note = Asp or Glu
  VARIANT 463: note = Arg, Lys or His
  VARIANT 464: note = Ile, Leu or Val
  VARIANT 464: note = Ile, Leu or Val
  VARIANT 469..471: note = Arg, Lys or His
  VARIANT 472: note = Xaa can be any naturally occurring amino acid
  VARIANT 473..474: note = Asp or Glu
  VARIANT 475: note = Ile, Leu or Val
  VARIANT 476: note = Ser or Thr
  VARIANT 477: note = Ile, Leu or Val
  VARIANT 478: note = Ser or Thr
  VARIANT 480: note = Ile, Leu or Val
  VARIANT 481..482: note = Ser or Thr
  VARIANT 483..484: note = Ile, Leu or Val
  VARIANT 485: note = Ser or Thr
  VARIANT 487: note = Arg, Lys or His
  VARIANT 489: note = Ser or Thr
  VARIANT 490: note = Ile, Leu or Val
  VARIANT 492: note = Ile, Leu or Val
  VARIANT 493: note = Ser or Thr
  VARIANT 495: note = Ile, Leu or Val
  VARIANT 496: note = Arg, Lys or His
  VARIANT 498..499: note = Ser or Thr
  VARIANT 501: note = Ile, Leu or Val
  VARIANT 504: note = Arg, Lys or His
  VARIANT 505: note = Asp or Glu
  VARIANT 508..509: note = Arg, Lys or His
  VARIANT 510: note = Ile, Leu or Val
  VARIANT 511: note = Arg, Lys or His
  VARIANT 516: note = Ser or Thr
  VARIANT 517: note = Asp or Glu
  VARIANT 518: note = Ser or Thr
  VARIANT 519: note = Asp or Glu
  VARIANT 520: note = Ile, Leu or Val
  VARIANT 521: note = Arg, Lys or His
  VARIANT 524: note = Ile, Leu or Val
  VARIANT 525..527: note = Arg, Lys or His
  VARIANT 528: note = Asp or Glu
  VARIANT 530: note = Arg, Lys or His
  VARIANT 532..534: note = Xaa can be any naturally occurring amino acid or absent
  VARIANT 535: note = Xaa can be any naturally occurring amino acid
  VARIANT 537: note = Xaa can be any naturally occurring amino acid or absent
  VARIANT 538..539: note = Xaa can be any naturally occurring amino acid
  VARIANT 540: note = Ile, Leu or Val
  VARIANT 541: note = Arg, Lys or His
  VARIANT 542: note = Ile, Leu or Val
  VARIANT 543: note = Ser or Thr
  VARIANT 543: note = Ser or Thr
  VARIANT 544..545: note = Arg, Lys, or His
  VARIANT 546: note = Asp or Glu
  VARIANT 547..548: note = Ile, Leu or Val
  VARIANT 549..550: note = Arg, Lys or His
  VARIANT 552: note = Ser or Thr
  VARIANT 554: note = Ile, Leu or Val
  VARIANT 556: note = Asp or Glu
  VARIANT 557: note = Ser or Thr
  VARIANT 558..559: note = Arg, Lys or His
  VARIANT 560: note = Asp or Glu
  VARIANT 562: note = Ile, Leu or Val
  VARIANT 564: note = Ile, Leu or Val
  VARIANT 565: note = Asp or Glu
  VARIANT 566: note = Ser or Thr
  VARIANT 567..568: note = Ile, Leu or Val
  VARIANT 569: note = Arg, Lys or His
  VARIANT 570..572: note = Ile, Leu or Val
  VARIANT 573: note = Asp or Glu
  VARIANT 574..575: note = Arg, Lys or His
  VARIANT 577: note = Ser or Thr
  VARIANT 578..580: note = Asp or Glu
  VARIANT 582: note = Xaa can be any naturally occurring amino acid
  VARIANT 585: note = Ile, Leu or Val
  VARIANT 586: note = Arg, Lys or His
  VARIANT 588: note = Arg, Lys or His
  VARIANT 589..590: note = Xaa can be any naturally occurring amino acid
  VARIANT 591: note = Xaa can be any naturally occurring amino acid or absent
  VARIANT 592: note = Xaa can be any naturally occurring amino acid
  VARIANT 593..598: note = Xaa can be any naturally occurring amino acid or absent
XXXXXXXXXXXXXXXXMXXXXXXXAQXXXXXXXXXXXXXXXGFXXXXXXXXXXXXXXXXXXXXXXXXXXXXXXXXXXXXXXXXXXXXXXXAQXNXXXXXXVXFXXQXXARXXXLNRPXKLNALNXXMXXXXFXXLNEYXKSXXXNXXXXXSXNQPRXXCAGGDVAXXAXXNXXXXFXXXXXXXFFXXXYSXNFQXATYXKPXXXXMXGITMGGGVGXXXHXPFRXATENTXWAMPEMDIGFFPDVGXXFAXPXXXXXANXXXQXAXYLCXTGXXXXGXXAYXXGXASHYXXXXNXXXLXXRLGEXXPXXXXXXXXXXXQXXXFFXXXNXXXXEFXXPXXPXXYXFXYXNXXLXVIXXCFXXXXXXXXXXXXXXLXXXXXXXYXGXXXAXXFAXXXXXXLXXKSPXSXQXAXXXXXXNXXXXXXXAXXXDLXTAXNMCXNXXXXQXXXXEFXXAXXXKLXXKQXXPYPWXXXXXXXXXXQXXXXXXPXPXXPXXLXXNXXNXTWXXYPXXXXYQLPXXXXXXQYXXXXXNXNXXXXGXXXXXXXXXXXXXXFXNXNXXXXXKXGXXXXXXXXXXXXCXXXXAXGGXXWXXXXXXXXXXX

SEQ ID NO: 8 - protein, 351 aa, synthetic construct
  VARIANT 4: note = Arg, Lys or His
  VARIANT 9: note = Ile, Leu or Val
  VARIANT 10: note = Ser or Thr
  VARIANT 11: note = Asp or Glu
  VARIANT 16: note = Ser or Thr
  VARIANT 31: note = Asp or Glu
  VARIANT 45: note = Arg, Lys or His
  VARIANT 46..47: note = Asp or Glu
  VARIANT 48: note = Arg, Lys or His
  VARIANT 51: note = Ile, Leu or Val
  VARIANT 59: note = Ser or Thr
  VARIANT 79: note = Ile, Leu or Val
  VARIANT 83: note = Arg, Lys or His
  VARIANT 86: note = Arg, Lys or His
  VARIANT 89: note = Asp or Glu
  VARIANT 91: note = Ala, Asp, Arg, His, Lys, Ser, Thr, Asn, Gln, Tyr
  VARIANT 93: note = Asp or Glu
  VARIANT 98: note = Ile, Leu or Val
  VARIANT 102: note = Arg, Lys or His
  VARIANT 125: note = Arg, Lys or His
  VARIANT 131: note = Asp or Glu
  VARIANT 132: note = Arg, Lys or His
  VARIANT 156: note = Arg, Lys or His
  VARIANT 161: note = Ser or Thr
  VARIANT 163: note = Arg, Lys or His
  VARIANT 168: note = Ser or Thr
  VARIANT 170: note = Ser or Thr
  VARIANT 171: note = Ile, Leu or Val
  VARIANT 175: note = Ser or Thr
  VARIANT 188..189: note = Ser or Thr
  VARIANT 190: note = Asp or Glu
  VARIANT 191: note = Ser or Thr
  VARIANT 194: note = Arg, Lys or His
  VARIANT 197: note = Ser or Thr
  VARIANT 198: note = Asp or Glu
  VARIANT 199: note = Ile, Leu or Val
  VARIANT 200: note = Asp or Glu
  VARIANT 200: note = Ser or Thr
  VARIANT 201: note = Ser or Thr
  VARIANT 202: note = Ile, Leu or Val
  VARIANT 205..206: note = Arg, Lys or His
  VARIANT 207..209: note = Asp or Glu
  VARIANT 211: note = Arg, Lys or His
  VARIANT 212: note = Ser or Thr
  VARIANT 213: note = Arg, Lys or His
  VARIANT 216: note = Asp or Glu
  VARIANT 217..218: note = Ile, Leu or Val
  VARIANT 219: note = Arg, Lys or His
  VARIANT 220: note = Ser or Thr
  VARIANT 223..226: note = Ser or Thr
  VARIANT 227: note = Ile, Leu or Val
  VARIANT 228: note = Asp or Glu
  VARIANT 229: note = Ser or Thr
  VARIANT 230: note = Asp or Glu
  VARIANT 232..233: note = Ser or Thr
  VARIANT 234..235: note = Ile, Leu or Val
  VARIANT 237: note = Asp or Glu
  VARIANT 238: note = Ile, Leu or Val
  VARIANT 240: note = Ser or Thr
  VARIANT 245: note = Asp or Glu
  VARIANT 246: note = Ser or Thr
  VARIANT 247: note = Ile, Leu or Val
  VARIANT 249: note = Asp or Glu
  VARIANT 252: note = Arg, Lys or His
  VARIANT 256: note = Arg, Lys or His
  VARIANT 257: note = Asp or Glu
  VARIANT 259..260: note = Ser or Thr
  VARIANT 263: note = Ile, Leu or Val
  VARIANT 264: note = Arg, Lys or His
  VARIANT 265: note = Ser or Thr
  VARIANT 266: note = Arg, Lys or His
  VARIANT 267: note = Asp or Glu
  VARIANT 268: note = Ser or Thr
  VARIANT 275: note = Ile, Leu or Val
  VARIANT 285: note = Ile, Leu or Val
  VARIANT 286: note = Asp or Glu
  VARIANT 288: note = Arg, Lys or His
  VARIANT 289: note = Asp or Glu
  VARIANT 292: note = Ile, Leu or Val
  VARIANT 294: note = Asp or Glu
  VARIANT 301: note = Ile, Leu or Val
  VARIANT 310: note = Asp or Glu
  VARIANT 319: note = Ile, Leu or Val
  VARIANT 321: note = Ile, Leu or Val
  VARIANT 332: note = Arg, Lys or His
  VARIANT 334: note = Ile, Leu or Val
  VARIANT 335: note = Ser or Thr
  VARIANT 338: note = Ser or Thr
  VARIANT 339..341: note = Asp or Glu
  VARIANT 344: note = Arg, Lys or His
MTEXVLFSXXXNGVAXITLNRPKALNSLSYXMLQPIGQKLKEWEXXXXIAXIVLKGAGXKGFCAGGDIKTLYEARSNEXALQXAEXFFXEXYXIDTYXYQYXKPIIACLDGIVMGGGVGLTNGAXYRIVTXXTKWAMPEMNIGFFPDVGAAYFLNXAPGYXGXYVALXAXXLKAXDVLFINAADYFMXXXXLPXFLXXXXXXNWXXXXXVXXXLKXXXXXFAXXXXXXXXLXXXXEXXNXHFAFXXXEXIIXSLEXXQXXFAXXXXXXLLSKSPXSLKVTLKQFXXGXXKSXEXCFATDLXLAKNFMRHXDFFEGVRSXVXDKDQNPNYKYXQXXDVXXXXVNXFFNLLNA

SEQ ID NO: 9 - protein, 370 aa, synthetic construct
  VARIANT 3: note = Ile, Leu or Val
  VARIANT 4: note = Arg, Lys or His
  VARIANT 7: note = Asp or Glu
  VARIANT 8: note = Arg, Lys or His
  VARIANT 9..10: note = Ser or Thr
  VARIANT 11: note = Ile, Leu or Val
  VARIANT 12: note = Arg, Lys or His
  VARIANT 18: note = Ile, Leu or Val
  VARIANT 20: note = Ser or Thr
  VARIANT 24: note = Asp or Glu
  VARIANT 25: note = Arg, Lys or His
  VARIANT 26: note = Ser or Thr
  VARIANT 31: note = Ser or Thr
  VARIANT 36: note = Asp or Glu
  VARIANT 37: note = Ile, Leu or Val
  VARIANT 40: note = Asp or Glu
  VARIANT 41: note = Arg, Lys or His
  VARIANT 42: note = Ile, Leu or Val
  VARIANT 43: note = Arg, Lys or His
  VARIANT 47: note = Arg, Lys or His
  VARIANT 48: note = Asp or Glu
  VARIANT 51..52: note = Ile, Leu or Val
  VARIANT 55: note = Ile, Leu or Val
  VARIANT 69: note = Asp or Glu
  VARIANT 71..72: note = Arg, Lys or His
  VARIANT 74: note = Ile, Leu or Val
  VARIANT 75: note = Asp or Glu
  VARIANT 78: note = Arg, Lys or His
  VARIANT 79: note = Asp or Glu
  VARIANT 80: note = Arg, Lys or His
  VARIANT 83: note = Asp or Glu
  VARIANT 84: note = Ile, Leu or Val
  VARIANT 90: note = Arg, Lys or His
  VARIANT 94: note = Asp or Glu
  VARIANT 95: note = Ala, Asp, Arg, His, Lys, Ser, Thr, Asn, Gln, or Tyr
  VARIANT 99: note = Asp or Glu
  VARIANT 100: note = Arg, Lys or His
  VARIANT 101: note = Arg, Lys or His
  VARIANT 102: note = Ile, Leu or Val
  VARIANT 104: note = Ser or Thr
  VARIANT 109..110: note = Ile, Leu or Val
  VARIANT 116: note = Arg, Lys or His
  VARIANT 118: note = Ile, Leu or Val
  VARIANT 125: note = Ile, Leu or Val
  VARIANT 129: note = Ser or Thr
  VARIANT 130: note = Ile, Leu or Val
  VARIANT 136..137: note = Ser or Thr
  VARIANT 144: note = Ile, Leu or Val
  VARIANT 145: note = Ser or Thr
  VARIANT 159: note = Ser or Thr
  VARIANT 161: note = Ile, Leu or Val
  VARIANT 164: note = Arg, Lys or His
  VARIANT 169: note = Ile, Leu or Val
  VARIANT 172: note = Ser or Thr
  VARIANT 175: note = Arg, Lys or His
  VARIANT 176: note = Ile, Leu or Val
  VARIANT 179: note = Arg, Lys or His
  VARIANT 182: note = Ile, Leu or Val
  VARIANT 185: note = Asp or Glu
  VARIANT 191: note = Ile, Leu or Val
  VARIANT 192: note = Arg, Lys or His
  VARIANT 193: note = Asp or Glu
  VARIANT 194: note = Arg, Lys or His
  VARIANT 197..198: note = Asp or Glu
  VARIANT 200: note = Ile, Leu or Val
  VARIANT 201..202: note = Asp or Glu
  VARIANT 204: note = Ile, Leu or Val
  VARIANT 206: note = Ile, Leu or Val
  VARIANT 212: note = Ser or Thr
  VARIANT 213: note = Asp or Glu
  VARIANT 214: note = Ile, Leu or Val
  VARIANT 217: note = Arg, Lys or His
  VARIANT 220: note = Ile, Leu or Val
  VARIANT 221: note = Arg, Lys or His
  VARIANT 223..224: note = Asp or Glu
  VARIANT 225: note = Arg, Lys or His
  VARIANT 226: note = Arg, Lys or His
  VARIANT 228: note = Arg, Lys or His
  VARIANT 229..230: note = Asp or Glu
  VARIANT 231: note = Ile, Leu or Val
  VARIANT 233: note = Asp or Glu
  VARIANT 236: note = Ile, Leu or Val
  VARIANT 242: note = Arg, Lys or His
  VARIANT 243: note = Ile, Leu or Val
  VARIANT 245: note = Asp or Glu
  VARIANT 246: note = Ile, Leu or Val
  VARIANT 249: note = Ile, Leu or Val
  VARIANT 251: note = Asp or Glu
  VARIANT 252: note = Ile, Leu or Val
  VARIANT 254: note = Ser or Thr
  VARIANT 257: note = Arg, Lys or His
  VARIANT 259: note = Ile, Leu or Val
  VARIANT 260: note = Asp or Glu
  VARIANT 262: note = Ile, Leu or Val
  VARIANT 263: note = Arg, Lys or His
  VARIANT 264: note = Asp or Glu
  VARIANT 265: note = Arg, Lys or His
  VARIANT 266: note = Asp or Glu
  VARIANT 270: note = Ile, Leu or Val
  VARIANT 272: note = Arg, Lys or His
  VARIANT 275..276: note = Arg, Lys or His
  VARIANT 277: note = Ile, Leu or Val
  VARIANT 278: note = Arg, Lys or His
  VARIANT 279: note = Asp or Glu
  VARIANT 283: note = Ile, Leu or Val
  VARIANT 284: note = Ser or Thr
  VARIANT 286: note = Arg, Lys or His
  VARIANT 287: note = Ile, Leu or Val
  VARIANT 290: note = Asp or Glu
  VARIANT 292: note = Ile, Leu or Val
  VARIANT 293: note = Arg, Lys or His
  VARIANT 297: note = Arg, Lys or His
  VARIANT 302: note = Asp or Glu
  VARIANT 303: note = Ile, Leu or Val
  VARIANT 305: note = Arg, Lys or His
  VARIANT 309: note = Ser or Thr
  VARIANT 310: note = Ile, Leu or Val
  VARIANT 319: note = Asp or Glu
  VARIANT 321: note = Ser or Thr
  VARIANT 329: note = Ile, Leu or Val
  VARIANT 331: note = Arg, Lys or His
  VARIANT 333: note = Arg, Lys or His
  VARIANT 336: note = Arg, Lys or His
  VARIANT 338: note = Arg, Lys or His
  VARIANT 341: note = Asp or Glu
  VARIANT 342: note = Ile, Leu or Val
  VARIANT 345: note = Ile, Leu or Val
  VARIANT 347: note = Asp or Glu
  VARIANT 349..350: note = Ile, Leu or Val
  VARIANT 350: note = Ile, Leu or Val
  VARIANT 351: note = Asp or Glu
  VARIANT 355: note = Asp or Glu
  VARIANT 356: note = Arg, Lys or His
  VARIANT 357: note = Ile, Leu or Val
  VARIANT 357: note = Ile, Leu or Val
  VARIANT 359: note = Asp or Glu
  VARIANT 361..362: note = Arg, Lys or His
  VARIANT 364: note = Ile, Leu or Val
  VARIANT 364: note = Ile, Leu or Val
  VARIANT 366: note = Asp or Glu
  VARIANT 367: note = Ile, Leu or Val
  VARIANT 367: note = Ile, Leu or Val
  VARIANT 368: note = Ser or Thr
  VARIANT 369..370: note = Xaa can be any naturally occurring amino acid
MNXXFEXXXXXXGARIGXAXLDAXXXLNALXLPMIXXLGXXXXAWAXXPGXXCVXLRGNGAKAFCAGGXVXXLXXACXXXPGXXPPLAAXFFAXXYRLXXXXHXYPKPXXCWGHGXVXGGGMGLXQGAXXRIVTPXXRLAMPEXXIGLYPDVGASWFLXRXPGXLGLFXGLXGAXXNAXDAXDLXLADRFXXXXQQXXLXXXLXQXNWQEQXXXQLXSLXXAXXXXAXXXXPXAQXLPRRQXXDXXLDXAXXAXAWXAXXAXXXXXDPLXAXAAXXXXXGCPXXAXXVWXQXXRARXLSLAXXFXMEYXXSLNCCRHPXFXEGVRARLXDXDXQPXWXWPXXAQXPXAXXXAHFXXXWXGXXPXAXXXXX

SEQ ID NO: 10 - protein, 505 aa, synthetic construct
  VARIANT 1..10: note = Xaa can be any naturally occurring amino acid or absent
  VARIANT 11: note = Xaa can be any naturally occurring amino acid
  VARIANT 12..23: note = Xaa can be any naturally occurring amino acid or absent
  VARIANT 24: note = Xaa can be any naturally occurring amino acid
  VARIANT 25..30: note = Xaa can be any naturally occurring amino acid or absent
  VARIANT 32: note = Xaa can be any naturally occurring amino acid
  VARIANT 34..35: note = Xaa can be any naturally occurring amino acid or absent
  VARIANT 39..40: note = Ile, Leu or Val
  VARIANT 42: note = Ser or Thr
  VARIANT 43: note = Ile, Leu or Val
  VARIANT 48: note = Ile, Leu or Val
  VARIANT 52: note = Ser or Thr
  VARIANT 57: note = Arg, Lys or His
  VARIANT 63: note = Ser or Thr
  VARIANT 67: note = Ile, Leu or Val
  VARIANT 70: note = Ile, Leu or Val
  VARIANT 73: note = Arg, Lys or His
  VARIANT 74: note = Ile, Leu or Val
  VARIANT 75: note = Arg, Lys or His
  VARIANT 80: note = Asp or Glu
  VARIANT 83: note = Ile, Leu or Val
  VARIANT 86..87: note = Ile, Leu or Val
  VARIANT 89: note = Arg, Lys or His
  VARIANT 90: note = Xaa can be any naturally occurring amino acid or absent
  VARIANT 95: note = Arg, Lys or His
  VARIANT 102: note = Asp or Glu
  VARIANT 103: note = Ile, Leu or Val
  VARIANT 104: note = Arg, Lys or His
  VARIANT 105: note = Ser or Thr
  VARIANT 106: note = Ile, Leu or Val
  VARIANT 108: note = Asp or Glu
  VARIANT 116: note = Xaa can be any naturally occurring amino acid
  VARIANT 127: note = Asp or Glu
  VARIANT 128: note = Xaa can be any naturally occurring amino acid
  VARIANT 128: note = Ala, Asp, Arg, His, Lys, Ser, Thr, Asn, Glu, Tyr
  VARIANT 131: note = Ile, Leu or Val
  VARIANT 132: note = Asp or Glu
  VARIANT 135: note = Ile, Leu or Val
  VARIANT 142..143: note = Ile, Leu or Val
  VARIANT 150: note = Ile, Leu or Val
  VARIANT 164: note = Arg, Lys or His
  VARIANT 165..166: note = Ile, Leu or Val
  VARIANT 170: note = Ser or Thr
  VARIANT 171: note = Arg, Lys or His
  VARIANT 193: note = Arg, Lys or His
  VARIANT 195..200: note = Xaa can be any naturally occurring amino acid or absent
  VARIANT 208: note = Ile, Leu or Val
  VARIANT 211: note = Ser or Thr
  VARIANT 215: note = Ile, Leu or Val
  VARIANT 221: note = Ile, Leu or Val
  VARIANT 223: note = Ile, Leu or Val
  VARIANT 227: note = Asp or Glu
  VARIANT 233: note = Asp or Glu
  VARIANT 239: note = Ile, Leu or Val
  VARIANT 242: note = Ile, Leu or Val
  VARIANT 245: note = Ile, Leu or Val
  VARIANT 252: note = Asp or Glu
  VARIANT 253: note = Ile, Leu or Val
  VARIANT 261: note = Xaa can be any naturally occurring amino acid
  VARIANT 262: note = Xaa can be any naturally occurring amino acid or absent
  VARIANT 263: note = Xaa can be any naturally occurring amino acid
  VARIANT 265: note = Ile, Leu or Val
  VARIANT 275: note = Asp or Glu
  VARIANT 278: note = Ile, Leu or Val
  VARIANT 281..284: note = Xaa can be any naturally occurring amino acid or absent
  VARIANT 289: note = Ile, Leu or Val
  VARIANT 296..298: note = Xaa can be any naturally occurring amino acid or absent
  VARIANT 301: note = Ile, Leu or Val
  VARIANT 306: note = Xaa can be any naturally occurring amino acid
  VARIANT 308: note = Ile, Leu or Val
  VARIANT 311: note = Ile, Leu or Val
  VARIANT 313..314: note = Xaa can be any naturally occurring amino acid or absent
  VARIANT 316: note = Asp or Glu
  VARIANT 328: note = Ile, Leu or Val
  VARIANT 332: note = Ser or Thr
  VARIANT 334: note = Ile, Leu or Val
  VARIANT 335: note = Ser or Thr
  VARIANT 337: note = Arg, Lys or His
  VARIANT 338: note = Ile, Leu or Val
  VARIANT 350: note = Ser or Thr
  VARIANT 351: note = Ile, Leu or Val
  VARIANT 353: note = Asp or Glu
  VARIANT 358: note = Asp or Glu
  VARIANT 361: note = Ile, Leu or Val
  VARIANT 367..368: note = Arg, Lys or His
  VARIANT 369..372: note = Xaa can be any naturally occurring amino acid or absent
  VARIANT 374: note = Asp or Glu
  VARIANT 379: note = Ile, Leu or Val
  VARIANT 380: note = Arg, Lys or His
  VARIANT 383..384: note = Ile, Leu or Val
  VARIANT 386: note = Arg, Lys or His
  VARIANT 387: note = Asp or Glu
  VARIANT 393: note = Arg, Lys or His
  VARIANT 397: note = Ile, Leu or Val
  VARIANT 400: note = Ile, Leu or Val
  VARIANT 402: note = Asp or Glu
  VARIANT 405..419: note = Xaa can be any naturally occurring amino acid or absent
  VARIANT 420: note = Ile, Leu or Val
  VARIANT 427: note = Ile, Leu or Val
  VARIANT 431: note = Xaa can be any naturally occurring amino acid
  VARIANT 433..434: note = Xaa can be any naturally occurring amino acid or absent
  VARIANT 440: note = Xaa can be any naturally occurring amino acid
  VARIANT 441..446: note = Xaa can be any naturally occurring amino acid or absent
  VARIANT 447..448: note = Xaa can be any naturally occurring amino acid
  VARIANT 449..505: note = Xaa can be any naturally occurring amino acid or absent
XXXXXXXXXXXXXXXXXXXXXXXXXXXXXXMXMXXTEHXXFXXSENGXASIXLNRPXALNSLXYDMXQPXGQXXXEWENXERXALXXLXXGAGTXGFCAGGXXXXXYXARSNEPGXALQHAERFFEXXYEXXTYXYQYKKPXXACLDGIXMGGGVGLTNGAKYXXXTERXXWAMPEMNIGFFPDVGAAYFLNXAXXXXXXPGYLGRYXALXASIXKASDVXFXNAAXYFMTSXSLPAFXTEXESXNWHKEDXXHTHLLKEXXXVXRTFATAPNLXSEXAPXXXXSLEEXNSHFAFXXXDTXEEIWXAXHSXEXXKXQSSFALKTKETXLSKXPXXLXXTLKQFIDGRDKXXEXCFATXLVXAKNFMXXXXXXEXFFEGXXSVXXDXXQNPNYXYKQXSDXSXEDXXXXXXXXXXXXXXXXNRFFNLXNAGXHXXPLADLXXXXXXXXXXXXXXXXXXXXXXXXXXXXXXXXXXXXXXXXXXXXXXXXXXXXXXXXXXXXXXXXXX

SEQ ID NO: 11 - DNA, 6801 bp, synthetic construct
  source 1..6801: mol_type = other DNA
atgagattgcagttgagaaccttgactagaaggtttttctctatggcttctggttcttctactccagatgttgctccattggttgatccaaatattcataagggtttggcctctcactttttcggtttgaactctgttcatactgctaagccatctaaggtcaaagaatttgttgcttctcatggtggtcataccgttatcaacaaagttttgattgccaacaacggtattgctgccgtaaaagaaattaggtctgttagaaaatgggcctacgaaacttttggtgacgaaagagctatttccttcactgttatggctacacctgaagatttggctgctaatgctgattacattagaatggccgatcaatacgttgaagttccaggtggtacaaacaacaacaattacgctaacgttgaattgatcgttgatgtcgctgaaagatttggtgttgatgctgtttgggctggttggggtcatgcttctgaaaatccattattgccagaatctttggctgcttctccaagaaagatcgtttttattggtccaccaggtgctgctatgagatcattgggtgataagatttcttctaccatcgttgctcaacatgctaaggttccatgtattccatggtctggtactggtgttgatgaagttgttgttgataagtccaccaacttggtttccgtttctgaagaagtttacactaagggttgtactactggtccaaaacaaggtttggaaaaggctaagcaaattggtttcccagttatgattaaggcttctgaaggtggtggtggtaaaggtattagaaaggttgaaagggaagaagatttcgaagctgcttaccatcaagttgaaggtgaaattccaggttccccaattttcattatgcaattggctggtaacgccagacatttggaagttcaattattggctgatcagtacggcaacaacatttctttgtttggtagagattgctccgttcaaagaaggcatcaaaagattattgaagaagccccagttactgttgctggtcaacaaacttttactgctatggaaaaagctgccgtcagattgggtaaattggttggttatgtttctgccggtactgtcgaatacttgtactctcatgaagatgacaagttctacttcttggaattgaacccaagattgcaagttgaacatccaactactgaaatggttaccggtgttaatttgccagctgctcaattgcaaattgctatgggtattccattggacagaatcaaggatattaggttgttctacggtgttaacccacataccactactccaattgatttcgatttctctggtgaagatgctgacaaaactcaaagaaggccagttccaagaggtcatacaactgcttgtagaattacttctgaagatccaggtgaaggttttaaaccatctggtggtactatgcacgaattgaactttaggtcctcttctaatgtttggggttacttctctgttggtaatcaaggtggtatccactctttctctgattctcaattcggtcatattttcgcctttggtgaaaacagatccgcctcaagaaaacatatggttgttgccttgaaagaattgtccatcagaggtgatttcagaaccactgttgaatacttgatcaaattattggaaaccccagacttcgaggataacactattactactggttggttggacgagttgatctctaacaaattgactgctgaaagaccagattccttcttggctgttgtttgtggtgctgctacaaaagctcatagagcttcagaagattctatcgctacttacatggcttctttggaaaaaggtcaagttccagccagagacattttgaaaactttgttcccagttgacttcatctacgaaggtcaaagatacaagtttaccgctaccagatcatccgaagattcttacactttgttcatcaacggttccagatgcgatattggtgttagaccattgtctgatggtggtattttgtgtttggttggtggtagatcccataacgtttattggaaagaagaagttggcgctaccagattgtctgttgattctaaaacttgcttgttggaagtcgaaaacgatccaactcaattgagatcaccatctccaggtaagttggttaagttcttggttgaaaacggtgatcacgttagagctaatcaaccatacgctgaaatcgaagtcatgaagatgtacatgactttgaccgctcaagaagatggtatcgttcaattgatgaagcaaccaggttctactattgaagccggtgatattttgggtattttggctttggatgatccatccaaggtcaaacatgctaaaccatttgaaggtcagttgccagaattgggtccaccaacattgtctggtaacaaaccacatcaaagatacgaacattgccaaaacgtcttgcacaacattttgttgggtttcgataaccaggttgtcatgaagtctacattgcaagaaatggtcggtttgttgagaaatccagaattgccatacttgcaatgggctcatcaagtttcttcattgcatacaagaatgtccgctaagttggatgctactttggctggtttgattgataaggctaaacaaaggggtggtgaatttccagctaagcaattattgagagccttggaaaaagaagcttcatctggcgaagttgatgctttgtttcaacaaacattggcccctttgtttgatttggctagagaatatcaagatggtttggccatccatgaattgcaagttgctgctggtttgttgcaagcttattatgattctgaagctagattctgcggtccaaacgttagagatgaagatgttatcttgaagttgagggaagaaaacagggactctttgagaaaagttgttatggcccaattgtcccattcaagagttggtgctaaaaacaacttggttttggctttgttggatgagtacaaggttgctgatcaagctggtactgattctccagcttctaatgttcatgttgctaaatacttgaggccagtcttgagaaagattgtcgaattggaatcaagagcttccgctaaggtttctttgaaggctagagaaattttgatccaatgcgctttgccatccttgaaagaaagaactgatcaattggaacacatcttgagatcctctgttgttgaatcaagatacggtgaagttggtttggaacatagaactccaagagctgacatcttgaaagaagttgttgactccaagtacatcgtgttcgatgttttggctcaattcttcgctcatgatgatccatggatagttttggctgctttggagttgtatattagaagggcttgtaaggcctactccattttggatattaactaccaccaagactctgatttgccaccagttatttcttggagattcagattgccaactatgtcatctgccttgtacaactctgttgtttcttctggttctaagactccaacttctccatctgtttcaagagctgattctgtttccgatttctcttacaccgttgaaagagattctgctccagctagaactggtgctatagttgctgttccacatttggatgatttggaagatgctttgaccagagtcttggaaaatttgccaaaaagaggtgctggtttggctatttctgttggtgcttctaacaaatcagctgctgcttctgctagagatgctgctgctgcagctgcttcttctgttgatactggtttgtctaacatctgcaacgttatgatcggtagagttgatgaatccgatgatgatgataccttgatcgccagaatttcccaagttatcgaagatttcaaagaggacttcgaagcttgctccttgagaagaattactttctcattcggtaactccagaggtacttacccaaagtactttacttttagaggtccagcctatgaagaagatccaaccattagacatattgaaccagctttggcctttcaattggaattggctagattgtctaacttcgacatcaagccagttcataccgataacagaaacatccatgtttacgaagctactggtaagaatgctgcttccgataagagatttttcaccagaggtatagttagaccaggtagattgagagaaaacatcccaacatccgagtacttgatttctgaagctgatagattgatgtccgatattttggatgccttggaagttattggtactaccaactctgatttgaaccacatcttcattaacttctccgctgtttttgctttgaagccagaagaagttgaagctgcttttggtggttttttggaaagattcggtagaagattgtggagattgagagttactggtgccgaaattagaatgatggtttctgatccagaaactggttctgcttttccattgagagctatgatcaacaacgtttccggttacgttgtccaatctgaattatacgctgaagccaagaatgataagggtcaatggatctttaagtccttgggtaaaccaggttcaatgcatatgagatccattaacactccataccctaccaaagaatggttgcaacctaaaagatacaaggcccatttgatgggtactacctactgttatgatttcccagagttgttcagacagtccattgaatctgattggaaaaagtacgatggtaaggctccagatgatttgatgacttgcaacgaattgatcttggacgaagattctggtgaattgcaagaagttaatagagaaccaggtgctaacaacgttggtatggttgcttggaaatttgaagctaagactccagaatatccaaggggtagaagttttatcgttgttgccaacgatattaccttccagattggttcttttggtccagctgaagatcaattcttcttcaaggttactgaattggccagaaagttgggtattccaagaatctacttgtctgctaattccggtgctagaattggtattgctgatgaattggtcggtaagtacaaagttgcttggaatgacgaaactgatccatctaagggtttcaagtacttgtacttcactccagaatcattggctaccttgaaaccagatactgttgttaccaccgaaattgaagaagaaggtccaaacggcgttgaaaagagacatgttattgattacatcgtcggtgaaaaggatggtttgggtgttgaatgtttgagaggttctggtttaattgctggtgctacttcaagagcttacaaggatattttcaccttgaccttggttacctgtagatcagttggtattggtgcttacttggttagattgggtcaaagagccattcaaattgaaggtcagccaattatcttgactggtgctccagctattaacaagttgttgggtagagaagtctactcctctaacttgcaattgggtggtactcaaatcatgtacaacaacggtgtttctcatttgaccgctagagatgatttgaacggtgttcataagatcatgcagtggttgtcttatattccagcttcaagaggtttgccagttccagttttgccacataagactgatgtttgggatagagatgttaccttccaaccagttagaggtgaacaatatgatgtcagatggttgatttctggtaggactttggaagatggtgcttttgaatctggtttgttcgataaggactctttccaagaaactttatctggttgggctaagggtgttgttgttggtagagctagattgggtggtattccatttggtgttattggtgttgaaactgccactgttgataacactactccagctgatccagctaatccagattctattgaaatgtctacttccgaagctggtcaagtttggtatccaaattctgctttcaagacctcccaagccattaacgattttaatcatggtgaagccttgccattgatgattttggctaattggagaggtttttccggtggtcaaagagatatgtacaacgaagttttgaagtacggctcctttattgtcgatgctttggttgattacaagcagccaattatggtttacattccaccaactggtgaattgagaggtggttcttgggttgttgttgacccaactattaactccgatatgatggaaatgtacgccgatgttgaaagtagaggtggtgttttggaaccagaaggtatggttggtattaagtacagaagagacaagttgttagataccatggccagattagatccagagtactcttccttgaaaaaacaattggaagagtccccagactccgaagaattgaaagttaagttgtccgtcagggaaaagtctttgatgccaatctaccaacaaatctccgttcaatttgctgacttgcatgatagagctggtagaatggaagctaaaggtgttattagagaagccttggtttggaaggatgctagaagatttttcttttggaggatcagaagaaggttggtcgaggaatatttgatcaccaagatcaactccattttgccatcttgtaccagattggaatgtttggctagaatcaaatcttggaagccagctactttggatcaaggttctgatagaggtgttgctgaatggtttgacgaaaattctgatgctgtttctgccaggttgtctgaattgaaaaaagatgcttctgctcagtccttcgcttcccaattgagaaaagatagacaaggtacattgcagggtatgaagcaagctttggcttctttgtctgaagctgaaagagctgaattattgaagggcttgtaa

SEQ ID NO: 12 - DNA, 7891 bp, synthetic construct
  source 1..7891: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacgcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctccaccgcggtggcggccgcataggccactagtggatctgatatcatcgatgaattcgagctcgttttcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactgattattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttacccatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggcaagctaaacagatctcccgggtagtttaaacaatgaatgtcacctttgaagaaagagccagtttacacggttacagaatcggtatcgcatccttagacgccccagcatccttgaacgctttgtccttaccaatgattgatgctttacaagacagattgagagcttgggcagaagatgcagacatagcctgtgtcttgttacgtggtaatggttctaaagcattttgcgcaggtggtgacgttgtccaattagctaagaaatgtttggcctccccaggtgaagcccctgaattggctgaaagatttttcgctagaagttacagattagatcattatttgcacacatacccaaagcctttgatatgctgggctcatggtcacgttttaggtggtggtatgggtttgttacaaggtgctggtattagaatagtcaccccatcttcaagattggcaatgcctgaaatctctattggtttatttccagatgttggtggttcccatttcttaagtagattgcctggtaaattgggtttatttttcggtttaaccgcttcaccattgaatgccagagatgctttggacttaaacttggctgatagattcttgttagatactcaacaagacgcattgatcgatggtttgatccaattgaactggagagaacaaccagatttgcaattgcattccttgttaaaggctttagaacaacaagcaagaagtgaattaccagctgcacaatggttgcctagaagagaaagattagacgccttgttagatcaagctactttaccattgtcttggcaagccttagcttcattggaaaacgatgaagacgcattgttagccaaagccgctaagactatgttgggtggttcacctttaacaggtcatttggtctggggtcaaattagaagagctagacacttatctttggcacaagtatttcaaatggaatacggcatgtcattgaattgttgcagacatccagaatttgcagaaggtgtaagagccagattaatcgataaagaccacgctccacattggcactggcctgacgtaaaccaagttcctgaagccgttattgcagcccattttgcaccattagatgaccaccctttagctgatttggcataagtttaaactcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggcctctagcagcttttgttccctttagtgagggttaatttcgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 13 - DNA, 60 bp, synthetic construct
  source 1..60: mol_type = other DNA
ccaatatataataaaatatggaggaatgcgatgctcagaaatacgctaaaatgtgcccaa

SEQ ID NO: 14 - DNA, 60 bp, synthetic construct
  source 1..60: mol_type = other DNA
tgcctggagatccttactcgagttggatccttatttccatcttaagccatcgttaacttc

SEQ ID NO: 15 - DNA, 37 bp, synthetic construct
  source 1..37: mol_type = other DNA
ttttactgatgcgtattctttgaattttcaaatagca

SEQ ID NO: 16 - DNA, 34 bp, synthetic construct
  source 1..34: mol_type = other DNA
tcaaagaatacgcatcagtaaaaaatttgatgga

SEQ ID NO: 17 - DNA, 41 bp, synthetic construct
  source 1..41: mol_type = other DNA
ttttactgatgtttattctttgaattttcaaatagcaactt

SEQ ID NO: 18 - DNA, 39 bp, synthetic construct
  source 1..39: mol_type = other DNA
tcaaagaataaacatcagtaaaaaatttgatggacttgg

SEQ ID NO: 19 - DNA, 39 bp, synthetic construct
  source 1..39: mol_type = other DNA
ttttactgattcgtattctttgaattttcaaatagcaac

SEQ ID NO: 20 - DNA, 36 bp, synthetic construct
  source 1..36: mol_type = other DNA
tcaaagaatacgaatcagtaaaaaatttgatggact

SEQ ID NO: 21 - DNA, 55 bp, synthetic construct
  source 1..55: mol_type = other DNA
ccaatatataataaaatatggaggaatgcgatgtctacaacacataacgtccctc

SEQ ID NO: 22 - DNA, 53 bp, synthetic construct
  source 1..53: mol_type = other DNA
tgcctggagatccttactcgagttggatccttactcaacaggtaaggcgcgag

SEQ ID NO: 23 - DNA, 61 bp, synthetic construct
  source 1..61: mol_type = other DNA
ctaattacatgactcgaggtcgacggtatcgttatttccatcttaagccatcgttaacttc

SEQ ID NO: 24 - DNA, 67 bp, synthetic construct
  source 1..67: mol_type = other DNA
cattagaaagaaagcatagcaatctaatctaagtttaaaacaatgactactcaaccccagctaaatg

SEQ ID NO: 25 - DNA, 60 bp, synthetic construct
  source 1..60: mol_type = other DNA
gaaagcatagcaatctaatctaagtttaaaacaatgaccgaacaagtcttattctcagta

SEQ ID NO: 26 - DNA, 59 bp, synthetic construct
  source 1..59: mol_type = other DNA
ctaattacatgactcgaggtcgacggtatcgttaagcgttcaacaaattgaaaaatctg

SEQ ID NO: 27 - DNA, 58 bp, synthetic construct
  source 1..58: mol_type = other DNA
gaaagcatagcaatctaatctaagtttaaaacaatgaccgaacatgtattattctcag

SEQ ID NO: 28 - DNA, 57 bp, synthetic construct
  source 1..57: mol_type = other DNA
ctaattacatgactcgaggtcgacggtatcgttaagcgtttaacaaattgaaaaatc

SEQ ID NO: 29 - DNA, 57 bp, synthetic construct
  source 1..57: mol_type = other DNA
gaaagcatagcaatctaatctaagtttaaaacaatgagaagatacatcagaggtggt

SEQ ID NO: 30 - DNA, 57 bp, synthetic construct
  source 1..57: mol_type = other DNA
ctaattacatgactcgaggtcgacggtatcgttatgcagcgttcaacaaattgaaaa

SEQ ID NO: 31 - DNA, 58 bp, synthetic construct
  source 1..58: mol_type = other DNA
gaaagcatagcaatctaatctaagtttaaaacaatgaccgaacaagtcttattctcag

SEQ ID NO: 32 - DNA, 58 bp, synthetic construct
  source 1..58: mol_type = other DNA
ctaattacatgactcgaggtcgacggtatcgttaagcgttcaacaaattgaaaaatct

SEQ ID NO: 33 - DNA, 60 bp, synthetic construct
  source 1..60: mol_type = other DNA
gaaagcatagcaatctaatctaagtttaaaacaatgaacttacaatttgaagaaagacca

SEQ ID NO: 34 - DNA, 57 bp, synthetic construct
  source 1..57: mol_type = other DNA
ctaattacatgactcgaggtcgacggtatcgttacaaatcagctaaagggtgttcac

SEQ ID NO: 35 - DNA, 59 bp, synthetic construct
  source 1..59: mol_type = other DNA
gaaagcatagcaatctaatctaagtttaaaacaatgaacttacactttgaagaattgac

SEQ ID NO: 36 - DNA, 57 bp, synthetic construct
  source 1..57: mol_type = other DNA
ctaattacatgactcgaggtcgacggtatcgttagtagtcagacaaatctgctaaag

SEQ ID NO: 37 - DNA, 59 bp, synthetic construct
  source 1..59: mol_type = other DNA
gaaagcatagcaatctaatctaagtttaaaacaatgacaatccactgtgaagtattaac

SEQ ID NO: 38 - DNA, 55 bp, synthetic construct
  source 1..55: mol_type = other DNA
ctaattacatgactcgaggtcgacggtatcgttaaccaacgtcagccaaagggtg

SEQ ID NO: 39 - DNA, 58 bp, synthetic construct
  source 1..58: mol_type = other DNA
gaaagcatagcaatctaatctaagtttaaaacaatgaatgtcacctttgaagaaagag

SEQ ID NO: 40 - DNA, 55 bp, synthetic construct
  source 1..55: mol_type = other DNA
ctaattacatgactcgaggtcgacggtatcgttatgccaaatcagctaaagggtg

SEQ ID NO: 41 - DNA, 4431 bp, synthetic construct
  source 1..4431: mol_type = other DNA
cgaaaccctatgctctgttgttcggatttgaaattttaaaactacattaatgtgttagtttttctttctttctttctttgtcttgacgtgatttggacttctgtcttgcattcgcgtccattcatctgacccaatattccttttggttttgttatccttataaaaagaaaggaagcttcttagagggaaaaaaatgatgaagagtaatgccaaaatataaataaataaataaatatgaaaatcattttctatttttaatagaataagaagagcatcttaagattacaatttcaagaaatagtttacacagtatatccaataactccaataaactactttcctatacaaatttctatggtgggattaatagtaaaacttctgtacttctctaattcaccaagaaattaaggtaaacatctggtaagcactatccagctttttgctattacacatatggcttttctgcaatcatttcttcccattttgtctcaagccgttagtcttgaaaccacaggcggagtagagttacttgatgcggtattttacatgccttttttcactgcaaaaaaaatgaaatacatatttacacgatttgcaggacagtttacgatagtgagtatgcagaatagttaacacctttgttttatccttttgtgtcttaattatatgatataaaggcgcctggcaaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacagatgcgtaaggagaaaataccgcatcaggaaattgtaaacgttaatattttgttaaaattcgcgttaaatttttgttaaatcagctcattttttaaccaataggccgatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaattttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagtttaaaacaatgaatgtcacctttgaagaaagagccagtttacacggttacagaatcggtatcgcatccttagacgccccagcatccttgaacgctttgtccttaccaatgattgatgctttacaagacagattgagagcttgggcagaagatgcagacatagcctgtgtcttgttacgtggtaatggttctaaagcattttgcgcaggtggtgacgttgtccaattagctaagaaatgtttggcctccccaggtgaagcccctgaattggctgaaagatttttcgctagaagttacagattagatcattatttgcacacatacccaaagcctttgatatgctgggctcatggtcacgttttaggtggtggtatgggtttgttacaaggtgctggtattagaatagtcaccccatcttcaagattggcaatgcctgaaatctctattggtttatttccagatgttggtggttcccatttcttaagtagattgcctggtaaattgggtttatttttcggtttaaccgcttcaccattgaatgccagagatgctttggacttaaacttggctgatagattcttgttagatactcaacaagacgcattgatcgatggtttgatccaattgaactggagagaacaaccagatttgcaattgcattccttgttaaaggctttagaacaacaagcaagaagtgaattaccagctgcacaatggttgcctagaagagaaagattagacgccttgttagatcaagctactttaccattgtcttggcaagccttagcttcattggaaaacgatgaagacgcattgttagccaaagccgctaagactatgttgggtggttcacctttaacaggtcatttggtctggggtcaaattagaagagctagacacttatctttggcacaagtatttcaaatggaatacggcatgtcattgaattgttgcagacatccagaatttgcagaaggtgtaagagccagattaatcgataaagaccacgctccacattggcactggcctgacgtaaaccaagttcctgaagccgttattgcagcccattttgcaccattagatgaccaccctttagctgatttggcataacgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccataaagcagccgctaccaaacagacaagattcagtatgtaaggtaaatacctttttgcacagttaaactacccaaacttattaaagcttgataaattactgaaattccacctttcagttagattcaggcctcatatagattagatatagggtacgtaacattctgtcaaccaagttgttggaatgaaagtctaaaatgtcatctattcggtagcactcatgttactagtatactgtcacatgcggtgtaacgtggggacataaaacagacatcaaatataatggaagctgaaatgcaaagatcgataatgtaataggaatgaaacatataaaacgaaaggagaagtaatggtaatattagtatgtagaaataccgattcaattttggggattcttatattctcgagagaatttctagtataatctgtatacataatattataggctttaccaacaatggaatttcgacaattatcatattattcaccaattaatcacaagttggtaatgagtttgataacaagttactttcttaacaacgttagtatcgtcaaaacactcggttttactcgagcttgtagcacaataataccgtgtagagttctgtattgttcttcttagtgcttgtatatgctcatcccgaccttccatt

SEQ ID NO: 42 - DNA, 5356 bp, synthetic construct
  source 1..5356: mol_type = other DNA
accggagcttggatatgataaacgaaatattcttgaatcgtgagatcgcctgttttcaaaaccgttggaggcagaaacaattttgtcacaagatgggcattctaccccatccttgctgtattattgtagtctcgctttcttttatgctggacaaatgagactactgcacatttttatacgttcttggttttttttaaaggtgtggtttcggcattatcctgccgcacgtttcttggataattcatcctgattctctattttaaacgcttcagcctatcaggatttggttttgatacatactgcaagagtgtatctcgggaacagtcatttattccgcaacaaacttaattgcggaacgcgttaggcgatttctagcatatatcaaataccgttcgcgatttcttctgggttcgtctcttttcttttaaatacttattaacgtactcaaacaactacacttcgttgtatctcagaatgagatccctcagtatgacaatacatcattctaaacgttcgtaaaacacatatgaaacaactttataacaaagcgaacaaaatgggcaacatgagatgaaactccgcgtcccttagctgaactacccaaacgtacgaatgcctgaacaattagtttagatccgagattccgcgcttccatcatttagtataatccatattttatataatatataggataagtaacagcccgcgaatcgaggagaacttctagtatatccacatacctaatattattgccttattaaaaatggaatcccaacaattacatcaaaatccacattctcttcaaaatcaattgtcctgtacttccttgttcatgtgtgttcaaaaacgttatatttataggataattatactctatttctcaacaagtaattggttgtttggccgagcggtctaaggcgcctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtggctggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatcttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatagtaaaggaatacaggtaagcaaattgatactaatggctcaacgtgataaggaaaaagaattgcactttaacattaatattgacaaggaggagggcaccacacaaaaagttaggtgtaacagaaaatcatgaaactacgattcctaatttgatattggaggattttctctaaaaaaaaaaaaatacaacaaataaaaaacactcaatgacctgaccatttgatggagtttaagtcaataccttcttgaagcatttcccataatggtgaaagttccctcaagaattttactctgtcagaaacggcctatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaattttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagtttaaaacaatgaatgtcacctttgaagaaagagccagtttacacggttacagaatcggtatcgcatccttagacgccccagcatccttgaacgctttgtccttaccaatgattgatgctttacaagacagattgagagcttgggcagaagatgcagacatagcctgtgtcttgttacgtggtaatggttctaaagcattttgcgcaggtggtgacgttgtccaattagctaagaaatgtttggcctccccaggtgaagcccctgaattggctgaaagatttttcgctagaagttacagattagatcattatttgcacacatacccaaagcctttgatatgctgggctcatggtcacgttttaggtggtggtatgggtttgttacaaggtgctggtattagaatagtcaccccatcttcaagattggcaatgcctgaaatctctattggtttatttccagatgttggtggttcccatttcttaagtagattgcctggtaaattgggtttatttttcggtttaaccgcttcaccattgaatgccagagatgctttggacttaaacttggctgatagattcttgttagatactcaacaagacgcattgatcgatggtttgatccaattgaactggagagaacaaccagatttgcaattgcattccttgttaaaggctttagaacaacaagcaagaagtgaattaccagctgcacaatggttgcctagaagagaaagattagacgccttgttagatcaagctactttaccattgtcttggcaagccttagcttcattggaaaacgatgaagacgcattgttagccaaagccgctaagactatgttgggtggttcacctttaacaggtcatttggtctggggtcaaattagaagagctagacacttatctttggcacaagtatttcaaatggaatacggcatgtcattgaattgttgcagacatccagaatttgcagaaggtgtaagagccagattaatcgataaagaccacgctccacattggcactggcctgacgtaaaccaagttcctgaagccgttattgcagcccattttgcaccattagatgaccaccctttagctgatttggcataacgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggaccaactatcatccgctaattactgacattaccaaatgagatctgtgaatgggcaagataaaaaacaaaaattgaaatgtttgacgttatgtaaaactattaattccttcgctttcggcggtcacagaatttgcgtgtagctgactcttgttcaatcaatatcatttgttactttatttgaaagtctgtattactgcgcctattgtcatccgtaccaaagaacgtcaaaaagaaacaagataatttttgtgcttacaccatttatagatcactgagcccagaatatcgctggagctcagtgtaagtggcatgaacacaactctgactgatcgcacatattgccgttatcataaatactagttgtacttgtcaatgcgacgaatggcatcatgcctattattacgttcctctttttccgtttcatgtttccagaatgctattgaatctaacacttcaattataaaaaagaataaatccgcaataattttaggctaattgttgtactgtcaagcgaacctaatggttaaaattcagaggaaccttcgacgtagtctgatcgctacttctatatcttatgttcccagtcaatcaaaagttgatactataatagctgccatttatacctgttagttatggcgatcgtttatcacg

SEQ ID NO: 43 - DNA, 3492 bp, synthetic construct
  source 1..3492: mol_type = other DNA
aactcaatcgtgctaagcgacaaactgaaaaggtatatcaaccagagatgaaatcggaaattagcggggatagcactctgaaacgtgttcaagtcaccttccacgggaatggtcgggtaaataaaaagaaaaagaaggttcctccacggaaagatggtatcaagttccgttgattgaaaatttttcttttcaagcgatgaggttagcgaagttgttcgaacaagcccaaatatgtttagaaggcagtacgaattgataccctgaactgttactacataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaattttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagtttaaaacaatgaatgtcacctttgaagaaagagccagtttacacggttacagaatcggtatcgcatccttagacgccccagcatccttgaacgctttgtccttaccaatgattgatgctttacaagacagattgagagcttgggcagaagatgcagacatagcctgtgtcttgttacgtggtaatggttctaaagcattttgcgcaggtggtgacgttgtccaattagctaagaaatgtttggcctccccaggtgaagcccctgaattggctgaaagatttttcgctagaagttacagattagatcattatttgcacacatacccaaagcctttgatatgctgggctcatggtcacgttttaggtggtggtatgggtttgttacaaggtgctggtattagaatagtcaccccatcttcaagattggcaatgcctgaaatctctattggtttatttccagatgttggtggttcccatttcttaagtagattgcctggtaaattgggtttatttttcggtttaaccgcttcaccattgaatgccagagatgctttggacttaaacttggctgatagattcttgttagatactcaacaagacgcattgatcgatggtttgatccaattgaactggagagaacaaccagatttgcaattgcattccttgttaaaggctttagaacaacaagcaagaagtgaattaccagctgcacaatggttgcctagaagagaaagattagacgccttgttagatcaagctactttaccattgtcttggcaagccttagcttcattggaaaacgatgaagacgcattgttagccaaagccgctaagactatgttgggtggttcacctttaacaggtcatttggtctggggtcaaattagaagagctagacacttatctttggcacaagtatttcaaatggaatacggcatgtcattgaattgttgcagacatccagaatttgcagaaggtgtaagagccagattaatcgataaagaccacgctccacattggcactggcctgacgtaaaccaagttcctgaagccgttattgcagcccattttgcaccattagatgaccaccctttagctgatttggcataacgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccgtacaatcacaaaattaatcataatgttcatttatctataatcaaacaatgagaatatatactgaagtttggtaggttattcttcagagatgcagtacgatatcaccaacggtatgcagacacctatcaaaacataaacggattcatcatgaggtaaaacgacttcctgttcaacgctgagatcgtgatcatgagaatctccaccaacaaatgctgtgaaagacgcatatagtaaactgccaccactcataagcagaagatttccacttat

SEQ ID NO: 44 - DNA, 1959 bp, synthetic construct
  source 1..1959: mol_type = other DNA
atgtcccaaactcataagcacgctattccagctaatattgctgatagatgcttgatcaacccagaacaatacgaaactaagtacaagcaatccatcaacgatccagatactttttggggtgaacaaggtaagattttggattggattaccccataccaaaaggtcaagaatacttcttttgctccaggtaacgtttccatcaaatggtatgaagatggtactttgaacttggctgctaactgtttggatagacacttgcaagaaaacggtgatagaaccgctattatttgggaaggtgatgatgcctctcaatccaaacatatctcttacagagaattgcacagagatgtctgtagattcgctaacactttgttggatttgggtattaagaagggtgacgttgttgctatctatatgccaatggttcctgaagctgctgttgctatgttggcttgtgctagaattggtgctgttcattctgttattttcggtggtttttcaccagaagctgttgccggtagaattatcgattcttcatccagattggttatcaccgctgatgaaggtgttagagctggtagatcaattccattgaaaaagaacgttgatgacgccttgaagaacccaaatgttacttctgttgaacacgtcatcgttttgaagagaaccgattctgatattgactggcaagaaggtagagatttgtggtggagagatttgattgaaaaggcttctccagaacatcaaccagaagcaatgaacgctgaagatcctttgtttatcttgtacacttctggttctactggtaagccaaaaggtgttttacacactactggtggttatttggtttacgctgctactactttcaagtacgttttcgattatcatccaggtgacatctattggtgtactgctgatgttggttgggttactggtcattcttatttgttgtatggtccattagcttgtggtgctactaccttgatgttcgaaggtgttccaaattggccaactccagctagaatgtgtcaagttgttgacaaacaccaagtcaacatcttgtatactgctccaactgctattagagctttgatggctgaaggtgataaggctattgaaggtactgatagatcctccttgagaatcttgggttctgttggtgaacctattaaccctgaagcttgggaatggtactggaagaaaattggtaaagaaaagtgcccagttgttgatacttggtggcaaactgaaacaggtggttttatgattactccattgccaggtgctattgaattgaaagctggttctgctactagaccattttttggtgttcaaccagccttggttgataatgaaggtcatccacaagaaggtgctactgaaggtaatttggttattactgattcttggccaggtcaagctagaactttgtttggtgatcacgaaagattcgaacaaacctacttttccaccttcaagaacatgtacttttctggtgatggtgctagaagagatgaagatggttactattggattaccggtagagttgatgatgtcttgaatgtttctggtcacagattgggtacagccgaaattgaatctgctttggttgctcatccaaagattgctgaagcagcagttgttggtattccacatgctattaagggtcaagcaatctacgcttacgttaccttgaatcatggtgaagaaccatccccagaattatacgctgaagttagaaactgggtcagaaaagaaattggtccattggctactccagatgttttacattggacagattctttgccaaagaccagatcaggtaagatcatgagaagaatcttgagaaaaattgctgccggtgatacttctaacttgggtgatacctctactttggctgatccaggtgttgttgaaaagccattagaagaaaagcaagccattgccatgccatcttaa

SEQ ID NO: 45 - protein, 366 aa, Pseudomonas mendocina
  VARIANT 95: note = A, D, R, H, K, S, T, N, Q, Y, or a subset thereof
MNLQFEERPSLHGYRIGIASLDAEKSLNALTLPMIQALDARLQAWAEDPTIACVMLRGNGPKAFCAGGDWQLVQQCREHPGEVPPLARRFFADXYRLDHRIHSYPKPFICWAHGHVLGGGMGLMQGAGVRIVTPSSRLGMPEINIGLYPDVGGSWFLARLPGRLGLFLGLTAASINARDALDLNLADRFLRDDQQDALLEGLVQLNWREQPAAQLHSLLRALENEARGELPAAQWLPRRERIDELLDVADLPAAVQAISALQQDDDALLARAAKTLAHGCPLTAHLVWQQIRRARHLSLAEVFRMEYAMSLNCCRHPDFPEGVRARLIDKDQTPHWHWPDVAAIPEAVIEAHFAPAWEGEHPLADL

SEQ ID NO: 46 - protein, 350 aa, Bacillus cereus
  VARIANT 91: note = A, D, R, H, K, S, T, N, Q, Y, or a subset thereof
MTEHVLFSVSENGVASITLNRPKALNSLSYDMLQPIGQKLKEWENDERIALIVLKGAGTKGFCAGGDIKTLYEARSNEAALQHAERFFEEXYEIDTYIYQYKKPIIACLDGIVMGGGVGLTNGAKYRIVTERTKWAMPEMNIGFFPDVGAAYFLNKAPGYAGRYVALTASILKASDVLFINAADYFIASDSLPNFLTELESVNWSKEDDVHTHLKEVIRTFATAPTLESELAPSLEEINSHFAFDTIEEIIHSLEKDQSSFSLKAKETLLSKSPISLKVTLKQFIDGQNKSVEECFATDLVLAKNFMRHEDFFEGVRSVWDKDQNPNYKYKQLSDVSEEDVNRFFNLLNA

SEQ ID NO: 47 - protein, 359 aa, Bacillus mycoides
  VARIANT 101: note = A, D, R, H, K, S, T, N, Q, Y, or a subset thereof
MRRYIRGGFKMTENVLFSIHENGVASITLNRPKALNSLSYDMLHPIGQKLKEWEKDDRIAWILKGAGTKGFCAGGDIKTLYEARSNEVALQHAEHFFEEXYEIDTYIYHYPKPIIACLDGIVMGGGVGLTNGAKYRIVTDRTKWAMPEMNIGFFPDVGAAYFLNKAPGQTGRYVALTASVLKAADVLYIKAADHYMPSETLPTFLDAIEKVNWHDQNIHITLKELINKYETAPSVESELVSLLEEIDQHFSFHTVEDIIHSLDNADGSFASKTKETLLSKSPFSLKVTLKQLIDGKEKSIEECFATDLVLAKNFMRHEDFFEGVRSVVVDKDQNPKYKYKQLSDVSDEDVNRFFNLLNA

SEQ ID NO: 48 - protein, 370 aa, Pseudomonas fluorescens
  VARIANT 95: note = A, D, R, H, K, S, T, N, Q, Y, or a subset thereof
MNLHFEELTGSDGARIGIASLDAEKSLNALSLPMILALGDRLDAWAKDPNIVCVLLRGNGPKAFCAGGEVRSLALACREQPGEVPALAAQFFAAXYRLDYRLHTFPKPLICWGHGYVLGGGMGLLQSAAVRIVTPSSRLAMPEISIGLYPDVGASWFLSRLPGKLGLFLGLTGAHVNGRDALDLGLADRFLRDDQQDELIEGLLQLNWQEQTAMQLNSLFKALAQEAVDQLPEAQWLPRRAQIDEWLDVGDVRSAWRALSQLRDHADPLFSRAGKTLSEGCPLTAHLVWEQIQRARHLSLAQVFQMEYTLSLNCCRHPEFSEGVRARLIDKDQTPRWHWPDVHTLPDAVVQAHFNKAWEGRHPLADLSDY

SEQ ID NO: 49 - protein, 351 aa, Bacillus thuringiensis
  source 1..351: note = subsp. finitimus (strain YBT-020)
  VARIANT 91: note = A, D, R, H, K, S, T, N, Q, Y, or a subset thereof
MTEHVLFSVSENGVASITLNRPKALNSLSYDMLQPIGQKLKEWEHDERIALIVLKGAGTKGFCAGGDIKTLYEARSNEAALQHAERFFEEXYEIDTYIYQYKKPIIACLDGIVMGGGVGLTNGAKYRIVTERTKWAMPEMNIGFFPDVGAAYFLNKAPGYAGRYVALTASILKAADVLFINAADYFIASDSLPNFLTELESVNWPKKDDVHTHLKEVIRTFATAPTLESELAPSLEEINSHFAFDTIEEIIHSLEKDQSSFALKAKETLLSKSPISLKVTLKQFIDGQNKSVEECFATDLVLAKNFMRHEDFFEGVRSVVVDKDQNPDYKYKQLSDVSEEDVNRFFNLLNA

SEQ ID NO: 50 - protein, 366 aa, Pseudomonas fulva
  VARIANT 95: note = A, D, R, H, K, S, T, N, Q, Y, or a subset thereof
MNVTFEERASLHGYRIGIASLDAPASLNALSLPMIDALQDRLRAWAEDADIACVLLRGNGSKAFCAGGDVVQLAKKCLASPGEAPELAERFFARXYRLDHYLHTYPKPLICWAHGHVLGGGMGLLQGAGIRIVTPSSRLAMPEISIGLFPDVGGSHFLSRLPGKLGLFFGLTASPLNARDALDLNLADRFLLDTQQDALIDGLIQLNWREQPDLQLHSLLKALEQQARSELPAAQWLPRRERLDALLDQATLPLSWQALASLENDEDALLAKAAKTMLGGSPLTGHLVWGQIRRARHLSLAQVFQMEYGMSLNCCRHPEFAEGVRARLIDKDHAPHWHWPDVNQVPEQVIAAHFAPLDDHPLADLA

SEQ ID NO: 51 - protein, 351 aa, Bacillus cereus
  source 1..351: note = (strain ZK / E33L)
  VARIANT 91: note = A, D, R, H, K, S, T, N, Q, Y, or a subset thereof
MTEHVLFSVSENGVASITLNRPKALNSLSYEMLQPIGKKLKEWENDEQIALIVLKGAGTKGFCAGGDIKTLYEARSNEIALQHAERFFEEXYEIDTYIYQYKKPIIACLDGIVMGGGVGLTNGAKYRIVTERTKWAMPEMNIGFFPDVGAAYFLNKAPGFAGRYVALTASILKASDVLFINAADYFMTSDSLPKFLTELESVNWHKGDDVHIHLKEVIRTFATTSNLESELAPLLEEINAHFAFDTIEEIIHSLEKDQSSFALKTKKTLLSKSPISLKVTLKQFIDGHDKSVEECFATDLVLAKNFMRHEDFFEGVRSVVVDKDQNPNYKYKQLSDVSDEDVNRFFNLLNA

SEQ ID NO: 52 - protein, 351 aa, Bacillus cereus
  source 1..351: note = (strain ATCC 14579 / DSM 31)
  VARIANT 91: note = A, D, R, H, K, S, T, N, Q, Y, or a subset thereof
MTEQVLFSVSENGVATITLNRPKALNSLSYDMLQPIGQKLKEWEHDERIALIVLKGAGTKGFCAGGDIKTLYEARSNEVALQHAERFFEEXYEIDTYIYQYTKPIIACLDGIVMGGGVGLTNGAKYRIVTERTKWAMPEMNIGFFPDVGAAYFLNKAPGYTGRFVALTASILKASDVLFINAADYFMTSDSLPEFLTELESVNWHKEDDVHTNLKEVIRTFATAPNLESELAPSLEVINSHFAFDTIEEIIHSLEKDESSFALKTKEILLSKSPISLKVTLKQFIDGQDKSVEECFATDLILAKNFMRHEDFFEGVRSVVVDKDQNPNYKYKQLSDVSEEDVNRFFNLLNA

SEQ ID NO: 53 - protein, 309 aa, Escherichia coli [sequence omitted]